SEQUENCE LISTING (101 sequences)

SEQ ID NO: 1 - DNA, 27 bp, Artificial Sequence
  Synthetic Construct
cgttttccaaatctgtaacagactggg

SEQ ID NO: 2 - DNA, 25 bp, Artificial Sequence
  Synthetic Construct
aggacgttgataggttggatgtgga

SEQ ID NO: 3 - DNA, 31 bp, Artificial Sequence
  Synthetic Construct
ggtagctatgtagggaagggataaacgctga

SEQ ID NO: 4 - DNA, 28 bp, Artificial Sequence
  Synthetic Construct
gcgctaaggagcttaacttctgtgttcg

SEQ ID NO: 5 - DNA, 22 bp, Artificial Sequence
  Synthetic Construct
gacggttgtcccggtttaagca

SEQ ID NO: 6 - DNA, 21 bp, Artificial Sequence
  Synthetic Construct
gctggtatcttcgactggtct

SEQ ID NO: 7 - DNA, 22 bp, Artificial Sequence
  Synthetic Construct
aggctgggtgtgtaagcgttgt

SEQ ID NO: 8 - DNA, 25 bp, Artificial Sequence
  Synthetic Construct
caagcaattcggttggatatccgtt

SEQ ID NO: 9 - DNA, 35 bp, Artificial Sequence
  Synthetic Construct
  misc_feature (16)..(16): n is 2,6-Diaminopurine
ggtaatgaattacctntctctgctggtttcttctt

SEQ ID NO: 10 - DNA, 27 bp, Artificial Sequence
  Synthetic Construct
  misc_feature (13)..(13): n is 2,6-Diaminopurine
accagcatcttcngcatcttctgtaaa

SEQ ID NO: 11 - DNA, 34 bp, Artificial Sequence
  Synthetic Construct
  misc_feature (13)..(13): n is 2,6-Diaminopurine
gaagttatgtttnctattcgaatcgtggtccagt

SEQ ID NO: 12 - DNA, 27 bp, Artificial Sequence
  Synthetic Construct
gttgtaaagccatgatgctcgtaacca

SEQ ID NO: 13 - DNA, 20 bp, Artificial Sequence
  Synthetic Construct
ggcatgcctgtttgagcgtc

SEQ ID NO: 14 - DNA, 29 bp, Artificial Sequence
  Synthetic Construct
gcttattgatatgcttaagttcagcgggt

SEQ ID NO: 15 - DNA, 22 bp, Artificial Sequence
  Synthetic Construct
tgaggcttgactatacaacacc

SEQ ID NO: 16 - DNA, 29 bp, Artificial Sequence
  Synthetic Construct
ctaaaatgaacagataaagtaagattcaa

SEQ ID NO: 17 - DNA, 29 bp, Artificial Sequence
  Synthetic Construct
aaaacttatatgacttcaaatccagtttt

SEQ ID NO: 18 - DNA, 27 bp, Artificial Sequence
  Synthetic Construct
tttactcaataaaagataacaccacag

SEQ ID NO: 19 - DNA, 24 bp, Artificial Sequence
  Synthetic Construct
tggataagtaaaagcaacttggtt

SEQ ID NO: 20 - DNA, 30 bp, Artificial Sequence
  Synthetic Construct
aatgaagattcaactcaataagaaacaaca

SEQ ID NO: 21 - DNA, 23 bp, Artificial Sequence
  Synthetic Construct
taccaaggcgcttgagagaactc

SEQ ID NO: 22 - DNA, 19 bp, Artificial Sequence
  Synthetic Construct
ctggtgtgtaggtgaagtc

SEQ ID NO: 23 - DNA, 23 bp, Artificial Sequence
  Synthetic Construct
gtgtgttgtagggtgaagtcgac

SEQ ID NO: 24 - DNA, 22 bp, Artificial Sequence
  Synthetic Construct
caccttgaaatcacatacctga

SEQ ID NO: 25 - DNA, 23 bp, Artificial Sequence
  Synthetic Construct
ccatttgaagttgtttattatgc

SEQ ID NO: 26 - DNA, 27 bp, Artificial Sequence
  Synthetic Construct
gggaaatgattaattatgcattaaatc

SEQ ID NO: 27 - DNA, 26 bp, Artificial Sequence
  Synthetic Construct
tttttcagatttaggattagttgatt

SEQ ID NO: 28 - DNA, 22 bp, Artificial Sequence
  Synthetic Construct
gatccgtattggttatatcatc

SEQ ID NO: 29 - DNA, 20 bp, Artificial Sequence
  Synthetic Construct
ggaataatacgccgaccagc

SEQ ID NO: 30 - DNA, 25 bp, Artificial Sequence
  Synthetic Construct
aaggatctatttcagtatgatgcag

SEQ ID NO: 31 - DNA, 200 bp, Artificial Sequence
  Synthetic Construct
tgccgaagcgttttccaaatctgtaacagactgggctgattgaatcttactttatctgttcattttagctagaggtataactaaatcaagttgtcttgcatatttaagaatcgattgatgctttatatacaactgcttgggtgttgtatagtcaagcctcacgagcaattagtattggtcagcttcacatatcactatgc

SEQ ID NO: 32 - DNA, 200 bp, Artificial Sequence
  Synthetic Construct
gcatgggaacaggtgtatccttctcgctatcgccaccacactgggtgttgtttcttattgagttgaatcttcattcactcaaaactggattgaagtttgaatcaaaataaccaagttgcttttacttatccattctttggttaagtcctcgaccgattagtattggtccgctccaactatcactagccttccacttccaa

SEQ ID NO: 33 - DNA, 200 bp, Artificial Sequence
  Synthetic Construct
gcatggttacaggtgtatccttctcgctatcgccaccacactgtggtgttatcttttattgagtaaattttgttcactcaaaactggatttgaagtcatataagtttttttccgagttcttttcttttaacctattggttaagtcctcgatcgattagtatcagtccgctccatacatcactgtacttccactcctgacc

SEQ ID NO: 34 - DNA, 200 bp, Artificial Sequence
  Synthetic Construct
cagctccatccgcagggacttcacctacacaccagcgtgccttctcccgaagttacggcaccattttgcctagttccttcacccgagttctctcaagcgccttggtattctctacctgaccacctgtgtcggtttggggtacgatttgatgttacctgatgcttagaggcttttcctggaagcagggcatttgttacttc

SEQ ID NO: 35 - DNA, 169 bp, Artificial Sequence
  Synthetic Construct
cgcttgggcttacgtctatccggattcaggtatgtgatttcaaggtgttttgcggttcatgcgaactttcggttcgtcgacttcaccttacaacacacaatcgtcagattgtttgggtgttatatggtcaagcctcacgggcaattagtactggttagctcaacgcctc

SEQ ID NO: 36 - DNA, 151 bp, Artificial Sequence
  Synthetic Construct
tttaccactaacaccatagaaattataacggtcaatgccatgatttaatgcataattaatcatttcccattgcactgcataacttccggcaaaatgacggaatgcatttgatgtaccaccagcataataaacaacttcaaatgggttgata

SEQ ID NO: 37 - DNA, 186 bp, Artificial Sequence
  Synthetic Construct
tgtgatttaaacaagtttactaaggcatcatttttctcgcgaccttcaaatggcacgatatctttatcatatagatgatataaccaatacggatctaatttaacatataaacattgatgttgctgtaaatatttatctaactcttttaaataataatcaactaatcctaaatctgaaaaatccatt

SEQ ID NO: 38 - DNA, 25 bp, Artificial Sequence
  Synthetic Construct
gcattaatcgacggtatggttgacc

SEQ ID NO: 39 - DNA, 26 bp, Artificial Sequence
  Synthetic Construct
cctgctgaaacaggttttcccacata

SEQ ID NO: 40 - DNA, 25 bp, Artificial Sequence
  Synthetic Construct
agtgatgatgagttgtttgccagtg

SEQ ID NO: 41 - DNA, 22 bp, Artificial Sequence
  Synthetic Construct
tgaattgtcgccgcgtgaccag

SEQ ID NO: 42 - DNA, 23 bp, Artificial Sequence
  Synthetic Construct
acccagcggtttgagggagaaac

SEQ ID NO: 43 - DNA, 30 bp, Artificial Sequence
  Synthetic Construct
aaagtttgaagatatacgtggtggacgtta

SEQ ID NO: 44 - DNA, 21 bp, Artificial Sequence
  Synthetic Construct
cgcacgcgcaagatggaaacg

SEQ ID NO: 45 - DNA, 23 bp, Artificial Sequence
  Synthetic Construct
aagttcagcgggtattcctacct

SEQ ID NO: 46 - DNA, 27 bp, Artificial Sequence
  Synthetic Construct
agctttttgttgtctcgcaacactcgc

SEQ ID NO: 47 - DNA, 26 bp, Artificial Sequence
  Synthetic Construct
ctaccaaacacaatgtgtttgagaag

SEQ ID NO: 48 - DNA, 31 bp, Artificial Sequence
  Synthetic Construct
cctgatttgaggtcaaacttaaagacgtctg

SEQ ID NO: 49 - DNA, 24 bp, Artificial Sequence
  Synthetic Construct
  misc_feature (22)..(22): n is 5′ 5-Nitroindole
agtcctacctgatttgaggtcnaa

SEQ ID NO: 50 - DNA, 21 bp, Artificial Sequence
  Synthetic Construct
  misc_feature (4)..(4): n is 5′ 5-Nitroindole
ccgngggtttgagggagaaat

SEQ ID NO: 51 - DNA, 33 bp, Artificial Sequence
  Synthetic Construct
aaagttatgaaataaattgtggtggccactagc

SEQ ID NO: 52 - DNA, 22 bp, Artificial Sequence
  Synthetic Construct
acccgggggtttgagggagaaa

SEQ ID NO: 53 - DNA, 24 bp, Artificial Sequence
  Synthetic Construct
agtcctacctgatttgaggtcgaa

SEQ ID NO: 54 - DNA, 21 bp, Artificial Sequence
  Synthetic Construct
ccgagggtttgagggagaaat

SEQ ID NO: 55 - DNA, 25 bp, Artificial Sequence
  Synthetic Construct
ggaagggatcaggtggttcactctt

SEQ ID NO: 56 - DNA, 29 bp, Artificial Sequence
  Synthetic Construct
aaaacttatgtgacttcaaatccagtttt

SEQ ID NO: 57 - DNA, 23 bp, Artificial Sequence
  Synthetic Construct
tctgacgattgtgtgttgtaagg

SEQ ID NO: 58 - DNA, 20 bp, Artificial Sequence
  Synthetic Construct
ggatagacgtaagcccaagc

SEQ ID NO: 59 - DNA, 198 bp, Artificial Sequence
  Synthetic Construct
gcatggttacaggtgtatccttctcgctatcgccaccacactgtggtgttatcttttattgagtaaattttgttcactcaaaactggatttgaagtcatataagtttttttccgagttcttttcttttaacctattggttaagtcctcgatcgattagtatcagtccgctccatacatcactgtacttccactcctga

SEQ ID NO: 60 - DNA, 22 bp, Artificial Sequence
  Synthetic Construct
ggttaagtcccgcaacgagcgc

SEQ ID NO: 61 - DNA, 19 bp, Artificial Sequence
  Synthetic Construct
aggaggtgatccaaccgca

SEQ ID NO: 62 - DNA, 20 bp, Artificial Sequence
  Synthetic Construct
gggcatgcctgtttgagcgt

SEQ ID NO: 63 - DNA, 27 bp, Artificial Sequence
  Synthetic Construct
ctacctgatttgaggcgacaacaaaac

SEQ ID NO: 64 - DNA, 24 bp, Artificial Sequence
  Synthetic Construct
ccgcgaagattggtgagaagacat

SEQ ID NO: 65 - DNA, 26 bp, Artificial Sequence
  Synthetic Construct
cctacctgatttgagggcgaaatgtc

SEQ ID NO: 66 - DNA, 19 bp, Artificial Sequence
  Synthetic Construct
ggagcaacgcctaaccggg

SEQ ID NO: 67 - DNA, 27 bp, Artificial Sequence
  Synthetic Construct
gtcctacctgatttgaggggaaaaagc

SEQ ID NO: 68 - DNA, 28 bp, Artificial Sequence
  Synthetic Construct
aacaaatccaccaacggtgagaagatat

SEQ ID NO: 69 - DNA, 20 bp, Artificial Sequence
  Synthetic Construct
gcgtagacttcgctgcggat

SEQ ID NO: 70 - DNA, 19 bp, Artificial Sequence
  Synthetic Construct
cgtagacttcgctgcggat

SEQ ID NO: 71 - DNA, 20 bp, Artificial Sequence
  Synthetic Construct
ctgggcggtgagaagaaatc

SEQ ID NO: 72 - DNA, 21 bp, Artificial Sequence
  Synthetic Construct
gcgtagacttcgctgctggaa

SEQ ID NO: 73 - DNA, 20 bp, Artificial Sequence
  Synthetic Construct
ccgtgcggtgagaagaaatc

SEQ ID NO: 74 - DNA, 28 bp, Artificial Sequence
  Synthetic Construct
tcctacctgatttgaggaaatagcatgg

SEQ ID NO: 75 - DNA, 22 bp, Artificial Sequence
  Synthetic Construct
atttagcggatgcaaaaccacc

SEQ ID NO: 76 - DNA, 24 bp, Artificial Sequence
  Synthetic Construct
ggaaatctaacgagagagcatgct

SEQ ID NO: 77 - DNA, 19 bp, Artificial Sequence
  Synthetic Construct
cgatgcgtgacacccaggc

SEQ ID NO: 78 - DNA, 26 bp, Artificial Sequence
  Synthetic Construct
caaggtgcaatgactttgtttgggca

SEQ ID NO: 79 - DNA, 31 bp, Artificial Sequence
  Synthetic Construct
gcaacttcaaagtgtacagtattggtatccc

SEQ ID NO: 80 - DNA, 25 bp, Artificial Sequence
  Synthetic Construct
agctgtagtttaaggcaaatgttgg

SEQ ID NO: 81 - DNA, 25 bp, Artificial Sequence
  Synthetic Construct
aggatcgcaaaatcaaccacaaaca

SEQ ID NO: 82 - DNA, 28 bp, Artificial Sequence
  Synthetic Construct
cctaaatgttaaaccccttgacaaccca

SEQ ID NO: 83 - DNA, 23 bp, Artificial Sequence
  Synthetic Construct
cccatcaggatatccagcttcgg

SEQ ID NO: 84 - DNA, 32 bp, Artificial Sequence
  Synthetic Construct
ctttaccgatacttcaatttcaccgagctcca

SEQ ID NO: 85 - DNA, 29 bp, Artificial Sequence
  Synthetic Construct
cacaggtctctgcaaatctgtaaagagaa

SEQ ID NO: 86 - DNA, 26 bp, Artificial Sequence
  Synthetic Construct
cgatggttcacgggattctgcaattc

SEQ ID NO: 87 - DNA, 30 bp, Artificial Sequence
  Synthetic Construct
gagacgttttggatacatgtgaaagaaggc

SEQ ID NO: 88 - DNA, 25 bp, Artificial Sequence
  Synthetic Construct
ttgtagaacaatctgggctttttgg

SEQ ID NO: 89 - DNA, 24 bp, Artificial Sequence
  Synthetic Construct
ggagaactcatatcaggagcacaa

SEQ ID NO: 90 - DNA, 27 bp, Artificial Sequence
  Synthetic Construct
gctatttctgctgttaaaagttcttgt

SEQ ID NO: 91 - DNA, 26 bp, Artificial Sequence
  Synthetic Construct
ctaaaacttaagctttgcaattgtgg

SEQ ID NO: 92 - DNA, 25 bp, Artificial Sequence
  Synthetic Construct
tctagcggttgacagagaaacattg

SEQ ID NO: 93 - DNA, 28 bp, Artificial Sequence
  Synthetic Construct
aaaaaattaaaaccatataacccacgaa

SEQ ID NO: 94 - DNA, 25 bp, Artificial Sequence
  Synthetic Construct
gagacagcgtccaaatcgttacacc

SEQ ID NO: 95 - DNA, 23 bp, Artificial Sequence
  Synthetic Construct
tcttaaccttccagcaccgggca

SEQ ID NO: 96 - DNA, 26 bp, Artificial Sequence
  Synthetic Construct
ctatgtagggaagggataaacgctga

SEQ ID NO: 97 - DNA, 28 bp, Artificial Sequence
  Synthetic Construct
gcgctaaggagcttaacttctgtgttcg

SEQ ID NO: 98 - DNA, 20 bp, Artificial Sequence
  Synthetic Construct
ctcctacgggaggcagcagt

SEQ ID NO: 99 - DNA, 21 bp, Artificial Sequence
  Synthetic Construct
gtattaccgcggctgctggca

SEQ ID NO: 100 - DNA, 53 bp, Artificial Sequence
  Synthetic Construct
tcgtcggcagcgtcagatgtgtataagagacagctcctacgggaggcagcagt

SEQ ID NO: 101 - DNA, 55 bp, Artificial Sequence
  Synthetic Construct
gtctcgtgggctcggagatgtgtataagagacaggtattaccgcggctgctggca

(19) United States
(12) Patent Application Publication
(10) Pub. No.: US 2022/0282310 A1
(43) Pub. Date: Sep. 8, 2022

(54) METHODS AND COMPOSITIONS FOR HIGH SENSITIVITY SEQUENCING IN COMPLEX SAMPLES

(71) Applicant: T2 Biosystems, Inc., Lexington, MA (US)

(72) Inventors: Jessica Lee SNYDER, Woburn, MA (US); Brendan John MANNING, Arlington, MA (US); Christopher STEELE, Framingham, MA (US); Roger SMITH, Westwood, MA (US); Heidi Susanne GIESE, Newburyport, MA (US); Yin Shan Cathy WONG, Newton, MA (US); Daniel GAMERO, Medford, MA (US); Thomas J. LOWERY, Jr., Belmont, MA (US)

(73) Assignees: T2 Biosystems, Inc., Lexington, MA (US); T2 Biosystems, Inc., Lexington, MA (US)

(21) Appl. No.: 17/274,911

(22) PCT Filed: Sep. 10, 2019
(86) PCT No.: PCT/US19/50439

§ 371 Date: Mar. 10, 2021

Related U.S. Application Data
(60) Provisional application No. 62/729,373, filed on Sep. 10, 2018.
(60) Provisional application No. 62/860,907, filed on Jun. 13, 2019.

(51) Int. Cl. C12Q 1/6825; C12Q 1/6869; C12Q 1/6806

(52) U.S. Cl. CPC C12Q 1/6825; C12Q 1/6869; C12Q 1/6806

(57) ABSTRACT

Provided herein are methods of detecting and sequencing target nucleic acids in complex samples (e.g., blood), as well as related panels and compositions (e.g., systems, cartridges, and kits).

### CROSS-REFERENCE TO RELATED APPLICATIONS

This application claims benefit to U.S. Provisional Application No. 62/729,373, filed on Sep. 10, 2018, and U.S. Provisional Application No. 62/860,907, filed on Jun. 13, 2019, each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Sep. 9, 2019, is named 50713-125WO3_Sequence_Listing_9.9.19_ST25 and is 21,836 bytes in size.

### FIELD OF THE INVENTION

The invention features methods and compositions for sequencing target nucleic acids (e.g., DNA) in complex samples containing cells and/or cell debris, for example, blood samples (e.g., whole blood). The methods and compositions can be used for detecting the presence and sequence of target nucleic acids, including those from pathogens, which can be used, e.g., to inform treatment decisions.

### BACKGROUND OF THE INVENTION

Sequencing of nucleic acids (e.g., DNA) is an important tool that can be used for many approaches, including identifying and classifying organisms (e.g., pathogens) and identifying the presence of genetic variants (e.g., single nucleotide polymorphisms (SNPs) or other mutations) associated with resistance to antimicrobial agents, which can be used to provide information for diagnosis and treatment. Current sequencing approaches (e.g., massively parallel sequencing) typically require isolation of target nucleic acids from biological or environmental samples prior to sequencing. However, nucleic acid isolation is time-consuming, costly, and prone to contamination. Further, nucleic acids that are present in low copy numbers, such as microbial target DNA, may be lost during isolation, which can reduce sensitivity. Therefore, minimal processing of complex samples before sequencing assays is desirable for high-sensitivity approaches.

Sequencing of target nucleic acids in complex biological or environmental samples remains challenging, in part due to the presence of interfering substances including cells, cell debris (for example, heme compounds in blood samples), and the presence of high concentrations of non-target or host (e.g., human) nucleic acids within the sample. For example, one milliliter of human blood contains approximately 3 to 6 million white blood cells. Since one human cell contains approximately 6 pg of nuclear DNA, 18 to 36 μg of human DNA is contained in one milliliter of crude blood lysate. In contrast, 10 bacterial cells contain 33 fg of DNA (based on a 2 Mbase genome). Thus, an approximate 8.4 billion-fold excess of human DNA over the microbial DNA of interest can exist. Additionally, amplification in complex samples can result in the production of non-specific amplicons which interfere with sequencing of the desired target nucleic acid(s), and may necessitate time-consuming and difficult data analysis to remove non-specific sequences.

Thus, there remains a need in the art for improved methods and compositions for sequencing target nucleic acids directly in complex samples containing cells, cell debris, and/or non-target or host cell nucleic acids (e.g., DNA).

### SUMMARY OF THE INVENTION

The invention features methods and compositions (e.g., systems, cartridges, and kits) for sequencing target nucleic acids in complex samples.

In one aspect, the invention features a method for detecting a target nucleic acid in a biological sample obtained from a subject, wherein the biological sample includes subject-derived cells or cell debris, the method including: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of detecting a concentration of about 10 copies/mL of the target nucleic acid in the biological sample. In some embodiments, the environmental or biological sample has a volume of about 0.2 mL to about 20 mL, about 0.2 mL to about 15 mL, about 0.2 mL to about 10 mL, about 0.2 mL to about 5 mL, about 0.2 mL to about 2 mL, about 0.4 mL to about 20 mL, about 0.4 mL to about 15 mL, about 0.4 mL to about 10 mL, about 0.4 mL to about 5 mL, about 0.4 mL to about 2 mL, about 0.6 mL to about 20 mL, about 0.6 mL to about 15 mL, about 0.6 mL to about 10 mL, about 0.6 mL to about 5 mL, about 0.6 mL to about 2 mL, about 0.8 mL to about 20 mL, about 0.8 mL to about 15 mL, about 0.8 mL to about 10 mL, about 0.8 mL to about 5 mL, about 0.8 mL to about 2 mL, about 1 mL to about 20 mL, about 1 mL to about 15 mL, about 1 mL to about 10 mL, about 1 mL to about 5 mL, about 1 mL to about 4 mL, about 1 mL to about 3 mL, about 1 mL to about 2 mL, about 1.5 mL to about 20 mL, about 1.5 mL to about 15 mL, about 1.5 mL to about 10 mL, about 1.5 mL to about 5 mL, about 1.5 mL to about 4 mL, about 1.5 mL to about 3 mL, about 1.5 mL to about 2 mL, about 2 mL to about 20 mL, about 2 mL to about 15 mL, about 2 mL to about 10 mL, about 2 mL to about 5 mL, about 2 mL to about 4 mL, about 2 mL to about 3 mL, about 3 mL to about 20 mL, about 3 mL to about 15 mL, about 3 mL to about 10 mL, about 3 mL to about 5 mL, about 3 mL to about 4 mL, about 4 mL to about 20 mL, about 4 mL to about 15 mL, about 4 mL to about 10 mL, about 4 mL to about 5 mL, about 5 mL to about 20 mL, about 5 mL to about 15 mL, about 5 mL to about 10 mL, about 6 mL to about 20 mL, about 6 mL to about 15 mL, about 6 mL to about 10 mL, about 7 mL to about 20 mL, about 7 mL to about 15 mL, about 7 mL to about 10 mL, about 8 mL to about 20 mL, about 8 mL to about 15 mL, about 8 mL to about 10 mL, about 9 mL to about 20 mL, about 9 mL to about 15 mL, about 9 mL to about 10 mL, about 10 mL to about 20 mL, or about 10 mL to about 15 mL. In some embodiments, the biological sample has a volume of about 0.2 mL to about 5 mL. In some embodiments, the biological sample has a volume of about 2 mL. In some embodiments, the biological sample is selected from the group consisting of blood, bloody fluids, tissue samples, urine, cerebrospinal fluid (CSF), synovial fluid (SF), and sputum. In some embodiments, the blood is whole blood, a crude blood lysate, serum, or plasma. In some embodiments, the whole blood is ethylenediaminetetraacetic acid (EDTA) whole blood, sodium citrate whole blood, sodium heparin whole blood, lithium heparin whole blood, or potassium oxylate/sodium fluoride whole blood. In some embodiments, the bloody fluid is wound exudate, phlegm, or bile. In some embodiments, the tissue sample is a tissue biopsy. In some embodiments, the tissue biopsy is a skin biopsy, muscle biopsy, or lymph node biopsy. In some embodiments, the tissue sample is a homogenized tissue sample. In some embodiments, the target nucleic acid is characteristic of a pathogen.

In some embodiments of the preceding aspect, step (a) includes amplifying the target nucleic acid in a lysate produced by lysing cells in the biological sample. In some embodiments, the lysate has at least about a 2:1 higher concentration of cell debris relative to the biological sample. In some embodiments, the lysate has at least about a 5:1 higher concentration of cell debris relative to the biological sample. In some embodiments, the lysate has about a 10:1 higher concentration of cell debris relative to the biological sample. In some embodiments, the lysate has about a 20:1 higher concentration of cell debris relative to the biological sample. In some embodiments, the lysate has about a 40:1 higher concentration of cell debris relative to the biological sample. In some embodiments, the lysate has about a 60:1 higher concentration of cell debris relative to the biological sample. In some embodiments, the cell debris is solid material.

In another aspect, the invention features a method for detecting a target pathogen nucleic acid in a whole blood sample, the method including: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and resuspending the pellet to form an extract, optionally washing the pellet one or more times prior to resuspending the pellet; (d) lysing the remaining cells in the extract of step (c) to form a lysate, the lysate containing both subject cell nucleic acid and pathogen nucleic acid; (e) amplifying pathogen nucleic acids in the lysate of step (d) to form an amplified lysate solution including an amplified target pathogen nucleic acid; and (f) sequencing the amplified target pathogen nucleic acid, thereby detecting the target pathogen nucleic acid in the sample. In some embodiments, step (c) includes washing the pellet one time prior to resuspending the pellet. In some embodiments, the washing or resuspending is performed with a wash buffer solution. In some embodiments, the wash buffer solution is Tris-EDTA (TE) buffer. In some embodiments, the washing is performed with a wash buffer solution having a volume of about 100 μL to about 500 μL. In some embodiments, the volume is about 150 μL. In some embodiments, the resuspending of step (c) is performed with a wash buffer solution having a volume of about 50 μL to about 150 μL. In some embodiments, the volume is about 100 μL. In some embodiments, the wash buffer solution further includes an amplification control nucleic acid. In some embodiments, step (a) further includes adding a total process control (TPC) to the whole blood sample. In some embodiments, the TPC is an engineered cell including a control target nucleic acid.

In another aspect, the invention features a method for detecting a target pathogen nucleic acid in a whole blood sample, the method including: (a) providing an amplified lysate solution that has been produced by: (i) contacting a whole blood sample suspected of containing one or more pathogen cells with an erythrocyte lysis agent, thereby lysing red blood cells; (ii) centrifuging the product of step (a)(i) to form a supernatant and a pellet; (iii) discarding some or all of the supernatant of step (a)(ii) and resuspending the pellet to form an extract, optionally washing the pellet one or more times prior to resuspending the pellet; (iv) lysing the remaining cells in the extract of step (a)(iii) to form a lysate, the lysate containing both subject cell nucleic acid and pathogen nucleic acid; (v) amplifying pathogen nucleic acids in the lysate of step (a)(iv) to form an amplified lysate solution including an amplified target pathogen nucleic acid; and (b) sequencing the amplified target pathogen nucleic acid, thereby detecting the target pathogen nucleic acid in the sample. In some embodiments, step (a)(iii) includes washing the pellet one time prior to resuspending the pellet. In some embodiments, the washing or resuspending is performed with a wash buffer solution. In some embodiments, the wash buffer solution is TE buffer. In some embodiments, the washing is performed with a wash buffer solution having a volume of about 100 μL to about 500 μL. In some embodiments, the volume is about 150 μL. In some embodiments, the resuspending of step (a)(iii) is performed with a wash buffer solution having a volume of about 50 μL to about 150 μL. In some embodiments, the volume is about 100 μL. In some embodiments, the wash buffer solution further includes an amplification control nucleic acid. In some embodiments, step (a)(i) further includes adding a TPC to the whole blood sample. In some embodiments, the TPC is an engineered cell including a control target nucleic acid.

In another aspect, the invention features a method for detecting a target pathogen nucleic acid in a whole blood sample, the method including: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and washing the pellet once; (d) centrifuging the product of step (c) to form a supernatant and a pellet; (e) discarding some or all of the supernatant of step (d) and mixing the pellet of (d) with a buffer solution; (f) combining the product of step (e) with beads to form a mixture and agitating the mixture to form a lysate, said lysate containing both subject cell nucleic acid and pathogen nucleic acid; (g) amplifying pathogen nucleic acids in the lysate of step (f) to form an amplified lysate solution including an amplified target pathogen nucleic acid; and (h) sequencing the amplified target pathogen nucleic acid, thereby detecting the target pathogen nucleic acid in the sample. In some embodiments, the washing of step (c) is performed with a wash buffer solution. In some embodiments, the wash buffer solution is TE buffer. In some embodiments, the washing is performed with a wash buffer solution having a volume of about 100 μL to about 500 μL. In some embodiments, the volume is about 150 μL. In some embodiments, step (e) includes mixing the pellet with a buffer solution having a volume of about 50 μL to about 150 μL. In some embodiments, the volume is about 100 μL. In some embodiments, the buffer solution is TE buffer. In some embodiments, the buffer solution and/or the wash buffer solution further includes an amplification control nucleic acid. In some embodiments, step (a) further includes adding a TPC to the whole blood sample. In some embodiments, the TPC is an engineered cell including a control target nucleic acid. In some embodiments, the lysate or the amplified lysate solution has at least about a 2:1 higher concentration of subject cell DNA and/or cell debris relative to the whole blood sample. In some embodiments, the lysate or the amplified lysate solution has at least about a 5:1 higher concentration of subject cell DNA and/or cell debris relative to the whole blood sample. In some embodiments, the lysate or the amplified lysate solution has about a 10:1 higher concentration of subject cell DNA and/or cell debris relative to the whole blood sample. In some embodiments, the amplified lysate solution has a 20:1 higher concentration of subject cell DNA and/or cell debris relative to the whole blood sample. In some embodiments, the lysate or the amplified lysate solution has a 40:1 higher concentration of subject cell DNA and/or cell debris relative to the whole blood sample. In some embodiments, the lysate or the amplified lysate solution has a 60:1 higher concentration of subject cell DNA and/or cell debris relative to the whole blood sample. In some embodiments, the cell debris is solid material.

In some embodiments of any of the preceding methods, the amplifying is performed in the presence of from about 0.5 μg to about 100 μg of subject cell DNA. In some embodiments, the amplifying is performed in the presence of from about 20 to about 80 μg of subject cell DNA. In some embodiments, the amplifying is performed in the presence of about 60 μg of subject cell DNA. In some embodiments, at least a portion of the subject DNA is from white blood cells of the subject.

In some embodiments of any of the preceding methods, the amplifying includes polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM). In some embodiments, the amplifying includes PCR. In some embodiments, the PCR is symmetric PCR or asymmetric PCR.

In some embodiments of any of the preceding methods, the amplifying includes: (i) adding to the lysate an amplification buffer solution including a buffering agent to form a reaction mixture; (ii) heating the reaction mixture to form a denatured reaction mixture; and (iii) adding a thermostable nucleic acid polymerase to the denatured reaction mixture under conditions and for a time sufficient for amplification of the target nucleic acid. In some embodiments, the method further includes centrifuging the denatured reaction mixture to form a pellet and a supernatant prior to step (iii). In some embodiments, step (iii) includes adding the thermostable nucleic acid polymerase to the supernatant.

In other embodiments of any of the preceding methods, the amplifying includes: (i) adding to the lysate an amplification buffer solution including a buffering agent and a thermostable nucleic acid polymerase to form a reaction mixture under conditions and for a time sufficient for amplification of the target nucleic acid; (ii) heating the reaction mixture to form a denatured reaction mixture; and (iii) centrifuging the denatured reaction mixture to form a pellet and a supernatant.

In some embodiments of any of the preceding methods, the amplification buffer solution has a moderately alkaline pH at ambient temperature. In some embodiments, the moderately alkaline pH at ambient temperature is about pH 8.7. In some embodiments, the pH of the buffer solution remains approximately at or above a neutral pH at 95° C. In some embodiments, the pH of the buffer solution is about 6.5 to about 9.0 at 95° C. In some embodiments, the pH of the buffer solution is about 7.0 to about 8.5 at 95° C. In some embodiments, the pH of the buffer solution is about 7.0 to about 7.5 at 95° C. In some embodiments, the pH of the buffer solution is about 7.2 at 95° C.

In some embodiments of any of the preceding methods, the final concentration of the thermostable nucleic acid polymerase in step (iii) is at least about 0.02 units per microliter of the denatured reaction mixture. In some embodiments, the final concentration of the thermostable nucleic acid polymerase in step (i) is at least about 0.02 units per microliter of the reaction mixture. In some embodiments, the final concentration of the thermostable nucleic acid polymerase ranges from about 0.125 to about 0.5 units/μL. In some embodiments, the final concentration of the thermostable nucleic acid ranges from about 0.125 to about 0.25 units/μL.

In some embodiments of any of the preceding methods, step (iii) includes adding to the denatured reaction mixture at least about 2.4×10⁻⁵ micrograms of a thermostable nucleic acid polymerase per microliter of denatured reaction mixture. In some embodiments, the final concentration of thermostable nucleic acid polymerase is from about 2.4×10⁻⁵ micrograms to about 0.01 micrograms per microliter of denatured reaction mixture or reaction mixture. In some embodiments, the final concentration of thermostable nucleic acid polymerase is from about 2.4×10⁻⁵ micrograms to about 0.0001 micrograms per microliter of denatured reaction mixture or reaction mixture.

In other embodiments of any of the preceding methods, step (i) includes adding at least about 2.4×10⁻⁵ micrograms of a thermostable nucleic acid polymerase per microliter of reaction mixture. In some embodiments, the final concentration of thermostable nucleic acid polymerase is from about 2.4×10⁻⁵ micrograms to about 0.01 micrograms per microliter of denatured reaction mixture or reaction mixture. In some embodiments, the final concentration of thermostable nucleic acid polymerase is from about 2.4×10⁻⁵ micrograms to about 0.0001 micrograms per microliter of denatured reaction mixture or reaction mixture.

In some embodiments of any of the preceding methods, the thermostable nucleic acid polymerase is a thermostable DNA polymerase. In some embodiments, the thermostable DNA polymerase is a wild-type thermostable DNA polymerase or a mutant thermostable DNA polymerase. In some embodiments, the wild-type thermostable DNA polymerase is Thermus aquaticus (Taq) DNA polymerase, Thermus thermophilus (Tth) DNA polymerase, Thermus filiformis (Tfi) DNA polymerase, Thermus flavus (Tfl) DNA polymerase, Thermatoga maritima (Tma) DNA polymerase, Thermus spp. Z05 DNA polymerase, or an archaeal polymerase. In some embodiments, the mutant thermostable DNA polymerase is selected from the group consisting of Klentaq®1, Klentaq® LA, Cesium Klentaq® AC, Cesium Klentaq® AC LA, Cesium Klentaq® C, Cesium Klentaq® C LA, Omni Klentaq®, Omni Klentaq® 2, Omni Klentaq® LA, Omni Taq, OmniTaq LA, Omni Taq 2, Omni Taq 3, Hemo KlenTaq®, KAPA Blood DNA polymerase, KAPA3G Plant DNA polymerase, KAPA 3G Robust DNA polymerase, MyTaq™ Blood, PHUSION® Blood II DNA polymerase, AmpliTaq® (Taq G46D), AmpliTaq® Gold, RealTaq, ExcelTaq™, and BioReady Taq. In some embodiments, the mutant thermostable DNA polymerase is a hot start thermostable DNA polymerase. In some embodiments, the hot start thermostable DNA polymerase is APTATAQ™, Hawk Z05, or PHUSION® Blood II DNA polymerase. In some embodiments, the thermostable nucleic acid polymerase is inhibited by the presence of subject-derived cells or cell debris under normal reaction conditions. In some embodiments, the thermostable nucleic acid polymerase is inhibited by the presence of whole blood under normal reaction conditions. In some embodiments, the thermostable nucleic acid polymerase is inhibited by 1% (v/v) whole blood under normal reaction conditions. In some embodiments, the thermostable nucleic acid polymerase is inhibited by 8% (v/v) whole blood under normal reaction conditions. In some embodiments, the normal reaction conditions are the reaction conditions recommended by the manufacturer of the thermostable DNA polymerase.

In some embodiments of any of the preceding methods, the method includes adding deoxynucleotide triphosphates (dNTPs), magnesium, a forward primer, and/or a reverse primer during step (i) or during step (iii).

In some embodiments of any of the preceding methods, the whole blood sample has a volume of about 0.2 mL to about 20 mL, about 0.2 mL to about 15 mL, about 0.2 mL to about 10 mL, about 0.2 mL to about 5 mL, about 0.2 mL to about 2 mL, about 0.4 mL to about 20 mL, about 0.4 mL to about 15 mL, about 0.4 mL to about 10 mL, about 0.4 mL to about 5 mL, about 0.4 mL to about 2 mL, about 0.6 mL to about 20 mL, about 0.6 mL to about 15 mL, about 0.6 mL to about 10 mL, about 0.6 mL to about 5 mL, about 0.6 mL to about 2 mL, about 0.8 mL to about 20 mL, about 0.8 mL to about 15 mL, about 0.8 mL to about 10 mL, about 0.8 mL to about 5 mL, about 0.8 mL to about 2 mL, about 1 mL to about 20 mL, about 1 mL to about 15 mL, about 1 mL to about 10 mL, about 1 mL to about 5 mL, about 1 mL to about 4 mL, about 1 mL to about 3 mL, about 1 mL to about 2 mL, about 1.5 mL to about 20 mL, about 1.5 mL to about 15 mL, about 1.5 mL to about 10 mL, about 1.5 mL to about 5 mL, about 1.5 mL to about 4 mL, about 1.5 mL to about 3 mL, about 1.5 mL to about 2 mL, about 2 mL to about 20 mL, about 2 mL to about 15 mL, about 2 mL to about 10 mL, about 2 mL to about 5 mL, about 2 mL to about 4 mL, about 2 mL to about 3 mL, about 3 mL to about 20 mL, about 3 mL to about 15 mL, about 3 mL to about 10 mL, about 3 mL to about 5 mL, about 3 mL to about 4 mL, about 4 mL to about 20 mL, about 4 mL to about 15 mL, about 4 mL to about 10 mL, about 4 mL to about 5 mL, about 5 mL to about 20 mL, about 5 mL to about 15 mL, about 5 mL to about 10 mL, about 6 mL to about 20 mL, about 6 mL to about 15 mL, about 6 mL to about 10 mL, about 7 mL to about 20 mL, about 7 mL to about 15 mL, about 7 mL to about 10 mL, about 8 mL to about 20 mL, about 8 mL to about 15 mL, about 8 mL to about 10 mL, about 9 mL to about 20 mL, about 9 mL to about 15 mL, about 9 mL to about 10 mL, about 10 mL to about 20 mL, or about 10 mL to about 15 mL. In some embodiments of any of the preceding methods, the whole blood sample has a volume of about 0.2 mL to about 5 mL. In some embodiments, the volume is about 2 mL.

In some embodiments of any of the preceding methods, the lysate produced from the whole blood sample has a volume of about 10 μL to about 500 μL (e.g., about 10 μL, about 20 μL about 30 μL, about 40 μL, about 50 μL, about 60 μL, about 70 μL, about 80 μL, about 90 μL, about 100 μL, about 125 μL, about 150 μL, about 175 μL, about 200 μL, about 225 μL, about 250 μL, about 275 μL, about 300 μL, about 325 μL, about 350 μL, about 375 μL, about 400 μL, about 425 μL, about 450 μL, about 475 μL, or about 500 μL). In some embodiments, the lysate produced from the whole blood sample has a volume of about 25 μL to about 200 μL. In some embodiments, the lysate produced from the whole blood sample has a volume of about 50 μL.

In some embodiments of any of the preceding methods, the reaction mixture of step (i) contains about 1% to about 70% lysate (e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, or about 70% lysate). In some embodiments, the reaction mixture of step (i) contains about 50% lysate.

In some embodiments of any of the preceding methods, the method does not include extracting or purifying the amplified target nucleic acid prior to the sequencing. In some embodiments, the extracting includes chloroform or phenol/chloroform extraction, nuclease digestion, salting out, ion exchange extraction, binding to silica or other solid phase materials, or gel extraction.

In some embodiments of any of the preceding methods, the method further includes cleaning up the amplified target nucleic acid prior to the sequencing. In some embodiments, the cleaning up includes magnetic bead purification, enzymatic clean-up, or column clean-up.

In some embodiments of any of the preceding methods, the sequencing includes massively parallel sequencing, Sanger sequencing, or single-molecule sequencing. In some embodiments, the massively parallel sequencing includes sequencing by synthesis or sequencing by ligation. In some embodiments, the massively parallel sequencing includes sequencing by synthesis. In some embodiments, the sequencing by synthesis includes ILLUMINA™ dye sequencing, ion semiconductor sequencing, or pyrosequencing. In some embodiments, the sequencing by synthesis includes ILLUMINA™ dye sequencing. In some embodiments, the sequencing by ligation includes sequencing by oligonucleotide ligation and detection (SOLiD™) sequencing or polony-based sequencing. In some embodiments, the single-molecule sequencing is nanopore sequencing, single-molecule real-time (SMRT™) sequencing, or Helicos™ sequencing.

In some embodiments, the massively parallel sequencing comprises use of a synthetic control DNA to normalize read counts, wherein the target nucleic acid is detected in the sample if the normalized read count for the target nucleic acid is at or above a reference read count. In some embodiments, the reference read count is 3 standard deviations above the average normalized read count of the highest contaminating sequence from a negative sample.

In some embodiments of any of the preceding methods, the method further includes amplifying one or more additional target nucleic acids in a multiplexed amplification reaction to generate one or more additional amplicons.

In some embodiments of any of the preceding methods, the method identifies the genus of the pathogen.

In some embodiments of any of the preceding methods, the method identifies the species of the pathogen.

In some embodiments of any of the preceding methods, the method further includes detecting the amplified target nucleic acid using T2 magnetic resonance (T2MR) prior to the sequencing. In some embodiments, the method includes the following steps: (i) adding magnetic particles to a portion of the amplified solution or amplified lysate solution to form a detection mixture, wherein the magnetic particles have binding moieties on their surface, the binding moieties operative to alter aggregation of the magnetic particles in the presence of the amplified target nucleic acid, and (ii) detecting the presence of the amplified target nucleic acid by measuring the aggregation of the magnetic particles using T2MR. In some embodiments, step (ii) includes the following steps: (a) providing the detection mixture in a detection tube within a device, the device including a support defining a well for holding the detection tube including the mixture, and having an RF coil configured to detect a signal produced by exposing the mixture to a bias magnetic field created using one or more magnets and an RF pulse sequence; (b) exposing the detection mixture to a bias magnetic field and an RF pulse sequence; (c) following step (b), measuring the signal from the detection tube; and (d) on the basis of the result of step (c), detecting the amplified target nucleic acid. In some embodiments, the magnetic particles include a first population of magnetic particles conjugated to a first probe, and a second population of magnetic particles conjugated to a second probe, the first probe operative to bind to a first segment of the amplified target nucleic acid and the second probe operative to bind to a second segment of the amplified target nucleic acid, wherein the magnetic particles form aggregates in the presence of the amplified target nucleic acid. In some embodiments, from 1×10⁶ to 1×10¹³ magnetic particles are added per milliliter of the sample or the amplified solution. In some embodiments, the magnetic particles have a mean diameter of from about 650 nm to about 950 nm. In some embodiments, the magnetic particles have a mean diameter of from about 670 nm to about 890 nm. In some embodiments, the magnetic particles have a T₂ relaxivity per particle of from 1×10⁹ to 1×10¹² mM⁻¹s⁻¹. In some embodiments, the magnetic particles are substantially monodisperse. In some embodiments, detecting the amplified target nucleic acid using T2MR results in a species or group-level identification of the target nucleic acid by T2MR. In some embodiments, the group-level identification identifies the organism from which the target nucleic acid is obtained as pan-Gram positive, pan-Gram negative, Enterobacteriaceae, an Enterobacter spp., an Enterobacter cloacae complex, a Citrobacter spp., an Enterococcus spp., a Streptococcus spp., a Staphylococcus spp., an Acinetobacter spp., a Corynebacterium spp., a Mycobacterium spp., pan-fungal, or a Candida spp. In some embodiments, the Staphylococcus spp. is a coagulase-negative Staphylococcus spp. In some embodiments, detecting the amplified target nucleic acid using T2MR results in the identification of a sequence of an antimicrobial resistance gene or a toxin gene or a fragment thereof. In some embodiments, the antimicrobial resistance gene is bla_{KPC}, blaZ, bla_{NDM}, bla_{IMP}, bla_{VIM}, bla_{OXA} (e.g., bla_{OXA-48}), bla_{CMY}, bla_{DHA}, bla_{TEM}, bla_{SHV}, bla_{CTX-M}, bla_{SME}, bl/a_{FOX}, bla_{MIR}, femA, femB, mecA, mecC, macB, fosA, vanA, vanB, vanC, vanD, vanE, vanG, mefA, metE, ermA, ermB, tetA, tetB, tetX, tetR, qnrA, qnrB, qnrS, FKS1, FKS2, ERG11, or PDR1. In some embodiments, the toxin gene is a B. anthracis toxin or capsule gene, an enteropathogenic E. coli Tir gene, a C. difficile toxin gene, or a C. botulinum toxin gene. In some embodiments, the toxin gene is selected from the group consisting of Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G. In some embodiments, sequencing the amplified target nucleic acid results in a species-level or variant-level identification of the target nucleic acid. In some embodiments, the species level is a taxonomic species, a taxonomic subspecies, a strain, or a nucleic acid variant. In some embodiments, the nucleic acid variant includes a single nucleotide polymorphism (SNP), an insertion/deletion (indel), a repetitive element, or a microsatellite repeat. In some embodiments, the group-level identification by T2MR is pan-Gram positive, and the species-level identification by sequencing is Enterococcus faecium, Enterococcus faecalis, Streptococcus pneumoniae, Streptococcus pyogenes, a viridans Streptococcus, or Staphylococcus aureus. In some embodiments, the group-level identification by T2MR is pan-Gram negative, and the species-level identification by sequencing is Acinetobacter baumannii, Escherichia coli, Haemophilus influenzae, Klebsiella pneumoniae, or Pseudomonas aeruginosa. In some embodiments, the identification by T2MR is an antimicrobial resistance gene, and the variant-level identification by sequencing is a nucleic acid variant of the antimicrobial resistance gene. In some embodiments, the antimicrobial resistance gene is bla_{KPC}, blaZ, bla_{NDM}, bla_{IMP}, bla_{VIM}, bla_{OXA} (e.g., bla_{OXA-48}), bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, metE, ermA, ermB, tetA, tetB, tetX, tetR, qnrA, qnrB, qnrS, FKS1, FKS2, ERG11, or PDR1. In some embodiments: (i) the identification by T2MR is bla_{KPC}, and the variant-level identification by sequencing is KPC-1, KPC-2, KPC-3, KPC-4, KPC-5, KPC-6, KPC-7, KPC-8, KPC-10, KPC-11, KPC-12, KPC-13, KPC-14, KPC-15, KPC-16, KPC-17, KPC-18, KPC-19, KPC-21, KPC-22, KPC-23, KPC-24, KPC-25, KPC-26, KPC-27, KPC-28, KPC-29, KPC-30, KPC-31, KPC-32, KPC-33, KPC-34, or KPC-35; (ii) the identification by T2MR is bla_{CTX-M}, and the variant-level identification by sequencing is CTX-M-1, CTX-M-2, CTX-M-3, CTX-M-4, CTX-M-5, CTX-M-6, CTX-M-7, CTX-M-8, CTX-M-9, CTX-M-10, CTX-M-12, CTX-M-13, CTX-M-14, CTX-M-15, CTX-M-16, CTX-M-17, CTX-M-19, CTX-M-20, CTX-M-21, CTX-M-22, CTX-M-23, CTX-M-24, CTX-M-25, CTX-M-26, CTX-M-27, CTX-M-28, CTX-M-29, CTX-M-30, CTX-M-31, CTX-M-32, CTX-M-33, CTX-M-34, CTX-M-35, CTX-M-36, CTX-M-37, CTX-M-38, CTX-M-39, CTX-M-40, CTX-M-41, CTX-M-42, CTX-M-43, CTX-M-44, CTX-M-46, CTX-M-47, CTX-M-48, CTX-M-49, CTX-M-50, CTX-M-51, CTX-M-52, CTX-M-53, CTX-M-54, CTX-M-55, CTX-M-56, CTX-M-58, CTX-M-59, CTX-M-60, CTX-M-61, CTX-M-62, CTX-M-63, CTX-M-64, CTX-M-65, CTX-M-66, CTX-M-67, CTX-M-68, CTX-M-69, CTX-M-71, CTX-M-72, CTX-M-73, CTX-M-74, CTX-M-75, CTX-M-76, CTX-M-77, CTX-M-78, CTX-M-79, CTX-M-80, CTX-M-81, CTX-M-82, CTX-M-83, CTX-M-84, CTX-M-85, CTX-M-86, CTX-M-87, CTX-M-88, CTX-M-89, CTX-M-90, CTX-M-91, CTX-M-92, CTX-M-93, CTX-M-94, CTX-M-95, CTX-M-96, CTX-M-97, CTX-M-98, CTX-M-99, CTX-M-100, CTX-M-101, CTX-M-102, CTX-M-103, CTX-M-104, CTX-M-105, CTX-M-110, CTX-M-111, CTX-M-112, CTX-M-113, CTX-M-114, CTX-M-115, CTX-M-116, CTX-M-117, CTX-M-121, CTX-M-122, CTX-M-123, CTX-M-124, CTX-M-125, CTX-M-126, CTX-M-127, CTX-M-129, CTX-M-130, CTX-M-131, CTX-M-132, CTX-M-134, CTX-M-136, CTX-M-137, CTX-M-138, CTX-M-139, CTX-M-141, CTX-M-142, CTX-M-144, CTX-M-146, CTX-M-147, CTX-M-148, CTX-M-150, CTX-M-151, CTX-M-152, CTX-M-155, CTX-M-156, CTX-M-157, CTX-M-158, CTX-M-159, CTX-M-160, CTX-M-161, CTX-M-162, CTX-M-163, CTX-M-164, CTX-M-165, CTX-M-166, CTX-M-167, CTX-M-168, CTX-M-169, CTX-M-170, CTX-M-171, CTX-M-172, CTX-M-173, CTX-M-174, CTX-M-175, CTX-M-176, CTX-M-177, CTX-M-178, CTX-M-179, CTX-M-180, CTX-M-181, CTX-M-182, CTX-M-183, CTX-M-184, CTX-M-185, CTX-M-186, CTX-M-187, CTX-M-188, CTX-M-189, CTX-M-190, CTX-M-191, CTX-M-192, CTX-M-193, CTX-M-194, CTX-M-195, CTX-M-196, CTX-M-197, CTX-M-198, CTX-M-199, CTX-M-200, CTX-M-201, CTX-M-202, CTX-M-203, CTX-M-204, CTX-M-205, CTX-M-206, CTX-M-207, CTX-M-208, CTX-M-209, CTX-M-210, CTX-M-211, CTX-M-212, CTX-M-213, CTX-M-214, CTX-M-216, CTX-M-217, CTX-M-218, CTX-M-219, or CTX-M-220; or (iii) the identification by T2MR is bla_{NDM}, and the variant-level identification by sequencing is NDM-1, NDM-2, NDM-3, NDM-4, NDM-5, NDM-6, NDM-7, NDM-8, NDM-9, NDM-10, NDM-11, NDM-12, NDM-13, NDM-14, NDM-15, NDM-16, NDM-17, NDM-18, NDM-19, NDM-20, NDM-21, NDM-22, NDM-23, or NDM-24. In some embodiments, the group-level identification by T2MR is pan-fungal or a Candida spp., and the species-level identification by T2MR is Candida albicans, Candida guilliermondii, Candida glabrata, Candida krusei, Candida lusitaniae, Candida parapsilosis, Candida metapsilosis, Candida orthopsilosis, Candida dublinensis, Candida tropicalis, Candida auris, Candida haemulonii, Candida duobushaemulonii, Candida pseudohaemulonii, an Aspergillus spp., or a Cryptococcus spp. In some embodiments, the detecting by T2MR is completed within 5 hours of amplifying the target nucleic acid. In some embodiments, the detecting by T2MR is completed within 3 hours of amplifying the target nucleic acid.

In some embodiments of any of the preceding methods, the pathogen is a fungal pathogen, a bacterial pathogen, a protozoan pathogen, or a viral pathogen. In some embodiments, the pathogen is a fungal pathogen. In some embodiments, the fungal pathogen is a Candida spp., an Aspergillus spp., or a Cryptococcus spp. In some embodiments, the Candida spp. is selected from the group consisting of Candida albicans, Candida guilliermondii, Candida glabrata, Candida krusei, Candida lusitaniae, Candida parapsilosis, Candida metapsilosis, Candida orthopsilosis, Candida dublinensis, Candida tropicalis, Candida auris, Candida haemulonii, Candida duobushaemulonii, or Candida pseudohaemulonii. In some embodiments, the Candida spp. is selected from the group consisting of Candida albicans, Candida guilliermondii, Candida glabrata, Candida krusei, Candida lusitaniae, Candida parapsilosis, and Candida tropicalis. In some embodiments, the pathogen is a bacterial pathogen. In some embodiments, the amplifying includes amplifying a pan-bacterial amplicon. In some embodiments, the pan-bacterial amplicon is a 16S rRNA amplicon. In some embodiments, the amplifying includes amplifying the 16S rRNA amplicon in the presence of a forward primer including the nucleic acid sequence of 5′-GGTTAAGTCCCGCAACGAGCGC-3′ (SEQ ID NO: 60) and a reverse primer including the nucleic acid sequence of 5′-AGGAGGTGATCCAACCGCA-3′ (SEQ ID NO: 61). In some embodiments, the bacterial pathogen is a Gram positive bacterium, a Gram negative bacterium, an Enterobacteriaceae family bacterium, an Enterobacter spp., a Citrobacter spp., an Enterococcus spp., a Streptococcus spp., a Staphylococcus spp., an Acinetobacter spp., a Corynebacterium spp., Enterobacter cloacae complex, or a Mycobacterium spp. In some embodiments, the Staphylococcus spp. is a coagulase-negative Staphylococcus spp. In some embodiments, the Streptococcus spp. is a viridans Streptococcus. In some embodiments, the bacterial pathogen is selected from the group consisting of Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, and Neisseria meningitides. In some embodiments, the bacterial pathogen is selected from the group consisting of Acinetobacter baumannii, Enterococcus faecium, Staphylococcus aureus, Klebsiella pneumoniae, Pseudomonas aeruginosa, and Escherichia coli. In some embodiments, the bacterial pathogen is selected from Borrelia burgdorferi, Borrelia afzelii, and Borrelia garinii. In some embodiments, the protozoan pathogen is Babesia microti or Babesia divergens.

In some embodiments of any of the preceding methods, the method is capable of detecting a concentration of about 10 colony-forming units (CFU)/mL of the pathogen species in the whole blood sample or lower (e.g., about 1, about 2, about 3, about 4, about 5, about 6, about 7, about 8, about 9, or about 10 CFU/mL). In some embodiments, the method is capable of detecting a concentration of about 1 CFU/mL to about 10 CFU/mL of the pathogen species in the whole blood sample.

In some embodiments of any of the preceding methods, the target nucleic acid is an antimicrobial resistance gene. In some embodiments, the antimicrobial resistance gene is bla_{KPC}, blaZ, bla_{NDM}, bla_{IMP}, bla_{VIM}, bla_{OXA} (e.g., bla_{OXA-48}), bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, mefE, ermA, ermB, tetA, tetB, tetX, tetR, qnrA, qnrB, qnrS, FKS1, FKS2, ERG11, or PDR1.

In some embodiments of any of the preceding methods, the method further includes diagnosing the subject based on the detection of the target nucleic acid, wherein the presence of the target nucleic indicates that the subject is suffering from a disease associated with the pathogen. In some embodiments, the method further includes administering to the subject a suitable therapy.

In another aspect, the invention features a method for detecting a target nucleic acid in a biological sample obtained from a subject, wherein the biological sample includes subject-derived cells or cell debris, the method including: (a) amplifying a target nucleic acid in the biological sample to form an amplified solution including an amplified target nucleic acid; (b) detecting the amplified target nucleic acid using T2MR to provide a group-level identification of the target nucleic acid; and (c) sequencing the amplified target nucleic acid to provide a species-level or variant-level identification of the target nucleic acid, wherein the method is capable of detecting a concentration of about 10 copies/mL of the target nucleic acid in the biological sample.

Other features and advantages of the invention will be apparent from the following detailed description, drawings, and the claims.

### BRIEF DESCRIPTION OF THE DRAWINGS

FIG. 1 is a schematic diagram of a T2 magnetic resonance (T2MR) and Sanger sequencing process.

FIG. 2 is a graph showing read counts from three next-generation sequencing (NGS)-analyzed samples spiked at 2-3× limit of detection (LoD). The read counts of the spiked species are greater than the sum of all background contaminants.

FIG. 3 is a graph showing normalized read counts for 5, 10, 25, and 50 colony-forming unit (CFU)/mL E. faecium in whole blood samples. Read counts were normalized by a synthetic control. A cutoff was established from blank samples (dotted line). All E. faecium samples have normalized reads above the cutoff, and the presumptive LoD is ≤5 CFU/mL. Average normalized reads for contaminating species with the highest read count were consistently under the cutoff. A linear relationship between normalized read counts and sample concentration was observed for E. faecium spikes (solid line).

### DETAILED DESCRIPTION OF EMBODIMENTS OF THE INVENTION

The invention provides, inter alia, methods, systems, cartridges, kits, and panels for sequencing of one or more target nucleic acids in complex biological or environmental samples containing cells, cell debris (e.g., blood), or non-specific nucleic acids (e.g., subject (e.g., host) cell DNA). The present invention is based, at least in part, on the unexpected discovery of sample preparation approaches that allow for direct sequencing of target nucleic acids in complex samples without prior nucleic acid extraction or purification. Surprisingly, sequencing was successfully performed using amplified lysate samples that contain concentrated cell debris and subject-cell derived nucleic acids (e.g., DNA) relative to the original biological sample (e.g., blood) from a subject. As an example, 2 mL of a biological sample (e.g., blood) concentrated down to 0.1 mL in a lysate corresponds to a 20:1 higher concentration of debris compared to the original sample. If the lysate is diluted 1:1 for amplification, the amplification is performed in a lysate (e.g., an amplified lysate solution) that represents a 10:1 concentration of the debris in the original sample. The present approaches allow for high-sensitivity and specific sequencing-based detection of target nucleic acids, including those from low-titer pathogens (e.g., less than 10 cells/mL, e.g., 1, 2, 3, 4, 5, 6, 7, 8, 9, or 10 cells/mL). Further, the present approaches can provide group-level, species-level, and/or variant-level information regarding target nucleic acids, e.g., pathogen-associated target nucleic acids, antibiotic resistance genes, or toxins.

In some embodiments, sequencing of the target nucleic acid amplicon(s) allows for rapid, accurate, and high sensitivity detection and identification of a microbial pathogen present in a biological or environmental sample containing host cells, cell debris, and/or host cell nucleic acids (e.g., DNA), including but not limited to whole blood, processed whole blood (e.g., a crude whole blood lysate), serum, plasma, or other blood derivatives; bloody fluids such as wound exudate, phlegm, bile, and the like; tissue samples (e.g., tissue biopsies); and sputum (e.g., purulent sputum and bloody sputum)), which may be used, for example, for diagnosis of a disease (e.g., sepsis, bloodstream infections (BSIs) (e.g., bacteremia, fungemia (e.g., Candidemia), and viremia), endocarditis, transplant-associated infection, Lyme disease (e.g., for pan-tick-borne pathogen identification), septic shock, and diseases that may manifest with similar symptoms to diseases caused by or associated with microbial pathogens, e.g., systemic inflammatory response syndrome (SIRS)).

In some embodiments, the methods, systems, cartridges, kits, and panels can be used in combination with T2MR detection of target nucleic acids. For example, in some embodiments, the T2MR detection can provide group-level information that is used to direct or narrow sequencing in a sample. In some embodiments, the T2MR detection approaches employ magnetic particles. In some embodiments, the methods and systems employ an NMR unit, optionally one or more magnetic assisted agglomeration (MAA) units, optionally one or more incubation stations at different temperatures, optionally one or more vortexers, optionally one or more centrifuges, optionally a fluidic manipulation station, optionally a robotic system, and optionally one or more modular cartridges, as described in International Patent Application Publication No. WO 2012/054639, which is incorporated herein by reference in its entirety. In some embodiments, the methods of the invention are performed using a fully-automated system, e.g., which may contain a sequencing unit and, optionally, a NMR unit.

Definitions

The terms “amplification” or “amplify” or derivatives thereof, as used herein, mean one or more methods known in the art for copying a target or template nucleic acid, thereby increasing the number of copies of a selected nucleic acid sequence. Amplification may be exponential or linear. A “target nucleic acid” refers to a nucleic acid or a portion thereof that is to be amplified, detected, and/or sequenced. A target or template nucleic acid may be any nucleic acid, including DNA or RNA. The sequences amplified in this manner form an “amplified target nucleic acid,” “amplified region,” or “amplicon,” which are used interchangeably herein. Primers and/or probes can be readily designed to target a specific template nucleic acid sequence. Exemplary amplification approaches include but are not limited to polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, and ramification amplification (RAM).

As used herein, the terms “unit” or “units,” when used in reference to thermostable nucleic acid polymerases, refer to an amount of the thermostable nucleic acid polymerase (e.g., thermostable DNA polymerase). Typically a unit is defined as the amount of enzyme that will incorporate a particular amount of dNTPs (e.g., 10-20 nmol) into acid-insoluble material in 30-60 min at 65° C.-75° C. under particular assay conditions, although each manufacturer may define units differently. Unit definitions and assay conditions for commercially-available thermostable nucleic acid polymerases are known in the art. In some embodiments, one unit of thermostable nucleic acid polymerase (e.g., Taq DNA polymerase) may be the amount of enzyme that will incorporate 15 nmol of dNTP into acid-insoluble material in 30 min at 75° C. in an assay containing 1× ThermoPol® Reaction Buffer (New England Biosciences), 200 μM dNTPs including [³H]-dTTP, and 15 nM primed M13 DNA.

The term “sequencing” refers to any method for determining the nucleotide order of a nucleic acid (e.g., DNA), such as a target nucleic acid or an amplified target nucleic acid. Exemplary sequencing approaches include but are not limited to massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing)), long-read or single-molecule sequencing (e.g., Helicos™ sequencing, single-molecule real-time (SMRT™) sequencing, and nanopore sequencing) and Sanger sequencing. Massively parallel sequencing is also referred to in the art as next-generation or second-generation sequencing, and typically involves parallel sequencing of a large number (e.g., thousands, millions, or billions) of spatially-separated, clonally amplified templates or single nucleic acid molecules. Short reads are often used in massively parallel sequencing. See, e.g., Metzker, Nature Reviews Genetics 11:31-36, 2010. Long-read sequencing and/or single-molecule sequencing are sometimes referred to as third-generation sequencing. Hybrid approaches (e.g., massively parallel and single molecule approaches or massively parallel and long-read approaches) can also be used. It is to be understood that some approaches may fall into more than one category, for example, some approaches may be considered both second-generation and third-generation approaches, and some sources refer to both second and third generation sequencing as “next-generation” sequencing.

By “analyte” is meant a substance or a constituent of a sample to be analyzed. Exemplary analytes include one or more species of one or more of the following: a nucleic acid (e.g., DNA or RNA (e.g., mRNA)), an oligonucleotide, a protein, a peptide, a polypeptide, an amino acid, an antibody, a carbohydrate, a polysaccharide, glucose, a lipid, a gas (e.g., oxygen or carbon dioxide), an electrolyte (e.g., sodium, potassium, chloride, bicarbonate, blood urea nitrogen (BUN), magnesium, phosphate, calcium, ammonia, lactate), a lipoprotein, cholesterol, a fatty acid, a glycoprotein, a proteoglycan, a lipopolysaccharide, a cell surface marker (e.g., a cell surface protein of a pathogen), a cytoplasmic marker (e.g., CD4/CD8 or CD4/viral load), a therapeutic agent, a metabolite of a therapeutic agent, a marker for the detection of a weapon (e.g., a chemical or biological weapon), an organism, a pathogen, a pathogen byproduct, a parasite (e.g., a protozoan or a helminth), a protist, a fungus (e.g., yeast (e.g., a Candida species (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis)) or mold), a bacterium (e.g., Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, or Neisseria meningitides), an actinomycete, a cell (e.g., a whole cell, a tumor cell, a stem cell, a white blood cell, a T cell (e.g., displaying CD3, CD4, CD8, IL2R, CD35, or other surface markers), or another cell identified with one or more specific markers), a virus, a prion, a plant component, a plant by-product, algae, an algae by-product, plant growth hormone, an insecticide, a man-made toxin, an environmental toxin, an oil component, and components derived therefrom. In particular embodiments, the analyte is a nucleic acid (e.g., DNA or RNA (e.g., mRNA)), such as a target nucleic acid or an amplified target nucleic acid.

A “biological sample” is a sample obtained from a subject including but not limited to blood (e.g., whole blood, processed whole blood (e.g., a crude whole blood lysate), serum, plasma, and other blood derivatives), bloody fluids (e.g., wound exudate, phlegm, bile, and the like), urine, cerebrospinal fluid (CSF), synovial fluid (SF), breast milk, sweat, tears, saliva, semen, feces, vaginal fluid or tissue, sputum (e.g., purulent sputum and bloody sputum), nasopharyngeal aspirate or swab, lacrimal fluid, mucous, epithelial swab (e.g., a buccal swab, an axilla swab, a groin swab, an axilla/groin swab, or an ear swab), tissues (e.g., tissue biopsies (e.g., skin biopsies (e.g., from wounds, burns, or tick bites), muscle biopsies, or lymph node biopsies)), including tissue homogenates), organs, bones, teeth, or culture media (e.g., BHI, SABHI, SDA, LB, and the like), among others. In some embodiments, the biological sample is whole blood, which may contain an anticoagulant (e.g., EDTA, sodium citrate, sodium heparin, lithium heparin, and/or potassium oxylate/sodium fluoride). In several embodiments, the biological sample contains cells, cell debris, and/or nucleic acids (e.g., DNA) derived from the subject from which the sample was obtained. In particular embodiments, the subject is a host of a pathogen, and the biological sample obtained from the subject includes subject (host)-derived cells, cell debris, and nucleic acids (e.g., DNA), as well as one or more pathogen cells. In some embodiments, the swab buffer diluent or swab transport medium is, without limitation, PBST, Amies Buffer, Amies Buffer+10% (v/v) 10× PBST, Cary Blair Media, or Liquid Stuart Swabs (which may include addition of 10% (v/v) 10× PBST). The biological sample may be a liquid sample.

As used herein, an “environmental sample” is a sample obtained from an environment, e.g., a surface swab sample, a sample from a building or a container, an air sample, a water sample, a soil sample, and the like. The environmental sample may contain any analyte described herein, e.g., a pathogen such as a bacterial (e.g., Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, or Neisseria meningitides), fungal (e.g., a Candida species (e.g., Candida auris, Candida lusitaniae, Candida haemulonii, Candida duobushaemulonii, Candida pseudohaemulonii, Candida guilliermondii, Candida albicans, Candida glabrata, Candida krusei, C. parapsilosis, and/or C. tropicalis)), protozoan, or viral organism or pathogen. In some embodiments, an environmental sample is from a hospital or other healthcare facility. In some embodiments, the environmental sample is a swab, e.g., swab buffer diluent or swab transport medium is, without limitation, PBST, Amies Buffer, Amies Buffer+10% (v/v) 10× PBST, Cary Blair Media, or Liquid Stuart Swabs (which may include addition of 10% (v/v) 10× PBST). The environmental sample may be a liquid sample.

A “biomarker” is a biological substance that can be used as an indicator of a particular disease state or particular physiological state of an organism, generally a biomarker is a protein or other native compound measured in bodily fluid whose concentration reflects the presence or severity or staging of a disease state or dysfunction, can be used to monitor therapeutic progress of treatment of a disease or disorder or dysfunction, or can be used as a surrogate measure of clinical outcome or progression. In some embodiments, the biomarker is a nucleic acid (e.g., DNA or RNA (e.g., mRNA)).

The term “cell debris” refers to any materials released from cells that have been lysed or otherwise died. Cell debris may include any material that is contained within a cell, e.g., nucleic acids, proteins (e.g., hemoglobin), lipids, glycolipids, small molecules, carbohydrates, heme compounds, membranes, and the like. In several embodiments, the cell debris is or includes solid material, such as solid material that can be concentrated with a liquid-solid separation method (e.g., centrifugation or filtration). In some examples, the cell debris is the solid material present after centrifugation (such as solid material produced by the sample processing procedure described in Examples 1-6).

As used herein, the term “cells/mL” indicates the number of cells per milliliter of a biological or environmental sample. The number of cells may be determined using any suitable method, for example, hemocytometer, quantitative PCR, and/or automated cell counting. It is to be understood that in some embodiments, cells/mL may indicate the number of colony-forming units (CFU) per milliliter of a biological or environmental sample.

A “group,” as used herein, refers to a grouping of organisms, including pathogens. In some embodiments, a group may be a taxonomic classification, for instance, a taxonomic domain, a taxonomic kingdom, a taxonomic phylum, a taxonomic class, a taxonomic order, a taxonomic family, or a taxonomic genus. In other embodiments, a group may be defined by any desired or suitable characteristics such as, for example, resistance to an antimicrobial agent or Gram staining (e.g., Gram positive or Gram negative). For example, the group may be pan-Gram positive or pan-Gram negative. It is to be understood that, in some instances, a pathogen may belong to more than one group.

A “group-level” identification refers to identification of an analyte (e.g., a target nucleic acid) that provides information regarding a group from which the analyte was obtained (e.g., a taxonomic classification, for instance, a taxonomic domain, a taxonomic kingdom, a taxonomic phylum, a taxonomic class, a taxonomic order, a taxonomic family, or a taxonomic genus). In some embodiments, a group-level identification does not provide species-level identification.

The term “species,” as used herein, refers to a basic unit of biological classification as well as a taxonomic rank. A skilled artisan appreciates that a species may be defined based on a number of criteria, including, for example, DNA similarity, morphology, and ecological niche. The term encompasses any suitable species concept, including evolutionary species, phylogenetic species, typological species, genetic species, and reproductive species. The term also encompasses subspecies or strains.

A “species-level” identification refers to identification of an analyte (e.g., a target nucleic acid) that provides information regarding the species from which the analyte was obtained. With respect to target nucleic acids, in some embodiments, species-level identification provides information regarding nucleic acid variants (e.g., a single nucleotide polymorphism (SNP), an insertion/deletion (indel), a repetitive element, or a microsatellite repeat), which is also referred to herein as a “variant-level” identification. In some embodiments, a species-level or variant-level identification also provides a group-level identification.

A “pan-Bacterial” marker (e.g., target nucleic acid) is a marker that is characteristic of many or all forms of bacteria. The presence of a pan-Bacterial marker in a sample may indicate the presence of a bacterial organism (e.g., a pathogen) in the sample. Exemplary pan-Bacterial markers include, without limitation, 16S rRNA and 23S rRNA. In some embodiments, a pan-Bacterial marker is characteristic of about 90%, about 91%, about 92%, about 93%, about 94%, about 95%, about 96%, about 97%, about 98%, or about 99% of bacteria.

A “pan-Fungal” marker (e.g., target nucleic acid) is a marker that is characteristic of many or all forms of fungi. The presence of a pan-Fungal marker in a sample may indicate the presence of a fungal organism (e.g., a pathogen) in the sample. Exemplary pan-Fungal markers include, without limitation, Inverted Transcribed Spacer (ITS) rRNA (e.g., ITS1 or ITS2), 28S rRNA, 18S rRNA, 5.8S rRNA, or a combination thereof. In some embodiments, a pan-Fungal marker is characteristic of about 90%, about 91%, about 92%, about 93%, about 94%, about 95%, about 96%, about 97%, about 98%, or about 99% of fungi (e.g., medically-relevant fungi, e.g., fungal pathogens).

A “pathogen” means an agent causing disease or illness to its host, such as an organism or infectious particle, capable of producing a disease in another organism, and includes but is not limited to bacteria (e.g., Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, or Neisseria meningitides), viruses, protozoa, prions, fungi (e.g., yeast (e.g., Candida species), or pathogen by-products. “Pathogen by-products” are those biological substances arising from the pathogen that can be deleterious to the host or stimulate an excessive host immune response, for example pathogen nucleic acids, antigen(s), metabolic substances, enzymes, biological substances, or toxins (e.g., Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G).

By “pathogen-associated analyte” is meant an analyte characteristic of the presence of a pathogen (e.g., a bacterium, fungus, or virus) in a sample. The pathogen-associated analyte can be a particular substance derived from a pathogen (e.g., a nucleic acid (e.g., DNA or RNA (e.g., mRNA)), a protein, lipid, polysaccharide, or any other material produced by a pathogen) or a mixture derived from a pathogen (e.g., whole cells, or whole viruses). In certain instances, the pathogen-associated analyte is selected to be characteristic of the genus, species, or specific strain of pathogen being detected. Alternatively, the pathogen-associated analyte is selected to ascertain a property of the pathogen, such as resistance to a particular therapy. In some embodiments, a pathogen-associated analyte may be a target nucleic acid that has been amplified. In other embodiments, a pathogen-associated analyte may be a host antibody or other immune system protein that is expressed in response to an infection by a pathogen (e.g., an IgM antibody, an IgA antibody, an IgG antibody, or a major histocompatibility complex (MHC) protein).

A “subject” is an animal, preferably a mammal (including, for example, rodents (e.g., mice or rats), farm animals (e.g., cows, sheep, horses, and donkeys), pets (e.g., cats and dogs), or primates (e.g., humans and non-human primates (e.g., monkeys, chimpanzees, and gorillas)). In particular embodiments, the subject is a human. A subject may be a patient (e.g., a patient having or suspected of having a disease associated with or caused by a pathogen). In some embodiments, a subject is a host of one or more pathogens.

By “pharmaceutical composition” is meant any composition that contains a therapeutically or biologically active agent (e.g., an antifungal agent) that is suitable for administration to a subject.

As used herein, by “administering” is meant a method of giving a dosage of a composition (e.g., a pharmaceutical composition) described herein (e.g., a composition comprising an antimicrobial agent) to a subject. The compositions utilized in the methods described herein can be administered by any suitable route, e.g., parenteral (for example, intravenous, intramuscular, intra-arterial, intracardiac, subcutaneous, or intraperitoneal), dermal, transdermal, ocular, inhalation, buccal, sublingual, perilingual, nasal, rectal, topical, and oral. The compositions utilized in the methods described herein can also be administered locally or systemically. The preferred method of administration can vary depending on various factors (e.g., the components of the composition being administered and the severity of the condition being treated).

As used herein, “linked” means attached or bound by covalent bonds, non-covalent bonds, and/or linked via Van der Waals forces, hydrogen bonds, and/or other intermolecular forces.

The term “magnetic particle” refers to particles including materials of high positive magnetic susceptibility such as paramagnetic compounds, superparamagnetic compounds, and magnetite, gamma ferric oxide, or metallic iron.

The terms “aggregation,” “agglomeration,” and “clustering” are used interchangeably in the context of the magnetic particles described herein and mean the binding of two or more magnetic particles to one another, for example, via a multivalent analyte, multimeric form of analyte, antibody, nucleic acid molecule, or other binding molecule or entity. In some instances, magnetic particle agglomeration is reversible. Such aggregation may lead to the formation of “aggregates,” which may include amplicons and magnetic particles bearing binding moieties.

As used herein, “nonspecific reversibility” refers to the colloidal stability and robustness of magnetic particles against non-specific aggregation in a liquid sample and can be determined by subjecting the particles to the intended assay conditions in the absence of a specific clustering moiety (i.e., an analyte or an agglomerator). For example, nonspecific reversibility can be determined by measuring the T₂ values of a solution of magnetic particles before and after incubation in a uniform magnetic field (defined as <5000 ppm) at 0.45 T for 3 minutes at 37° C. Magnetic particles are deemed to have nonspecific reversibility if the difference in T₂ values before and after subjecting the magnetic particles to the intended assay conditions vary by less than 10% (e.g., vary by less than 9%, 8%, 6%, 4%, 3%, 2%, or 1%). If the difference is greater than 10%, then the particles exhibit irreversibility in the buffer, diluents, and matrix tested, and manipulation of particle and matrix properties (e.g., coating and buffer formulation) may be required to produce a system in which the particles have nonspecific reversibility. In another example, the test can be applied by measuring the T₂ values of a solution of magnetic particles before and after incubation in a gradient magnetic field 1 Gauss/mm-10000 Gauss/mm.

As used herein, the term “NMR relaxation rate” refers to a measuring any of the following in a sample T₁, T₂, T₁/T₂ hybrid, T₁ᵣₕₒ, T₂ᵣₕₒ, and T₂*. The systems and methods of the invention are designed to produce an NMR relaxation rate characteristic of whether an analyte is present in the liquid sample. In some instances, the NMR relaxation rate is characteristic of the quantity of analyte present in the liquid sample.

As used herein, the term “T₁/T₂ hybrid” refers to any detection method that combines a T₁ and a T₂ measurement. For example, the value of a T₁/T₂ hybrid can be a composite signal obtained through the combination of, ratio, or difference between two or more different T₁ and T₂ measurements. The T₁/T₂ hybrid can be obtained, for example, by using a pulse sequence in which T₁ and T₂ are alternatively measured or acquired in an interleaved fashion. Additionally, the T₁/T₂ hybrid signal can be acquired with a pulse sequence that measures a relaxation rate that is comprised of both T₁ and T₂ relaxation rates or mechanisms.

By “pulse sequence” or “RF pulse sequence” is meant one or more radio frequency pulses to be applied to a sample and designed to measure, e.g., certain NMR relaxation rates, such as spin echo sequences. A pulse sequence may also include the acquisition of a signal following one or more pulses to minimize noise and improve accuracy in the resulting signal value.

As used herein, the term “signal” refers to an NMR relaxation rate, frequency shift, susceptibility measurement, diffusion measurement, or correlation measurements.

As used herein, reference to the “size” of a magnetic particle refers to the average diameter for a mixture of the magnetic particles as determined by microscopy, light scattering, or other methods.

As used herein, the term “substantially monodisperse” refers to a mixture of magnetic particles having a polydispersity in size distribution as determined by the shape of the distribution curve of particle size in light scattering measurements. The FWHM (full width half max) of the particle distribution curve less than 25% of the peak position is considered substantially monodisperse. In addition, only one peak should be observed in the light scattering experiments and the peak position should be within one standard deviation of a population of known monodisperse particles.

By “T₂ relaxivity per particle” is meant the average T₂ relaxivity per particle in a population of magnetic particles.

As used herein, “unfractionated” refers to an assay in which none of the components of the sample being tested are removed following the addition of magnetic particles to the sample and prior to the NMR relaxation measurement.

A “reference read count” refers to a read count that is used for comparison purposes. For example, a massively parallel sequencing approach described herein may involve use of a synthetic control DNA to normalize read counts, and a target nucleic acid is detected if the normalized read count for the target nucleic acid is at or above a reference read count. Any suitable cutoff may be used as a reference read count. For example, in some embodiments, the reference read count is 3 standard deviations above the average normalized read count of the highest contaminating sequence from a negative sample (e.g., a control sample that does not contain a target nucleic acid).

It is contemplated that units, methods, systems, cartridges, kits, panels, and processes of the claimed invention encompass variations and adaptations developed using information from the embodiments described herein. Throughout the description, where units, systems, cartridges, kits, or panels are described as having, including, or including specific components, or where processes and methods are described as having, including, or including specific steps, it is contemplated that, additionally, there are units, systems, cartridges, kits, or panels of the present invention that consist essentially of, or consist of, the recited components, and that there are processes and methods according to the present invention that consist essentially of, or consist of, the recited processing steps. It should be understood that the order of steps or order for performing certain actions is immaterial, unless otherwise specified, so long as the invention remains operable. Moreover, in many instances two or more steps or actions may be conducted simultaneously.

Methods of Sequencing Target Nucleic Acids in Complex Samples

The invention provides methods for sequencing target nucleic acids in complex samples (e.g., biological or environmental samples) containing cells, cell debris (e.g., solid material), or nucleic acids (e.g., DNA or RNA (e.g., mRNA), e.g., non-target and/or subject-derived nucleic acids). In several embodiments, the sample contains cells and/or cell debris derived from a mammalian host subject and one or more pathogen cells. In several embodiments, the sample contains nucleic acids (e.g., DNA or RNA (e.g., mRNA)) derived from a mammalian host subject and one or more pathogen cells.

For example, provided herein is a method for detecting a target nucleic acid in an environmental sample or a biological sample obtained from a subject, wherein the environmental or biological sample includes cells (e.g., subject-derived cells) or cell debris, the method including one or two of the following steps: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of detecting a concentration of about 10 copies/mL (or less, e.g., 1, 2, 3, 4, 5, 6, 7, 8, or 9 copies/mL) of the target nucleic acid in the biological sample.

Also provided herein is a method for determining the sequence of a target nucleic acid in an environmental sample or a biological sample obtained from a subject, wherein the environmental or biological sample includes cells (e.g., subject-derived cells) or cell debris, the method including one or two of the following steps: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of determining the sequence of the target nucleic acid at a concentration of about 10 copies/mL (or less, e.g., 1, 2, 3, 4, 5, 6, 7, 8, or 9 copies/mL) of the target nucleic acid in the biological sample.

Further provided herein is a method for detecting a target nucleic acid in an environmental sample or a biological sample obtained from a subject, wherein the environmental or biological sample includes cells (e.g., subject-derived cells) or cell debris, the method including sequencing an amplified target nucleic acid to detect whether the target nucleic acid is present in the biological sample, wherein the amplified target nucleic acid has been amplified in the environmental or biological sample, and the method is capable of detecting a concentration of about 10 copies/mL (or less, e.g., 1, 2, 3, 4, 5, 6, 7, 8, or 9 copies/mL) of the target nucleic acid in the biological sample.

Also provided herein is a method for determining the sequence of a target nucleic acid in an environmental sample or a biological sample obtained from a subject, wherein the environmental or biological sample includes cells (e.g., subject-derived cells) or cell debris, the method including sequencing an amplified target nucleic acid to detect whether the target nucleic acid is present in the biological sample, wherein the amplified target nucleic acid has been amplified in the environmental or biological sample, and the method is capable of detecting a concentration of about 10 copies/mL (or less, e.g., 1, 2, 3, 4, 5, 6, 7, 8, or 9 copies/mL) of the target nucleic acid in the biological sample.

In any of the preceding methods, the environmental sample or biological sample may contain nucleic acids (e.g., DNA or RNA (e.g., mRNA)), such as host-derived nucleic acids or non-target nucleic acids present in the sample.

Further provided herein is a method for detecting a target nucleic acid in an environmental sample or a biological sample obtained from a subject, wherein the environmental or biological sample also includes non-target nucleic acids (e.g., DNA or RNA (e.g., mRNA)), the method including one or two of the following steps: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of detecting a concentration of about 10 copies/mL (or less, e.g., 1, 2, 3, 4, 5, 6, 7, 8, or 9 copies/mL) of the target nucleic acid in the biological sample.

Also provided herein is a method for determining the sequence of a target nucleic acid in an environmental sample or a biological sample obtained from a subject, wherein the environmental or biological sample also includes non-target nucleic acids (e.g., DNA or RNA (e.g., mRNA)), the method including one or two of the following steps: (a) amplifying a target nucleic acid in the biological sample to form an amplified solution including an amplified target nucleic acid; and (b) sequencing the amplified target nucleic acid to detect whether the target nucleic acid is present in the biological sample, wherein the method is capable of determining the sequence of the target nucleic acid at a concentration of about 10 copies/mL (or less, e.g., 1, 2, 3, 4, 5, 6, 7, 8, or 9 copies/mL) of the target nucleic acid in the biological sample.

In yet another example, provided herein is a method for detecting a target nucleic acid in an environmental sample or a biological sample obtained from a subject, wherein the environmental or biological sample also includes non-target nucleic acids (e.g., DNA or RNA (e.g., mRNA)), the method including sequencing an amplified target nucleic acid to detect whether the target nucleic acid is present in the biological sample, wherein the amplified target nucleic acid has been amplified in the environmental or biological sample, and the method is capable of detecting a concentration of about 10 copies/mL (or less, e.g., 1, 2, 3, 4, 5, 6, 7, 8, or 9 copies/mL) of the target nucleic acid in the biological sample.

Further provided herein is a method for determining the sequence of a target nucleic acid in an environmental sample or a biological sample obtained from a subject, wherein the environmental or biological sample also includes non-target nucleic acids (e.g., DNA or RNA (e.g., mRNA)), the method including sequencing an amplified target nucleic acid to detect whether the target nucleic acid is present in the biological sample, wherein the amplified target nucleic acid has been amplified in the environmental or biological sample, and the method is capable of detecting a concentration of about 10 copies/mL (or less, e.g., 1, 2, 3, 4, 5, 6, 7, 8, or 9 copies/mL) of the target nucleic acid in the biological sample.

The environmental or biological sample (e.g., blood (e.g., whole blood (e.g., ethylenediaminetetraacetic acid (EDTA) whole blood, sodium citrate whole blood, sodium heparin whole blood, lithium heparin whole blood, and/or potassium oxylate/sodium fluoride whole blood), a crude blood lysate, serum, or plasma)) can have any suitable volume. For example, in some embodiments, the environmental or biological sample has a volume of about 0.2 mL to about 50 mL, e.g., about 0.2 mL, about 0.3 mL, about 0.4 mL, about 0.5 mL, about 0.6 mL, about 0.7 mL, about 0.8 mL, about 0.9 mL, about 1 mL, about 2 mL, about 3 mL, about 4 mL, about 5 mL, about 6 mL, about 7 mL, about 8 mL, about 9 mL, about 10 mL, about 15 mL, about 20 mL, about 25 mL, about 30 mL, about 35 mL, about 40 mL, about 45 mL, or about 50 mL. In some embodiments, the environmental or biological sample has a volume of about 0.2 mL to about 20 mL, about 0.2 mL to about 15 mL, about 0.2 mL to about 10 mL, about 0.2 mL to about 5 mL, about 0.2 mL to about 2 mL, about 0.4 mL to about 20 mL, about 0.4 mL to about 15 mL, about 0.4 mL to about 10 mL, about 0.4 mL to about 5 mL, about 0.4 mL to about 2 mL, about 0.6 mL to about 20 mL, about 0.6 mL to about 15 mL, about 0.6 mL to about 10 mL, about 0.6 mL to about 5 mL, about 0.6 mL to about 2 mL, about 0.8 mL to about 20 mL, about 0.8 mL to about 15 mL, about 0.8 mL to about 10 mL, about 0.8 mL to about 5 mL, about 0.8 mL to about 2 mL, about 1 mL to about 20 mL, about 1 mL to about 15 mL, about 1 mL to about 10 mL, about 1 mL to about 5 mL, about 1 mL to about 4 mL, about 1 mL to about 3 mL, about 1 mL to about 2 mL, about 1.5 mL to about 20 mL, about 1.5 mL to about 15 mL, about 1.5 mL to about 10 mL, about 1.5 mL to about 5 mL, about 1.5 mL to about 4 mL, about 1.5 mL to about 3 mL, about 1.5 mL to about 2 mL, about 2 mL to about 20 mL, about 2 mL to about 15 mL, about 2 mL to about 10 mL, about 2 mL to about 5 mL, about 2 mL to about 4 mL, about 2 mL to about 3 mL, about 3 mL to about 20 mL, about 3 mL to about 15 mL, about 3 mL to about 10 mL, about 3 mL to about 5 mL, about 3 mL to about 4 mL, about 4 mL to about 20 mL, about 4 mL to about 15 mL, about 4 mL to about 10 mL, about 4 mL to about 5 mL, about 5 mL to about 20 mL, about 5 mL to about 15 mL, about 5 mL to about 10 mL, about 6 mL to about 20 mL, about 6 mL to about 15 mL, about 6 mL to about 10 mL, about 7 mL to about 20 mL, about 7 mL to about 15 mL, about 7 mL to about 10 mL, about 8 mL to about 20 mL, about 8 mL to about 15 mL, about 8 mL to about 10 mL, about 9 mL to about 20 mL, about 9 mL to about 15 mL, about 9 mL to about 10 mL, about 10 mL to about 20 mL, or about 10 mL to about 15 mL. In some embodiments, the environmental or biological sample has a volume of about 0.2 mL to about 20 mL, about 0.2 mL to about 15 mL, about 0.2 mL to about 10 mL, about 0.2 mL to about 5 mL, or about 0.2 mL to about 2 mL. In some embodiments, the environmental or biological sample has a volume of about 2 mL.

Any suitable environmental or biological sample can be used. For example, the biological sample can be selected from the group consisting of blood (e.g., whole blood (e.g., ethylenediaminetetraacetic acid (EDTA) whole blood, sodium citrate whole blood, sodium heparin whole blood, lithium heparin whole blood, and/or potassium oxylate/sodium fluoride whole blood), a crude blood lysate, serum, or plasma), bloody fluids (e.g., wound exudate, phlegm, or bile), tissue samples (e.g., a tissue biopsy such as a skin biopsy, muscle biopsy, or lymph node biopsy), urine, cerebrospinal fluid (CSF), synovial fluid (SF), and sputum. In some embodiments, the biological sample is blood (e.g., whole blood (e.g., EDTA whole blood, sodium citrate whole blood, sodium heparin whole blood, lithium heparin whole blood, and/or potassium oxylate/sodium fluoride whole blood), a crude blood lysate, serum, or plasma). In some embodiments, the tissue sample is a homogenized tissue sample. The environmental sample may be an environmental swab, e.g., a surface swab. In some embodiments, the swab buffer diluent or swab transport medium is, without limitation, phospho-buffered saline-TWEEN® (PBST), Amies Buffer, Amies Buffer+10% (v/v) 10× PBST, Cary Blair Media, or Liquid Stuart Swabs (which may include addition of 10% (v/v) 10× PBST).

In some embodiments of any of the preceding methods, the target nucleic acid is characteristic of a pathogen. In other embodiments, the target nucleic acid may be characteristic of a non-pathogenic organism.

In some embodiments of any of the preceding methods, step (a) includes amplifying the target nucleic acid in a lysate produced by lysing cells in the environmental or biological sample. The lysate may contain a concentration of cells, cell debris, and/or non-target or subject-cell derived nucleic acids (e.g., DNA) relative to the original environmental or biological sample. As an example, 2 mL of a biological sample concentrated down to 0.1 mL in a lysate corresponds to a 20:1 higher concentration of debris compared to the original sample. If the lysate is diluted 1:1 for amplification, the amplification is performed in a lysate that represents a 10:1 concentration of the debris in the original sample. In some embodiments, the lysate has at least about a 1.5:1 higher concentration of cell debris relative to the environmental or biological sample, e.g., about 1.5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, about 19:1, about 20:1, about 21:1, about 22:1, about 23:1, about 24:1, about 25:1, about 30:1, about 35:1, about 40:1, about 45:1, about 50:1, about 55:1, about 60:1, about 65:1, about 70:1, about 75:1, about 80:1, about 90:1, about 100:1, about 120:1, about 140:1, about 160:1, about 180:1, about 200:1, about 300:1, about 400:1, about 500:1, about 600:1, about 700:1, about 800:1, about 900:1, about 1000:1, or higher concentration of cell debris relative to the environmental or biological sample. In some embodiments, the lysate is not diluted prior to amplification. In other embodiments, the lysate is diluted to produce a diluted lysate (e.g., for use in amplification), and the diluted lysate has at least about a 1.5:1 higher concentration of cell debris relative to the environmental or biological sample, e.g., about 1.5:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, about 19:1, about 20:1, about 21:1, about 22:1, about 23:1, about 24:1, about 25:1, about 30:1, about 35:1, about 40:1, about 45:1, about 50:1, about 55:1, about 60:1, about 65:1, about 70:1, about 75:1, about 80:1, about 90:1, about 100:1, about 120:1, about 140:1, about 160:1, about 180:1, about 200:1, about 300:1, about 400:1, about 500:1, about 600:1, about 700:1, about 800:1, about 900:1, about 1000:1, or higher concentration of cell debris relative to the environmental or biological sample. In some embodiments, the lysate or the diluted lysate has about a 10:1 higher concentration of cell debris relative to the environmental or biological sample. In other embodiments, the lysate or the diluted lysate has about a 20:1 higher concentration of cell debris relative to the environmental or biological sample. In some embodiments, the cell debris is solid material (e.g., solid material that can be concentrated with a liquid-solid separation method (e.g., centrifugation or filtration). In some embodiments, the lysate or the amplified lysate solution is a super-saturated solution of cell debris (e.g., solid material).

Also provided herein is a method for detecting a target pathogen nucleic acid in a whole blood sample, the method including the following steps: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and resuspending the pellet to form an extract, optionally washing the pellet one or more times prior to resuspending the pellet; (d) lysing the remaining cells in the extract of step (c) to form a lysate, the lysate containing both subject cell nucleic acid and pathogen nucleic acid; (e) amplifying pathogen nucleic acids in the lysate of step (d) to form an amplified lysate solution including an amplified target pathogen nucleic acid; and (f) sequencing the amplified target pathogen nucleic acid, thereby detecting the target pathogen nucleic acid in the sample.

Further provided herein is a method for determining the sequence of a target pathogen nucleic acid in a whole blood sample, the method including the following steps: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and resuspending the pellet to form an extract, optionally washing the pellet one or more times prior to resuspending the pellet; (d) lysing the remaining cells in the extract of step (c) to form a lysate, the lysate containing both subject cell nucleic acid and pathogen nucleic acid; (e) amplifying pathogen nucleic acids in the lysate of step (d) to form an amplified lysate solution including an amplified target pathogen nucleic acid; and (f) sequencing the amplified target pathogen nucleic acid, thereby determining the sequence of the target pathogen nucleic acid in the sample.

In some embodiments of any of the preceding methods, step (c) can include washing the pellet one time prior to resuspending the pellet. In other embodiments, step (c) can include washing the pellet more than one time prior to resuspending the pellet, e.g., two, three, four, five, six, seven, eight, nine, or ten times. In some embodiments, the washing or resuspending is performed with a wash buffer solution (e.g., Tris-EDTA (TE) buffer). The wash buffer solution may have any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the washing is performed with a wash buffer solution having a volume of about 100 μL to about 500 μL. In some embodiments, the washing is performed with a wash buffer solution having a volume of about 100 μL to about 200 μL. In some embodiments, the volume is about 150 μL.

The resuspending of step (c) can be performed with a wash buffer solution having any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the resuspending is performed with a wash buffer solution having a volume of about 100 μL to about 500 μL or about 100 μL to about 200 μL. In some embodiments, the volume is about 100 μL.

In some embodiments of any of the preceding methods, the wash buffer solution further includes an amplification control nucleic acid. In some embodiments of any of the preceding methods, step (a) further includes adding a total process control (TPC) to the whole blood sample, e.g., an engineered cell including a control target nucleic acid.

In another example, provided herein is a method for detecting a target pathogen nucleic acid in a whole blood sample, the method including the following steps: (a) providing an amplified lysate solution that has been produced by: (i) contacting a whole blood sample suspected of containing one or more pathogen cells with an erythrocyte lysis agent, thereby lysing red blood cells; (ii) centrifuging the product of step (a)(i) to form a supernatant and a pellet; (iii) discarding some or all of the supernatant of step (a)(ii) and resuspending the pellet to form an extract, optionally washing the pellet one or more times prior to resuspending the pellet; (iv) lysing the remaining cells in the extract of step (a)(iii) to form a lysate, the lysate containing both subject cell nucleic acid and pathogen nucleic acid; (v) amplifying pathogen nucleic acids in the lysate of step (a)(iv) to form an amplified lysate solution including an amplified target pathogen nucleic acid; and (b) sequencing the amplified target pathogen nucleic acid, thereby detecting the target pathogen nucleic acid in the sample.

In a further example, provided herein is a method for determining the sequence of a target pathogen nucleic acid in a whole blood sample, the method including the following steps: (a) providing an amplified lysate solution that has been produced by: (i) contacting a whole blood sample suspected of containing one or more pathogen cells with an erythrocyte lysis agent, thereby lysing red blood cells; (ii) centrifuging the product of step (a)(i) to form a supernatant and a pellet; (iii) discarding some or all of the supernatant of step (a)(ii) and resuspending the pellet to form an extract, optionally washing the pellet one or more times prior to resuspending the pellet; (iv) lysing the remaining cells in the extract of step (a)(iii) to form a lysate, the lysate containing both subject cell nucleic acid and pathogen nucleic acid; (v) amplifying pathogen nucleic acids in the lysate of step (a)(iv) to form an amplified lysate solution including an amplified target pathogen nucleic acid; and (b) sequencing the amplified target pathogen nucleic acid, thereby determining the sequence of the target pathogen nucleic acid in the sample.

In some embodiments of any of the preceding methods, step (a)(iii) includes washing the pellet one time prior to resuspending the pellet. In other embodiments, step (a)(iii) includes washing the pellet more than one time prior to resuspending the pellet, e.g., two, three, four, five, six, seven, eight, nine, or ten times.

In some embodiments, the washing or resuspending is performed with a wash buffer solution (e.g., Tris-EDTA (TE) buffer). The wash buffer solution may have any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the washing is performed with a wash buffer solution having a volume of about 100 μL to about 500 μL. In some embodiments, the washing is performed with a wash buffer solution having a volume of about 100 μL to about 200 μL. In some embodiments, the volume is about 150 μL.

The resuspending of step (a)(iii) can be performed with a wash buffer solution having any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the resuspending is performed with a wash buffer solution having a volume of about 100 μL to about 500 μL or about 100 μL to about 200 μL. In some embodiments, the volume is about 100 μL.

In some embodiments of any of the preceding methods, the wash buffer solution further includes an amplification control nucleic acid. In some embodiments of any of the preceding methods, step (a)(i) further includes adding a total process control (TPC) to the whole blood sample, e.g., an engineered cell including a control target nucleic acid.

In a still further example, provided herein is a method for detecting a target pathogen nucleic acid in a whole blood sample, the method including the following steps: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and washing the pellet once; (d) centrifuging the product of step (c) to form a supernatant and a pellet; (e) discarding some or all of the supernatant of step (d) and mixing the pellet of (d) with a buffer solution; (f) combining the product of step (e) with beads to form a mixture and agitating the mixture to form a lysate, said lysate containing both subject cell nucleic acid and pathogen nucleic acid; (g) amplifying pathogen nucleic acids in the lysate of step (f) to form an amplified lysate solution including an amplified target pathogen nucleic acid; and (h) sequencing the amplified target pathogen nucleic acid, thereby detecting the target pathogen nucleic acid in the sample.

In yet another example, provided herein is a method for determining the sequence of a target pathogen nucleic acid in a whole blood sample, the method including the following steps: (a) contacting a whole blood sample suspected of containing one or more pathogen cells with an erythrocyte lysis agent, thereby lysing red blood cells; (b) centrifuging the product of step (a) to form a supernatant and a pellet; (c) discarding some or all of the supernatant of step (b) and washing the pellet once; (d) centrifuging the product of step (c) to form a supernatant and a pellet; (e) discarding some or all of the supernatant of step (d) and mixing the pellet of (d) with a buffer solution; (f) combining the product of step (e) with beads to form a mixture and agitating the mixture to form a lysate, said lysate containing both subject cell nucleic acid and pathogen nucleic acid; (g) amplifying pathogen nucleic acids in the lysate of step (f) to form an amplified lysate solution including an amplified target pathogen nucleic acid; and (h) sequencing the amplified target pathogen nucleic acid, thereby determining the sequence of the target pathogen nucleic acid in the sample.

In some embodiments of any of the preceding methods, the washing of step (c) is performed with a wash buffer solution (e.g., TE buffer). The wash buffer solution may have any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the washing is performed with a wash buffer solution having a volume of about 100 μL to about 500 μL. In some embodiments, the washing is performed with a wash buffer solution having a volume of about 100 μL to about 200 μL. In some embodiments, the volume is about 150 μL.

In some embodiments of any of the preceding methods, step (e) includes mixing the pellet with a buffer solution. The buffer solution (e.g., TE buffer) may have any suitable volume, e.g., about 25 μL to about 1 mL, e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 225 μL, about 250 μL, about 275 μL, about 300 μL, about 350 μL, about 400 μL, about 450 μL, about 500 μL, about 550 μL, about 600 μL, about 650 μL, about 700 μL, about 800 μL, about 850 μL, about 900 μL, about 950 μL, or about 1 mL. In some embodiments, the buffer solution has a volume of about 100 μL to about 200 μL. In some embodiments, the volume is about 100 μL.

In some embodiments of any of the preceding methods, the buffer solution and/or the wash buffer solution further includes an amplification control nucleic acid. In some embodiments of any of the preceding methods, step (a) further includes adding a TPC to the whole blood sample, e.g., an engineered cell including a control target nucleic acid.

The the lysate or the amplified lysate solution may contain a concentration of cells, cell debris, and/or non-target or subject-cell derived nucleic acids (e.g., DNA) relative to the original environmental or biological sample. As an example, 2 mL of a biological sample concentrated down to 0.1 mL in a lysate corresponds to a 20:1 higher concentration of debris compared to the original sample. If the lysate is diluted 1:1 for amplification, the amplification is performed in a lysate (e.g., an amplified lysate solution) that represents a 10:1 concentration of the debris in the original sample. In some embodiments of any of the preceding methods, the lysate or the amplified lysate solution has at least about a 1.5:1 higher concentration of subject cell DNA and/or cell debris relative to the whole blood sample, e.g., about 1.5:1. about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 18:1, about 19:1, about 20:1, about 21:1, about 22:1, about 23:1, about 24:1, about 25:1, about 30:1, about 35:1, about 40:1, about 45:1, about 50:1, about 55:1, about 60:1, about 65:1, about 70:1, about 75:1, about 80:1, about 90:1, about 100:1, about 120:1, about 140:1, about 160:1, about 180:1, about 200:1, about 300:1, about 400:1, about 500:1, about 600:1, about 700:1, about 800:1, about 900:1, about 1000:1, or higher concentration of subject cell DNA and/or cell debris relative to the whole blood sample. In some embodiments, the lysate or the amplified lysate solution has about a 10:1 higher concentration of subject cell DNA and/or cell debris relative to the whole blood sample. In some embodiments, the lysate or the amplified lysate solution has a 20:1 higher concentration of subject cell DNA and/or cell debris relative to the whole blood sample. In some embodiments, the lysate has about a 20:1 higher concentration of subject cell DNA and/or cell debris relative to the whole blood sample. In some embodiments, the amplified lysate solution has about a 10:1 higher concentration of subject cell DNA and/or cell debris relative to the whole blood sample. In some embodiments, the cell debris is solid material (e.g., solid material that can be concentrated with a liquid-solid separation method (e.g., centrifugation or filtration)). In some embodiments, the lysate or the amplified lysate solution is a super-saturated solution of cell debris (e.g., solid material).

In some embodiments of any of the preceding methods, an amplified target nucleic acid is produced in the presence of at least 1 μg of subject DNA, e.g., at least 1 μg of subject DNA, at least 5 μg of subject DNA, at least 10 μg of subject DNA, at least 15 μg of subject DNA, at least 20 μg of subject DNA, at least 25 μg of subject DNA, at least 30 μg of subject DNA, at least 35 μg of subject DNA, at least 40 μg of subject DNA, at least 45 μg of subject DNA, at least 50 μg of subject DNA, at least 55 μg of subject DNA, at least 60 μg of subject DNA, at least 80 μg of subject DNA, at least 90 μg of subject DNA, at least 100 μg of subject DNA, or more. For example, in some embodiments, the amplifying is performed in the presence of from about 0.5 μg to about 100 μg or about 20 to about 80 μg of subject cell DNA. In some embodiments, the amplifying is performed in the presence of about 60 μg of subject cell DNA. In some embodiments, at least a portion of the subject DNA is from white blood cells of the subject.

Any suitable amplification approach can be used. For example, in some embodiments, the amplifying includes polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM). In some embodiments, the amplifying includes PCR (e.g., symmetric PCR or asymmetric PCR).

In some embodiments of any of the preceding methods, the amplifying includes the following steps: (i) adding to the lysate an amplification buffer solution including a buffering agent to form a reaction mixture; (ii) heating the reaction mixture to form a denatured reaction mixture; and (iii) adding a thermostable nucleic acid polymerase to the denatured reaction mixture under conditions and for a time sufficient for amplification of the target nucleic acid. The method can further include centrifuging the denatured reaction mixture to form a pellet and a supernatant prior to step (iii). In some embodiments, step (iii) includes adding the thermostable nucleic acid polymerase to the supernatant.

In other embodiments of any of the preceding methods, the amplifying includes the following steps: (i) adding to the lysate an amplification buffer solution including a buffering agent and a thermostable nucleic acid polymerase to form a reaction mixture under conditions and for a time sufficient for amplification of the target nucleic acid; (ii) heating the reaction mixture to form a denatured reaction mixture; and (iii) centrifuging the denatured reaction mixture to form a pellet and a supernatant. In some embodiments, the final concentration of the thermostable nucleic acid polymerase in step (i) is at least about 0.02 units per microliter of the reaction mixture. In some embodiments, step (i) comprises adding at least about 2.4×10⁻⁵ micrograms of a thermostable nucleic acid polymerase per microliter of reaction mixture.

In some embodiments of any of the preceding methods, the amplification buffer solution has a moderately alkaline pH at ambient temperature. In some embodiments of any of the preceding methods, the moderately alkaline pH at ambient temperature is from about pH 7.1 to about pH 11.5 or higher (e.g., about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, about pH 10.0, about pH 10.1, about pH 10.2, about pH 10.3, about pH 10.4, about pH 10.5, about pH 10.6, about pH 10.7, about pH 10.8, about pH 10.9, about pH 11, about pH 11.1, about pH 11.2, about pH 11.3, about pH 11.3, about pH 11.4, about pH 11.5, or higher. In some embodiments, the moderately alkaline pH at ambient temperature is from about pH 7.1 to about pH 11.5, about pH 7.1 to about pH 11.0, about pH 7.1 to about pH 10.5, about pH 7.1 to about pH 10.0, about pH 7.1 to about pH 9.5, about pH 7.1 to about pH 9.0, about pH 7.1 to about pH 8.5, about pH 7.1 to about pH 8, about pH 7.1 to about pH 7.5, about pH 7.5 to about pH 11.5, about pH 7.5 to about pH 11.0, about pH 7.5 to about pH 10.5, about pH 7.5 to about pH 10.0, about pH 7.5 to about pH 9.5, about pH 7.5 to about pH 9.0, about pH 7.5 to about pH 8.5, about pH 7.5 to about pH 8.0, about pH 8.0 to about pH 11.5, about pH 8.0 to about pH 11.0, about pH 8.0 to about pH 10.5, about pH 8.0 to about pH 10.0, about pH 8.0 to about pH 9.5, about pH 8.0 to about pH 9.0, about pH 8.0 to about pH 9.0, about pH 8.0 to about pH 8.5, about pH 8.5 to about pH 11.5, about pH 8.5 to about pH 11.0, about pH 8.5 to about pH 10.0, about pH 8.5 to about pH 9.5, about pH 8.5 to about pH 9.0, about pH 9.0 to about pH 11.5, about pH 9.0 to about pH 11.0, about pH 9.0 to about pH 10.5, about pH 9.0 to about pH 10.0, about pH 9.0 to about pH 9.5, about pH 9.5 to about pH 11.5, about pH 9.5 to about pH 11.0, about pH 9.5 to about pH 10.5, or about pH 9.5 to about pH 10.0. In some embodiments, the moderately alkaline pH at ambient temperature is about pH 8.7. In some embodiments, ambient temperature is about 25° C. (e.g., about 20° C., about 21° C., about 22° C., about 23° C., about 24° C., about 25° C., about 26° C., about 27° C., about 28° C., about 29° C., about 30° C.). In some embodiments, ambient temperature is about 20° C. to about 30° C., about 20° C. to about 29° C., about 20° C. to about 28° C., about 20° C. to about 27° C., about 20° C. to about 26° C., about 20° C. to about 25° C., about 20° C. to about 24° C., about 20° C. to about 23° C., about 20° C. to about 22° C., about 20° C. to about 21° C., about 22° C. to about 30° C., about 22° C. to about 29° C., about 22° C. to about 28° C., about 22° C. to about 27° C., about 22° C. to about 26° C., about 22° C. to about 25° C., about 22° C. to about 24° C., about 22° C. to about 23° C., about 24° C. to about 30° C., about 24° C. to about 29° C., about 24° C. to about 28° C., about 24° C. to about 27° C., about 24° C. to about 26° C., or about 24° C. to about 25° C.

In some embodiments of any of the preceding methods, the pH of the buffer solution remains approximately at or above a neutral pH at 95° C. In some embodiments, the pH of the buffer solution is about pH 6.5 to about pH 10 (e.g., about pH 6.5, about pH 6.6, about pH 6.7, about pH 6.8, about pH 6.9, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, or about pH 10.0) at 95° C. For example, in some embodiments, the pH of the buffer solution at 95° C. is from about pH 6.5 to about pH 10.0, about pH 6.5 to about pH 9.5, about pH 6.5 to about pH 9.0, about pH 6.5 to about pH 8.5, about pH 6.5 to about pH 8.0, about pH 6.5 to about pH 7.5, about pH 7.0 to about pH 10.0, about pH 7.0 to about pH 9.5, about pH 7.0 to about pH 9.0, about pH 7.0 to about pH 8.5, about pH 7.0 to about pH 8.0, about pH 7.0 to about pH 7.5, about pH 7.5 to about pH 10.0, about pH 7.5 to about pH 9.5, about pH 7.5 to about pH 9.0, about pH 7.5 to about pH 8.5, about pH 7.5 to about pH 8.0, about pH 8.0 to about pH 10.0, about pH 8.0 to about pH 9.5, about pH 8.0 to about pH 9.0, about pH 8.0 to about pH 8.5, about pH 8.5 to about pH 10.0, about pH 8.5 to about pH 9.5, about pH 8.5 to about pH 9.0, about pH 9.0 to about pH 10.0, or about pH 9.5 to about pH 10.0. For example, in some embodiments, the pH of the buffer solution is about 6.5 to about 9.0, about 7.0 to about 8.5, or about 7.0 to about 7.5 at 95° C. In some embodiments, the pH of the buffer solution is about 7.2 at 95° C.

In some embodiments of any of the preceding methods, the final concentration of the thermostable nucleic acid polymerase in step (iii) is at least about 0.01 units (e.g., about 0.01 units, about 0.02 units, about 0.03 units, about 0.04 units, about 0.05 units, about 0.06 units, about 0.07 units, about 0.08 units, about 0.09 units, about 0.10 units, about 0.15 units about 0.2 units, about 0.25 units, about 0.3 units, about 0.35 units, about 0.4 units, about 0.45 units, about 0.5 units, about 0.6 units, about 0.65 units, about 0.7 units, about 0.8 units, about 0.9 units, about 1 unit, or more) per microliter of the denatured reaction mixture. In some embodiments, the final concentration of the thermostable nucleic acid polymerase may range from about 0.01 units to about 1 unit (e.g., about 0.01 units to about 1 unit, about 0.01 units to about 0.9 units, about 0.01 units to about 0.8 units, about 0.01 units to about 0.7 units, about 0.01 units to about 0.6 units, about 0.01 units to about 0.5 units, about 0.01 units to about 0.4 units, about 0.01 units to about 0.3 units, about 0.01 units to about 0.25 units, about 0.01 units to about 0.2 units, about 0.01 units to about 0.1 unit, about 0.02 units to about 1 unit, about 0.02 units to about 0.9 units, about 0.02 units to about 0.8 units, about 0.02 units to about 0.7 units, about 0.02 units to about 0.6 units, about 0.02 units to about 0.5 units, about 0.02 units to about 0.4 units, about 0.02 units to about 0.3 units, about 0.02 units to about 0.25 units, about 0.02 units to about 0.2 units, about 0.02 units to about 0.1 units, about 0.04 units to about 1 unit, about 0.04 unit to about 0.9 units, about 0.04 units to about 0.8 units, about 0.04 units to about 0.7 units, about 0.04 units to about 0.6 units, about 0.04 units to about 0.5 units, about 0.04 units to about 0.4 units, about 0.04 units to about 0.3 units, about 0.04 units to about 0.25 units, about 0.04 units to about 0.2 units, about 0.04 units to about 0.1 units, about 0.06 units to about 1 unit, about 0.06 units to about 0.9 units, about 0.06 units to about 0.8 units, about 0.06 units to about 0.7 units, about 0.06 units to about 0.6 units, about 0.06 units to about 0.5 units, about 0.06 units to about 0.4 units, about 0.06 units to about 0.3 units, about 0.06 units to about 0.25 units, about 0.06 units to about 0.2 units, about 0.06 units to about 0.1 units, about 0.08 units to about 1 unit, about 0.08 units to about 0.9 units, about 0.08 units to about 0.8 units, about 0.08 units to about 0.7 units, about 0.08 units to about 0.6 units, about 0.08 units to about 0.5 units, about 0.08 units to about 0.4 units, about 0.08 units to about 0.3 units, about 0.08 units to about 0.25 units, about 0.08 units to about 0.2 units, about 0.08 units to about 0.1 units, about 0.1 units to about 1 unit, about 0.1 units to about 0.9 units, about 0.1 units to about 0.8 units, about 0.1 units to about 0.7 units, about 0.1 units to about 0.6 units, about 0.1 units to about 0.5 units, about 0.1 units to about 0.4 units, about 0.1 units to about 0.3 units, about 0.1 units to about 0.25 units, about 0.1 units to about 0.2 units, about 0.2 units to about 1 unit, about 0.2 units to about 0.9 units, about 0.2 units to about 0.8 units, about 0.2 units to about 0.7 units, about 0.2 units to about 0.6 units, about 0.2 units to about 0.5 units, about 0.2 units to about 0.4 units, about 0.2 units to about 0.3 units, about 0.2 units to about 0.25 units, about 0.3 units to about 1 unit, about 0.3 units to about 0.9 units, about 0.3 units to about 0.8 units, about 0.3 units to about 0.7 units, about 0.3 units to about 0.6 units, about 0.3 units to about 0.5 units, about 0.3 units to about 0.4 units, about 0.4 units to about 1 unit, about 0.4 units to about 0.9 units, about 0.4 units to about 0.8 units, about 0.4 units to about 0.7 units, about 0.4 units to about 0.6 units, about 0.4 units to about 0.5 units, about 0.5 units to about 1 unit, about 0.5 units to about 0.9 units, about 0.5 units to about 0.8 units, about 0.5 units to about 0.7 units, about 0.5 units to about 0.6 units, about 0.6 units to about 1 unit, about 0.6 units to about 0.9 units, about 0.6 units to about 0.8 units, about 0.6 units to about 0.7 units, about 0.6 units to about 0.6 units, about 0.7 units to about 1 unit, about 0.7 units to about 0.9 units, about 0.7 units to about 0.8 units, about 0.8 units to about 1 unit, or about 0.8 units to about 0.9 units) per microliter of the mixture. In some embodiments, the final concentration of the thermostable nucleic acid polymerase ranges from about 0.125 to about 0.5 units/μL. In some embodiments, the final concentration of the thermostable nucleic acid ranges from about 0.125 to about 0.25 units/μL.

In some embodiments of any of the preceding methods, step (iii) includes adding to the denatured reaction mixture at least about 1×10⁻⁵ micrograms (e.g., about 1×10⁻⁵ micrograms, about 1.5×10⁻⁵ micrograms, about 2×10⁻⁵ micrograms, about 2.4×10⁻⁵ micrograms, about 2.5×10⁻⁵ micrograms, about 3×10⁻⁵ micrograms, about 4×10⁻⁵ micrograms, about 5×10⁻⁵ micrograms, about 6×10⁻⁵ micrograms, about 7×10⁻⁵ micrograms, about 8×10⁻⁵ micrograms, about 9×10⁻⁵ micrograms, about 1×10⁻⁴ micrograms, about 2×10⁻⁴ micrograms, about 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻⁴ micrograms, about 1×10⁻³ micrograms, about 2×10⁻³ micrograms, 3×10⁻³ micrograms, about 4×10⁻³ micrograms, about 5×10⁻³ micrograms, about 6×10⁻³ micrograms, about 7×10⁻³ micrograms, about 8×10⁻³ micrograms, about 9×10⁻³ micrograms, about 0.01 micrograms, about 0.02 micrograms, about 0.03 micrograms, about 0.04 micrograms, about 0.05 micrograms, or more) of a thermostable nucleic acid polymerase per microliter of denatured reaction mixture. For example, In some embodiments of any of the preceding methods, the mixture includes from about 1×10⁻⁵ micrograms to about 0.05 micrograms (e.g., about 1×10⁻⁵ micrograms to about 0.05 micrograms, about 1×10⁻⁵ micrograms to about 0.025 micrograms, about 1×10⁻⁵ micrograms to about 0.01 micrograms, about 1×10⁻⁵ micrograms to about 0.0075 micrograms, about 1×10⁻⁵ micrograms to about 0.005 micrograms, about 1×10⁻⁵ micrograms to about 0.0025 micrograms, about 1×10⁻⁵ micrograms to about 0.001 micrograms, about 1×10⁻⁵ micrograms to about 1×10⁻⁴ micrograms, about 2×10⁻⁵ micrograms to about 0.05 micrograms, about 2×10⁻⁵ micrograms to about 0.025 micrograms, about 2×10⁻⁵ micrograms to about 0.01 micrograms, about 2×10⁻⁵ micrograms to about 0.0075 micrograms, about 2×10⁻⁵ micrograms to about 0.005 micrograms, about 2×10⁻⁵ micrograms to about 0.0025 micrograms, about 2×10⁻⁵ micrograms to about 0.001 micrograms, about 2×10⁻⁵ micrograms to about 1×10⁻⁴ micrograms, about 2.4×10⁻⁵ micrograms to about 0.05 micrograms, about 2.4×10⁻⁵ micrograms to about 0.025 micrograms, about 2.4×10⁻⁵ micrograms to about 0.01 micrograms, about 2.4×10⁻⁵ micrograms to about 0.0075 micrograms, about 2.4×10⁻⁵ micrograms to about 0.005 micrograms, about 2.4×10⁻⁵ micrograms to about 0.0025 micrograms, about 2.4×10⁻⁵ micrograms to about 0.001 micrograms, about 2.4×10⁻⁵ micrograms to about 1×10⁻⁴ micrograms, about 5×10⁻⁵ micrograms to about 0.05 micrograms, about 5×10⁻⁵ micrograms to about 0.025 micrograms, about 5×10⁻⁵ micrograms to about 0.01 micrograms, about 5×10⁻⁵ micrograms to about 0.0075 micrograms, about 5×10⁻⁵ micrograms to about 0.005 micrograms, about 5×10⁻⁵ micrograms to about 0.0025 micrograms, about 5×10⁻⁵ micrograms to about 0.001 micrograms, about 5×10⁻⁵ micrograms to about 1×10⁻⁴ micrograms, about 8×10⁻⁵ micrograms to about 0.05 micrograms, about 8×10⁻⁵ micrograms to about 0.025 micrograms, about 8×10⁻⁵ micrograms to about 0.01 micrograms, about 8×10⁻⁵ micrograms to about 0.0075 micrograms, about 8×10⁻⁵ micrograms to about 0.005 micrograms, about 8×10⁻⁵ micrograms to about 0.0025 micrograms, about 8×10⁻⁵ micrograms to about 0.001 micrograms, about 8×10⁻⁵ micrograms to about 1×10⁻⁴ micrograms, about 1×10⁻⁴ micrograms to about 0.05 micrograms, about 1×10⁻⁴ micrograms to about 0.025 micrograms, about 1×10⁻⁴ micrograms to about 0.01 micrograms, about 1×10⁻⁴ micrograms to about 0.0075 micrograms, about 1×10⁴ micrograms to about 0.005 micrograms, about 1×10⁴ micrograms to about 0.0025 micrograms, about 1×10⁴ micrograms to about 0.001 micrograms, about 5×10⁻⁴ micrograms to about 0.05 micrograms, about 5×10⁻⁴ micrograms to about 0.025 micrograms, about 5×10⁻⁴ micrograms to about 0.01 micrograms, about 5×10⁻⁴ micrograms to about 0.0075 micrograms, about 5×10⁻⁴ micrograms to about 0.005 micrograms, about 5×10⁻⁴ micrograms to about 0.0025 micrograms, about 5×10⁻⁴ micrograms to about 0.001 micrograms, about 1×10⁻³ micrograms to about 0.05 micrograms, about 1×10⁻³ micrograms to about 0.025 micrograms, about 1×10⁻³ micrograms to about 0.01 micrograms, about 1×10⁻³ micrograms to about 0.0075 micrograms, about 1×10⁻³ micrograms to about 0.005 micrograms, or about 1×10⁻³ micrograms to about 0.0025 micrograms) of the thermostable nucleic acid polymerase per microliter of the mixture. In some embodiments, the final concentration of thermostable nucleic acid polymerase is from about 2.4×10⁻⁵ micrograms to about 0.01 micrograms per microliter of denatured reaction mixture or reaction mixture. In some embodiments, the final concentration of thermostable nucleic acid polymerase is from about 2.4×10⁻⁵ micrograms to about 0.0001 micrograms per microliter of denatured reaction mixture or reaction mixture.

In some embodiments of any of the preceding methods, step (ii) may include heating the reaction mixture to greater than about 55° C., e.g., 55° C., 60° C., 65° C., 70° C., 75° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C. In some embodiments, the temperature is about 95° C.

In some embodiments of any of the preceding methods, the thermostable nucleic acid polymerase is a thermostable DNA polymerase. Any suitable thermostable DNA polymerase may be used in the methods of the invention, for example, commercially available thermostable DNA polymerases, or any thermostable DNA polymerase described herein and/or known in the art. In some embodiments, the thermostable DNA polymerase is a wild-type thermostable DNA polymerase, e.g., Thermus aquaticus (Taq) DNA polymerase (see, e.g., U.S. Pat. No. 4,889,818), Thermus thermophilus (Tth) DNA polymerase (see, e.g., U.S. Pat. Nos. 5,192,674; 5,242,818; and 5,413,926), Thermus filiformis (Tfi) DNA polymerase, Thermus flavus (Tfl) DNA polymerase, Thermococcus litoralis (Tli) DNA polymerase (see, e.g., U.S. Pat. No. 5,332,785), Thermatoga maritima (Tma) DNA polymerase, Thermus spp. Z05 DNA polymerase, Tsp sps17 DNA polymerase derived from Thermus species sps17, now called Thermus oshimai (see, e.g., U.S. Pat. No. 5,405,774), Bacillus stearothermophilus (Bst) DNA polymerase (see, e.g., U.S. Pat. No. 5,747,298), an archaeal polymerase (e.g., thermostable DNA polymerases from hyperthermophylic archaeons Pyrococcus furiosus (e.g., Pfu; see, e.g., U.S. Pat. No. 5,948,663), KOD DNA polymerase derived from Pyrococcus sp. KOD1 (e.g., U.S. Pat. No. 6,033,859), Thermococcus litoralis (e.g., VENTR® (NEB)), and 9° N™ (NEB)), or a mutant, derivative, or fragment thereof having DNA polymerase activity (e.g., mutant DNA polymerases that include point mutations compared to a reference thermostable DNA polymerase sequence, e.g., Taq A271 F667Y, Tth A273 F668Y, and Taq A271 F667Y E681W; truncation mutants, e.g., KlenTAQ®, an N-terminal deletion variant of Taq lacking the first 280 amino acids; and mutants that include truncations and point mutations, e.g., Hemo KlenTaq®, an N-terminal deletion variant of Taq lacking the first 280 amino acids containing three internal point mutations that make it resistant to inhibitors in whole blood). For example, suitable DNA polymerases include, but are not limited to, Taq, Hemo KlenTaq®, Hawk Z05, APTATAQ™, Pfu, VENTR®, or higher fidelity DNA polymerases such as PHUSION® (Thermo Scientific), Q5® (NEB), KAPA HiFi™ (Roche), PfuUltra (Agilent), KOD XTREME™ (Millipore), HotStar HiFidelity (Qiagen), ACCUPRIME™ Pfx (Invitrogen), and PLATINUM™ Taq (Invitrogen).

In some embodiments, the thermostable DNA polymerase is a mutant thermostable DNA polymerase. In some embodiments, the mutant thermostable DNA polymerase is listed in Table B. In some embodiments, the mutant thermostable DNA polymerase is selected from the group consisting of Klentaq®1, Klentaq® LA, Cesium Klentaq® AC, Cesium Klentaq® AC LA, Cesium Klentaq® C, Cesium Klentaq® C LA, Omni Klentaq®, Omni Klentaq® 2, Omni Klentaq® LA, Omni Taq, OmniTaq LA, Omni Taq 2, Omni Taq 3, Hemo KlenTaq®, KAPA Blood DNA polymerase, KAPA3G Plant DNA polymerase, KAPA 3G Robust DNA polymerase, MyTaq™ Blood, PHUSION® Blood II DNA polymerase, AmpliTaq® (Taq G46D), AmpliTaq® Gold, RealTaq, ExcelTaq™, and BioReady Taq. In some embodiments, the thermostable DNA polymerase is a hot start thermostable DNA polymerase (e.g., APTATAQ™, Hawk Z05, or PHUSION® Blood II DNA polymerase).

In some embodiments, the thermostable nucleic acid polymerase (e.g., thermostable DNA polymerase) is inhibited by the presence of subject-derived cells or cell debris under normal reaction conditions. In some embodiments, the thermostable nucleic acid polymerase (e.g., thermostable DNA polymerase) is inhibited by whole blood under normal reaction conditions. In some embodiments, the thermostable nucleic acid polymerase (e.g., thermostable DNA polymerase) is inhibited by 1% (v/v) whole blood under normal reaction conditions. In some embodiments, the thermostable nucleic acid polymerase (e.g., thermostable DNA polymerase) is inhibited by 8% (v/v) whole blood under normal reaction conditions. In some embodiments, the normal reaction conditions are the reaction conditions recommended by the manufacturer of the thermostable DNA polymerase or reaction conditions that are commonly used in the art.

In some embodiments of any of the preceding methods, the method further includes amplifying one or more additional target nucleic acids in a multiplexed PCR reaction to generate one or more additional amplicons. In some embodiments, the multiplexed PCR reaction amplifies 2, 3, 4, 5, 6, 7, 8, 9, 10, 11, 12, 15, 20, 25, 30, 35, 40, 45, 50, 60, or more target nucleic acids.

In some embodiments of any of the preceding methods, the method further includes adding deoxynucleotide triphosphates (dNTPs), magnesium, one or more forward primers (e.g., 1, 2, 3, 4, 5, 6, 7, 8, 9, 10, 11, or 12 forward primers), and/or one or more reverse primers (e.g., 1, 2, 3, 4, 5, 6, 7, 8, 9, 10, 11, or 12 reverse primers) during step (i) or during step (iii).

In some embodiments of any of the preceding methods, the whole blood sample is about 0.2 mL to about 5 mL (e.g., about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.5 mL, about 3 mL, about 3.5 mL, about 4 mL, about 5 mL).

The invention allows use of a concentrated lysate prepared from a larger volume of whole blood. In some embodiments, a lysate produced from a whole blood sample of about 0.2 mL to about 10 mL has a volume of about 10 μL to about 1000 μL (e.g., about 10 μL, about 20 μL about 30 μL, about 40 μL, about 50 μL, about 60 μL, about 70 μL, about 80 μL, about 90 μL, about 100 μL, about 125 μL, about 150 μL, about 175 μL, about 200 μL, about 225 μL, about 250 μL, about 275 μL, about 300 μL, about 325 μL, about 350 μL, about 375 μL, about 400 μL, about 425 μL, about 450 μL, about 475 μL, about 500 μL, about 525 μL, about 550 μL, about 600 μL, about 625 μL, about 650 μL, about 675 μL, about 700 μL, about 725 μL, about 750 μL, about 775 μL, about 800 μL, about 825 μL, about 850 μL, about 875 μL, about 900 μL, about 925 μL, about 950 μL, about 975 μL, or about 1000 μL). In some embodiments, the lysate produced from the whole blood sample has a volume of about 25 μL to about 200 μL. In some embodiments, the lysate produced from the whole blood sample has a volume of about 50 μL. In some embodiments, the lysate is concentrated at least 2-fold, 3-fold, 4-fold, 5-fold, 6-fold, 7-fold, 8-fold, 9-fold, 10-fold, 20-fold, 30-fold, 40-fold, 50-fold, or more compared to the whole blood sample.

In some embodiments, the reaction mixture of step (i) contains about 1% to about 80% lysate (e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, or about 80% crude blood lysate In some embodiments, the reaction mixture of step (i) contains about 50% lysate.

In some embodiments of any of the preceding methods, the denatured reaction mixture has a volume ranging from about 0.1 μL to about 250 μL or more, e.g., about 1 μL, about 10 μL, about 20 μL, about 30 μL, about 40 μL, about 50 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, or more. In some embodiments, the volume of the denatured reaction mixture is about 100 μL.

In some embodiments of any of the preceding methods, the method does not include extracting or purifying the amplified target nucleic acid prior to the sequencing. In some embodiments, the extracting includes chloroform or phenol/chloroform extraction, nuclease digestion, salting out, ion exchange extraction, binding to silica or other solid phase materials, or gel extraction.

In some embodiments of any of the preceding methods, the method further includes cleaning up the amplified target nucleic acid prior to the sequencing. In some embodiments, the cleaning up includes magnetic bead purification, enzymatic clean-up, or column clean-up. In other embodiments, no clean-up step is performed.

Any suitable sequencing approach can be used in the methods described herein. For example, in some embodiments, the sequencing includes massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing)), single molecule sequencing (e.g., Helicos™ sequencing, single-molecule real-time (SMRT™) sequencing, and nanopore sequencing) and Sanger sequencing.

In some embodiments, the massively parallel sequencing comprises use of a synthetic control DNA to normalize read counts, wherein the target nucleic acid is detected in the sample if the normalized read count for the target nucleic acid is at or above a reference read count. Any suitable reference read count may be used. In some embodiments, the reference read count is 1, 2, 3, 4, 5, or 6 standard deviations above the average normalized read count of the highest contaminating sequence from a negative sample. In some embodiments, the reference read count is 3 standard deviations above the average normalized read count of the highest contaminating sequence from a negative sample.

In some embodiments of any of the preceding methods, the method further includes amplifying one or more additional target nucleic acids in a multiplexed amplification (e.g., multiplexed PCR) reaction to generate one or more additional amplicons.

In some embodiments of any of the preceding methods, the method identifies the genus of the pathogen. In some embodiments of any of the preceding methods, the method identifies the species of the pathogen.

Any of the methods described above may include detecting the amplified target nucleic acid using T2 magnetic resonance (T2MR). The detecting by T2MR can occur prior to, after, or concurrent with the sequencing. In particular embodiments, the detecting by T2MR occurs prior to the sequencing. For example, in some embodiments, the method includes the following steps: (i) adding magnetic particles to a portion of the amplified solution or amplified lysate solution to form a detection mixture, wherein the magnetic particles have binding moieties on their surface, the binding moieties operative to alter aggregation of the magnetic particles in the presence of the amplified target nucleic acid, and (ii) detecting the presence of the amplified target nucleic acid by measuring the aggregation of the magnetic particles using T2MR. In some embodiments, step (ii) includes the following steps: (a) providing the detection mixture in a detection tube within a device, the device including a support defining a well for holding the detection tube including the mixture, and having an RF coil configured to detect a signal produced by exposing the mixture to a bias magnetic field created using one or more magnets and an RF pulse sequence; (b) exposing the detection mixture to a bias magnetic field and an RF pulse sequence; (c) following step (b), measuring the signal from the detection tube; and (d) on the basis of the result of step (c), detecting the amplified target nucleic acid.

The magnetic particles may be any of the magnetic particles described herein or in International Patent Application Publication Nos. WO 2012/054639, WO 2016/118766, WO 2017/127731, or in International Patent Application No. PCT/US2018/033278, each of which is incorporated herein by reference in its entirety. In some embodiments, the magnetic particles include a first population of magnetic particles conjugated to a first probe, and a second population of magnetic particles conjugated to a second probe, the first probe operative to bind to a first segment of the amplified target nucleic acid and the second probe operative to bind to a second segment of the amplified target nucleic acid, wherein the magnetic particles form aggregates in the presence of the amplified target nucleic acid. The magnetic particles may be substantially monodisperse.

The magnetic particles may have any suitable size, for example, any size described below, or in International Patent Application Publication Nos. WO 2012/054639, WO 2016/118766, WO 2017/127731, or in International Patent Application No. PCT/US2018/033278. In some embodiments, the magnetic particles have a mean diameter of from 650 nm to 950 nm. In some embodiments, the magnetic particles have a mean diameter of from about 670 nm to about 890 nm.

The magnetic particles may have any T₂ relaxivity per particle, for example, T₂ relaxivity per particle described below. In some embodiments, the magnetic particles have a T₂ relaxivity per particle of from 1×10⁹ to 1×10¹² mM⁻¹s⁻¹.

Any suitable amount of magnetic particles can be added to the sample, for example, any amount described below. In some embodiments, from 1×10⁶ to 1×10¹³ magnetic particles are added per milliliter of the sample or the amplified solution.

In another example, provided herein is a method for detecting a target nucleic acid in a biological sample obtained from a subject, wherein the biological sample includes subject-derived cells or cell debris, the method including the following steps: (a) amplifying a target nucleic acid in the biological sample to form an amplified solution including an amplified target nucleic acid; (b) detecting the amplified target nucleic acid using T2MR to provide a group-level identification of the target nucleic acid; and (c) sequencing the amplified target nucleic acid to provide a species-level or variant-level identification of the target nucleic acid, wherein the method is capable of detecting a concentration of about 10 copies/mL of the target nucleic acid in the biological sample.

In any of the preceding methods, detecting the amplified target nucleic acid using T2MR can result in a group-level identification of the target nucleic acid by T2MR. The detecting can provide group-level information for any species described herein. In some embodiments, the group-level identification identifies the organism from which the target nucleic acid is obtained as pan-Gram positive, pan-Gram negative, Enterobacteriaceae, an Enterobacter spp., an Enterobacter cloacae complex, a Citrobacter spp., an Enterococcus spp., a Streptococcus spp., a Staphylococcus spp. (e.g., a coagulase-negative Staphylococcus spp.), an Acinetobacter spp., a Corynebacterium spp., a Mycobacterium spp., pan-fungal, a Candida spp., or a biothreat species (e.g., Bacillus anthracis, Francisella tularensis, Burkholderia spp. (e.g., B. mallei or B. pseudomallei), Yersinia pestis, or Rickettsia prowazekii). In some embodiments, the group-level identification identifies the target nucleic acid as including a sequence of an antimicrobial resistance gene or a toxin gene or a fragment thereof. In some embodiments, detecting the amplified target nucleic acid using T2MR results in the identification of a sequence of an antimicrobial resistance gene or a fragment thereof. Non-limiting examples of antimicrobial resistance genes include bla_{KPC}, blaZ, bla_{NDM}, bla_{IMP}, bla_{VIM}, bla_{OXA} (e.g., bla_{OXA-48}), bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, mefE, ermA, ermB, tetA, tetB, tetX, tetR, qnrA, qnrB, qnrS, FKS1, FKS2, ERG11, or PDR1, or variants thereof. In the literature, the enzymes encoded by these genes are typically spelled in capital letters, while the gene names are italicized. For example, the enzyme NDM is encoded by the bla_{NDM} gene. This convention generally holds for all of the beta lactamase genes (e.g., NDM, KPC, IMP, VIM, DHA, CMY, FOX, CTX-M, SHV, TEM, and OXA-48-like). In the present application, these terms are used interchangeably, and the capitalized shorthand terms, e.g., “NDM” may be used to refer to a nucleic acid for simplicity. Other resistance genes are typically italicized in the literature (e.g., mecA, mecC, vanA, vanB, mefA, mefE, ermA, and ermB), but in the present application, it is to be understood that italicized and non-italicized versions of these names are used interchangeably. In some embodiments, detecting the amplified target nucleic acid using T2MR results in the identification of a sequence of a toxin gene or a fragment thereof. Non-limiting examples of antimicrobial resistance genes include Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G.

In any of the preceding methods, sequencing the amplified target nucleic acid can result in a species-level or variant-level identification of the target nucleic acid. In some embodiments, the species level is a taxonomic species, a taxonomic subspecies, or a strain. In some embodients, the variant-level identification is a nucleic acid variant (e.g., a single nucleotide polymorphism (SNP), an insertion/deletion (indel), a repetitive element, or a microsatellite repeat).

For example, in some exemplary embodiments, the group-level identification by T2MR is pan-Gram positive, and the species-level identification by sequencing is Enterococcus faecium, Enterococcus faecalis, Streptococcus pneumoniae, Streptococcus pyogenes, a viridans Streptococcus, or Staphylococcus aureus. In other embodiments, the group-level identification by T2MR is pan-Gram negative, and the species-level identification by sequencing is Acinetobacter baumannii, Escherichia coli, Haemophilus influenzae, Klebsiella pneumoniae, or Pseudomonas aeruginosa. In still other embodiments, the group-level identification by T2MR is an antimicrobial resistance gene, and the species-level identification by sequencing is a nucleic acid variant of the antimicrobial resistance gene (e.g., bla_{KPC}, blaZ, bla_{NDM}, bla_{VIM}, bla_{OXA} (e.g., bla_{OXA-48}), bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, mefE, ermA, ermB, tetA, tetB, tetX, tetR, qnrA, qnrB, qnrS, FKS1, FKS2, ERG11, or PDR1). For example, in some embodiments, (i) the identification by T2MR is bla_{KPC}, and the variant-level identification by sequencing is KPC-1, KPC-2, KPC-3, KPC-4, KPC-5, KPC-6, KPC-7, KPC-8, KPC-10, KPC-11, KPC-12, KPC-13, KPC-14, KPC-15, KPC-16, KPC-17, KPC-18, KPC-19, KPC-21, KPC-22, KPC-23, KPC-24, KPC-25, KPC-26, KPC-27, KPC-28, KPC-29, KPC-30, KPC-31, KPC-32, KPC-33, KPC-34, or KPC-35; (ii) the identification by T2MR is bla_{crx-m}, and the variant-level identification by sequencing is CTX-M-1, CTX-M-2, CTX-M-3, CTX-M-4, CTX-M-5, CTX-M-6, CTX-M-7, CTX-M-8, CTX-M-9, CTX-M-10, CTX-M-12, CTX-M-13, CTX-M-14, CTX-M-15, CTX-M-16, CTX-M-17, CTX-M-19, CTX-M-20, CTX-M-21, CTX-M-22, CTX-M-23, CTX-M-24, CTX-M-25, CTX-M-26, CTX-M-27, CTX-M-28, CTX-M-29, CTX-M-30, CTX-M-31, CTX-M-32, CTX-M-33, CTX-M-34, CTX-M-35, CTX-M-36, CTX-M-37, CTX-M-38, CTX-M-39, CTX-M-40, CTX-M-41, CTX-M-42, CTX-M-43, CTX-M-44, CTX-M-46, CTX-M-47, CTX-M-48, CTX-M-49, CTX-M-50, CTX-M-51, CTX-M-52, CTX-M-53, CTX-M-54, CTX-M-55, CTX-M-56, CTX-M-58, CTX-M-59, CTX-M-60, CTX-M-61, CTX-M-62, CTX-M-63, CTX-M-64, CTX-M-65, CTX-M-66, CTX-M-67, CTX-M-68, CTX-M-69, CTX-M-71, CTX-M-72, CTX-M-73, CTX-M-74, CTX-M-75, CTX-M-76, CTX-M-77, CTX-M-78, CTX-M-79, CTX-M-80, CTX-M-81, CTX-M-82, CTX-M-83, CTX-M-84, CTX-M-85, CTX-M-86, CTX-M-87, CTX-M-88, CTX-M-89, CTX-M-90, CTX-M-91, CTX-M-92, CTX-M-93, CTX-M-94, CTX-M-95, CTX-M-96, CTX-M-97, CTX-M-98, CTX-M-99, CTX-M-100, CTX-M-101, CTX-M-102, CTX-M-103, CTX-M-104, CTX-M-105, CTX-M-110, CTX-M-111, CTX-M-112, CTX-M-113, CTX-M-114, CTX-M-115, CTX-M-116, CTX-M-117, CTX-M-121, CTX-M-122, CTX-M-123, CTX-M-124, CTX-M-125, CTX-M-126, CTX-M-127, CTX-M-129, CTX-M-130, CTX-M-131, CTX-M-132, CTX-M-134, CTX-M-136, CTX-M-137, CTX-M-138, CTX-M-139, CTX-M-141, CTX-M-142, CTX-M-144, CTX-M-146, CTX-M-147, CTX-M-148, CTX-M-150, CTX-M-151, CTX-M-152, CTX-M-155, CTX-M-156, CTX-M-157, CTX-M-158, CTX-M-159, CTX-M-160, CTX-M-161, CTX-M-162, CTX-M-163, CTX-M-164, CTX-M-165, CTX-M-166, CTX-M-167, CTX-M-168, CTX-M-169, CTX-M-170, CTX-M-171, CTX-M-172, CTX-M-173, CTX-M-174, CTX-M-175, CTX-M-176, CTX-M-177, CTX-M-178, CTX-M-179, CTX-M-180, CTX-M-181, CTX-M-182, CTX-M-183, CTX-M-184, CTX-M-185, CTX-M-186, CTX-M-187, CTX-M-188, CTX-M-189, CTX-M-190, CTX-M-191, CTX-M-192, CTX-M-193, CTX-M-194, CTX-M-195, CTX-M-196, CTX-M-197, CTX-M-198, CTX-M-199, CTX-M-200, CTX-M-201, CTX-M-202, CTX-M-203, CTX-M-204, CTX-M-205, CTX-M-206, CTX-M-207, CTX-M-208, CTX-M-209, CTX-M-210, CTX-M-211, CTX-M-212, CTX-M-213, CTX-M-214, CTX-M-216, CTX-M-217, CTX-M-218, CTX-M-219, or CTX-M-220; or (iii) the identification by T2MR is bla_{NDM}, and the variant-level identification by sequencing is NDM-1, NDM-2, NDM-3, NDM-4, NDM-5, NDM-6, NDM-7, NDM-8, NDM-9, NDM-10, NDM-11, NDM-12, NDM-13, NDM-14, NDM-15, NDM-16, NDM-17, NDM-18, NDM-19, NDM-20, NDM-21, NDM-22, NDM-23, or NDM-24.

In yet another example, in some embodiments, the group-level identification by T2MR is pan-fungal or a Candida spp., and the species-level identification by sequencing is Candida albicans, Candida guilliermondii, Candida glabrata, Candida krusei, Candida lusitaniae, Candida parapsilosis, Candida metapsilosis, Candida orthopsilosis, Candida dublinensis, Candida tropicalis, Candida auris, Candida haemulonii, Candida duobushaemulonii, or Candida pseudohaemulonii.

In any of the preceding methods, the detecting by T2MR can be completed within 5 hours of amplifying the target nucleic acid, e.g., within about 5, 4, 3, 2, or 1 hour.

Any suitable pathogen can be detected and/or sequenced using any of the methods described herein. For example, in some embodiments, the pathogen is a fungal pathogen, a bacterial pathogen, a protozoan pathogen, or a viral pathogen.

In some embodiments, the pathogen is a fungal pathogen (e.g., a Candida spp.). Any suitable fungal pathogen may be detected. In some embodiments, the amplifying includes amplifying a pan-fungal or pan-Candida spp. amplicon. In some embodiments, the Candida spp. is selected from the group consisting of Candida albicans, Candida guilliermondii, Candida glabrata, Candida krusei, Candida lusitaniae, Candida parapsilosis, Candida metapsilosis, Candida orthopsilosis, Candida dublinensis, Candida tropicalis, Candida auris, Candida haemulonii, Candida duobushaemulonii, Candida pseudohaemulonii, an Aspergillus spp., or a Cryptococcus spp. In some embodiments, the Candida spp. is selected from the group consisting of Candida albicans, Candida guilliermondii, Candida glabrata, Candida krusei, Candida lusitaniae, Candida parapsilosis, and Candida tropicalis. In other embodiments, the pathogen is a bacterial pathogen. Any suitable bacterial pathogen may be detected, including any described herein. In some embodiments, the amplifying includes amplifying a pan-bacterial amplicon (e.g., a 16S rRNA amplicon). Any suitable primer pair described herein or known in the art can be used. In some embodiments, the amplifying includes amplifying the 16S rRNA amplicon in the presence of a forward primer including the nucleic acid sequence of 5′-GGTTAAGTCCCGCAACGAGCGC-3′ (SEQ ID NO: 60) and a reverse primer including the nucleic acid sequence of 5′-AGGAGGTGATCCAACCGCA-3′ (SEQ ID NO: 61). In some embodiments, the bacterial pathogen is a Gram positive bacterium, a Gram negative bacterium, an Enterobacteriaceae family bacterium, an Enterobacter spp., a Citrobacter spp., a Enterococcus spp., a Streptococcus spp. (e.g., a viridans Streptococcus), a Staphylococcus spp. (e.g., a coagulase-negative Staphylococcus spp.), an Acinetobacter spp., a Corynebacterium spp., Enterobacter cloacae complex, or a Mycobacterium spp. In some embodiments, the bacterial pathogen is selected from the group consisting of Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, and Neisseria meningitides. In some embodiments, the bacterial pathogen is selected from the group consisting of Acinetobacter baumannii, Enterococcus faecium, Staphylococcus aureus, Klebsiella pneumoniae, Pseudomonas aeruginosa, and Escherichia coli. In other embodiments, the bacterial pathogen is selected from Borrelia burgdorferi, Borrelia afzelii, and Borrelia garinii.

In other embodiments, the pathogen is a protozoan pathogen. Any suitable protozoan pathogen may be detected, including any described herein, e.g., Babesia microti or Babesia divergens.

In some embodiments, the pathogen is a biothreat species, e.g., Bacillus anthracis, Francisella tularensis, Burkholderia spp. (e.g., B. mallei or B. pseudomallei), Yersinia pestis, or Rickettsia prowazekii.

In any of the preceding methods, the method can be capable of detecting a concentration of about 10 colony-forming units (CFU)/mL of the pathogen species in the whole blood sample or lower, e.g., about 1 CFU/mL to about 10 CFU/mL (e.g., about 1 CFU/mL, about 2 CFU/mL, about 3 CFU/mL, about 4 CFU/mL, about 5 CFU/mL, about 6 CFU/mL, about 7 CFU/mL, about 8 CFU/mL, about 9 CFU/mL, or about 10 CFU/mL) of the pathogen species in the whole blood sample.

In some embodiments of any of the preceding methods, the target nucleic acid may be an antimicrobial resistance gene. Any suitable antimicrobial resistance gene may be detected and/or sequenced using the methods described herein. Exemplary antimicrobial resistance genes include but are not limited to bla_{KPC}, blaZ, bla_{NDM}, bla_{OXA} (e.g., bla_{OXA-48}), bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, mefE, ermA, ermB, tetA, tetB, tetX, tetR, qnrA, qnrB, qnrS, FKS1, FKS2, ERG11, or PDR1.

In some embodiments of any of the preceding methods, the target nucleic acid may be a toxin gene. Any suitable toxin gene may be detected and/or sequenced using the methods described herein. Exemplary, non-limiting toxin genes include Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G.

Any of the methods described herein may further include diagnosing the subject based on the detection of the target nucleic acid, or the nucleotide sequence of the target nucleic acid, wherein the presence or sequence of the target nucleic indicates that the subject is suffering from a disease associated with the pathogen. The method may further include administering to the subject a suitable therapy, e.g., a therapy tailored to the identity of the pathogen based on the sequence of the target nucleic acid.

In another example, in some embodiments, the invention provides a method for sequencing a target nucleic acid in a sample including unprocessed whole blood, the method including: (a) providing a mixture including a buffer solution including a buffering agent, dNTPs, magnesium, a forward primer, a reverse primer, and a thermostable nucleic acid polymerase, wherein the buffer solution has a moderately alkaline pH at ambient temperature, and wherein the final concentration of the thermostable nucleic acid polymerase is at least about 0.01 units (e.g., about 0.01 units, about 0.02 units, about 0.03 units, about 0.04 units, about 0.05 units, about 0.06 units, about 0.07 units, about 0.08 units, about 0.09 units, about 0.10 units, about 0.15 units about 0.2 units, about 0.25 units, about 0.3 units, about 0.35 units, about 0.4 units, about 0.45 units, about 0.5 units, about 0.6 units, about 0.65 units, about 0.7 units, about 0.8 units, about 0.9 units, about 1 unit, or more) per microliter of the mixture; (b) adding to the mixture a portion of a whole blood sample obtained from a subject to form a reaction mixture; (c) amplifying the target nucleic acid to form an amplified solution including an amplicon; and (d) sequencing the amplicon. In some embodiments, the reaction mixture contains from about 1% to about 70% (v/v) whole blood, e.g., about 1%, about 2%, about 3%, about 4%, about 5%, about 10%, about 15%, about 20%, about 25%, about 30%, about 35%, about 40%, about 45%, about 50%, about 55%, about 60%, about 65%, or about 70% (v/v) whole blood). In some embodiments, the reaction mixture contains more than about 1%, more than about 2%, more than about 3%, more than about 4%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, or more than about 70% (v/v) whole blood.

In a still further example, in some embodiments, the invention provides a method for sequencing a target nucleic acid in a sample including whole blood, the method including: (a) providing a mixture, wherein the mixture includes a buffer solution including a buffering agent, dNTPs, magnesium, a forward primer, a reverse primer, and a thermostable nucleic acid polymerase, wherein the buffer solution has a moderately alkaline pH at ambient temperature, and wherein the mixture contains about at least about 1×10⁻⁵ micrograms (e.g., about 1×10⁻⁵ micrograms, about 1.5×10⁻⁵ micrograms, about 2×10⁻⁵ micrograms, about 2.4×10⁻⁵ micrograms, about 2.5×10⁻⁵ micrograms, about 3×10⁻⁵ micrograms, about 4×10⁻⁵ micrograms, about 5×10⁻⁵ micrograms, about 6×10⁻⁵ micrograms, about 7×10⁻⁵ micrograms, about 8×10⁻⁵ micrograms, about 9×10⁻⁵ micrograms, about 1×10⁻⁴ micrograms, about 2×10⁻⁴ micrograms, about 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻⁴ micrograms, about 1×10⁻³ micrograms, about 2×10⁻³ micrograms, 3×10⁻³ micrograms, about 4×10⁻³ micrograms, about 5×10⁻³ micrograms, about 6×10⁻³ micrograms, about 7×10⁻³ micrograms, about 8×10⁻³ micrograms, about 9×10⁻³ micrograms, about 0.01 micrograms, about 0.02 micrograms, about 0.03 micrograms, about 0.04 micrograms, about 0.05 micrograms, or more) of the thermostable nucleic acid polymerase per microliter of the mixture of the thermostable nucleic acid polymerase; (b) adding to the mixture a portion of a whole blood sample obtained from a subject to form a reaction mixture; (c) amplifying the target nucleic acid to form an amplified solution including an amplicon; and (d) sequencing the amplicon. In some embodiments, the reaction mixture contains from about 1% to about 70% (v/v) whole blood, e.g., about 1%, about 2%, about 3%, about 4%, about 5%, about 10%, about 15%, about 20%, about 25%, about 30%, about 35%, about 40%, about 45%, about 50%, about 55%, about 60%, about 65%, or about 70% (v/v) whole blood).

Any suitable buffering agent may be used in the methods of the invention. For example, in some embodiments, any buffer with a pKa ranging from about 7.0 to about 9.2 (e.g., about 7.0 to about 7.6; from about 7.6 to about 8.2; or about 8.2 to about 9.2) may be used. Exemplary buffering agents with a pKa ranging from about 7.0 to about 7.6 include but are not limited to: MOPS, BES, phosphoric acid, TES, HEPES, and DIPSO. Exemplary buffering agents with a pKa ranging from about 7.6 to about 8.2 include but are not limited to: TAPSO, TEA, n-ethylmorpholine, POPSO, EPPS, HEPPSO, Tris, and Tricine. Exemplary buffering agents with a pKa ranging from about 8.2 to about 9.2 include but are not limited to: glycylglycine, Bicine, TAPS, morpholine, n-methyldiethanolamine, AMPD (2-amino-2-methyl-1,3-propanediol), diethanolamine, and AMPSO. In some embodiments, a buffering agent with a pKa greater than 9.2 may be used. Exemplary buffering agents with a pKa greater than 9.2 include but are not limited to boric acid, CHES, glycine, CAPSO, ethanolamine, AMP (2-amino-2-methyl-1-propanol), piperazine, CAPS, 1,3-diaminopropane, CABS, and piperadine.

In some embodiments of any of the preceding methods, the method results in the production of at least 10⁵copies of the amplicon, e.g., at least 10⁵ copies, at least 10⁶ copies, at least 10⁷ copies, at least 10⁸ copies, at least 10⁹ copies, at least 10¹⁰ copies, at least 10¹¹ copies, at least 10¹² copies, at least 10¹³ copies, or at least 10¹⁴ copies of the amplicon. For example, in some embodiments, the method results in the production of at least 10⁸copies of the amplicon. In some embodiments, the method results in the production of at least 10⁹copies of the amplicon.

Any of the preceding methods can further include detecting one or more (e.g., 1, 2, 3, 4, 5, 6, 7, 8, 9, 10, or more) additional analytes (e.g., nucleic acids (e.g., DNA or RNA (e.g., mRNA)), proteins, cells, or the like. The detecting may be by any suitable approach, e.g., sequencing (e.g., massively-parallel, long-read, and/or Sanger sequencing), optical, fluorescent, mass, density, magnetic, chromatographic, and/or electrochemical measurement. In some embodiments, the detecting is performed by T2MR.

Further provided herein are systems for performing any of the methods described herein, as described further below.

Sample Preparation and Cell Lysis

The methods and systems of the invention may involve sample preparation and/or cell lysis. For example, an organism (e.g., a pathogen) present in a biological sample containing cells, cell debris, and/or nucleic acids (e.g., DNA or RNA (e.g., mRNA)), including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, CSF, SF, or sputum may be lysed prior to amplification of a target nucleic acid. Suitable lysis methods for lysing cells (e.g., pathogen cells) in a biological sample include, for example, mechanical lysis (e.g., beadbeating and sonication), heat lysis, and alkaline lysis.

In some embodiments, the lysis method is beadbeating. In some embodiments, beadbeating may be performed by adding glass beads (e.g., 0.5 mm glass beads, 0.6 mm glass beads, 0.7 mm glass beads, 0.8 mm glass beads, or 0.9 mm glass beads) to a biological sample to form a mixture and agitating the mixture. As an example, the sample preparation and cell lysis (e.g., beadbeating) may be performed using any of the approaches and methods described in WO 2012/054639. Following lysis, the sample may include cell debris or nucleic acids derived from mammalian host cells and/or from the pathogen cell(s) present in the sample.

In some embodiments, the methods of the invention may include preparing a tissue homogenate. Any suitable method or approach known in the art and/or described herein may be used, including but not limited to grinding (e.g., mortar and pestle grinding, cryogenic mortar and pestle grinding, or glass homogenizer), shearing (e.g., blender, rotor-stator, dounce homogenizer, or French press), beating (e.g., beadbeating), or sonication. In some embodiments, several approaches may be combined to prepare a tissue homogenate.

In some embodiments, the methods of the invention involve detection of one or more pathogen-associated analytes in a whole blood sample. In some embodiments, the methods may involve disruption of red blood cells (erythrocytes). In some embodiments, the disruption of the red blood cells can be carried out using an erythrocyte lysis agent (i.e., a lysis buffer, an isotonic lysis agent, or a nonionic detergent). Erythrocyte lysis buffers which can be used in the methods of the invention include, without limitation, isotonic solutions of ammonium chloride (optionally including carbonate buffer and/or EDTA), and hypotonic solutions. The basic mechanism of hemolysis using isotonic ammonium chloride is by diffusion of ammonia across red blood cell membranes. This influx of ammonium increases the intracellular concentration of hydroxyl ions, which in turn reacts with CO₂ to form hydrogen carbonate. Erythrocytes exchange excess hydrogen carbonate with chloride which is present in blood plasma via anion channels and subsequently increase in intracellular ammonium chloride concentrations. The resulting swelling of the cells eventually causes loss of membrane integrity.

Alternatively, the erythrocyte lysis agent can be an aqueous solution of nonionic detergents (e.g., nonyl phenoxypolyethoxylethanol (NP-40), 4-octylphenol polyethoxylate (TRITON™ X-100), BRIJ® 58, or related nonionic surfactants, and mixtures thereof). The erythrocyte lysis agent disrupts at least some of the red blood cells, allowing a large fraction of certain components of whole blood (e.g., certain whole blood proteins) to be separated (e.g., as supernatant following centrifugation) from the white blood cells or other cells (e.g., pathogen cells (e.g., bacterial cells and/or fungal cells)) present in the whole blood sample. Following erythrocyte lysis and centrifugation, the resulting pellet may be lysed, for example, as described above.

In some embodiments, the methods provided herein may include (a) providing a whole blood sample from a subject; (b) mixing the whole blood sample with an erythrocyte lysis agent solution to produce disrupted red blood cells; (c) following step (b), centrifuging the sample to form a supernatant and a pellet, discarding some or all of the supernatant, and resuspending the pellet to form an extract, (d) lysing cells of the extract (which may include white blood cells and/or pathogen cells) to form a lysate. In some embodiments, the method further comprises amplifying one or more target nucleic acids in the lysate. In some embodiments, the method further comprises sequencing one or more target nucleic acids in the lysate. In some embodiments, the sample of whole blood is from about 0.5 to about 10 mL of whole blood, for example, 0.5 mL, 1 mL, 2 mL, 3 mL, 4 mL, 5 mL, 6 mL, 7 mL, 8 mL, 9 mL, or 10 mL of whole blood. In some embodiments, the method may include washing the pellet (e.g., with a buffer such as TE buffer) prior to resuspending the pellet and optionally repeating step (c). In some embodiments, step (c) does not involve resuspending the pellet but instead includes adding a buffer solution to the pellet to form the extract. In some embodiments, the method may include 1, 2, 3, 4, 5, or more wash steps. In other embodiments, the method is performed without performing any wash step. In some embodiments, the amplifying is in the presence of whole blood proteins, non-target nucleic acids, or both. In some embodiments, the amplifying may be in the presence of from about 0.5 μg to about 200 μg (e.g., about 0.5 μg, 1 μg, 5 μg, 10 μg, 15 μg, 20 μg, 25 μg, 30 μg, 35 μg, 40 μg, 45 μg, 50 μg, 55 μg, 60 μg, 70 μg, 80 μg, 90 μg, 100 μg, 110μg, 120 μg, 130 μg, 140 μg, 150 μg, 160 μg, 170 μg, 180 μg, 190 μg, or 200 μg) of subject (i.e., host) DNA. In some embodiments, the amplifying may be in the presence of more than about 1-μg (e.g., more than about 10 μg, 15 μg, 20 μg, 25 μg, 30 μg, 35 μg, 40 μg, 45 μg, 50 μg, 55 μg, 60 μg, 70 μg, 80 μg, 90 μg, 100 μg, 110 μg, 120 μg, 130 μg, 140 μg, 150 μg, 160 μg, 170 μg, 180 μg, 190 μg, or 200 μg) of subject (i.e., host) DNA. In some embodiments, at least a portion of the subject (i.e., host) DNA is from white blood cells of the subject. In some embodiments, the subject (i.e., host) DNA is from white blood cells of the subject.

Amplification Approaches

In several embodiments, the methods and systems of the invention involve amplification of one or more nucleic acids. Amplification may be exponential or linear. A target or template nucleic acid may be any suitable nucleic acid. In some embodiments, the target nucleic acid is DNA or RNA (e.g., mRNA). The sequences amplified in this manner form an amplified target nucleic acid (also referred to herein as an amplicon). Primers and probes can be readily designed by those skilled in the art to target a specific template nucleic acid sequence. In certain preferred embodiments, resulting amplicons are short to allow for rapid cycling and generation of copies. The size of the amplicon can vary as needed, for example, to provide the ability to discriminate target nucleic acids from non-target nucleic acids. For example, amplicons can be less than about 1,000 nucleotides in length. In some embodiments, the amplicons are from 100 to 500 nucleotides in length (e.g., 100 to 200, 150 to 250, 300 to 400, 350 to 450, or 400 to 500 nucleotides in length). In other embodiments, the amplicons are greater than about 1,000 nucleotides in length, e.g., about 1,000, about 2,000, about 3,000, about 4,000, about 5,000, about 6,000, about 7,000, about 8,000, about 9,000, about 10,000, or more nucleotides in length. In some embodiments, more than one (e.g., 1, 2, 3, 4, 5, 6, 7, 8, 9, 10, or more than 10) target nucleic acids may be amplified in one reaction. In other embodiments, a single target nucleic acid may be amplified in one reaction. In some embodiments, the invention provides amplification-based nucleic acid detection assays conducted in complex samples containing cells and/or cell debris, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), urine, CSF, SF, or sputum (e.g., purulent sputum or bloody sputum). In several embodiments, the method provides methods for amplifying target nucleic acids in a biological sample that includes cells, cell debris, and/or nucleic acids (e.g., DNA or RNA (e.g., mRNA)) derived from both a host mammalian subject and from a microbial organism, particularly a microbial pathogen. The resulting amplified target nucleic acids, or portions or fragments thereof, can be sequenced according to any of the sequencing approaches known in the art and/or described herein.

Sample preparation typically involves removing or providing resistance for common PCR inhibitors found in complex samples containing cells and/or cell debris. Common inhibitors are listed in Table A (see also Wilson, Appl. Environ. Microbiol., 63:3741 (1997)). The “facilitators” in Table A indicate methodologies or compositions that may be used to reduce or overcome inhibition. Any of the facilitators may be used in the methods described herein. Inhibitors typically act by either prevention of cell lysis, degradation or sequestering a target nucleic acid, and/or inhibition of a polymerase activity. The most commonly employed polymerase, Taq, is typically inhibited by the presence of 0.1% blood in a reaction. Mutant Taq polymerases have been engineered that are resistant to common inhibitors (e.g., hemoglobin and/or humic acid) found in blood (see, e.g., Kermekchiev et al., Nucl. Acid. Res., 37(5): e40, (2009)). Manufacturer recommendations indicate these mutations enable direct amplification from up to 20% blood.

TABLE A
PCR inhibitors and facilitators for overcoming inhibition

| Sample or | | | |
|---|---|---|---|
| Specimen Type | Target | Inhibitor | Facilitator |
| feces | Escherichia coli | >10³ bacterial cells | ion-exchange column |
| CSF | Treponema | Cell debris causing nonspecific | nested primers |
| | pallidum | amplification | |
| whole blood | mammalian | >4 μl of blood/100-ml reaction | 1-2% blood per reaction |
| | tissue | mix | |
| | | (hemoglobin) | |
| feces | Rotavirus | unknown dilution | cellulose fiber |
| clinical | Cytomegalovirus | unidentified components | glass bead extraction |
| specimens | | | |
| human blood | human genes | DNA binding proteins | thermophilic protease from |
| and tissue | | | Thermus strain rt44A |
| mammalian | Mammalian | thermal cycler variations | formamide |
| tissue | tissue genetics | | |
| mammalian | Mammalian | thermal cycler variations | DMSO, glycerol, PEG, |
| tissue | tissue genetics | | organic solvents |
| clinical | Treponema | unknown factors | Various substrate-specific |
| specimens | pallidum | | physicochemical methods |
| forensic semen | Sperm | Genotyping errors; | |
| samples | | selective/total PCR inhibition by | |
| | | vaginal microorganisms | |
| feces | Salmonella | various body fluids | immunomagnetic |
| | enterica | | separation |
| feces | Various enteric | unknown | size exclusion |
| | viruses | | chromatography, |
| | | | physicochemical extraction |
| clinical | Herpes simplex | endogenous inhibitors, random | repurification, coamplified |
| specimens | virus | effects | positive control |
| feces | Escherichia coli | nonspecific inhibitors, urea, | additional primers and |
| | | hemoglobin, heparin, phenol, | reaction cyclers, booster |
| | | SDS | PCR |
| tissue culture | Cytomegalovirus | glove powder | |
| | HIV | | |
| suspensions, | Mycobacterium | mercury-based fixatives, | reduced fixation times, |
| skin biopsies | leprae | neutral buffered formaline | ethanol fixation |
| clinical | Mycobacterium | unknown inhibitors in pus, | physicochemical extraction |
| specimens | tuberculosis | tissue biopsies, sputum, pleural | |
| | | fluid | |
| mammalian | mammalian | unknown contaminant of | additional DNA |
| tissue | tissue genetics | reverse transcriptase | |
| formalin-fixed | Hepatitis C virus | ribonucleotide vanadyl | phenol/chloroform |
| paraffin tissue | | complexes | extraction |
| nasopharyngeal | Bordetella | unknown inhibitors | phenol/chloroform |
| aspirates and | pertussis | | extraction |
| swabs | | | |
| human | HIV type I | detergents | mineral oil |
| mononuclear | | | |
| blood cells | | | |
| bloodstain | human | unidentified heme compound, | BSA |
| | mitochondrial | hemin | |
| | DNA | | |
| blood | various | heparin | alternative polymerases |
| | | | and buffers, chelex, |
| | | | spermine, [Mg2+], glycerol, |
| | | | BSA, heparinase |
| sputum | Mycoplasma | N-acetyl-L-cysteine, | |
| | pneumoniae | dithiothreitol, mucolytic agents | |
| human tissue | HLA-DRB1 | pollen, glove powder, impure | |
| | genotyping | DNA, heparin, hemoglobin | |
| clinical | Mycobacterium | unknown | competitive internal control |
| specimens | tuberculosis | | |
| dental plaque | many | unknown | diatomaceous earth, |
| | | | guanidium isothiocyante, |
| | | | ethanol, acetone |
| ancient | Cytochrome b | unknown | ammonium acetate, |
| mammalian | gene | | ethidium bromide |
| tissues | | | |

Polymerase chain reaction amplification of DNA or cDNA is a tried and trusted methodology; however, as discussed above, polymerases are inhibited by agents contained in complex biological samples containing cells, cell debris, and/or nucleic acids (e.g., DNA or RNA)), including but not limited to commonly used anticoagulants and hemoglobin. Recently, mutant Taq polymerases have been engineered to harbor resistance to common inhibitors found in blood and soil. Currently available polymerases, e.g., HemoKlenTaq® (New England BioLabs, Inc., Ipswich, Mass.) as well as OmniTag® and OmniKlenTaq® (DNA Polymerase Technology, Inc., St. Louis, Mo.) are mutant (e.g., N-terminal truncation and/or point mutations) Taq polymerase that render them capable of amplifying DNA in the presence of up to 10%, 20% or 25% whole blood, depending on the product and reaction conditions (See, e.g., Kermekchiev et al. Nucl. Acids Res. 31:6139 (2003); and Kermekchiev et al., Nucl. Acid. Res., 37:e40 (2009); and see U.S. Pat. No. 7,462,475). Additionally, PHUSION® Blood Direct PCR Kits (Finnzymes Oy, Espoo, Finland), include a unique fusion DNA polymerase enzyme engineered to incorporate a double-stranded DNA binding domain, which allows amplification under conditions which are typically inhibitory to conventional polymerases such as Taq or Pfu, and allow for amplification of DNA in the presence of up to about 40% whole blood under certain reaction conditions. See Wang et al., Nucl. Acids Res. 32:1197 (2004); and see U.S. Pat. Nos. 5,352,778 and 5,500,363. Furthermore, Kapa Blood PCR Mixes (Kapa Biosystems, Woburn, Mass.), provide a genetically engineered DNA polymerase enzyme which allows for direct amplification of whole blood at up to about 20% of the reaction volume under certain reaction conditions. Despite these breakthroughs, direct optical detection of generated amplicons is typically not possible with existing methods since fluorescence, absorbance, and other light-based methods yield signals that are quenched by the presence of blood. See Kermekchiev et al., Nucl. Acid. Res., 37:e40 (2009).

Table B shows a list of mutant thermostable DNA polymerases that are compatible with many types of interfering substances and that may be used in the methods of the invention for amplification of target nucleic acids in biological samples containing cells and/or cell debris. In certain embodiments, the invention features the use of enzymes compatible with whole blood, e.g., mutant thermostable DNA polymerases including but not limited to NEB HemoKlenTaq™, DNAP OmniKlenTaq™, Kapa Biosystems whole blood enzyme, Thermo-Fisher Finnzymes PHUSION® enzyme, or any of the mutant thermostable DNA polymerases shown in Table B.

TABLE B
Exemplary mutant thermostable DNA polymerases

| Polymerase | Reference |
|---|---|
| Klentaq ®1 | Barnes, Proc Natl Acad Sci USA. |
| | 91(6): 2216-2220, 1994. |
| Klentaq ® LA | Barnes, Proc Natl Acad Sci USA. |
| | 91(6): 2216-2220, 1994. |
| Cesium Klentaq ® AC | Kermekchiev et al., Nuc. Acids Res. |
| | 31(21): 6139-6147, 2003. |
| Cesium Klentaq ® AC LA | Kermekchiev et al., Nuc. Acids Res. |
| | 31(21): 6139-6147, 2003. |
| Cesium Klentaq ® C | Kermekchiev et al., Nuc. Acids Res. |
| | 31(21): 6139-6147, 2003. |
| Cesium Klentaq ® C LA | Kermekchiev et al., Nuc. Acids Res. |
| | 31(21): 6139-6147, 2003. |
| Omni Klentaq ® | Kermekchiev et al. Nuc. Acids Res. |
| | 37(5): e40, 2009. |
| Omni Klentaq ® 2 | Kermekchiev et al. Nuc. Acids Res. |
| | 37(5): e40, 2009. |
| Omni Klentaq ® LA | Kermekchiev et al. Nuc. Acids Res. |
| | 37(5): e40, 2009. |
| Omni Taq | Kermekchiev et al. Nuc. Acids Res. |
| | 37(5): e40, 2009. |
| Omni Taq LA | Kermekchiev et al. Nuc. Acids Res. |
| | 37(5): e40, 2009. |
| Omni Taq 2 | Kermekchiev et al. Nuc. Acids Res. |
| | 37(5): e40, 2009. |
| Omni Taq 3 | Kermekchiev et al. Nuc. Acids Res. |
| | 37(5): e40, 2009. |
| Hemo KlenTaq ® | Kermekchiev et al. Nuc. Acids Res. |
| | 37(5): e40, 2009. |
| KAPA Blood DNA | KAPA Biosystems |
| Polymerase | |
| KAPA3G Plant DNA | KAPA Biosystems |
| Polymerase | |
| KAPA2G Robust DNA | KAPA Biosystems |
| Polymerase | |
| MyTaq ™ Blood-PCR Kit | Bioline |
| Phusion ® Blood DNA Kit | Thermo Scientific |
| with Hot Start Phusion II | Manage et al., Microfluid. Nanofluid. |
| | 10, 697-702, 2011. |

As described above, a variety of impurities and components of whole blood can be inhibitory to the polymerase and primer annealing. These inhibitors can sometimes lead to generation of false positives and low sensitivities. To reduce the generation of false positives and low sensitivities when amplifying and detecting nucleic acids in complex samples, it is desirable to utilize a thermal stable polymerase not inhibited by whole blood samples, for example as described above, and include one or more internal PCR assay controls (see Rosenstraus et al. J. Clin Microbiol. 36:191 (1998) and Hoofar et al., J. Clin. Microbiol. 42:1863 (2004)).

For example, the assay can include an internal control nucleic acid that contains primer binding regions identical to those of the target sequence to assure that clinical specimens are successfully amplified and detected. In some embodiments, the target nucleic acid and internal control can be selected such that each has a unique probe binding region that differentiates the internal control from the target nucleic acid. The internal control is, optionally, employed in combination with a processing positive control, a processing negative control, and a reagent control for the safe and accurate determination and identification of an infecting organism in, e.g., a whole blood clinical sample. The internal control can be an inhibition control that is designed to co-amplify with the nucleic acid target being detected. Failure of the internal inhibition control to be amplified is evidence of a reagent failure or process error. Universal primers can be designed such that the target sequence and the internal control sequence are amplified in the same reaction tube. Thus, using this format, if the target DNA is amplified but the internal control is not it is then assumed that the target DNA is present in a proportionally greater amount than the internal control and the positive result is valid as the internal control amplification is unnecessary. If, on the other hand, neither the internal control nor the target is amplified it is then assumed that inhibition of the PCR reaction has occurred and the test for that particular sample is not valid.

The assays of the invention can include one or more positive processing controls in which one or more target nucleic acids is included in the assay (e.g., each included with one or more cartridges) at 3× to 5× the limit of detection. If detected by T2MR, the measured T₂ for each of the positive processing controls must be above the pre-determined threshold indicating the presence of the target nucleic acid. The positive processing controls can detect all reagent failures in each step of the process (e.g., lysis, PCR, and T2MR detection), and can be used for quality control of the system. The assays of the invention can include one or more negative processing controls consisting of a solution free of target nucleic acid (e.g., buffer alone). If detected by T2MR, the T₂ measurements for the negative processing control should be below the threshold indicating a negative result while the T₂ measured for the internal control is above the decision threshold indicating an internal control positive result. The purpose of the negative control is to detect carry-over contamination and/or reagent contamination. The assays of the invention can include one or more reagent controls. The reagent control will detect reagent failures in the PCR stage of the reaction (i.e. incomplete transfer of master mix to the PCR tubes). The reagent controls can also detect gross failures in reagent transfer prior to T₂ detection.

The methods of the invention can also include use of a total process control (TPC), for example, an engineered cell (e.g., an engineered bacterium or fungus (e.g., yeast)) comprising a control target nucleic acid that has a known and defined sequence. The TPC may be added to the sample (e.g., environmental or biological sample) as a control to monitor steps including cell lysis, amplification, and sequencing.

In some embodiments, complex samples, which may be a liquid sample (including, for example, a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, CSF, SF, or sputum) can be directly amplified using about 5%, about 10%, about 20%, about 25%, about 30%, about 25%, about 40%, about 45%, about 50%, about 55%, about 60%, about 65%, about 70%, or more complex liquid sample in amplification reactions, and that the resulting amplicons can be directly detected from amplification reaction using, for example, sequencing (e.g., massively parallel, long-read, and/or Sanger sequencing) and/or magnetic resonance (MR) relaxation measurements (e.g., T2MR) upon the addition of conjugated magnetic particles bound to oligonucleotides complementary to the target nucleic acid sequence. Alternatively, the magnetic particles can be added to the sample prior to amplification. Thus, provided are methods for the use of nucleic acid amplification in a complex dirty sample, sequencing and/or hybridization of the resulting amplicon to paramagnetic particles, which may be followed by direct detection of hybridized magnetic particle conjugate and target amplicons using magnetic particle-based detection systems. In some embodiments, the detection is by sequencing only. In other embodiments, direct detection of hybridized magnetic particle conjugates and amplicons is via MR relaxation measurements (e.g., T₂, T₁, T₁/T₂ hybrid, T₂*, and the like). Further provided are methods which are kinetic, in order to quantify the original nucleic acid copy number within the sample (e.g., sampling and nucleic acid detection at pre-defined cycle numbers, comparison of endogenous internal control nucleic acid, use of exogenous spiked homologous competitive control nucleic acid). In some embodiments, the resulting amplicons are detected using a non-MR-based approach, for example, optical, fluorescent, mass, density, chromatographic, and/or electrochemical measurement.

While the exemplary methods described hereinafter relate to amplification using PCR, numerous other methods are known in the art for amplification of nucleic acids (e.g., isothermal methods, rolling circle methods, etc.). Those skilled in the art will understand that these other methods may be used either in place of, or together with, PCR methods. See, e.g., Saiki, “Amplification of Genomic DNA” in PCR Protocols, Innis et al., Eds., Academic Press, San Diego, Calif., pp 13-20 (1990); Wharam et al., Nucleic Acids Res. 29:E54 (2001); Hafner et al., Biotechniques, 30:852 (2001). Further amplification methods suitable for use with the present methods include, for example, reverse transcription PCR (RT-PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, ramification amplification (RAM), transcription based amplification system (TAS), transcription mediated amplification (TMA), the isothermal and chimeric primer-initiated amplification of nucleic acid (ICAN) method, and the smart amplification system (SMAP) method. These methods, as well as others are well known in the art and can be adapted for use in conjunction with provided methods of detection of amplified nucleic acid.

The PCR method is a technique for making many copies of a specific template DNA sequence. The PCR process is disclosed, for example, in U.S. Pat. Nos. 4,683,195; 4,683,202; and 4,965,188. One set of primers complementary to a template DNA are designed, and a region flanked by the primers is amplified by DNA polymerase in a reaction including multiple amplification cycles. Each amplification cycle includes an initial denaturation, and up to 50 cycles of annealing, strand elongation (or extension) and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan et al., Journal of Clinical Microbiology 33:556 (1995). Various modified PCR methods are available and well known in the art. Various modifications such as the “RT-PCR” method, in which DNA is synthesized from RNA using a reverse transcriptase before performing PCR; and the “TaqMan® PCR” method, in which only a specific allele is amplified and detected using a fluorescently labeled TagMan® probe, and Taq DNA polymerase, are known to those skilled in the art. RT-PCR and variations thereof have been described, for example, in U.S. Pat. Nos. 5,804,383; 5,407,800; 5,322,770; and 5,310,652, and references described therein; and Taq Man® PCR and related reagents for use in the method have been described, for example, in U.S. Pat. Nos. 5,210,015; 5,876,930; 5,538,848; 6,030,787; and 6,258,569.

In some embodiments, asymmetric PCR is performed to preferentially amplify one strand of a double-stranded DNA (dsDNA) template. Asymmetric PCR typically involves addition of an excess of the primer for the strand targeted for amplification. An exemplary asymmetric PCR condition is 300 nM of the excess primer and 75 nM of the limiting primer to favor single strand amplification. In other embodiments, 400 nM of the excess primer and 100 nM of the limiting primer may be used to favor single strand amplification. In other embodiments, symmetric PCR is performed.

In some embodiments, including embodiments that employ multiplexed PCR reactions, hot start PCR conditions may be used to reduce mis-priming, primer-dimer formation, improve yield, and/or and ensure high PCR specificity and sensitivity. A variety of approaches may be employed to achieve hot start PCR conditions, including hot start DNA polymerases (e.g., hot start DNA polymerases with aptamer-based inhibitors or with mutations that limit activity at lower temperatures) as well as hot start dNTPs (e.g., CLEANAMP™ dNTPs, TriLink Biotechnologies).

In some embodiments, a PCR reaction may include from about 20 cycles to about 55 cycles or more (e.g., about 20, 25, 30, 35, 40, 45, 50, or 55 cycles).

LCR is a method of DNA amplification similar to PCR, except that it uses four primers instead of two and uses the enzyme ligase to ligate or join two segments of DNA. Amplification can be performed in a thermal cycler (e.g., LCx of Abbott Labs, North Chicago, Ill.). LCR can be performed for example, as according to Moore et al., Journal of Clinical Microbiology 36:1028 (1998). LCR methods and variations have been described, for example, in European Patent Application Publication No. EP0320308, and U.S. Pat. No. 5,427,930.

The TAS method is a method for specifically amplifying a target RNA in which a transcript is obtained from a template RNA by a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS 86:1173 (1989). The TAS method has been described, for example, in International Patent Application Publication No. WO1988/010315.

Transcription mediated amplification (TMA) is a transcription-based isothermal amplification reaction that uses RNA transcription by RNA polymerase and DNA transcription by reverse transcriptase to produce an RNA amplicon from target nucleic acid. TMA methods are advantageous in that they can produce 100 to 1000 copies of amplicon per amplification cycle, as opposed to PCR or LCR methods that produce only 2 copies per cycle. TMA has been described, for example, in U.S. Pat. No. 5,399,491. NASBA is a transcription-based method which for specifically amplifying a target RNA from either an RNA or DNA template. NASBA is a method used for the continuous amplification of nucleic acids in a single mixture at one temperature. A transcript is obtained from a template RNA by a DNA-dependent RNA polymerase using a forward primer having a sequence identical to a target RNA and a reverse primer having a sequence complementary to the target RNA a on the 3′ side and a promoter sequence that recognizes T7 RNA polymerase on the 5′ side. A transcript is further synthesized using the obtained transcript as template. This method can be performed as according to Heim, et al., Nucleic Acids Res., 26:2250 (1998). The NASBA method has been described in U.S. Pat. No. 5,130,238.

The SDA method is an isothermal nucleic acid amplification method in which target DNA is amplified using a DNA strand substituted with a strand synthesized by a strand substitution type DNA polymerase lacking 5′→3′ exonuclease activity by a single stranded nick generated by a restriction enzyme as a template of the next replication. A primer containing a restriction site is annealed to template, and then amplification primers are annealed to 5′ adjacent sequences (forming a nick). Amplification is initiated at a fixed temperature. Newly synthesized DNA strands are nicked by a restriction enzyme and the polymerase amplification begins again, displacing the newly synthesized strands. SDA can be performed according to Walker, et al., PNAS, 89:392 (1992). SDA methods have been described in U.S. Pat. Nos. 5,455,166 and 5,457,027.

The LAMP method is an isothermal amplification method in which a loop is always formed at the 3′ end of a synthesized DNA, primers are annealed within the loop, and specific amplification of the target DNA is performed isothermally. LAMP can be performed according to Nagamine et al., Clinical Chemistry. 47:1742 (2001). LAMP methods have been described in U.S. Pat. Nos. 6,410,278; 6,974,670; and 7,175,985.

The ICAN method is anisothermal amplification method in which specific amplification of a target DNA is performed isothermally by a strand substitution reaction, a template exchange reaction, and a nick introduction reaction, using a chimeric primer including RNA-DNA and DNA polymerase having a strand substitution activity and RNase H. ICAN can be performed according to Mukai et al., J. Biochem. 142: 273(2007). The ICAN method has been described in U.S. Pat. No. 6,951,722.

The SMAP (MITANI) method is a method in which a target nucleic acid is continuously synthesized under isothermal conditions using a primer set including two kinds of primers and DNA or RNA as a template. The first primer included in the primer set includes, in the 3′ end region thereof, a sequence (Ac′) hybridizable with a sequence (A) in the 3′ end region of a target nucleic acid sequence as well as, on the 5′ side of the above-mentioned sequence (Ac′), a sequence (B′) hybridizable with a sequence (Bc) complementary to a sequence (B) existing on the 5′ side of the above-mentioned sequence (A) in the above-mentioned target nucleic acid sequence. The second primer includes, in the 3′ end region thereof, a sequence (Cc′) hybridizable with a sequence (C) in the 3′ end region of a sequence complementary to the above-mentioned target nucleic acid sequence as well as a loopback sequence (D-Dc′) including two nucleic acid sequences hybridizable with each other on an identical strand on the 5′ side of the above-mentioned sequence (Cc′). SMAP can be performed according to Mitani et al., Nat. Methods, 4(3): 257 (2007). SMAP methods have been described in U.S. Patent Application Publication Nos. 2006/0160084, 2007/0190531 and 2009/0042197.

The amplification reaction can be designed to produce a specific type of amplified product, such as nucleic acids that are double stranded; single stranded; double stranded with 3′ or 5′ overhangs; or double stranded with chemical ligands on the 5′ and 3′ ends. The amplified PCR product can be detected by: (i) sequencing; (ii) hybridization of the amplified product to magnetic particle bound complementary oligonucleotides, where two different oligonucleotides are used that hybridize to the amplified product such that the nucleic acid serves as an interparticle tether promoting particle agglomeration; (iii) hybridization mediated detection where the DNA of the amplified product must first be denatured; (iv) hybridization mediated detection where the particles hybridize to 5′ and 3′ overhangs of the amplified product; and/or (v) binding of the particles to the chemical or biochemical ligands on the termini of the amplified product, such as streptavidin functionalized particles binding to biotin functionalized amplified product.

Analytes

Embodiments of the invention include methods and systems for detecting and/or measuring the concentration of one or more analytes in a complex biological sample containing cells and/or cell debris, including but not limited to blood (e.g., whole blood, a crude whole blood lysate, serum, or plasma), bloody fluids (e.g., wound exudate, phlegm, bile, and the like), tissue samples (e.g., a tissue biopsy (e.g., a skin biopsy, muscle biopsy, or lymph node biopsy), including homogenized tissue samples), urine, cerebrospinal fluid (CSF), synovial fluid (SF), or sputum. In several embodiments, the analyte may be a nucleic acid derived from an organism. In some embodiments, the nucleic acid is a target nucleic acid derived from the organism that has been amplified to form an amplicon. In some embodiments, the organism is a plant, a mammal, or a microbial species. The nucleic acid can be detected by sequencing. In some embodiments, the nucleic acid may further be detected by other approaches, including T2MR.

In several embodiments, the analyte may be derived from a microbial pathogen. In such embodiments, the biological sample may include cells and/or cell debris from the host mammalian subject as well as one or more microbial pathogen cells. Exemplary analytes are described herein, e.g., in Table 24. For example, in some embodiments, the analyte is derived from a Gram-negative bacterium, a Gram-positive bacterium, a fungal pathogen (e.g., a yeast (e.g., Candida spp.) or Aspergillus spp.), a protozoan pathogen, or a viral pathogen. In some embodiments, the analyte is derived from a bacterial pathogen, including Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (e.g., K. pneumoniae with resistance marker KPC) and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter koseri), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter aerogenes and Enterobacter cloacae). In some embodiments, the analyte is an antimicrobial resistance marker. Exemplary non-limiting antimicrobial resistance markers include, e.g., bla_{KPC}, blaZ, bla_{NDM}, bla_{IMP}, bla_{VIM}, bla_{OXA} (e.g., bla_{OXA-48}), bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, mefE, ermA, ermB, tetA, tetB, tetX, tetR, qnrA, qnrB, qnrS, FKS1, FKS2, ERG11, or PDR1. In some embodiments, the analyte is derived from a fungal pathogen, for example, Candida spp. (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis) and Aspergillus spp. (e.g., Aspergillus fumigatus). In some embodiments, the analyte is derived from a protozoan pathogen such as a Babesia spp. (e.g., Babesia microti and Babesia divergens). In some embodiments, the analyte is derived from a viral pathogen (e.g., a retrovirus (e.g., HIV), an adeno-associated virus (AAV), an adenovirus, Ebolavirus, hepatitis (e.g., hepatitis A, B, C, or E), herpesvirus, human papillomavirus (HPV), rhinovirus, influenza, parainfluenza, measles, rotavirus, West Nile virus, zika virus, and the like). In some embodiments, the analyte is derived from a biothreat species e.g., Bacillus anthracis, Francisella tularensis, Burkholderia spp. (e.g., B. mallei or B. pseudomallei), Yersinia pestis, or Rickettsia prowazekii. In some embodiments, the analyte is a toxin gene, e.g., Bacillus anthracis toxin genes protective antigen (pagA), edema factor (cya), or lethal factor (lef); enteropathogenic E. coli translocated intimin receptor (Tir); Clostridium difficile toxins TcdA and TcdB; or Clostridium botulinum toxins BoNT/A, BoNT/B, BoNT/C, BoNT/D, BoNT/E, BoNT/F, or BoNT/G.

In some embodiments, a pathogen-associated analyte may be a nucleic acid derived from any of the organisms described above, for example, DNA or RNA (e.g., mRNA). In some embodiments, the nucleic acid is a target nucleic acid derived from the organism that has been amplified to form an amplicon. In some embodiments, the target nucleic acid may be a multi-copy locus. Use of a target nucleic acid derived from a multi-copy locus, in particular in methods involving amplification, may lead to an increase in sensitivity in the assay. Exemplary multi-copy loci may include, for example, ribosomal DNA (rDNA) operons and multi-copy plasmids. In other embodiments, the target nucleic acid may be a single-copy locus. In particular embodiments, the target nucleic acid may be derived from an essential locus, for example, an essential house-keeping gene. In particular embodiments, the target nucleic acid may be derived from a locus that is involved in virulence (e.g., a virulence gene). In any of the above embodiments, a locus may include a gene and/or an intragenic region, for example, an internally transcribed sequence (ITS) between rRNA genes (e.g., ITS1, between the 16S and 23S rRNA genes, or ITS2, between the 5S and 23S rRNA genes). In some embodiments, the target nucleic acid is a 16S rRNA target nucleic acid.

In some embodiments, a target nucleic acid may be (a) species-specific, (b) species-inclusive (in other words, present in all strains or subspecies of a given species), (c) compatible with an amplification/detection protocol, and/or (d) present in multiple copies. In some embodiments, a target nucleic acid may be group-specific or group-inclusive, e.g., genus-specific or genus inclusive. In particular embodiments, a target nucleic acid is chromosomally-encoded, which can help avoid loss by, for example, plasmid exchange and plasmid curing/transduction events.

The target nucleic acid may be a pan-Bacterial target nucleic acid. Any suitable pan-Bacterial nucleic acid can be used. For example, in some instances, the pan-Bacterial target nucleic acid is 16S rRNA.

The target nucleic acid may be a pan-Fungal target nucleic acid. Any suitable pan-Fungal nucleic acid can be used. For example, in some embidments, the pan-Fungal target nucleic acid is Internal Transcribed Spacer (ITS) rRNA (e.g., ITS1 or ITS2).

Below are exemplary, non-limiting target nucleic acids that can be used in the present invention. Any of the target nucleic acids described herein can be sequenced using the methods described herein. Any of the target nucleic acids described herein can further be detected, e.g., using T2MR, as described herein.

Acinetobacter Target Nucleic Acids

In some embodiments, a target nucleic acid may include sequence elements that are specific for an Acinetobacter spp., for example, Acinetobacter baumannii. For example, in some embodiments, an Acinetobacter baumannii target nucleic acid may be amplified in the presence of a forward primer and a reverse primer which are specific to Acinetobacter baumannii, as described below. Detection of such a target nucleic acid in a sample would typically indicate that an Acinetobacter baumannii bacterium was present in the sample. In other embodiments, a target nucleic acid of the invention may include sequence elements that are common to all Acinetobacter spp. For example, in some embodiments, an Acinetobacter spp. target nucleic acid may be amplified in the presence of a forward primer and a reverse primer, each of which is universal to all Acinetobacter spp. Detection of such a target nucleic acid in a sample typically would indicate that an Acinetobacter spp. bacterium was present in the sample. In yet other embodiments, these approaches may be combined.

In some embodiments, an Acinetobacter spp. target nucleic acid may be derived from a linear chromosome or a linear or circular plasmid (e.g., a single-, low-, or multi-copy plasmid). In some embodiments, an Acinetobacter spp. target nucleic acid may be derived from an essential locus (e.g., an essential housekeeping gene) or a locus involved in virulence (e.g., a gene essential for virulence). In some embodiments, an Acinetobacter spp. target nucleic acid may be derived from a multi-copy locus. In other embodiments, an Acinetobacter spp. target nucleic acid may be derived from a multi-copy plasmid.

In some embodiments, an Acinetobacter baumannii target nucleic acid is derived from a region that includes parts or all of the internally transcribed sequence (ITS) between the 5S and 23S rRNA genes (i.e., the ITS2 region). In particular embodiments, an Acinetobacter baumannii target nucleic acid may be amplified in the presence of a forward primer that includes the oligonucleotide sequence 5′-CGT TTT CCA AAT CTG TAA CAG ACT GGG-3′ (SEQ ID NO: 1) or 5′-GGA AGG GAT CAG GTG GTT CAC TCT T-3′ (SEQ ID NO: 55) and a reverse primer that includes the oligonucleotide sequence 5′-AGG ACG TTG ATA GG TTG GAT GTG GA-3′ (SEQ ID NO: 2). For example, in particular embodiments, an Acinetobacter baumannii target nucleic acid may be amplified in the presence of a forward primer that includes the oligonucleotide sequence 5′-GGA AGG GAT CAG GTG GTT CAC TCT T-3′ (SEQ ID NO: 55) and a reverse primer that includes the oligonucleotide sequence 5′-AGG ACG TTG ATA GG TTG GAT GTG GA-3′ (SEQ ID NO: 2). In some embodiments, an amplicon produced using these primers is detected by sequencing. In some embodiments, an amplicon produced using these primers is detected by hybridization using a 5′ capture probe that includes the oligonucleotide sequence 5′-TGA GGC TTG ACT ATA CAA CAC C-3′ (SEQ ID NO: 15) and/or a 3′ capture probe that includes the oligonucleotide sequence 5′-CTA AAA TGA ACA GAT AAA GTA AGA TTC AA-3′ (SEQ ID NO: 16) to detect the presence of Acinetobacter baumannii in a biological sample. In some embodiments, the 5′ capture probe and/or the 3′ capture probe is conjugated to a magnetic nanoparticle.

In some embodiments, a control target nucleic acid for A. baumannii may comprise the nucleic acid sequence of SEQ ID NO: 31.

Enterococcus Target Nucleic Acids

In some embodiments, a target nucleic acid may include sequence elements that are specific for an Enterococcus spp., for example, Enterococcus faecium or Enterococcus faecalis. For example, in some embodiments, an Enterococcus faecium target nucleic acid may be amplified in the presence of a forward primer and a reverse primer which are specific to Enterococcus faecium. Detection of such a target nucleic acid in a sample would typically indicate that an Enterococcus faecium bacterium was present in the sample. In other embodiments, a target nucleic acid may include sequence elements that are specific for multiple (e.g., 2, 3, 4, or 5) Enterococcus spp. For example, in some embodiments, a target nucleic acid may include sequence elements that are specific for Enterococcus faecium and Enterococcus faecalis, as described below. In other embodiments, a target nucleic acid of the invention may include sequence elements that are common to all Enterococcus spp. For example, in some embodiments, an Enterococcus spp. target nucleic acid may be amplified in the presence of a forward primer and a reverse primer, each of which is universal to all Enterococcus spp. Detection of such a target nucleic acid in a sample typically would indicate that an Enterococcus spp. bacterium was present in the sample. In yet other embodiments, these approaches may be combined.

In some embodiments, an Enterococcus spp. target nucleic acid may be derived from a linear chromosome or a linear or circular plasmid (e.g., a single-, low-, or multi-copy plasmid). In some embodiments, an Enterococcus spp. target nucleic acid may be derived from an essential locus (e.g., an essential housekeeping gene) or a locus involved in virulence (e.g., a gene essential for virulence). In some embodiments, an Enterococcus spp. target nucleic acid may be derived from a multi-copy locus. In particular embodiments, an Enterococcus spp. target nucleic acid may be derived from a multi-copy plasmid.

In some embodiments, an Enterococcus spp. target nucleic acid is derived from a region that includes parts or all of the ITS between the 23S and 5S rRNA genes. In particular embodiments, an target nucleic acid that is specific for Enterococcus faecium and Enterococcus faecalis may be amplified in the presence of a forward primer that includes the oligonucleotide sequence 5′-GGT AGC TAT GTA GGG AAG GGA TAA ACG CTG A-3′ (SEQ ID NO: 3) and a reverse primer that includes the oligonucleotide sequence 5′-GCG CTA AGG AGC TTA ACT TCT GTG TTC G-3′ (SEQ ID NO: 4). In some embodiments, an amplicon produced using these primers is detected by sequencing. In some embodiments, an amplicon produced using these primers is detected by hybridization using a 5′ capture probe that includes the oligonucleotide sequence 5′-AAA ACT TAT ATG ACT TCA AAT CCA GTT TT-3′ (SEQ ID NO: 17) or 5′-AAA ACT TAT GTG ACT TCA AAT CCA GTT TT-3′ (SEQ ID NO: 56) and/or a 3′ capture probe that includes the oligonucleotide sequence 5′-TTT ACT CAA TAA AAG ATA ACA CCA CAG-3′ (SEQ ID NO: 18) to detect the presence of Enterococcus faecium in a biological sample. In particular embodiments, an amplicon produced using these primers is detected by hybridization using a 5′ capture probe that includes the oligonucleotide sequence 5′-AAA ACT TAT GTG ACT TCA AAT CCA GTT TT-3′ (SEQ ID NO: 56) and/or a 3′ capture probe that includes the oligonucleotide sequence 5′-TTT ACT CAA TAA AAG ATA ACA CCA CAG T-3′ (SEQ ID NO: 18) to detect the presence of Enterococcus faecium in a biological sample. In some embodiments, an amplicon produced using these primers is detected by hybridization using a 5′ capture probe that includes the oligonucleotide sequence 5′-TGG ATA AGT AAA AGC AAC TTG GTT-3′ (SEQ ID NO: 19) and/or a 3′ capture probe that includes the oligonucleotide sequence 5′-AAT GAA GAT TCA ACT CAA TAA GAA ACA ACA-3′ (SEQ ID NO: 20) to detect the presence of Enterococcus faecalis in a biological sample. In some embodiments, the 5′ capture probe and/or the 3′ capture probe is conjugated to a magnetic nanoparticle.

In some embodiments, a control target nucleic acid for Enterococcus faecium may comprise the nucleic acid sequence of SEQ ID NO: 32. In other embodiments, a control target nucleic acid for Enterococcus faecium may comprise the nucleic acid sequence of SEQ ID NO: 59. In some embodiments, a control target nucleic acid for Enterococcus faecalis may comprise the nucleic acid sequence of SEQ ID NO: 33.

Klebsiella Target Nucleic Acids

In some embodiments, a target nucleic acid may include sequence elements that are specific for a Klebsiella spp., for example, Klebsiella pneumoniae. For example, in some embodiments, a Klebsiella pneumoniae target nucleic acid may be amplified in the presence of a forward primer and a reverse primer which are specific to Klebsiella pneumoniae, as described below. Detection of such a target nucleic acid in a sample would typically indicate that a Klebsiella pneumoniae bacterium was present in the sample. In other embodiments, a target nucleic acid of the invention may include sequence elements that are common to all Klebsiella spp. For example, in some embodiments, a Klebsiella spp. target nucleic acid may be amplified in the presence of a forward primer and a reverse primer, each of which is universal to all Klebsiella spp. Detection of such a target nucleic acid in a sample typically would indicate that a Klebsiella spp. bacterium was present in the sample. In yet other embodiments, these approaches may be combined.

In some embodiments, a Klebsiella spp. target nucleic acid may be derived from a linear chromosome or a linear or circular plasmid (e.g., a single-, low-, or multi-copy plasmid). In some embodiments, a Klebsiella spp. target nucleic acid may be derived from an essential locus (e.g., an essential housekeeping gene) or a locus involved in virulence (e.g., a gene essential for virulence). In some embodiments, a Klebsiella spp. target nucleic acid may be derived from a multi-copy locus. In particular embodiments, a Klebsiella spp. target nucleic acid may be derived from a multi-copy plasmid.

In some embodiments, a Klebsiella pneumoniae target nucleic acid is derived from a 23S rRNA gene. In particular embodiments, a Klebsiella pneumoniae target nucleic acid may be amplified in the presence of a forward primer that includes the oligonucleotide sequence 5′-GAC GGT TGT CCC GGT TTA AGC A-3′ (SEQ ID NO: 5) and a reverse primer that includes the oligonucleotide sequence 5′-GCT GGT ATC TTC GAC TGG TCT-3′ (SEQ ID NO: 6). In some embodiments, an amplicon produced using these primers is detected by sequencing. In some embodiments, an amplicon produced using these primers is detected by hybridization using a 5′ capture probe that includes the oligonucleotide sequence 5′-TAC CAA GGC GCT TGA GAG AAC TC-3′ (SEQ ID NO: 21) and/or a 3′ capture probe that includes the oligonucleotide sequence 5′-CTG GTG TGT AGG TGA AGT C-3′ (SEQ ID NO: 22) to detect the presence of Klebsiella pneumoniae in a biological sample. In some embodiments, the 5′ capture probe and/or the 3′ capture probe is conjugated to a magnetic nanoparticle.

In some embodiments, a control target nucleic acid for Klebsiella pneumoniae may comprise the nucleic acid sequence of SEQ ID NO: 34.

Pseudomonas Target Nucleic Acids

In some embodiments, a target nucleic acid may include sequence elements that are specific for a Pseudomonas spp., for example, Pseudomonas aeruginosa. For example, in some embodiments, a Pseudomonas aeruginosa target nucleic acid may be amplified in the presence of a forward primer and a reverse primer which are specific to Pseudomonas aeruginosa, as described below. Detection of such a target nucleic acid in a sample would typically indicate that a Pseudomonas aeruginosa bacterium was present in the sample. In other embodiments, a target nucleic acid of the invention may include sequence elements that are common to all Pseudomonas spp. For example, in some embodiments, a Pseudomonas spp. target nucleic acid may be amplified in the presence of a forward primer and a reverse primer, each of which is universal to all Pseudomonas spp. Detection of such a target nucleic acid in a sample typically would indicate that a Pseudomonas spp. bacterium was present in the sample. In yet other embodiments, these approaches may be combined.

In some embodiments, a Pseudomonas spp. target nucleic acid may be derived from a linear chromosome or a linear or circular plasmid (e.g., a single-, low-, or multi-copy plasmid). In some embodiments, a Pseudomonas spp. target nucleic acid may be derived from an essential locus (e.g., an essential housekeeping gene) or a locus involved in virulence (e.g., a gene essential for virulence). In some embodiments, a Pseudomonas spp. target nucleic acid may be derived from a multi-copy locus. In particular embodiments, a Pseudomonas spp. target nucleic acid may be derived from a multi-copy plasmid.

In some embodiments, a Pseudomonas aeruginosa target nucleic acid is derived from a region that includes parts or all of the ITS between the 23S and 5S rRNA genes. In particular embodiments, a Pseudomonas aeruginosa target nucleic acid may be amplified in the presence of a forward primer that includes the oligonucleotide sequence 5′-AGG CTG GGT GTG TAA GCG TTG T-3′ (SEQ ID NO: 7) and a reverse primer that includes the oligonucleotide sequence 5′-CAA GCA ATT CGG TTG GAT ATC CGT T-3′ (SEQ ID NO: 8). In some embodiments, an amplicon produced using these primers is detected by sequencing. In some embodiments, an amplicon produced using these primers is detected by hybridization using a 5′ capture probe that includes the oligonucleotide sequence 5′-GTG TGT TGT AGG GTG AAG TCG AC-3′ (SEQ ID NO: 23) or 5′-TCT GAC GAT TGT GTG TTG TAA GG-3′ (SEQ ID NO: 57) and/or a 3′ capture probe that includes the oligonucleotide sequence 5′-CAC CTT GAA ATC ACA TAC CTG A-3′ (SEQ ID NO: 24) or 5′-GGA TAG ACG TAA GCC CAA GC-3′ (SEQ ID NO: 58) to detect the presence of Pseudomonas aeruginosa in a biological sample. In particular embodiments, an amplicon produced using these primers is detected by hybridization using a 5′ capture probe that includes the oligonucleotide sequence 5′-TCT GAC GAT TGT GTG TTG TAA GG-3′ (SEQ ID NO: 57) and/or a 3′ capture probe that includes the oligonucleotide 5′-GGA TAG ACG TAA GCC CAA GC-3′ (SEQ ID NO: 58) to detect the presence of Pseudomonas aeruginosa in a biological sample. In some embodiments, the 5′ capture probe and/or the 3′ capture probe is conjugated to a magnetic nanoparticle.

In some embodiments, a control target nucleic acid for Pseudomonas aeruginosa may comprise the nucleic acid sequence of SEQ ID NO: 35.

Staphylococcus Target Nucleic Acids

In some embodiments, a target nucleic acid may include sequence elements that are specific for a Staphylococcus spp., for example, Staphylococcus aureus. For example, in some embodiments, a Staphylococcus aureus target nucleic acid may be amplified in the presence of a forward primer and a reverse primer which are specific to Staphylococcus aureus, as described below. Detection of such a target nucleic acid in a sample would typically indicate that a Staphylococcus aureus bacterium was present in the sample. In other embodiments, a target nucleic acid of the invention may include sequence elements that are common to all Staphylococcus spp. For example, in some embodiments, a Staphylococcus spp. target nucleic acid may be amplified in the presence of a forward primer and a reverse primer, each of which is universal to all Staphylococcus spp. Detection of such a target nucleic acid in a sample typically would indicate that a Staphylococcus spp. bacterium was present in the sample. In yet other embodiments, these approaches may be combined.

In some embodiments, a Staphylococcus spp. target nucleic acid may be derived from a linear chromosome or a linear or circular plasmid (e.g., a single-, low-, or multi-copy plasmid). In some embodiments, a Staphylococcus spp. target nucleic acid may be derived from an essential locus (e.g., an essential housekeeping gene), a locus involved in virulence (e.g., a gene essential for virulence), or a gene involved in antibiotic resistance (e.g., femA and femB). In some embodiments, a Staphylococcus spp. target nucleic acid may be derived from a multi-copy locus. In particular embodiments, a Staphylococcus spp. target nucleic acid may be derived from a multi-copy plasmid.

In some embodiments, a Staphylococcus aureus target nucleic acid is derived from the femAB operon. The femAB operon codes for two nearly identical approximately 50 kDa proteins involved in the formation of the Staphylococcal pentaglycine interpeptide bridge in peptidoglycan. These chromosomally-encoded proteins are considered as factors that influence the level of methicillin resistance and as essential housekeeping genes. femB is one gene in the femA/B operon, also referred to as graR, the two-component response regulator of methicillin resistance. femB encodes an aminoacyltransferase, whereas femA encodes a regulatory factor that is essential for expression of femB and therefore methicillin resistance expression. In some embodiments, a Staphylococcus aureus target nucleic acid is derived from the femA gene. For example, in particular embodiments, a Staphylococcus aureus target nucleic acid may be amplified in the presence of a forward primer that includes the oligonucleotide sequence 5′-GGT AAT GAATTA CCT/i6diPr/TC TCT GCT GGTTTC TTC TT-3′ (SEQ ID NO: 9) and a reverse primer that includes the oligonucleotide sequence 5′-ACC AGC ATC TTC/i6diPr/GC ATC TTC TGT AAA-3′ (SEQ ID NO: 10). Note that “/i6diPr/” indicates 2,6-Diaminopurine. In some embodiments, an amplicon produced using these primers is detected by sequencing. In some embodiments, an amplicon produced using these primers is detected by hybridization using a 5′ capture probe that includes the oligonucleotide sequence 5′-CCA TTT GAA GTT GTT TAT TAT GC-3′ (SEQ ID NO: 25) and/or a 3′ capture probe that includes the oligonucleotide sequence 5′-GGG AAA TGA TTA ATT ATG CAT TAA ATC-3′ (SEQ ID NO: 26) to detect the presence of Staphylococcus aureus in a biological sample. In some embodiments, an amplicon produced using these primers is detected by sequencing. In some embodiments, the 5′ capture probe and/or the 3′ capture probe is conjugated to a magnetic nanoparticle.

In some embodiments, a Staphylococcus aureus target nucleic acid is derived from the femB gene. For example, in other particular embodiments, a Staphylococcus aureus target nucleic acid may be amplified in the presence of a forward primer that includes the oligonucleotide sequence 5′-GAA GTT ATG TTT /i6diPr/CT ATT CGA ATC GTG GTC CAGT-3′ (SEQ ID NO: 11) and a reverse primer that includes the oligonucleotide sequence 5′-GTT GTA AAG CCA TGA TGC TCG TAA CCA-3′ (SEQ ID NO: 12). In some embodiments, an amplicon produced using these primers is detected by sequencing. In some embodiments, an amplicon produced using these primers is detected by hybridization using a 5′ capture probe that includes the oligonucleotide sequence 5′-TT TTT CAG ATT TAG GAT TAG TTG ATT-3′ (SEQ ID NO: 27) and/or a 3′ capture probe that includes the oligonucleotide sequence 5′-GAT CCG TAT TGG TTA TAT CAT C-3′ (SEQ ID NO: 28) to detect the presence of Staphylococcus aureus in a biological sample. In some embodiments, the 5′ capture probe and/or the 3′ capture probe is conjugated to a magnetic nanoparticle.

In some embodiments, a Staphylococcus aureus target nucleic acid includes all or a portion of both the femA gene and the femB gene.

In some embodiments, a control target nucleic acid for Staphylococcus aureus femA may comprise the nucleic acid sequence of SEQ ID NO: 36. In some embodiments, a control target nucleic acid for Staphylococcus aureus femB may comprise the nucleic acid sequence of SEQ ID NO: 37.

Escherichia Target Nucleic Acids

In some embodiments, a target nucleic acid may include sequence elements that are specific for an Escherichia spp., for example, Escherichia coli. For example, in some embodiments, an Escherichia coli target nucleic acid may be amplified in the presence of a forward primer and a reverse primer which are specific to Escherichia coli, as described below. Detection of such a target nucleic acid in a sample would typically indicate that an Escherichia coli bacterium was present in the sample. In other embodiments, a target nucleic acid of the invention may include sequence elements that are common to all Staphylococcus spp. For example, in some embodiments, an Escherichia spp. target nucleic acid may be amplified in the presence of a forward primer and a reverse primer, each of which is universal to all Escherichia spp. Detection of such a target nucleic acid in a sample typically would indicate that a Escherichia spp. bacterium was present in the sample. In yet other embodiments, these approaches may be combined.

In some embodiments, an Escherichia spp. target nucleic acid may be derived from a linear chromosome or a linear or circular plasmid (e.g., a single-, low-, or multi-copy plasmid). In some embodiments, an Escherichia spp. target nucleic acid may be derived from an essential locus (e.g., an essential housekeeping gene), a locus involved in virulence (e.g., a gene essential for virulence), or a gene involved in antibiotic resistance. In some embodiments, an Escherichia spp. target nucleic acid may be derived from a multi-copy locus. In particular embodiments, an Escherichia spp. target nucleic acid may be derived from a multi-copy plasmid.

In particular embodiments, an Escherichia coli target nucleic acid is derived from the yfcL gene. The yfcL gene is within an E. coli-specific Chaperone-Usher Fimbriae gene cluster (see, e.g., Wurpelet al. PLoS One Vol 8, e52835, 2013). For example, in other particular embodiments, Escherichia coli yfcL may be amplified in the presence of a forward primer that includes the oligonucleotide sequence 5′-GCA TTA ATC GAC GGT ATG GTT GAC C-3′ (SEQ ID NO: 38) and a reverse primer that includes the oligonucleotide sequence 5′-CCT GCT GAA ACA GGT TTT CCC ACA TA-3′ (SEQ ID NO: 39). In some embodiments, an amplicon produced using these primers is detected by sequencing. In some embodiments, an amplicon produced using these primers is detected by hybridization using a 5′ capture probe that includes the oligonucleotide sequence 5′-AGT GAT GAT GAG TTG TTT GCC AGT G-3′ (SEQ ID NO: 40) and/or a 3′ capture probe that includes the oligonucleotide sequence 5′-TGA ATT GTC GCC GCG TGA CCA G-3′ (SEQ ID NO: 41) to detect the presence of Escherichia coli in a biological sample. In some embodiments, the 5′ capture probe and/or the 3′ capture probe is conjugated to a magnetic nanoparticle.

Candida Target Nucleic Acids

In some embodiments, a target nucleic acid may include sequence elements that are specific for a Candida spp. (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis). For example, in some embodiments, a Candida albicans target nucleic acid may be amplified in the presence of a forward primer and a reverse primer which are specific to Candida albicans. Detection of such a target nucleic acid in a sample would typically indicate that a Candida albicans cell was present in the sample. In other embodiments, a target nucleic acid of the invention may include sequence elements that are common to all Candida spp. For example, in some embodiments, a Candida spp. target nucleic acid may be amplified in the presence of a forward primer and a reverse primer, each of which is universal to all Candida spp., as described below. Detection of such a target nucleic acid in a sample typically would indicate that a Candida spp. cell was present in the sample. In yet other embodiments, these approaches may be combined.

In some embodiments, a Candida spp. target nucleic acid may be derived from a linear chromosome or a linear or circular plasmid (e.g., a single-, low-, or multi-copy plasmid). In some embodiments, a Candida spp. target nucleic acid may be derived from an essential locus (e.g., an essential housekeeping gene) or a locus involved in virulence (e.g., a gene essential for virulence). In some embodiments, a Candida spp. target nucleic acid may be derived from a multi-copy locus. For example, in some embodiments, a Candida spp. target nucleic acid may be derived from a ribosomal DNA operon.

Detection of a Candida species can be performed as described, for example, in International Patent Application Publication No. WO 2012/054639, which is incorporated herein by reference in its entirety. In particular embodiments, a Candida spp. target nucleic acid may be amplified in the presence of a forward primer that includes the oligonucleotide sequence 5′-GGC ATG CCT GTT TGA GCG TC-3′ (SEQ ID NO: 13) or 5′-GGG CAT GCC TGT TTG AGC GT-3′ (SEQ ID NO: 62) and a reverse primer that includes the oligonucleotide sequence 5′-GCT TAT TGA TAT GCT TAA GTT CAG CGG GT-3′ (SEQ ID NO: 14). In some embodiments, an amplicon produced using these primers is detected by sequencing. For example, in some embodiments, a Candida species target nucleic acid may be amplified in the presence of a forward primer that includes the oligonucleotide sequence 5′-GGC ATG CCT GTT TGA GCG T-3′ (SEQ ID NO: 13) and a reverse primer that includes the oligonucleotide sequence 5′-GCT TAT TGA TAT GCT TAA GTT CAG CGG GT-3′ (SEQ ID NO: 14). In other embodiments, a Candida species target nucleic acid may be amplified in the presence of a forward primer that includes the oligonucleotide sequence 5′-GGG CAT GCC TGT TTG AGC GT-3′ (SEQ ID NO: 62) and a reverse primer that includes the oligonucleotide sequence 5′-GCT TAT TGA TAT GCT TAA GTT CAG CGG GT-3′ (SEQ ID NO: 14). The capture probes listed in Table D can be used for detection (e.g., T2MR detection) of an amplicon produced by these primers to identify the presence of the indicated Candida species. The dual target probe pair will detect either or both targets present in a sample.

TABLE D
Capture Probes for Detection of Candida spp.

| Candida Capture Probes | Sequence |
|---|---|
| Candida albicans Probe #1 | 5′-ACC CAG CGG TTT GAG GGA GAA AC-3′ (SEQ ID NO: 42) |
| Candida albicans Probe #2 | 5′-AAA GTT TGA AGA TAT ACG TGG TGG ACG TTA-3′ (SEQ ID NO: |
| | 43) |
| Candida krusei Probe #1 | 5′-CGC ACG CGC AAG ATG GAA ACG-3′ (SEQ ID NO: 44) |
| Candida krusei Probe #2 | 5′-AAG TTC AGC GGG TAT TCC TAC CT-3′ (SEQ ID NO: 45) |
| Candida krusei probe | 5′-AGC TTT TTG TTG TCT CGC AAC ACT CGC-3′ (SEQ ID NO: 46) |
| Candida glabrata Probe #1 | 5′-CTA CCA AAC ACA ATG TGT TTG AGA AG-3′ (SEQ ID NO: 47) |
| Candida glabrata Probe #2 | 5′-CCT GAT TTG AGG TCA AAC TTA AAG ACG TCT G-3′ (SEQ ID NO: |
| | 48) |
| Candida | 5′-AGT OCT ACC TGA TTT GAG GTC NitInd¹AA-3′ (SEQ ID NO: 49) |
| parapsilosis/tropicalis Probe | |
| #1 | |
| Candida | 5′-CCG NitInd¹GG GTT TGA GGG AGA AAT-3′ (SEQ ID NO: 50) |
| parapsilosis/tropicalis Probe | |
| #2 | |
| Candida tropicalis | 5′-AAA GTT ATG AAATAA ATT GTG GTG GCC ACT AGC-3′ (SEQ ID |
| | NO: 51) |
| Candida tropicalis | 5′-ACC CGG GGGTTT GAG GGA GAA A-3′ (SEQ ID NO: 52) |
| Candida parapsilosis | 5′-AGT CCT ACC TGA TTT GAG GTC GAA-3′ (SEQ ID NO: 53) |
| Candida parapsilosis | 5′-CCG AGG GTT TGA GGG AGA AAT-3′ (SEQ ID NO: 54) |
| Candida auris 5′ Capture | 5′-CTA CCT GAT TTG AGG CGA CAA CAA AAC-3′ (SEQ ID NO: 63) |
| probe | |
| Candida auris 3′ Capture | 5′-CCG CGA AGA TTG GTG AGA AGA CAT-3′ (SEQ ID NO: 64) |
| probe | |
| Candida lusitaniae 5′ | 5′-CCT ACC TGA TTT GAG GGC GAA ATG TC-3′ (SEQ ID NO: 65) |
| Capture probe | |
| Candida lusitaniae 3′ | 5′-GGA GCA ACG CCT AAC CGG G-3′ (SEQ ID NO: 66) |
| Capture probe | |
| Candida haemulonii 5′ | 5′-GTC CTA CCT GAT TTG AGG GGA AAA AGC-3′ (SEQ ID NO: 67) |
| Capture probe | |
| Candida haemulonii 3′ | 5′-AAC AAA TCC ACC AAC GGT GAG AAG ATA T-3′ (SEQ ID NO: 68) |
| Capture probe | |
| Candida duobushaemulonii | 5′-GCG TAG ACT TCG CTG CGG AT-3′ (SEQ ID NO: 69) |
| 5′ Capture probe | |
| Alternate Candida | 5′-CGT AGA CTT CGC TGC GGA T-3′ (SEQ ID NO: 70) |
| duobushaemulonii 5′ | |
| Capture probe | |
| Candida duobushaemulonii | 5′-CTG GGC GGT GAG AAG AAA TC-3′ (SEQ ID NO: 71) |
| 3′ Capture probe | |
| Candida pseudohaemulonii | 5′-GCG TAG ACT TCG CTG CTG GAA-3′ (SEQ ID NO: 72) |
| 5′ Capture probe | |
| Candida pseudohaemulonii | 5′-CCG TGC GGT GAG AAG AAA TC-3′ (SEQ ID NO: 73) |
| 3′ Capture probe | |
| Candida duobushaemulonii | 5′-TCC TAC CTG ATT TGA GGA AAT AGC ATG G-3′ (SEQ ID NO: 74) |
| and Candida | |
| pseudohaemulonii (dual | |
| target) 5′ Capture probe | |
| Candida duobushaemulonii | 5′-ATT TAG CGG ATG CAA AAC CAC C-3′ (SEQ ID NO: 75) |
| and Candida | |
| pseudohaemulonii (dual | |
| target) 3′ Capture probe | |

¹NitInd is 5′ 5-Nitroindole, a base that is capable of annealing with any of the four DNA bases.

In some methods, a Candida amplicon produced by amplification of a Candida target nucleic acid in the presence of a forward primer comprising the oligonucleotide sequence 5′-GGC ATG CCT GTT TGA GCG TC-3′ (SEQ ID NO: 13) and a reverse primer that includes the oligonucleotide sequence 5′-GCT TAT TGA TAT GCT TAA GTT CAG CGG GT-3′ (SEQ ID NO: 14) is detected by hybridization a first nucleic acid probe and a second nucleic acid probe conjugated to one or more populations of magnetic particles. For example, certain embodiments, (i) the Candida species is Candida albicans, the first probe includes the oligonucleotide sequence 5′-ACC CAG CGG TTT GAG GGA GAA AC-3′ (SEQ ID NO: 42), and the second probe includes the oligonucleotide sequence 5′-AAA GTT TGA AGA TAT ACG TGG TGG ACG TTA-3′ (SEQ ID NO: 43); (ii) the Candida species is Candida krusei and the first probe and the second probe include an oligonucleotide sequence selected from: 5′-CGC ACG CGC AAG ATG GAA ACG-3′ (SEQ ID NO: 44), 5′-AAG TTC AGC GGG TAT TCC TAC CT-3′ (SEQ ID NO: 45), and 5′-AGC TTT TTG TTG TCT CGC AAC ACT CGC-3′ (SEQ ID NO: 46); (iii) the Candida species is Candida glabrata, the first probe includes the oligonucleotide sequence: 5′-CTA CCA AAC ACA ATG TGT TTG AGA AG-3′ (SEQ ID NO: 47), and the second probe includes the oligonucleotide sequence: 5′-CCT GAT TTG AGG TCA AAC TTA AAG ACG TCT G-3′ (SEQ ID NO: 48); and (iv) the Candida species is Candida parapsilosis or Candida tropicalis and the first probe and the second probe include an oligonucleotide sequence selected from: 5′-AGT CCT ACC TGA TTT GAG GTCNitIndAA-3′ (SEQ ID NO: 49), 5′-CCG NitlndGG GTT TGA GGG AGA AAT-3′ (SEQ ID NO: 50), 5′-AAA GTT ATG AAATAA ATT GTG GTG GCC ACT AGC-3′ (SEQ ID NO: 51), 5′-ACC CGG GGGTTT GAG GGA GAA A-3′ (SEQ ID NO: 52), 5′-AGT CCT ACC TGA TTT GAG GTC GAA-3′ (SEQ ID NO: 53), and 5′-CCG AGG GTT TGA GGG AGA AAT-3′ (SEQ ID NO: 54). In some embodiments, the first probe comprises the oligonucleotide sequence of SEQ ID NO: 29 and the second probe comprises the oligonucleotide sequence of SEQ ID NO: 30.

In some methods, a Candida species amplicon produced by amplification of a Candida species target nucleic acid in the presence of a forward primer comprising the oligonucleotide sequence 5′-GGC ATG CCT GTT TGA GCG T-3′ (SEQ ID NO: 13) or 5′-GGG CAT GCC TGT TTG AGC GT-3′ (SEQ ID NO: 62) and a reverse primer that includes the oligonucleotide sequence 5′-GCT TAT TGA TAT GCT TAA GTT CAG CGG GT-3′ (SEQ ID NO: 14) is detected by hybridization a first nucleic acid probe and a second nucleic acid probe conjugated to one or more populations of magnetic particles. For example, in certain embodiments, (i) the Candida species is Candida auris, the first probe includes the oligonucleotide sequence 5′-CTA CCT GAT TTG AGG CGA CAA CAA AAC-3′ (SEQ ID NO: 63), and the second probe includes the oligonucleotide sequence 5′-CCG CGA AGA TTG GTG AGA AGA CAT-3′ (SEQ ID NO: 64); (ii) the Candida species is Candida lusitaniae, the first probe includes the oligonucleotide sequence 5′-CCT ACC TGA TTT GAG GGC GAA ATG TC-3′ (SEQ ID NO: 65), and the second probe includes the oligonucleotide sequence 5′-GGA GCA ACG CCT AAC CGG G-3′ (SEQ ID NO: 66); (iii) the Candida species is Candida haemulonii, the first probe includes the oligonucleotide sequence: 5′-GTC CTA CCT GAT TTG AGG GGA AAA AGC-3′ (SEQ ID NO: 67), and the second probe includes the oligonucleotide sequence: 5′-AAC AAA TCC ACC AAC GGT GAG AAG ATA T-3′ (SEQ ID NO: 68); (iv) the Candida species is Candida duobushaemulonii, the first probe includes the oligonucleotide sequence: 5′-CGT AGA CTT CGC TGC GGA T-3′ (SEQ ID NO: 70) or 5′-GCG TAG ACT TCG CTG CGG AT-3′ (SEQ ID NO: 69), and the second probe includes the oligonucleotide sequence: 5′-CTG GGC GGT GAG AAG AAA TC-3′ (SEQ ID NO: 71); (v) the Candida species is Candida pseudohaemulonii, the first probe includes the oligonucleotide sequence: 5′-GCG TAG ACT TCG CTG CTG GAA-3′ (SEQ ID NO: 72), and the second probe includes the oligonucleotide sequence: 5′-CCG TGC GGT GAG AAG AAA TC-3′ (SEQ ID NO: 73); and/or (vi) the Candida species is Candida duobushaemulonii or Candida pseudohaemulonii, the first probe includes the oligonucleotide sequence: 5′-TCC TAC CTG ATT TGA GGA AAT AGC ATG G-3′ (SEQ ID NO: 74), and the second probe includes the oligonucleotide sequence: 5′-ATT TAG CGG ATG CAA AAC CAC C-3′ (SEQ ID NO: 75).

Borrelia Target Nucleic Acids

In some embodiments, a target nucleic acid may include sequence elements that are specific for a Borrelia spp. (e.g., B. burgdorferi, B. afzelii, and B. garinii). For example, in some embodiments, a Borrelia burgdorferi target nucleic acid may be amplified in the presence of a forward primer and a reverse primer which are specific to Borrelia burgdorferi. Detection of such a target nucleic acid in a sample would typically indicate that a Borrelia burgdorferi cell was present in the sample. In other embodiments, a target nucleic acid of the invention may include sequence elements that are common to all Borrelia spp. For example, in some embodiments, a Borrelia spp. target nucleic acid may be amplified in the presence of a forward primer and a reverse primer, each of which is universal to all Borrelia spp. Detection of such a target nucleic acid in a sample typically would indicate that a Borrelia spp. cell was present in the sample. In yet other embodiments, these approaches may be combined.

In some embodiments, a Borrelia spp. target nucleic acid may be derived from a linear chromosome or a linear or circular plasmid (e.g., a single-, low-, or multi-copy plasmid). In some embodiments, a Borrelia spp. target nucleic acid may be derived from an essential locus (e.g., an essential housekeeping gene) or a locus involved in virulence (e.g., a gene essential for virulence). In some embodiments, a Borrelia spp. target nucleic acid may be derived from a multi-copy locus. For example, in some embodiments, a Candida spp. target nucleic acid may be derived from a ribosomal DNA operon.

Detection of a Candida species can be performed as described, for example, in International Patent Application Publication No. WO 2016/118766, which is incorporated herein by reference in its entirety. Any of the primers and probes described in WO 2016/118766 can be used in the present invention. In some embodiments, the primer(s) and/or probe(s) are described in Table E.

TABLE E
Primers and Probes for Amplification and Sequencing of Borrelia spp.

| Sequence | SEQ ID NO: | Species |
|---|---|---|
| 5′-GGAAATCTAACGAGAGAGCATGCT-3′ | 76 | OIC control F |
| 5′-CGATGCGTGACACCCAGGC-3′ | 77 | OIC control R |
| 5′-CAAGGTGCAATGACTTTGTTTGGGCA-3′ | 78 | B. afzelii F |
| 5′-GCAACTTCAAAGTGTACAGTATTGGTATCCC-3′ | 79 | B. afzeffi R |
| 5′-AGCTGTAGTTTAAGGCAAATGTTGG-3′ | 80 | B. burgdorferi F |
| 5′-AGGATCGCAAAATCAACCACAAACA-3′ | 81 | B. burgdorferi R |
| 5′-CCTAAATGTTAAACCCCTTGACAACCCA-3′ | 82 | B. garinii F |
| 5′-CCCATCAGGATATCCAGCTTCGG-3′ | 83 | B. garinii R |
| 5′-CTTTACCGATACTTCAATTTCACCGAGCTCCA-3′ | 84 | Pan Borrelia F |
| 5′-CACAGGTCTCTGCAAATCTGTAAAGAGAA-3′ | 85 | Pan Borrelia R |
| 5′-CGATGGTTCACGGGATTCTGCAATTC-3′ | 86 | OIC control 5′ |
| 5′-GAGACGTTTTGGATACATGTGAAAGAAGGC-3′ | 87 | OIC control 3′ |
| 5′-TTGTAGAACAATCTGGGCTTTTTGG-3′ | 88 | B. afzelii 5′ |
| 5′-GGAGAACTCATATCAGGAGCACAA-3′ | 89 | B. afzelii 3′ |
| 5′-GCTATTTCTGCTGTTAAAAGTTCTTGT-3′ | 90 | B. burgdorferi 5′ |
| 5′-CTAAAACTTAAGCTTTGCAATTGTGG-3′ | 91 | B. burgdorferi 3′ |
| 5′-TCTAGCGGTTGACAGAGAAACATTG-3′ | 92 | B. garinii 5′ |
| 5′-AAAAAATTAAAACCATATAACCCACGAA-3′ | 93 | B. garinii 3′ |
| 5′-GAGACAGCGTCCAAATCGTTACACC-3′ | 94 | Pan Borrelia 5′ |
| 5′-TCTTAACCTTCCAGCACCGGGCA-3′ | 95 | Pan Borrelia 3′ |

Variant Primers and Probes

In some embodiments, the invention features a primer that has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any of the preceding forward or reverse primers, or any forward or reverse primer described in the application. Such primers can be used in any of the methods of the invention described herein.

In some embodiments, the invention features a probe that has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100%) identity with any of the preceding probes.

In some embodiments, any of the preceding primers or probes may include one or more modified bases, for example, 2,6-Diaminopurine (abbreviated herein as “/i6diPr/”), deoxyinosine (abbreviated herein as “/ideoxyl/”), nitroindole (abbreviated herein as /35NiTInd/ or NitInd) or other modified bases known in the art.

Panels

The methods and compositions (e.g., systems, devices, or cartridges) described herein can be configured to detect and/or sequence target nucleic acids from a predetermined panel of pathogens. In some embodiments, the panel may be configured to individually detect between 1 and 18 (e.g., 1, 2, 3, 4, 5, 6, 7, 8, 9, 10, 11, 12, 13, 14, 15, 16, 17, or 18) pathogens selected from the following: Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (including, e.g., K. pneumoniae with resistance marker KPC) and Klebsiella oxytoca), Pseudomonas spp. (e.g., Pseudomonas aeruginosa), Staphylococcus spp. (including, e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter kosen), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexnen), and Enterobacter spp. (e.g., Enterobacter aerogenes and Enterobacter cloacae), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida)), Streptococcus spp. (including Streptococcus pneumonia), and Neisseria spp. (including Neisseria meningitidis). In some embodiments, the bacterial pathogen panel is further configured to detect a fungal pathogen, for example, Candida spp. (e.g., Candida albicans, Candida guilliermondii, Candida glabrata, Candida krusei, Candida lusitaniae, Candida parapsilosis, Candida dublinensis, and Candida tropicalis) and Aspergillus spp. (e.g., Aspergillus fumigatus). In some embodiments, the pathogen panel is further configured to detect a Candida spp. (including Candida albicans, Candida guilliermondii, Candida glabrata, Candida krusei, Candida lusitaniae, Candida parapsilosis, Candida dublinensis, and Candida tropicalis). In cases where multiple species of a genus are detected, the species may be detected using individual target nucleic acids or using target nucleic acids that are universal to all of the species, for example, target nucleic acids amplified using universal primers.

In some embodiments, the panel may be configured to individually detect one or more (e.g., 1, 2, 3, 4, 5, 6, or 7) of Acinetobacter baumannii, Enterococcus faecium, Enterococcus faecalis, Klebsiella pneumoniae, Pseudomonas aeruginosa, Escherichia coli, and Staphylococcus aureus.

In some embodiments, the panel may be configured to individually detect one or more (e.g., 1, 2, 3, 4, 5, 6, or 8) Candida spp. (e.g., Candida albicans, Candida guilliermondii, Candida glabrata, Candida krusei, Candida lusitaniae, Candida parapsilosis, Candida dublinensis, and Candida tropicalis).

In some embodiments, the panel can be a Lyme disease pathogen panel configured to individually detect one, two, or three Borrelia burgdorferi sensu lato (Borrelia burgdorferi, Borrelia afzelii, and Borrelia garinii) species. These species may be detected using individual target nucleic acids or using target nucleic acids that are universal to all three species, for example, target nucleic acids amplified using universal primers. In some embodiments, the panel is configured to detect Borrelia burgdorferi. In some embodiments, the panel is configured to detect Borrelia afzelii. In some embodiments, the panel is configured to detect Borrelia garinii. In some embodiments, the panel is configured to detect Borrelia burgdorferi and Borrelia afzelii. In some embodiments, the panel is configured to detect Borrelia burgdorferi and Borrelia garinii. In some embodiments, the panel is configured to detect Borrelia afzelii and Borrelia garinii. In some embodiments, the panel is configured to detect Borrelia burgdorferi, Borrelia afzelii and Borrelia garinii. In some embodiments, the panel may be configured to individually detect one or more (e.g., 1, 2, 3, 4, 5, or 6) of Rickettsia rickettsii, Coxiella burnettii, Ehrlichia chaffeensis, Babesia microti, Francisella tularensis, and Anaplasma phagocytophilum.

In some embodiments, the panel is a panel described in Table 24 below.

In some embodiments, the panel is a biothreat panel configured to detect one or more (e.g., 1, 2, 3, 4, 5, or 6) of Bacillus anthracis, Francisella tularensis, Burkholderia spp. (e.g., B. mallei or B. pseudomallei), Yersinia pestis, and Rickettsia prowazekii.

In any of the above embodiments, the panel may be configured to detect a marker that is characteristic of a genus, for example, a pan-bacterial marker, a pan-Candida marker, or a pan-Borrelia marker. In any of the above panels, the analyte may be a nucleic acid (e.g., an amplified target nucleic acid, as described above), or a polypeptide (e.g., a polypeptide derived from the pathogen or a pathogen-specific antibody produced by a host subject, for example, an IgM or IgG antibody). In some embodiments, multiple analytes (e.g., multiple amplicons) are used to detect a pathogen. In any of the above panels, the biological sample may be a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), or sputum. In some embodiments, the biological sample is blood (e.g., whole blood, a crude whole blood lysate, serum, or plasma). Such panels may be used, for example, to diagnose bloodstream infections. In some embodiments, the biological sample may be a tissue sample, for example, a homogenized tissue sample. Such panels may be used, for example, to detect infections present in tissue, e.g., tissue biopsies of skin at the site of a tick bite to identify Borrelia spp. for diagnosis of Lyme disease.

For example, in some embodiments, the panel may include a Pan-Bacterila marker (e.g., 16S) and/or a Pan-Fungal marker (e.g., ITS). Such a panel may further include one or more resistance genes (e.g., 1, 2, 3, 4, 5, 6, 7, 8, 9, 10, 11, 12, 13, 14, 15, 16, 17, 18, 19, 20, or more resistance genes). Any resistance gene may be included in the panel. For example, in some embodiments, the panel includes one or more of mecA, mecC, vanA, vanB, KPC, OXA-48, VIM, IMP, NDM, CMY, DHA, and CTX-M. In some embodiments, the panel includes one ore more (e.g., 1, 2, 3, 4, 5, 6, 7, 8, 9, 10, 11, 12, 13, or all 14) of the following: a Pan-Bacterial marker (e.g., 16S), a Pan-Fungal marker (e.g., ITS), mecA, mecC, vanA, vanB, KPC, OXA-48, VIM, IMP, NDM, CMY, DHA, and CTX-M. In some embodiments, the panel is a panel described in Table 30 below. In some embodiments, the panel is a toxin gene panel. For example, in some embodiments, the toxin gene panel includes on or more of Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G.

Medical Conditions

The methods of the invention can also be used diagnose and monitor diseases and other medical conditions. In some embodiments, the methods of the invention may be used diagnose and monitor diseases in a multiplexed, automated, no sample preparation system.

The methods and systems of the invention can be used to identify and monitor the pathogenesis of disease in a subject, to select therapeutic interventions, and to monitor the effectiveness of the selected treatment. For example, for a patient having or at risk of bacteremia and/or sepsis, the methods and systems of the invention can be used to identify the infectious pathogen, pathogen load, and to monitor white blood cell count and/or biomarkers indicative of the status of the infection. The identity of the pathogen (e.g., at a group-level and/or a species-level) can be used to select an appropriate therapy. In some embodiments, the methods may further include administering a therapeutic agent following monitoring or diagnosing an infectious disease. The therapeutic intervention (e.g., a particular antibiotic agent) can be monitored as well to correlate the treatment regimen to the circulating concentration of antibiotic agent and pathogen load to ensure that the patient is responding to treatment.

Exemplary diseases that can be diagnosed and/or monitored by the methods and systems of the invention include diseases caused by or associated with microbial pathogens (e.g., bacterial infection or fungal infection), endocarditis, transplant-associated infection, Lyme disease, bloodstream infection (e.g., bacteremia or fungemia), pneumonia, peritonitis, osteomyeletis, meningitis, empyema, urinary tract infection, sepsis, septic shock, and septic arthritis) and diseases that may manifest with similar symptoms to diseases caused by or associated with microbial pathogens (e.g., SIRS).

For example, the methods and systems of the invention may be used to diagnose and/or monitor a disease caused by the following non-limiting examples of pathogens: bacterial pathogens, including Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (e.g., K. pneumoniae with resistance marker KPC) and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter kosen), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexnen), and Enterobacter spp. (e.g., Enterobacter aerogenes and Enterobacter cloacae); and fungal pathogens including but not limited to Candida spp. (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis) and Aspergillus spp. (e.g., Aspergillus fumigatus). In some embodiments, the pathogen may be a Borrelia spp., including Borrelia burgdorferi sensu lato (Borrelia burgdorferi, Borrelia afzelii, and Borrelia garinii) species, Borrelia americana, Borrelia andersonii, Borrelia bavariensis, Borrelia bissettii, Borrelia carolinensis, Borrelia californiensis, Borrelia chilensis, Borrelia genomosp. 1 and 2, Borrelia japonica, Borrelia kurtenbachii, Borrelia lusitaniae, Borrelia myomatoii, Borrelia sinica, Borrelia spielmanii, Borrelia tanukii, Borrelia turdi, Borrelia valaisiana and unclassified Borrelia spp. In other embodiments, the pathogen may be selected from the following: Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella bumetii), Babesia spp. (including Babesia microti and Babesia divergens), Anaplasma spp. (including Anaplasma phagocytophilum), Francisella spp., (including Francisella tularensis (including Francisella tularensis subspp. holarctica, mediasiatica, and novicida)), Streptococcus spp. (including Streptococcus pneumonia), and Neisseria spp. (including Neisseria meningitidis). In some embodiments, the pathogen is a viral pathogen (e.g., a retrovirus (e.g., HIV), an adeno-associated virus (AAV), an adenovirus, Ebolavirus, hepatitis (e.g., hepatitis A, B, C, or E), herpesvirus, human papillomavirus (HPV), rhinovirus, influenza, parainfluenza, measles, rotavirus, West Nile virus, zika virus, and the like). In some embodiments, the pathogen is a biothreat species, e.g., Bacillus anthracis, Francisella tularensis, Burkholderia spp. (e.g., B. mallei or B. pseudomallei), Yersinia pestis, or Rickettsia prowazekii.

Treatment

In some embodiments, the methods further include administering a therapeutic agent or a composition thereof (e.g., a pharmaceutical composition) to a subject following a diagnosis. Typically, the identification of a particular pathogen in a biological sample obtained from the subject (e.g., a complex sample containing host cells and/or cell debris, e.g., blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), urine, CSF, SF, or sputum) will guide the selection of the appropriate therapeutic agent (e.g., antimicrobial agent, e.g., an antibiotic, an anti-fungal agent, and the like).

For example, for a bacterial infection (e.g., bacteremia), a therapy may include an antibiotic. In some instances, an antibiotic may be administered orally. In other instances, the antibiotic may be administered intravenously. Exemplary non-limiting antibiotics that may be used in the methods of the invention include but are not limited to, acrosoxacin, amifioxacin, amikacin, amoxycillin, ampicillin, aspoxicillin, azidocillin, azithromycin, aztreonam, balofloxacin, benzylpenicillin, biapenem, brodimoprim, cefaclor, cefadroxil, cefatrizine, cefcapene, cefdinir, cefetamet, ceftmetazole, cefoxitin, cefprozil, cefroxadine, ceftarolin, ceftazidime, ceftibuten, ceftobiprole, cefuroxime, cephalexin, cephalonium, cephaloridine, cephamandole, cephazolin, cephradine, chlorquinaldol, chlortetracycline, ciclacillin, cinoxacin, ciprofloxacin, clarithromycin, clavulanic acid, clindamycin, clofazimine, cloxacillin, colistin, danofloxacin, dapsone, daptomycin, demeclocycline, dicloxacillin, difloxacin, doripenem, doxycycline, enoxacin, enrofloxacin, erythromycin, fleroxacin, flomoxef, flucloxacillin, flumequine, fosfomycin, gentamycin, isoniazid, imipenem, kanamycin, levofloxacin, linezolid, mandelic acid, mecillinam, meropenem, metronidazole, minocycline, moxalactam, mupirocin, nadifloxacin, nafcillin, nalidixic acid, netilmycin, netromycin, nifuirtoinol, nitrofurantoin, nitroxoline, norfloxacin, ofloxacin, oxacillin, oxytetracycline, panipenem, pefloxacin, phenoxymethylpenicillin, pipemidic acid, piromidic acid, pivampicillin, pivmecillinam, polymixin-b, prulifloxacin, rufloxacin, sparfloxacin, sulbactam, sulfabenzamide, sulfacytine, sulfametopyrazine, sulphacetamide, sulphadiazine, sulphadimidine, sulphamethizole, sulphamethoxazole, sulphanilamide, sulphasomidine, sulphathiazole, teicoplanin, temafioxacin, tetracycline, tetroxoprim, tigecycline, tinidazole, tobramycin, tosufloxacin, trimethoprim, vancomycin, and pharmaceutically acceptable salts or esters thereof.

In another example, for a fungal infection, a treatment may include an antifungal agent. Exemplary antifungal agents include, but are not limited to, polyenes (e.g., amphotericin B, candicidin, filipin, hamycin, natamycin, nystatin, and rimocidin), azoles (e.g., imidazoles such as bifonazole, butoconazole, clotrimazole, eberconazole, econazole, fenticonazole, flutrimazole, isoconazole, ketoconazole, luliconazole, miconazole, omoconazole, oxiconazole, sertaconazole, sulconazole, and tioconazole; triazoles such as albaconazole, efinaconazole, epoxiconazole, fluconazole, isavuconazole, itraconazole, posaconazole, propiconazole, ravuconazole, terconazole, and voriconazole; and thiazoles such as abafungin), allylamines (e.g., amorolfin, butenafine, naftifine, and terbinafine), echinocandins (e.g., anidulafungin, caspofungin, and micafungin), and other antifungal agents including but not limited to benzoic acid, ciclopirox olamine, 5-flucytosin, griseofulvin, haloprogin, tolnaftate, aminocandin, chlordantoin, chlorphenesin, nifuroxime, undecylenic acid, crystal violet, and pharmaceutically acceptable salts or esters thereof.

In some embodiments, a method of treatment may include administering a treatment to an asymptomatic patient, for example, based on the detection and/or identification of a pathogen present in a biological sample derived from the patient by the methods of the invention. In other embodiments, a method of treatment may include administering a treatment to a symptomatic patient based on the detection of identification of a pathogen present in a biological sample derived from the patient by the methods of the invention. In several embodiments, the biological sample may contain cells, cell debris, and/or nucleic acids (e.g., DNA or RNA (e.g., mRNA)) derived from both the host subject and a pathogen, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), CSF, SF, or sputum (e.g., purulent sputum or bloody sputum). In some embodiments, the biological sample is blood (e.g., whole blood, a crude whole blood lysate, serum, or plasma) or a bloody fluid (e.g., wound exudate, phlegm, bile, and the like). In particular embodiments, the biological sample is whole blood. In other particular embodiments, the biological sample is a crude whole blood lysate.

In some embodiments, the treatment selected for a patient is based on the detection and/or identification of a pathogen by the methods of the invention. Appropriate treatments for different pathogen species are known in the art. In one example, if a Gram positive bacterium is detected in a biological derived from a patient, a method of treatment may involve administration of vancomycin. In another example, if a Gram negative bacterium is detected in a biological derived from a patient, a method of treatment may involve administration of pipercillin-tazobactam. In another example, in some embodiments, if an Acinetobacter spp. (e.g., Acinetobacter baumannii) is detected in a biological sample derived from a patient, a method of treatment may involve administration of colistin, meropenem, and/or gentamicin. In another example, in some embodiments, if a Klebsiella spp. (e.g., Klebsiella pneumoniae) is detected in a biological sample derived from a patient, a method of treatment may involve administration of meropenem. In yet another example, in some embodiments, if a Pseudomonas spp. (e.g., Pseudomonas aeruginosa) is detected in a biological sample derived from a patient, a method of treatment may involve administration of pipercillin-tazobactam. In a further example, in some embodiments, if an Escherichia spp. (e.g., Escherichia coli) is detected in a biological sample derived from a patient, a method of treatment may involve administration of meropenem. In another example, in some embodiments, if an Enterococcus spp. (e.g., Enterococcus faecium) is detected in a biological sample derived from a patient, a method of treatment may involve administration of daptomycin.

Systems and Cartridges

The invention provides systems for carrying out the methods of the invention. For example, in some embodiments, the systems include one or more sequencing units. In other embodiments, the system results in production of a sample that can be sequenced separately, for example, a sample that requires one or more further steps for sequencing (e.g., adaptor ligation and tagging). Such systems may further include other components for carrying out an automated assay of the invention, such as a thermocycling unit for the amplification of oligonucleotides; a centrifuge, a robotic arm for delivery an liquid sample from unit to unit within the system; one or more incubation units; a fluid transfer unit (i.e., pipetting device) for combining assay reagents and a biological sample (e.g., a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, CSF, SF, or sputum) to form the liquid sample; a computer with a programmable processor for storing data, processing data, and for controlling the activation and deactivation of the various units according to a one or more preset protocols; and a cartridge insertion system for delivering pre-filled cartridges to the system, optionally with instructions to the computer identifying the reagents and protocol to be used in conjunction with the cartridge. The systems may also include one or more NMR units, MAA units, cartridge units, and agitation units, as described in WO 2012/054639. Any of the systems described in WO 2012/054639 may be used for embodiments that involve T2MR detection, e.g., for providing group-level information to focus or narrow subsequent sequencing. For example, FIG. 42 of WO 2012/054639 depicts a system that can be used for embodiments involving T2MR detection. In some embodiments, the system stores a sample containing one or more amplified target nucleic acids for downstream sequencing.

The sequencing unit may include any system or device that is known in the art for sequencing, e.g., massively parallel sequencing, long-read sequencing, or Sanger sequencing. Exemplary sequencing devices include but are not limited to ILLUMINA® systems (e.g., the ILLUMINA® iSeq 100 system, MiniSeq® system, MiSeq® systems, NextSeq® series platforms, HiSeq® series platforms, HiSeq X® series platforms, and NovaSeq® 6000 system); the BGISEQ-500 system; the 10× Genomics Chromium™ system; Ion Torrent sequencing systems (e.g., Ion PGM™, Ion Proton™, Ion S5™, and Ion S5 XL); Oxford Nanopore systems (e.g., MinION and PromethiON); Pacific Biosystems systems (e.g., PacBio RS II or PacBio Sequel); and the Roche 454 system. Other sequencing systems are known in the art.

The systems of the invention can provide an effective means for high throughput detection and/or sequencing of analytes present in sample, e.g., an environmental sample or a biological sample from a subject. The detection methods may be used in a wide variety of circumstances including, without limitation, sequencing of nucleic acids, identification and/or quantification of analytes that are associated with specific biological processes, physiological conditions, disorders or stages of disorders. As such, the systems have a broad spectrum of utility in, for example, disease diagnosis, parental and forensic identification, disease onset and recurrence, individual response to treatment versus population bases, and monitoring of therapy. The devices and systems can provide a flexible system for personalized medicine. The system of the invention can be changed or interchanged along with a protocol or instructions to a programmable processor of the system to perform a wide variety of assays as described herein. The systems of the invention offer many advantages of a laboratory setting contained in a desk-top or smaller size automated instrument.

The invention provides methods and systems that may involve one or more cartridge units to provide a convenient method for placing all of the assay reagents (e.g., sequencing reagents) and consumables onto the system. For example, the cartridge units can include reagents for sequencing. Such reagents include, e.g., library preparation reagents (e.g., tagmentation reagents such as NEXTERA® XT library preparation reagents), buffers, adaptors, primers, enzymes (e.g., thermostable polymerases), and the like. The system can include a replaceable and/or interchangeable cartridge containing an array of wells pre-loaded, e.g., with sequencing reagents or magnetic particles, and designed for detection and/or sequencing of a particular analyte, e.g., a particular target nucleic acid. Alternatively, the system may be usable with different cartridges, each designed for detection and/or concentration measurements of different analytes, or configured with separate cartridge modules for reagent and detection for a given assay. The cartridge may be sized to facilitate insertion into and ejection from a housing for the preparation of a liquid sample which is transferred to other units in the system (e.g., a sequencing unit or an NMR unit). Any of the cartridges described in WO 2012/054639 can be used in the methods and systems described herein.

A modular cartridge can provide a simple means for cross contamination control during certain assays, including but not limited to distribution of amplification (e.g., PCR) products into multiple detection or sequencing aliquots. In addition, a modular cartridge can be compatible with automated fluid dispensing, and provides a way to hold reagents at very small volumes for long periods of time (in excess of a year). Finally, pre-dispensing these reagents allows concentration and volumetric accuracy to be set by the manufacturing process and provides for a point of care use instrument that is more convenient as it can require much less precise pipetting.

The modular cartridge can be designed for a multiplexed assay. The challenge in multiplexing assays is combining multiple assays which have incompatible assay requirements (i.e., different incubation times and/or temperatures) on one cartridge. The cartridge format depicted in FIGS. 14A-14C of WO 2012/054639 allows for the combination of different assays with dramatically different assay requirements. The cartridge features two main components: (i) a reagent module (i.e., the reagent strip portion) that contains all of the individual reagents required for the full assay panel (for example, a panel as described below), and (ii) the detection module. In some embodiments, a cartridge may be configured to detect and/or sequence target nucleic acids from 2 to 24 or more pathogens (e.g., 2, 3, 4, 5, 6, 7, 8, 9, 10, 11, 12, 13, 14, 15, 16, 17, 18, 19, 20, 21, 22, 23, 24, or more pathogens). The detection modules contain only the parts of the cartridge that carry through the incubation, and can carry single assays or several assays, as needed.

The cartridge units can further include one or more populations of magnetic particles, either as a liquid suspension or dried magnetic particles which are reconstituted prior to use. For example, the cartridge units of the invention can include a compartment including from 1×10⁶ to 1×10¹³ magnetic particles (e.g., from 1×10⁶ to 1×10⁸, 1×10⁷ to 1×10⁹, 1×10⁸ to 1×10¹⁰, 1×10⁹ to 1×10¹¹, 1×10¹⁰ to 1×10¹², 1×10¹¹ to 1×10¹³, or from 1×10⁷ to 5×10⁸ magnetic particles) for assaying a single liquid sample.

Assay Reagents

The methods and compositions (e.g., systems, devices, or cartridges) described herein may include any suitable reagents, for example, surfactants, buffer components, additives, chelating agents, and the like. The surfactant may be selected from a wide variety of soluble non-ionic surface active agents including surfactants that are generally commercially available under the IGEPAL® trade name from GAF Company. The IGEPAL® liquid non-ionic surfactants are polyethylene glycol p-isooctylphenyl ether compounds and are available in various molecular weight designations, for example, IGEPAL® CA720, IGEPAL® CA630, and IGEPAL® CA890. Other suitable non-ionic surfactants include those available under the trade name TETRONIC® 909 from BASF Corporation. This material is a tetra-functional block copolymer surfactant terminating in primary hydroxyl groups. Suitable non-ionic surfactants are also available under the ALPHONIC® trade name from Vista Chemical Company and such materials are ethoxylates that are non-ionic biodegradables derived from linear primary alcohol blends of various molecular weights. The surfactant may also be selected from poloxamers, such as polyoxyethylene-polyoxypropylene block copolymers, such as those available under the trade names SYNPERONIC® PE series (ICI), PLURONIC® series (BASF), Supronic, MONOLAN®, PLURACARE®, and PLURODAC®, polysorbate surfactants, such as TWEEN® 20 (PEG-20 sorbitan monolaurate), and glycols such as ethylene glycol and propylene glycol.

Such non-ionic surfactants may be selected to provide an appropriate amount of detergency for an assay without having a deleterious effect on assay reactions. In particular, surfactants may be included in a reaction mixture for the purpose of suppressing non-specific interactions among various ingredients of the aggregation assays of the invention. The non-ionic surfactants are typically added to the liquid sample prior in an amount from 0.01% (w/w) to 5% (w/w).

The non-ionic surfactants may be used in combination with one or more proteins (e.g., albumin, fish skin gelatin, lysozyme, or transferrin) also added to the liquid sample prior in an amount from 0.01% (w/w) to 5% (w/w).

Furthermore, the assays, methods, and cartridge units of the invention can include additional suitable buffer components (e.g., Tris base, selected to provide a pH of about 7.8 to 8.2 in the reaction milieu); and chelating agents to scavenge cations (e.g., ethylene diamine tetraacetic acid (EDTA), EDTA disodium, citric acid, tartaric acid, glucuronic acid, saccharic acid or suitable salts thereof).

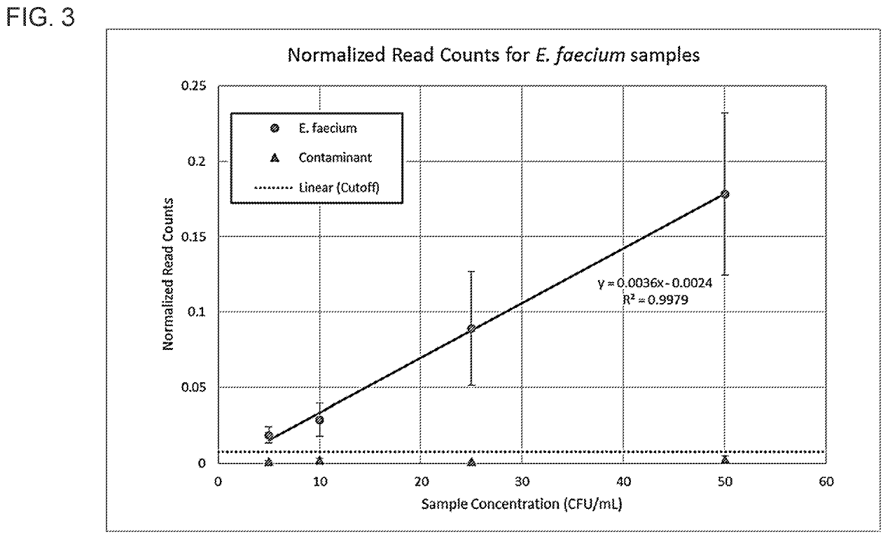

In some embodiments, the methods and systems of the invention may involve use of magnetic particles and NMR (e.g., T2MR). For example, T2MR can be used, for example, to obtain group-level information regarding a target nucleic acid, which can be used to narrow or focus sequencing analysis. The magnetic particles can be coated with a binding moiety (e.g., oligonucleotide, antibody, and the like) such that in the presence of analyte, or multivalent binding agent, aggregates are formed. Aggregation depletes portions of the sample from the microscopic magnetic non-uniformities that disrupt the solvent's T₂ signal, leading to an increase in T₂ relaxation (see, e.g., FIG. 3 of International Patent Application Publication No. WO 2012/054639, which is incorporated herein by reference in its entirety). Any NMR-based detection approach described in WO 2012/054639 may be used in the methods and systems described herein.

The T₂ measurement is a single measure of all spins in the ensemble, measurements lasting typically 1-10 seconds, which allows the solvent to travel hundreds of microns, a long distance relative to the microscopic non-uniformities in the liquid sample. Each solvent molecule samples a volume in the liquid sample and the T₂ signal is an average (net total signal) of all (nuclear spins) on solvent molecules in the sample; in other words, the T₂ measurement is a net measurement of the entire environment experienced by a solvent molecule, and is an average measurement of all microscopic non-uniformities in the sample.

The observed T₂ relaxation rate for the solvent molecules in the liquid sample is dominated by the magnetic particles, which in the presence of a magnetic field form high magnetic dipole moments. In the absence of magnetic particles, the observed T₂ relaxation rates for a liquid sample are typically long (i.e., T₂ (water)=approximately 2000 ms, T₂ (blood)=approximately 1500 ms). As particle concentration increases, the microscopic non-uniformities in the sample increase and the diffusion of solvent through these microscopic non-uniformities leads to an increase in spin decoherence and a decrease in the T₂ value. The observed T₂ value depends upon the particle concentration in a non-linear fashion, and on the relaxivity per particle parameter.

In embodiments that involve NMR detection, e.g., to provide initial group-level information, the number of magnetic particles, and if present the number of agglomerant particles, remain constant during the assay. The spatial distribution of the particles changes when the particles cluster. Aggregation changes the average “experience” of a solvent molecule because particle localization into clusters is promoted rather than more even particle distributions. At a high degree of aggregation, many solvent molecules do not experience microscopic non-uniformities created by magnetic particles and the T₂ approaches that of solvent. As the fraction of aggregated magnetic particles increases in a liquid sample, the observed T₂ is the average of the non-uniform suspension of aggregated and single (unaggregated) magnetic particles. The assays of the invention are designed to maximize the change in T₂ with aggregation to increase the sensitivity of the assay to the presence of analytes, and to differences in analyte concentration.

In some embodiments, the methods of the invention involve contacting a solution (e.g., a sample, e.g., a liquid sample, that includes whole blood or a crude whole blood lysate) with between from 1×10⁶ to 1×10¹³ magnetic particles per milliliter of the liquid sample (e.g., from 1×10⁶ to 1×10⁸, 1×10⁷ to 1×10⁸, 1×10⁷ to 1×10⁹, 1×10⁸ to 1×10¹⁰, 1×10⁹ to 1×10¹¹, or 1×10¹⁰ to 1×10¹³ magnetic particles per milliliter).

In some embodiments, the magnetic particles used in the methods and systems of the invention have a mean diameter of from 150 nm to 1200 nm (e.g., from 150 to 250, 200 to 350, 250 to 450, 300 to 500, 450 to 650, 500 to 700 nm, 700 to 850, 800 to 950, 900 to 1050, or from 1000 to 1200 nm). For example, in some embodiments, the magnetic particles used in the methods of the invention may have a mean diameter of from 150 nm to 699 nm (e.g., from 150 to 250, 200 to 350, 250 to 450, 300 to 500, 450 to 650, or from 500 to 699 nm). In other embodiments, the magnetic particles used in the methods of the invention may have a mean diameter of from 700 nm to 1200 nm (e.g., from 700 to 850, 800 to 950, 900 to 1050, or from 1000 to 1200 nm). In particular embodiments, the magnetic particles may have a mean diameter of from 700 nm to 950 nm (e.g., from 700 to 750, 700 to 800, 700 to 850, or from 700 to 900 nm).

In some embodiments, the magnetic particles used in the methods of the invention may have a T₂ relaxivity per particle of from 1×10⁸ to 1×10¹² mM⁻¹s⁻¹ (e.g., from 1×10⁸ to 1×10⁹, 1×10⁸ to 1×10¹⁰, 1×10⁹ to 1×10¹⁰, 1×10⁹ to 1×10¹¹, or from 1×10¹° to 1×10¹² mM⁻¹s⁻¹). In some embodiments, the magnetic particles have a T₂ relaxivity per particle of from 1×10⁹ to 1×10¹² mM⁻¹s⁻¹ (e.g., from 1×10⁹ to 1×10¹⁰, 1×10⁹ to 1×10¹¹, or from 1×10¹⁰ to 1×10¹² mM⁻¹s⁻¹).

In some embodiments, the magnetic particles may be substantially monodisperse. In some embodiments, the magnetic particles in a liquid sample (e.g., a biological sample containing cells and/or cell debris, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), or sputum) may exhibit nonspecific reversibility in the absence of the one or more analytes and/or multivalent binding agent. In some embodiments, the magnetic particles may further include a surface decorated with a blocking agent selected from albumin, fish skin gelatin, gamma globulin, lysozyme, casein, peptidase, and an amine-bearing moiety (e.g., amino polyethyleneglycol, glycine, ethylenediamine, or amino dextran.

The above methods can be used with any of the following categories of detection of aggregation or disaggregation described herein, including those described in WO 2012/054639, e.g., at pages 110-111.

Contamination Control

One potential problem in the use of amplification methods such as PCR as an analytical tool is the risk of having new reactions contaminated with old, amplified products. Such contamination could potentially affect downstream sequencing results as well. Potential sources of contamination include a) large numbers of target organisms in clinical specimens that may result in cross-contamination, b) plasmid clones derived from organisms that have been previously analyzed and that may be present in larger numbers in the laboratory environment, and c) repeated amplification of the same target sequence leading to accumulation of amplification products in the laboratory environment. A common source of the accumulation of the PCR amplicon is aerosolization of the product. Typically, if uncontrolled aerosolization occurs, the amplicon will contaminate laboratory reagents, equipment, and ventilation systems. When this happens, all reactions will be positive, and it is not possible to distinguish between amplified products from the contamination or a true, positive sample. In addition to taking precautions to avoid or control this carry-over of old products, preferred embodiments include a blank reference reaction in every PCR experiment to check for carry-over. For example, carry-over contamination will be visible on the agarose gel as faint bands or fluorescent signal when TaqMan® probes, MolBeacons®, or intercalating dyes, among others, are employed as detection mechanisms. Furthermore, it is preferred to include a positive sample. As an example, in some embodiments, contamination control is performed using any of the approaches and methods described in WO 2012/054639. In some embodiments, a bleach solution is used to neutralize potential amplicons, for example, in a reaction tube of a T2Dx® device being used to perform a method of the invention. In some embodiments, contamination control includes the use of ethylene oxide (EtO) treatment, for example, of cartridge components.

Typically, the instrumentation and processing areas for samples that undergo amplification are split into pre- and post-amplification zones. This minimizes the chances of contamination of samples with amplicon prior to amplification. For example, the T2Dx® instrument design is such that the pre- and post-amplification instrumentation and processing areas are integrated into a single instrument. This is made possible as described in the sections below.

Amplifying Multiple Amplicons Characteristic of a Species for Improved Sensitivity and/or Specificity

In some embodiments, the methods of the invention may involve amplification and detection of more than one amplicon characteristic of a species in a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), urine, CSF, SF, or sputum. In some embodiments, amplification of more than one target nucleic acid characteristic of a species increases the total amount of amplicons characteristic of the species in an assay (in other words, the amount of analyte is increased in the assay). This increase may allow, for example, an increase in sensitivity and/or specificity of detection of the species compared to a method that involves amplification and detection of a single amplicon characteristic of a species, e.g., for T2MR detection. In some embodiments, the methods of the invention may involve amplifying 2, 3, 4, 5, 6, 7, 8, 9, or 10 amplicons characteristic of a species.

In some embodiments, multiple (e.g., 2, 3, 4, 5, 6, 7, 8, 9, or 10) single-copy loci from a species are amplified and detected. In some embodiments, 2 single-copy loci from a species are amplified and detected. In some embodiments, amplification and detection of multiple single-copy loci from a species may allow for a sensitivity of detection comparable with methods that involve detecting an amplicon that is derived from a multi-copy locus. In some embodiments, methods involving detection of multiple single-copy loci amplified from a microbial species can detect from about 1-10 cells/mL (e.g., 1, 2, 3, 4, 5, 6, 7, 8, 9 or 10 cells/mL) of the microbial species in a liquid sample. In some embodiments, methods involving detection of multiple single-copy loci amplified from a microbial species have at least 95% correct detection when the microbial species is present in the liquid sample at a frequency of less than or equal to 5 cells/mL (e.g., 1, 2, 3, 4, or 5 cells/mL) of liquid sample.

The invention also provides embodiments in which at least three amplicons are produced by amplification of two target nucleic acids, each of which is characteristic of a species. For example, in some embodiments, a first target nucleic acid and a second target nucleic acid to be amplified may be separated (for example, on a chromosome or on a plasmid) by a distance ranging from about 50 base pairs to about 1000 1500 base pairs (bp), e.g., about 50, 100, 150, 200, 250, 300, 400, 500, 600, 700, 800, 900, or 1000, 1100, 1200, 1300, 1400, or 1500 bp base pairs. In some embodiments, a first target nucleic acid and a second target nucleic acid to be amplified may be separated (for example, on a chromosome or on a plasmid) by a distance ranging from about 50 bp to about 1000 bp (e.g., about 50, 100, 150, 200, 250, 300, 400, 500, 600, 700, 800, 900, or 1000 bp). In some embodiments the first target nucleic acid and the second target nucleic acid to be amplified may be separated by a distance ranging from about 50 bp to about 1500 bp, from about 50 bp to about 1400 bp, from about 50 bp to about 1300 bp, from about 50 bp to about 1200 bp, from about 50 bp to about 1100 bp, from about 50 bp to about 1000 bp, from about 50 bp to about 950 bp, from about 50 bp to about 900 bp, from about 50 bp to about 850 bp, from about 50 bp to about 800 bp, from about 50 bp to about 800 bp, from about 50 bp to about 750 bp, from about 50 bp to about 700 bp, from about 50 bp to about 650 bp, from about 50 bp to about 600 bp, from about 50 bp to about 550 bp, from about 50 bp to about 500 bp, from about 50 bp to about 500 bp, from about 50 bp to about 450 bp, from about 50 bp to about 400 bp, from about 50 bp to about 350 bp, from about 50 bp to about 300 bp, from about 50 bp to about 250 bp, from about 50 bp to about 200 bp, from about 50 bp to about 150 bp, or from about 50 bp to about 100 bp. In some embodiments, amplification of the first and second target nucleic acids using individual primer pairs (each having a forward and a reverse primer) may lead to amplification of an amplicon that includes the first target nucleic acid, an amplicon that includes the second target nucleic acid, and an amplicon that contains both the first and the second target nucleic acid. This may result in an increase in sensitivity of detection of the species compared to samples in which the third amplicon is not present. In any of the preceding embodiments, amplification may be by asymmetric PCR.

The invention provides magnetic particles decorated with nucleic acid probes to detect two or more amplicons characteristic of a species. For example, in some embodiments, the magnetic particles include two populations, wherein each population is conjugated to probes such that the magnetic particle that can operably bind each of the two or more amplicons. For instance, in embodiments where two target nucleic acids have been amplified to form a first amplicon and a second amplicon, a pair of particles each of which have a mix of capture probes on their surface may be used. In some embodiments, the first population of magnetic particles may be conjugated to a nucleic acid probe that operably binds a first segment of the first amplicon and a nucleic acid probe that operably binds a first segment of the second amplicon, and the second population of magnetic particles may be conjugated to a nucleic acid probe that operably binds a second segment of the first amplicon and a nucleic acid probe that operably binds a second segment of the second amplicon. For instance, one particle population may be conjugated with a 5′ capture probe specific to the first amplicon and a 5′ capture probe specific to second amplicon, and the other particle population may be conjugated with a 3′ capture probe specific to the first amplicon and a 3′ capture probe specific to the second amplicon.

In such embodiments, the magnetic particles may aggregate in the presence of the first amplicon and aggregate in the presence of the second amplicon. Aggregation may occur to a greater extent when both amplicons are present.

In some embodiments, a magnetic particle may be conjugated to two, three, four, five, six, seven, eight, nine, or ten nucleic acid probes, each of which operably binds a segment of a distinct target nucleic acid. In some embodiments, a magnetic particle may be conjugated to a first nucleic acid probe and a second nucleic acid probe, wherein the first nucleic acid probe operably binds to a first target nucleic acid, and the second nucleic acid probe operably binds to a second target nucleic acid. In other embodiments, a magnetic particle may be conjugated to a first nucleic acid probe that operably binds a first target nucleic acid, a second nucleic acid probe that operably binds a second target nucleic acid, and a third nucleic acid that operably binds a third target nucleic acid. In yet other embodiments, a magnetic particle may be conjugated to a first nucleic acid probe that operably binds a first target nucleic acid, a second nucleic acid probe that operably binds a second target nucleic acid, a third nucleic acid that operably binds a third target nucleic acid, and a fourth nucleic acid probe that operably binds a fourth target nucleic acid. In still other embodiments, a magnetic particle may be conjugated to a first nucleic acid probe that operably binds a first target nucleic acid, a second nucleic acid probe that operably binds a second target nucleic acid, a third nucleic acid that operably binds a third target nucleic acid, a fourth nucleic acid probe that operably binds a fourth target nucleic acid, and a fifth nucleic acid probe that operably binds a fifth target nucleic acid. In some embodiments, one population of magnetic particles includes the 5′ capture probe for each amplicon to be detected, and the other population of magnetic particles includes the 3′ capture probe for each amplicon to be detected.

Kits

The invention provides kits and articles of manufacture that can be used for carrying out the methods described herein. The kit may include one or more containers for holding the components of the kit (e.g., tubes (e.g., microcentrifuge tubes), plates (e.g., microtiter plates), trays, packaging materials (e.g., boxes), and the like. The kit may also include instructions (e.g., printed instructions for using the kit).

For example, a kit may include one or more, or all, of the following: one or more containers (e.g., tubes) that contain erythrocyte lysis buffers, one or more containers containing buffers or buffered solutions (e.g., TE buffer); one or more containers that contain primers (e.g., any of the primers described herein), one or more containers that contain control nucleic acids or total process controls, one or more containers containing lysis reagents (e.g., beads for beadbeating), and/or one or more containers containing amplification reagents (e.g., buffers, thermostable DNA polymerases, nucleotides, magnesium (e.g., MgCl₂), and the like). The kit may further include reagents for sequencing (e.g., buffers, library preparation reagents, enzymes, adaptors, and the like). The kit may further include reagents for T2MR detection (e.g., magnetic particles, probes, conjugated magnetic particles, and the like).

### EXAMPLES

The following examples are put forth so as to provide those of ordinary skill in the art with a complete disclosure and description of how the devices, systems, and methods described herein are performed, made, and evaluated, and are intended to be purely exemplary of the invention and are not intended to limit the scope of what the inventors regard as their invention.

### Example 1

### Sequencing Assay for Analysis of Amplicons in Complex Samples

A method was developed to use sequencing (e.g., Sanger, massively parallel sequencing, and/or single-molecule sequencing) for analysis of amplicons (e.g., species-specific amplicons from pathogens) from complex samples such as whole blood patient test samples. Such a method can be used, for example, for obtaining more specific sequence-based information from an amplicon after identifying that the amplicon is present in a sample, or to confirm or validate the identity of an amplicon detected by T2MR. The method described in this study uses optimized singleplex (single primer pair) reaction mixtures for the amplification of T2BACTERIA® panel species from blood samples (e.g., patient blood samples) and subsequent identification with Sanger sequencing using the T2BACTERIA® species-specific primers.

A. Evaluation of T2BACTERIA® Species-Specific Primers

To test the detection of each of the T2BACTERIA® panel members with the respective species-specific primers, blood lysate was prepared from whole blood spiked with 2-50 CFU/mL of one species using the sample processing procedure, as described below. The samples were processed using the sample processing and amplification procedures described below, and the generated double-stranded amplification products were sent to GENEWIZ for purification and sequencing.

Sequencing of the Acinetobacter baumannii (Ab), Efm, and Eci amplicons with the respective species-specific primers resulted in good quality reads and species identification (Table 1). These data demonstrate that sequencing (e.g., Sanger sequencing, massively parallel sequencing, and/or single-molecule sequencing) can be performed on amplicons produced in whole blood lysate, including using species-specific primers that are used to produce amplicons for T2MR-detection based approaches, such as the T2CANDIDA® and T2BACTERIA® panels.

TABLE 1
Sequencing of Ab, Efm, and Eci amplicon with species-specific primers

| | | Bases | Bases | QS | QS | Avg | | | | |
|---|---|---|---|---|---|---|---|---|---|---|
| | | before | after | before | after | Peak | Identity | Coverage | Taxonomy | Sequencing |
| Species | Primer | trimming | trimming | trimming | trimming | Height | (%) | (%) | Result | result |
| Ab | F | 279 | 242 | 44 | 50 | 1099 | 99 | 100 | 10/10 | (+) |
| | R | 276 | 236 | 44 | 52 | 1101 | 99 | 100 | 10/10 | (+) |
| | F | 278 | 237 | 41 | 48 | 1118 | 99 | 100 | 10/10 | (+) |
| | R | 277 | 231 | 43 | 52 | 1227 | 99 | 100 | 10/10 | (+) |
| | F | 276 | 226 | 37 | 44 | 1151 | 99 | 100 | 10/10 | (+) |
| | R | 274 | 227 | 41 | 50 | 1221 | 99 | 100 | 10/10 | (+) |
| | F | 275 | 237 | 37 | 43 | 1177 | 99 | 100 | 10/10 | (+) |
| | R | 280 | 236 | 41 | 48 | 1157 | 98 | 99 | 10/10 | (+) |
| Eci | F | 210 | 175 | 48 | 57 | 817 | 99 | 100 | 10/10 | (+) |
| | R | 206 | 168 | 50 | 61 | 812 | 100 | 100 | 10/10 | (+) |
| | F | 209 | 180 | 48 | 56 | 868 | 100 | 100 | 10/10 | (+) |
| | R | 206 | 175 | 50 | 59 | 833 | 100 | 100 | 10/10 | (+) |
| | F | 206 | 182 | 49 | 55 | 904 | 100 | 100 | 10/10 | (+) |
| | R | 200 | 181 | 53 | 58 | 874 | 99 | 100 | 10/10 | (+) |
| | F | 206 | 180 | 50 | 57 | 826 | 100 | 100 | 10/10 | (+) |
| | R | 202 | 174 | 52 | 60 | 816 | 100 | 100 | 10/10 | (+) |
| Efm | F | 312 | 279 | 46 | 51 | 815 | 99 | 99 | 10/10 | (+) |
| | R | 316 | 291 | 40 | 43 | 663 | 99 | 98 | 10/10 | (+) |
| | F | 324 | 263 | 46 | 56 | 866 | 96 | 94 | 10/10 | (+) |
| | R | 319 | 265 | 44 | 52 | 689 | 97 | 96 | 10/10 | (+) |
| | F | 317 | 281 | 46 | 52 | 850 | 97 | 96 | 10/10 | (+) |
| | R | 316 | 281 | 42 | 47 | 701 | 99 | 96 | 10/10 | (+) |
| | F | 311 | 293 | 51 | 54 | 813 | 99 | 99 | 10/10 | (+) |
| | R | 313 | 291 | 47 | 50 | 690 | 99 | 99 | 10/10 | (+) |

B. Evaluation of Amplicon Clean-Up Kits for Sequencing

An evaluation was performed on various post-amplification clean-up methods to evaluate their effect on obtaining high-quality sequencing reads of the amplified T2BACTERIA® panel members. These methods included the GENEWIZ in-house cleanup procedure and five commercially available kits:

1. AGENCOURT® AMPURE® XP PCR Purification Kit

2. ChargeSwitch®-Pro PCR Clean-up Kit

3. Thermo Scientific® GeneJET® PCR Purification Kit

4. QIAGEN® QIAquick® PCR Purification Kit

5. QIAGEN® QIAquick® Gel Extraction Kit

GENEWIZ uses an enzymatic-based clean-up process. The AMPURE® approach involves binding 100 bp and larger DNA molecules to paramagnetic beads for purification. The ChargeSwitch®, GeneJet®, and both QIAGEN® kits are column-based purification methods. Amplicons generated with asymmetric reaction mixtures for Ab, Kp, and Pa were cleaned up with each of the purification methods outlined above according to the manufacturer's instructions and then sequenced at GENEWIZ. The QS values for each of the sequencing reads were then compared to determine the optimal PCR product purification method. The results are summarized in Table 2.

TABLE 2
Quality Scores and read lengths for each of the different clean-up methods by species

| | | | | | | | Thermo | | | | | |
|---|---|---|---|---|---|---|---|---|---|---|---|---|
| | | | AGENCOURT ® | | | | Scientific ® | | QIAGEN ® | | QIAGEN ® | |
| | | | AMPURE ® | | | | GeneJET ® | | QIAquick ® | | QIAquick ® | |
| | | | XP PCR | | ChargeSwitch ®- | | PCR | | PCR | | Gel | |
| | GENEWIZ | | Purification | | Pro PCR Clean- | | Purification | | Purification | | Extraction | |
| | Clean-Up | | Kit | | up Kit | | Kit | | Kit | | Kit | |
| | QualityScore | CRL | QualityScore | CRL | QualityScore | CRL | QualityScore | CRL | QualityScore | CRL | QualityScore | CRL |
| | Ab Amplicon | | | | | | | | | | | |
| 1 | 43 | 201 | 19 | 85 | 35 | 199 | 44 | 239 | 45 | 241 | 43 | 236 |
| Forward | | | | | | | | | | | | |
| 1 | 46 | 210 | 32 | 209 | 38 | 208 | 49 | 235 | 50 | 235 | 48 | 237 |
| Reverse | | | | | | | | | | | | |
| 2 | 39 | 201 | 27 | 211 | 12 | 1 | | | | | | |
| Forward | | | | | | | | | | | | |
| 2 | 43 | 210 | 26 | 211 | 11 | 1 | | | | | | |
| Reverse | | | | | | | | | | | | |
| | Kp Amplicon | | | | | | | | | | | |
| 1 | 53 | 203 | 33 | 204 | 35 | 206 | 52 | 248 | 54 | 245 | 51 | 246 |
| Forward | | | | | | | | | | | | |
| 1 | 52 | 218 | 32 | 211 | 31 | 207 | 54 | 244 | 54 | 243 | 54 | 205 |
| Reverse | | | | | | | | | | | | |
| 2 | 15 | 2 | 31 | 204 | 38 | 205 | | | | | | |
| Forward | | | | | | | | | | | | |
| 2 | 52 | 215 | 25 | 173 | 36 | 215 | | | | | | |
| Reverse | | | | | | | | | | | | |
| | Pa Amplicon | | | | | | | | | | | |
| 1 | 12 | 1 | 12 | 1 | 12 | 1 | 26 | 105 | 26 | 105 | 26 | 104 |
| Forward | | | | | | | | | | | | |
| 1 | 22 | 66 | 17 | 52 | 12 | 1 | 13 | 1 | 13 | 1 | 12 | 1 |
| Reverse | | | | | | | | | | | | |
| 2 | 12 | 1 | 12 | 1 | 11 | 1 | | | | | | |
| Forward | | | | | | | | | | | | |
| 2 | 22 | 70 | 17 | 52 | 9 | 1 | | | | | | |
| Reverse | | | | | | | | | | | | |

GENEWIZ, GeneJet®, and both QIAGEN® kits produced very comparable quality scores and read lengths to each other, while AMPURE® and ChargeSwitch® produced poorer quality scores and read lengths. These data indicate that a variety of PCR clean-up kits can be used prior to sequencing. For ease-of-use, in subsequent experiments described in this Example, the GENEWIZ in-house purification method was used for PCR purification prior to sequencing.

C. Titration Sensitivity Experiment at Titers as Low as 2-4 CFU/mL

(i) T2BACTERIA® Species Identification—Concentration Series

Species identification by T2MR and bi-directional Sanger sequencing was performed for each T2BACTERIA® panel member at different blood spike concentrations in a titration experiment. Spiked whole blood samples at 2-50 CFU/mL for each T2BACTERIA® panel species were processed with the sample processing procedure as described below. Negative samples, consisting of unspiked whole blood, were tested with each set of species-specific primers. In addition, 4 clinical samples were also tested.

The lysate from each sample was amplified in 2 parallel reactions which were combined after PCR completion. Amplified samples were analyzed by T2MR and sent for purification and sequencing at GENEWIZ as described in Section B above and in Section D below.

(ii) Data Analysis

Data from sequencing, BLAST search, and T2MR results were collected. Low quality ends of the sequence were trimmed using the default settings (window length=18 bases, good number of bases per window=75%, quality value cutoff=25). Trimming properties (e.g., number of bases, quality scores, and peak height) were exported for each sequence. Trimmed sequences were converted to FASTA files and a BLASTn search of the nucleotide (nt) database was performed on each sequence with NCBI default parameters and exclusion of models and environmental samples. Hit tables and taxonomy (sometimes abbreviated herein as “tax”) reports were exported for each search.

Samples having a low number of bases after trimming (<10 bp) could not be run with a BLAST search and were considered to be negatives in terms of sequencing. Many of these samples had low quality scores and overlapping fluorescence traces.

BLAST hit tables indicated up to 100 hits for a given sequence. In many cases, these hits contained the expected species and several near neighbors, with the expected species having the highest identity and coverage percentages. These tables could be biased by the number of a given species in the database. For instance, the NCBI database contains 10,401 genomic assemblies for Escherichia coli while only 868 assemblies for Enterococcus faecium. Hit tables for E. coli contained predominantly strains of E. coli while hit tables for E. faecium contained many near Enterococcus neighbors due to the relative paucity of E. faecium in the database. Thus, scoring the BLAST matches by identity and coverage may give more meaningful results in some cases than the taxonomic makeup of the search results.

Sequencing of negative samples in some cases resulted in the potential amplification of species other than T2BACTERIA® panel members or background human DNA. These cases are paired with a negative T₂ result and while the sequencing results may contain the T2BACTERIA® target within the hit table, either low identity and/or coverage provides evidence for a sample lacking a recognized target. For example, a near neighbor of a T2BACTERIA® target may be present in the sample and BLAST hit tables may contain the target, but the identity and coverage will be higher for the near neighbor than the T2BACTERIA® target. The targets may also appear in the hit table when an amplicon is made from background human DNA and contains the primer associated with the T2BACTERIA® target, but in this case the coverage will be low and of the primer region alone.

(iii) Results

All data (T2MR and sequencing results) are summarized in Tables 3 and 4. These data tables also contain the results from the testing of the spiked samples for each T2BACTERIA® panel member that were processed with the reaction conditions as described for the final method above.

TABLE 3
Positive Spiked Samples

| | | | Bases | Bases | QS | QS | | | | | | | | |
|---|---|---|---|---|---|---|---|---|---|---|---|---|---|---|
| | | | before | after | before | after | Avg | | | | | | | |
| Spe- | | | trim- | trim- | trim- | trim- | Peak | T2 | Identity | Coverage | Tax | Blast | T2 | |
| cies | Primer | Conc | ming | ming | ming | ming | height | (ms) | (%) | (%) | Result | Notes | Result | Sequencing |
| Ab | F | 9-15 | 279 | 250 | 42 | 46 | 769 | 183 | 99 | 100 | 94/100 | | (+) | (+) |
| | | CFU/mL | | | | | | | | | | | | |
| | R | 9-15 | 282 | 247 | 43 | 49 | 649 | | 100 | 100 | 94/100 | | | |
| | | CFU/mL | | | | | | | | | | | | |
| Ab | F | 9-15 | 279 | 250 | 43 | 47 | 794 | 184 | 98-99 | 100 | 94/100 | | (+) | (+) |
| | | CFU/mL | | | | | | | | | | | | |
| | R | 9-15 | 285 | 249 | 43 | 49 | 817 | | 100 | 100 | 94/100 | | | |
| | | CFU/mL | | | | | | | | | | | | |
| Ab | F | 6-9 | 276 | 2 | 11 | 0 | 722 | 130 | n/a | n/a | n/a | | (+) | (+) |
| | | CFU/mL | | | | | | | | | | | | |
| | R | 6-9 | 275 | 237 | 40 | 46 | 694 | | 100 | 100 | 94/100 | | | |
| | | CFU/mL | | | | | | | | | | | | |
| Ab | F | 6-9 | 279 | 25 | 11 | 29 | 765 | 144 | 100 | 100 | 84/100 | short input | (+) | (+) |
| | | CFU/mL | | | | | | | | | | parameters | | |
| | R | 6-9 | 277 | 237 | 41 | 47 | 635 | | 100 | 100 | 94/100 | | | |
| | | CFU/mL | | | | | | | | | | | | |
| Ab | F | 3-6 | 277 | 78 | 16 | 29 | 774 | 126 | 100 | 100 | 89/100 | | (+) | (+) |
| | | CFU/mL | | | | | | | | | | | | |
| | R | 3-6 | 276 | 240 | 43 | 48 | 661 | | 100 | 100 | 94/100 | | | |
| | | CFU/mL | | | | | | | | | | | | |
| Ab | F | 3-6 | 276 | 2 | 12 | 0 | 794 | 127 | n/a | n/a | n/a | | (+) | (+) |
| | | CFU/mL | | | | | | | | | | | | |
| | R | 3-6 | 276 | 245 | 41 | 46 | 588 | | 100 | 100 | 94/100 | | | |
| | | CFU/mL | | | | | | | | | | | | |
| Eci | F | 30-50 | 208 | 178 | 42 | 49 | 711 | 325 | 100 | 100 | 99/100 | | (+) | (+) |
| | | CFU/mL | | | | | | | | | | | | |
| | R | 30-50 | 205 | 169 | 44 | 52 | 891 | | 100 | 100 | 99/100 | | | |
| | | CFU/mL | | | | | | | | | | | | |
| Eci | F | 30-50 | 210 | 178 | 44 | 51 | 936 | 290 | 100 | 100 | 99/100 | | (+) | (+) |
| | | CFU/mL | | | | | | | | | | | | |
| | R | 30-50 | 205 | 174 | 44 | 52 | 923 | | 100 | 100 | 99/100 | | | |
| | | CFU/mL | | | | | | | | | | | | |
| Eci | F | 20-30 | 211 | 156 | 39 | 50 | 600 | 315 | 100 | 100 | 99/100 | | (+) | (+) |
| | | CFU/mL | | | | | | | | | | | | |
| | R | 20-30 | 208 | 169 | 40 | 48 | 669 | | 100 | 100 | 99/100 | | | |
| | | CFU/mL | | | | | | | | | | | | |
| Eci | F | 20-30 | 209 | 156 | 40 | 50 | 573 | 302 | 100 | 100 | 99/100 | | (+) | (+) |
| | | CFU/mL | | | | | | | | | | | | |
| | R | 20-30 | 204 | 169 | 40 | 48 | 695 | | 100 | 100 | 99/100 | | | |
| | | CFU/mL | | | | | | | | | | | | |
| Eci | F | 10-20 | 210 | 165 | 41 | 50 | 622 | 255 | 100 | 100 | 99/100 | | (+) | (+) |
| | | CFU/mL | | | | | | | | | | | | |
| | R | 10-20 | 205 | 168 | 38 | 46 | 676 | | 100 | 100 | 99/100 | | | |
| | | CFU/mL | | | | | | | | | | | | |
| Eci | F | 10-20 | 213 | 163 | 39 | 49 | 584 | 264 | 100 | 100 | 99/100 | | (+) | (+) |
| | | CFU/mL | | | | | | | | | | | | |
| | R | 10-20 | 205 | 169 | 43 | 51 | 620 | | 100 | 100 | 99/100 | | | |
| | | CFU/mL | | | | | | | | | | | | |
| Efm | F | 15-25 | 321 | 267 | 43 | 50 | 772 | 235 | 99 | 100 | 44/100 | | (+) | (+) |
| | | CFU/mL | | | | | | | | | | | | |
| | R | 15-25 | 319 | 277 | 41 | 47 | 685 | | 99 | 100 | 49/100 | | | |
| | | CFU/mL | | | | | | | | | | | | |
| Efm | F | 15-25 | 323 | 252 | 40 | 49 | 892 | 226 | 99 | 100 | 44/100 | | (+) | (+) |
| | | CFU/mL | | | | | | | | | | | | |
| | R | 15-25 | 323 | 279 | 37 | 42 | 787 | | 98 | 100 | 49/100 | | | |
| | | CFU/mL | | | | | | | | | | | | |
| Efm | F | 10-15 | 314 | 277 | 42 | 47 | 884 | 151 | 99-100 | 100 | 45/100 | | (+) | (+) |
| | | CFU/mL | | | | | | | | | | | | |
| | R | 10-15 | 320 | 277 | 38 | 43 | 738 | | 99 | 100 | 50/100 | | | |
| | | CFU/mL | | | | | | | | | | | | |
| Efm | F | 10-15 | 315 | 260 | 43 | 50 | 885 | 177 | 99-100 | 100 | 45/100 | | (+) | (+) |
| | | CFU/mL | | | | | | | | | | | | |
| | R | 10-15 | 320 | 284 | 39 | 43 | 587 | | 98-99 | 100 | 50/100 | | | |
| | | CFU/mL | | | | | | | | | | | | |
| Efm | F | 5-10 | 314 | 271 | 40 | 46 | 846 | 177 | 99-100 | 100 | 45/100 | | (+) | (+) |
| | | CFU/mL | | | | | | | | | | | | |
| | R | 5-10 | 319 | 272 | 34 | 39 | 587 | | 99 | 100 | 50/100 | | | |
| | | CFU/mL | | | | | | | | | | | | |
| Efm | F | 5-10 | 321 | 276 | 43 | 48 | 877 | 169 | 99 | 100 | 45/100 | | (+) | (+) |
| | | CFU/mL | | | | | | | | | | | | |
| | R | 5-10 | 316 | 273 | 42 | 47 | 708 | | 99-100 | 100 | 50/100 | | | |
| | | CFU/mL | | | | | | | | | | | | |
| Kp | F | 6-10 | 284 | 249 | 48 | 54 | 837 | 476 | 99 | 100 | 95/100 | | (+) | (+) |
| | | CFU/mL | | | | | | | | | | | | |
| | R | 6-10 | 287 | 257 | 50 | 56 | 818 | | 98-100 | 100 | 100/100 | | | |
| | | CFU/mL | | | | | | | | | | | | |
| Kp | F | 6-10 | 285 | 250 | 50 | 57 | 897 | 544 | 99 | 100 | 95/100 | | (+) | (+) |
| | | CFU/mL | | | | | | | | | | | | |
| | R | 6-10 | 285 | 256 | 51 | 57 | 831 | | 98-99 | 100 | 100/100 | | | |
| | | CFU/mL | | | | | | | | | | | | |
| Kp | F | 4-6 | 285 | 241 | 43 | 49 | 767 | 519 | 99 | 100 | 95/100 | | (+) | (+) |
| | | CFU/mL | | | | | | | | | | | | |
| | R | 4-6 | 306 | 2 | 7 | 0 | 769 | | n/a | n/a | n/a | | | |
| | | CFU/mL | | | | | | | | | | | | |
| Kp | F | 4-6 | 286 | 226 | 39 | 47 | 712 | 541 | 99 | 100 | 95/100 | | (+) | (+) |
| | | CFU/mL | | | | | | | | | | | | |
| | R | 4-6 | 312 | 2 | 15 | 0 | 723 | | n/a | n/a | n/a | | | |
| | | CFU/mL | | | | | | | | | | | | |
| Kp | F | 2-4 | 285 | 233 | 36 | 42 | 757 | 520 | 99-100 | 100 | 95/100 | | (+) | (+) |
| | | CFU/mL | | | | | | | | | | | | |
| | R | 2-4 | 287 | 237 | 38 | 44 | 793 | | 99-100 | 100 | 100/100 | | | |
| | | CFU/mL | | | | | | | | | | | | |
| Kp | F | 2-4 | 287 | 242 | 42 | 48 | 665 | 531 | 99 | 100 | 95/100 | | (+) | (+) |
| | | CFU/mL | | | | | | | | | | | | |
| | R | 2-4 | 308 | 2 | 10 | 13 | 781 | | n/a | n/a | n/a | | | |
| | | CFU/mL | | | | | | | | | | | | |
| Pa | F | 15-25 | 187 | 94 | 30 | 46 | 826 | 397 | 98-100 | 100 | 99/100 | | (+) | (+) |
| | | CFU/mL | | | | | | | | | | | | |
| | R | 15-25 | 187 | 26 | 16 | 29 | 534 | | n/a | n/a | 0/100 | short input | | |
| | | CFU/mL | | | | | | | | | | parameters | | |
| Pa | F | 15-25 | 187 | 94 | 31 | 47 | 848 | 474 | 97-100 | 100 | 97/100 | | (+) | (+) |
| | | CFU/mL | | | | | | | | | | | | |
| | R | 15-25 | 188 | 26 | 17 | 30 | 547 | | n/a | n/a | 0/100 | short input | | |
| | | CFU/mL | | | | | | | | | | parameters | | |
| Pa | F | 10-15 | 186 | 75 | 25 | 39 | 959 | 447 | 97-100 | 100 | 99/100 | | (+) | (+) |
| | | CFU/mL | | | | | | | | | | | | |
| | R | 10-15 | 188 | 2 | 15 | 23 | 911 | | n/a | n/a | n/a | | | |
| | | CFU/mL | | | | | | | | | | | | |
| Pa | F | 10-15 | 188 | 85 | 26 | 40 | 838 | 504 | 96-100 | 92-100 | 98/107 | | (+) | (+) |
| | | CFU/mL | | | | | | | | | | | | |
| | R | 10-15 | 191 | 2 | 18 | 31 | 918 | | n/a | n/a | n/a | | | |
| | | CFU/mL | | | | | | | | | | | | |
| Pa | F | 5-10 | 187 | 75 | 24 | 41 | 856 | 475 | 98-100 | 92-100 | 98/107 | | (+) | (+) |
| | | CFU/mL | | | | | | | | | | | | |
| | R | 5-10 | 189 | 2 | 14 | 29 | 939 | | n/a | n/a | n/a | | | |
| | | CFU/mL | | | | | | | | | | | | |
| Pa | F | 5-10 | 189 | 82 | 24 | 42 | 972 | 456 | 98-100 | 94-100 | 97/107 | | (+) | (+) |
| | | CFU/mL | | | | | | | | | | | | |
| | R | 5-10 | 191 | 2 | 13 | 31 | 955 | | n/a | n/a | n/a | | | |
| | | CFU/mL | | | | | | | | | | | | |
| Sa | F | 6-10 | 179 | 150 | 45 | 53 | 1034 | 268 | 100 | 100 | 100/100 | | (+) | (+) |
| | | CFU/mL | | | | | | | | | | | | |
| | R | 6-10 | 187 | 152 | 45 | 55 | 980 | | 100 | 100 | 100/100 | | | |
| | | CFU/mL | | | | | | | | | | | | |
| Sa | F | 6-10 | 179 | 150 | 46 | 54 | 1032 | 261 | 100 | 100 | 100/100 | | (+) | (+) |
| | | CFU/mL | | | | | | | | | | | | |
| | R | 6-10 | 191 | 151 | 43 | 55 | 957 | | 100 | 100 | 100/100 | | | |
| | | CFU/mL | | | | | | | | | | | | |
| Sa | F | 4-6 | 178 | 135 | 42 | 51 | 795 | 307 | 100 | 100 | 100/100 | | (+) | (+) |
| | | CFU/mL | | | | | | | | | | | | |
| | R | 4-6 | 187 | 149 | 41 | 52 | 801 | | 100 | 100 | 100/100 | | | |
| | | CFU/mL | | | | | | | | | | | | |
| Sa | F | 4-6 | 177 | 136 | 41 | 50 | 831 | 316 | 100 | 100 | 100/100 | | (+) | (+) |
| | | CFU/mL | | | | | | | | | | | | |
| | R | 4-6 | 186 | 149 | 41 | 51 | 844 | | 100 | 100 | 100/100 | | | |
| | | CFU/mL | | | | | | | | | | | | |
| Sa | F | 2-4 | 178 | 136 | 43 | 53 | 794 | 296 | 100 | 100 | 100/100 | | (+) | (+) |
| | | CFU/mL | | | | | | | | | | | | |
| | R | 2-4 | 186 | 151 | 43 | 42 | 762 | | 98 | 100 | 100/100 | | | |
| | | CFU/mL | | | | | | | | | | | | |
| Sa | F | 2-4 | 178 | 139 | 42 | 50 | 765 | 296 | 100 | 100 | 100/100 | | (+) | (+) |
| | | CFU/mL | | | | | | | | | | | | |
| | R | 2-4 | 188 | 153 | 43 | 52 | 840 | | 98 | 100 | 100/100 | | | |
| | | CFU/mL | | | | | | | | | | | | |

TABLE 4
Negative Samples

| | | Bases | Bases | QS | QS | Avg | | | | | | | Se- |
|---|---|---|---|---|---|---|---|---|---|---|---|---|---|
| Spe- | | before | after | before | after | Peak | T2 | Identity | Coverage | Tax | Blast | T2 | quencing |
| cies | Primer | trimming | trimming | trimming | trimming | height | (ms) | (%) | (%) | Result | Notes | Result | Result |
| Ab | F | 516 | 2 | 3 | 4 | 532 | 184 | n/a | n/a | n/a | | (+) | (−) |
| | R | 533 | 2 | 3 | 0 | 550 | | n/a | n/a | n/a | | | |
| Ab | F | 263 | 2 | 2 | 0 | 805 | 28 | n/a | n/a | n/a | | (−) | (−) |
| | R | 341 | 2 | 4 | 8 | 734 | | n/a | n/a | n/a | | | |
| Ab | F | 256 | 2 | 2 | 0 | 795 | 28 | n/a | n/a | n/a | | (−) | (−) |
| | R | 339 | 2 | 4 | 0 | 824 | | n/a | n/a | n/a | | | |
| Eci | F | 203 | 2 | 6 | 27 | 209 | 29 | n/a | n/a | n/a | | (−) | (−) |
| | R | 208 | 2 | 6 | 0 | 224 | | n/a | n/a | n/a | | | |
| Eci | F | 315 | 2 | 4 | 0 | 753 | 28 | n/a | n/a | n/a | | (−) | (−) |
| | R | 809 | 2 | 8 | 0 | 793 | | n/a | n/a | n/a | | | |
| Eci | F | 313 | 2 | 4 | 0 | 342 | 28 | n/a | n/a | n/a | | (−) | (−) |
| | R | 366 | 2 | 5 | 0 | 486 | | n/a | n/a | n/a | | | |
| Efm | F | 257 | 224 | 50 | 56 | 417 | 29 | n/a | n/a | 0/100 | Human | (−) | (−) |
| | R | 257 | 218 | 50 | 58 | 975 | | 100 | 13 | 33/100 | Human, Efm | | |
| | | | | | | | | | | | primer | | |
| Efm | F | 301 | 256 | 38 | 43 | 815 | 30 | 83 | 99 | 41/100 | E. gallinarum, | (−) | (−) |
| | | | | | | | | | | | E. casseliflavus | | |
| | R | 306 | 262 | 41 | 47 | 742 | | 88 | 98 | 42/100 | E. gallinarum, | | |
| | | | | | | | | | | | E. casseliflavus | | |
| Efm | F | 290 | 2 | 8 | 10 | 749 | 29 | n/a | n/a | n/a | | (−) | (−) |
| | R | 263 | 217 | 32 | 38 | 781 | | 100 | 15 | 33/100 | Human, Efm | | |
| | | | | | | | | | | | primer | | |
| Pa | F | 723 | 2 | 5 | 0 | 229 | 30 | n/a | n/a | n/a | | (−) | (−) |
| | R | 354 | 2 | 4 | 0 | 474 | | n/a | n/a | n/a | | | |
| Pa | F | 326 | 2 | 5 | 21 | 773 | 29 | n/a | n/a | n/a | | (−) | (−) |
| | R | 356 | 2 | 5 | 0 | 713 | | n/a | n/a | n/a | | | |
| Pa | F | 277 | 2 | 4 | 0 | 606 | 27 | n/a | n/a | n/a | | (−) | (−) |
| | R | 270 | 2 | 4 | 0 | 964 | | n/a | n/a | n/a | | | |
| Sa | F | 265 | 26 | 19 | 29 | 3162 | 37 | n/a | n/a | 0/100 | Human | (−) | (−) |
| | R | 268 | 2 | 10 | 8 | 2772 | | n/a | n/a | n/a | | | |
| Sa | F | 50 | 2 | 2 | 1 | 5377 | 38 | n/a | n/a | n/a | | (−) | (−) |
| | R | 263 | 2 | 3 | 0 | 1572 | | n/a | n/a | n/a | | | |
| Sa | F | 242 | 134 | 28 | 49 | 1587 | 37 | n/a | n/a | 0/100 | Human | (−) | (−) |
| | R | 275 | 169 | 27 | 41 | 2080 | | 100 | 24 | 98/100 | Human, Sa | | |
| | | | | | | | | | | | primer | | |

All negative control samples were true T2MR negatives except for one negative sample analyzed with the Ab nominal reaction mix along with the 2-50 CFU/mL samples, which may have been contaminated during processing.

The sequencing results for the negative control samples returned traces with low QS values and short read lengths for all reaction buffers except 2 out of the 3 Efm DOE-optimal reaction mixtures and one sample amplified with the Sa DOE-optimal reaction buffer. Two Efm reaction buffer negatives appeared to have nearest neighbors to E. faecium, E. gallinarum, and E. casseliflavus, that match at 98-99% identity while Efm only matches at 83-88% identity. Since Efm was not detected by T2MR, it is likely that these near neighbors may be in the sample and are amplified by the Efm primers but are not detected by the T2MR probes. Three of the negatives matched sequences for human DNA, and while two of these had matches to Efm and Kp, the only matched region is the primer sequence with coverage below 30%. Therefore, these negatives were not considered positive for these species.

All positive spiked samples analyzed were true positives in T2MR. Sequencing results were obtained for samples spiked as low as 5 CFU/mL and agreement was found with T2MR. With only a few exceptions, the sequencing results showed QS values above 40 and good read lengths. In some cases, sequencing data was only obtained uni-directionally (2 Ab, 3 Kp, 4 Pa) but did identify the expected species.

T2MR and at least one direction of sequencing were concordant for 93% (14/15) negative samples and 100% (36/36) positive samples. Of the 4 clinical samples that were run, 100% (4/4) were concordant between T2MR and sequencing. These results indicate that T2MR and sequencing are complementary methods for determining the nature of a sample.

(iv) Qualitative T2MR/Sequencing Analysis Method

Based on these data, a data analysis methodology was developed to qualitatively determine if a sample is qualitatively positive or negative for a T2BACTERIA® target of interest. This method was applied to the data generated and results were reported as positive (+) or negative (−) in Tables 3 and 4.

T2MR

For the test result for a spiked sample to be considered T2MR positive, the T2MR signal for the bacterial target of interest must be greater than the defined cutoff. For the test result for a negative sample to be considered negative, the T2MR signal for the species of interest must be below the defined cutoff (Ab 130 ms; Eci 150 ms; Efm 65 ms; Kp 65 ms; Pa 65 ms; Sa 65 ms; and IC 85 ms).

Sequencing

Using Chromatogram Explorer Lite v5.0.2, ab1 files were converted to FASTA files with removal of low-quality ends enabled. The settings of low-quality ends trimmings are as follows: Good bases no: 75%, Window length: 18 bases, Good base: 25 QV, Apply to all: Enabled. For this analysis, sequencing was considered negative if the length of the trimmed amplicons was less than the preliminary cutoffs shown in Table 5.

TABLE 5
Trimmed Amplicon Cutoffs

| | | | Avg - 5x | |
|---|---|---|---|---|
| | Avg Trimmed | | Standard | Preliminary |
| Species | length | Stdev | Deviation | Cutoff |
| Ab | 242 | 11.45 | 185 | 150 |
| Eci | 166 | 6.75 | 133 | 100 |
| Efm | 271 | 7.71 | 232 | 200 |
| Kp | 240 | 11.47 | 182 | 150 |
| Pa | 88 | 8.65 | 45 | 45 |
| Sa | 147 | 6.48 | 115 | 100 |

If an amplicon met the minimum length requirement, Blastn (NCBI) searches were performed for each FASTA sequence generated using the “Nucleotide collection (nr/nt)” database.

All sequences were selected and saved as a hit table in a .txt or .csv file, saving the taxonomy report. The hit table and taxonomy report were filtered, with all hits with an identity and coverage below 90% considered negative. The remaining number of this per species divided by the total number of remaining hits was calculated. A species was considered positively identified if it made up above 80% of the remaining hits for either the sense or antisense (unidirectional) amplicon.

Conclusion

Unexpectedly, the methods described herein allowed sequencing from lysates that contain concentrated amounts of cell debris and host cell nucleic acids such as DNA. The lysates prepared according to the sample processing procedure described below resulted in a highly concentrated blood lysate that can be characterized as a super-saturated solution of debris. The method developed in this Example to identify T2BACTERIA® panel species by sequencing has been shown to be species-specific and sensitive. Therefore, the methods described herein can be used to sequence target nucleic acids in complex samples such as blood.

D. Materials and Methods

Sample Processing Procedure

Samples/specimens (whole blood or other sample matrix) were obtained. A desired number of T2BACTERIA® 2.8 mL lysis tube assemblies containing erythrocyte lysis buffer and glass beads were obtained, and centrifuged for 5 seconds at 2000×g to collect the lysis buffer and beads to the bottom of the tube. See, e.g., International Patent Application Nos. WO 2012/054639 and WO 2017/127731. The sample was inverted 5-10 times to mix, and 2 mL of the sample was added to the lysis tube by dispensing against the side of the tube. The sample was mixed by pipetting up and down. The tubes were capped and the samples were allowed to incubate for 5 minutes at room temperature (RT) to ensure complete lysis of red blood cells. The tubes were centrifuged for 5 minutes at 6000×g at RT. The cell pellet was located and the supernatant was removed. Next, 150 μL of 1× TE was added to wash the pellet, and the tubes were re-capped and pulse vortexed twice briefly to dislodge the pellet, followed by centrifuging the tube for 4 min at 6000×g at RT. All of the supernatant was removed by pipetting with the tip in the center of the bead bed without disturbing the cell pellet. Next, 110 μL of 1× TE was added to the beads and pellet, and the tubes were recapped. The tubes were loaded into a vortexer and bead beat at 3200 rpm for 5 minutes. The tubes were removed from the vortexer and centrifuged for 2 min at 6000×g to get the sample and beads to the bottom of tubes. This procedure results in a concentrated blood lysate that is a super-saturated solution of cell debris (including solid material).

Amplfiication Procedure

A pre-chilled 96-well cold block and desired number of EPPENDORF® 0.1 mL strip tubes and caps, 2 wells per 2.8 mL lysis tube sample was obtained, the strip tubes were labelled and placed into the cold block. Next, 50 μL of lysate was added to the corresponding well in the strip tube, and the remaining 50 μL of lysate was added to another well. Appropriate amounts of reaction buffer singleplex mixes were obtained from the 2-8° C. storage and 30 μL of the appropriate reaction buffer (singleplex formulation containing the respective target primers) was added to each well containing the 50 μL lysate. Reaction buffers as described in International Patent Application Publication No. WO 2017/127731 were used for some experiments (e.g., as described in Example 3 of WO 2017/127731). In other experiments, the reaction buffer included 0.2 μM F primer, 0.4 μM R primer, 4 mM MgCl₂, pH 8.3, 0.2 mM dNTPs, and 15 μL DNA polymerase. The samples were securely capped and placed in the PCR Block of the thermal cycler. The “denature” program on the thermal cycler was then run (95° C. for 5 minutes followed by cooling to 25° C.). The tubes were removed and placed in a centrifuge fitted with a PCR Strip Tube rotor. The samples were centrifuged for 5 minutes at 8000×g and then placed into a pre-chilled 96 well cold block. Next, 20 μL of thermostable DNA polymerase mix was added to the appropriate samples. The tubes were capped and loaded on the thermocycler (MASTERCYCLER® PRO) and the samples were amplified. The PCR cycling parameters included 1 cycle of 95° C. for 3 min, followed by 40-46 cycles of 95° C. for 20 sec, 58° C. for 30 sec, 68° C. for 30 sec, and 1 cycle of 68° C. for 1 min, followed by holding at 4° C. Upon completion of cycling, reactions were detected by the T2MR detection procedure below.

T2MR Detection Procedure:

Upon completion of cycling, the reactions were detected as follows. Prior to setting up detection reactions, tube racks were placed into a 62° C. benchtop oven and allowed to incubate for at least 1 h. Hybridization reactions were transferred to the pre-heated racks just before placing them into the EPPENDORF® THERMOMIXER® for the 30 minute hybridization. The appropriate number (1 per amplified sample) of individual 0.2 mL dome-capped Eppendorf PCR tubes were placed on clean racks and labelled. Next, 9 μL of the amplicon was transferred to the 0.2 mL tube. To each of the 0.2 mL tubes containing amplicon, 6.3 μL of 1× TE was added, and the samples were mixed by pipetting 4 times. Appropriate volumes of each particle (Ab, Efm, Eci, Kp, Pa, Sa, and OIC) were obtained from the 2-8° C. storage. The particle bulk was vortexed to ensure homogenous mixing of particles. The particle bulk was vortexed. Next, 15 μL of the species-specific particle bulk was added into respective individual dome-capped Eppendorf PCR tubes, and the 0.2 mL individual PCR tubes were transferred to a 96-well metal block. The individual 0.2 mL PCR tubes were loaded into a THERMOMIXER® that was set to 62° C. to minimize cooling. The samples were hybridized for 30 min at 62° C. and 1,400 rpm in the THERMOMIXER®. After hybridization was completed, the plates were removed and transferred to a96-well tube holding block and loaded into a T2MR unit for T2MR reading.

### Example 2

### T2Bacteria Amplicon Sequencing Validation

A method was designed to amplify bacterial targets on the T2BACTERIA® Panel in singleplex, detect with T2MR and confirm the T₂ result by sequencing. The design and development of this method is described in Example 1. Detection can be carried out with both T2BACTERIA® magnetic particles and by Sanger sequencing. This assay can be used to confirm the presence a bacterial species from the T2Bacteria panel (Acinetobacter baumannii, Enterococcus faecium, Escherichia coli, Klebsiella pneumoniae, Pseudomonas aeruginosa and Staphylococcus aureus) spiked in or present in whole blood from sequencing and from T2MR detection. This method can also be performed to identify the subspecies or strain of the bacterial species, or to provide additional information regarding the amplicon that is detected by T2MR (e.g., sequence information to determine the genotype at a single nucleotide polymorphism (SNP)).

A. Materials and Methods

Frozen samples were allowed to equilibrate to room temperature for 90 min±30 min. Samples were mixed by inversion 8-10 times and visually inspected prior to starting the assay. If a noticeable clot was observed, the sample was discarded in the biohazard trash and a replacement sample was used. Samples were processed according to the sample processing procedure as described in Example 1 to produce a concentrated blood lysate. Reaction buffer containing the primers in Table 6 was added to the lysate, and the sample was denatured prior to addition of thermostable polymerase. Following amplification of the samples, T2MR detection and Sanger sequencing was performed as described in Example 1.

Sequencing reactions were prepared as follows. Primers were diluted from stock, aliquoted, and labeled according to the desired sequencing method. The samples were sequenced bi-directionally with a forward and reverse primer, according to Table 6.

TABLE 6
Stock primers used for sequencing

| Target | Forward Primer | Reverse Primer |
|---|---|---|
| A. baumannii | SEQ ID NO: 1 | SEQ ID NO: 2 |
| E. coli | SEQ ID NO: 38 | SEQ ID NO: 39 |
| E. faecium | SEQ ID NO: 3 | SEQ ID NO: 4 |
| K. pneumoniae | SEQ ID NO: 5 | SEQ ID NO: 6 |
| P. aeruginosa | SEQ ID NO: 7 | SEQ ID NO: 8 |
| S. aureus | SEQ ID NO: 9 | SEQ ID NO: 10 |

The samples were then sent to a sequencing vendor for sequencing (e.g., GENEWIZ). Remaining amplicon not used in the T2MR detection or in sequencing was stored at −20° C. for three months to allow for repeat testing.

Single species spiked specimens were prepared in K2EDTA treated whole blood at 1-2× LoD (which corresponded to 1-16 CFU/mL, depending on the species) and 2-3× LoD (which corresponded to 2-24 CFU/mL, depending on the species).

Amplified materials were prepared for sequencing according to GENEWIZ's sample preparation guidelines; T2BACTERIA® primers were first diluted down to 100 μM from 1mM stock in 1× TE. From the 100 μM stock, the primers were further diluted down to 5 μM in sterile water. 45 μL of the amplified samples were aliquoted in labeled 1.7 mL centrifuge tubes with sample number, sample ID, species identification, and protocol number. The GENEWIZ Sanger sequencing submission form for unpurified PCR product was filled with sample name for the name of the appropriate Forward and Reverse primers.

Samples were processed by GENEWIZ and Sanger sequenced. An enzyme cleanup with Exonuclease I, Shrimp Alkaline Phosphatase, and buffer was used to degrade excess enzyme, dNTPs, and primers. The digestion product was then diluted and used as template for Sanger sequencing. Primer extension sequencing was performed using Applied Biosystems BIG DYE® v3.1. The reactions were then run on Applied Biosystem's 3730xl DNA Analyzer. The results were provided as .abi, .seq, and .phd files.

Based on the original cutoff criteria, the 1-2× LoD Efm sample failed to meet the acceptance criteria for two samples. The original acceptance criteria was based off the results of samples run in the development of the test. The control length is the first step in distinguishing between a positive and negative sample and is designed, primarily, to prevent BLAST searching short non-specific sequences. The trimmed amplicon is still required to meet a specification of 95% identity and coverage to a target species, which are believed to facilitate the specificity of the test. The cutoff was adjusted for Ab, Ed, Efm, Kp, and Sa, see Table 7. This change will prevent or reduce repeat testing and re-sequencing of samples and will prevent false negative results in clinical samples with untested genomic variability.

TABLE 7
Trimmed Amplicon Cutoff

| | | | Original Trimmed | | New Trimmed | |
|---|---|---|---|---|---|---|
| | Avg | | Amplicon Length | | Amplicon Length | |
| | Trimmed | Std. | (Approx. Avg - | | (Approx. Avg - | |
| Species | Length | Dev. | 5x Std. Dev.) | | 15x Std. Dev.) | |
| Ab | 242 | 11 | 150 | bp | 70 | bp |
| Eci | 166 | 7 | 100 | bp | 65 | bp |
| Efm | 271 | 8 | 200 | bp | 155 | bp |
| Kp | 240 | 11 | 150 | bp | 70 | bp |
| Pa | 88 | 9 | 45 | bp | 45 | bp* |
| Sa | 147 | 6 | 100 | bp | 50 | bp |

As a result of this change in cutoff, 4 negative samples required BLAST searching and 2 Efm samples (1 repeat) were identified as positive. All summary and final results shown are with the new cutoff.

B. Results

Six whole blood samples per target species (36 positive samples total) were tested at two concentrations, 1-2× and 2-3× LoD (which corresponded to 1-16 CFU/mL and 2-24 CFU/mL, respectively, depending on the species). Two negative whole blood samples were tested per target reaction mixture (12 negative samples total), a summary of sequencing and T2MR results is provided in Table 8. Thirty-six spiked positive samples at 1-2 and 2-3 times the T2BACTERIA® panel LoD's were determined to be positive by both T2MR and sequencing. Twelve negative samples were determined to be negative by both T2MR and sequencing. There were no discordant samples observed. These results demonstrate the analytical sensitivity and specificity of the T2MR sequencing methodology in samples containing concentrated amounts of cell debris and subject cell nucleic acid (e.g., DNA).

TABLE 8
Summary of T2MR and Sequencing Results

| | | Bases | Bases | | | | |
|---|---|---|---|---|---|---|---|
| | | before | after | T2 | | | Sequencing |
| Sample | Target | trimming | trimming | Results | Identity | Coverage | Result |
| 13_Ab_1-2x_PRO-00904- | Ab | 586 | 253 | 126 | 99 | 100 | 10/10 |
| Ab_F | | | | | | | |
| 13_Ab_1-2x_PRO-00904- | Ab | 500 | 255 | 126 | 99 | 98 | 10/10 |
| Ab_R | | | | | | | |
| 14_Ab_2-3x_PRO-00904- | Ab | 577 | 254 | 123 | 99 | 99 | 10/10 |
| Ab_F | | | | | | | |
| 14_Ab_2-3x_PRO-00904- | Ab | 352 | 250 | 123 | 99 | 100 | 10/10 |
| Ab_R | | | | | | | |
| 19_Ab_1-2x_PRO-00904- | Ab | 578 | 248 | 135 | 99 | 99 | 10/10 |
| Ab_F | | | | | | | |
| 19_Ab_1-2x_PRO-00904- | Ab | 537 | 252 | 135 | 98-99 | 99 | 10/10 |
| Ab_R | | | | | | | |
| 20_Ab_2-3x_PRO-00904- | Ab | 579 | 211 | 115 | 99 | 99 | 10/10 |
| Ab_F | | | | | | | |
| 20_Ab_2-3x_PRO-00904- | Ab | 542 | 250 | 115 | 98-99 | 99 | 10/10 |
| Ab_R | | | | | | | |
| 25_Ab_1-2x_PRO-00904- | Ab | 579 | 247 | 130 | 99-100 | 100 | 10/10 |
| Ab_F | | | | | | | |
| 25_Ab_1-2x_PRO-00904- | Ab | 370 | 194 | 130 | 99-100 | 100 | 10/10 |
| Ab_R | | | | | | | |
| 26_Ab_2-3x_PRO-00904- | Ab | 583 | 250 | 136 | 99-100 | 100 | 10/10 |
| Ab_F | | | | | | | |
| 26_Ab_2-3x_PRO-00904- | Ab | 431 | 249 | 136 | 98-99 | 100 | 10/10 |
| Ab_R | | | | | | | |
| 15_Eci_1-2x_PRO-00904- | Eci | 276 | 188 | 528 | 100 | 99 | 9/10 |
| Eci_F | | | | | | | |
| 15_Eci_1-2x_PRO-00904- | Eci | 277 | 184 | 528 | 99 | 100 | 9/10 |
| Eci_R | | | | | | | |
| 16_Eci_2-3x_PRO-00904- | Eci | 307 | 183 | 483 | 100 | 99 | 9/10 |
| Eci_F | | | | | | | |
| 16_Eci_2-3x_PRO-00904- | Eci | 280 | 185 | 483 | 100 | 99 | 9/10 |
| Eci_R | | | | | | | |
| 21_Eci_1-2x_PRO-00904- | Eci | 316 | 180 | 540 | 100 | 99 | 9/10 |
| Eci_F | | | | | | | |
| 21_Eci_1-2x_PRO-00904- | Eci | 313 | 170 | 540 | 99 | 99 | 9/10 |
| Eci_R | | | | | | | |
| 22_Eci_2-3x_PRO-00904- | Eci | 315 | 183 | 491 | 100 | 99 | 9/10 |
| Eci_F | | | | | | | |
| 22_Eci_2-3x_PRO-00904- | Eci | 298 | 178 | 491 | 99 | 99 | 9/10 |
| Eci_R | | | | | | | |
| 27_Eci_1-2x_PRO-00904- | Eci | 326 | 188 | 401 | 100 | 99 | 9/10 |
| Eci_F | | | | | | | |
| 27_Eci_1-2x_PRO-00904- | Eci | 315 | 179 | 401 | 100 | 99 | 9/10 |
| Eci_R | | | | | | | |
| 28_Eci_2-3x_PRO-00904- | Eci | 307 | 183 | 371 | 100 | 99 | 9/10 |
| Eci_F | | | | | | | |
| 28_Eci_2-3x_PRO-00904- | Eci | 327 | 185 | 371 | 99 | 98 | 9/10 |
| Eci_R | | | | | | | |
| 17_Efm_1-2x_PRO-00904- | Efm | 310 | 156 | 162 | 99-100 | 100 | 10/10 |
| Efm_F | | | | | | | |
| 17_Efm_1-2x_PRO-00904- | Efm | 312 | 87 | 162 | 97-98 | 100 | 10/10 |
| Efm_R | | | | | | | |
| 18_Efm_2-3x_PRO-00904- | Efm | 312 | 280 | 142 | 99-100 | 100 | 10/10 |
| Efm_F | | | | | | | |
| 18_Efm_2-3x_PRO-00904- | Efm | 303 | 283 | 142 | 99-100 | 100 | 10/10 |
| Efm_R | | | | | | | |
| 23_Efm_1-2x_PRO-00904- | Efm | 311 | 265 | 153 | 99-100 | 100 | 10/10 |
| Efm_F | | | | | | | |
| 23_Efm_1-2x_PRO-00904- | Efm | 308 | 285 | 153 | 99 | 99 | 10/10 |
| Efm_R | | | | | | | |
| 24_Efm_2-3x_PRO-00904- | Efm | 311 | 280 | 176 | 99-100 | 100 | 10/10 |
| Efm_F | | | | | | | |
| 24_Efm_2-3x_PRO-00904- | Efm | 302 | 282 | 176 | 99-100 | 100 | 10/10 |
| Efm_R | | | | | | | |
| 01_Efm_1-2X_PRO-00904- | Efm | 311 | 274 | 147 | 99-100 | 100 | 10/10 |
| Efm_F | | | | | | | |
| 01_Efm_1-2X_PRO-00904- | Efm | 315 | 278 | 147 | 99 | 99 | 10/10 |
| Efm_R | | | | | | | |
| 30_Efm_2-3x_PRO-00904- | Efm | 311 | 280 | 176 | 99-100 | 100 | 10/10 |
| Efm_F | | | | | | | |
| 30_Efm_2-3x_PRO-00904- | Efm | 305 | 285 | 176 | 99-100 | 99 | 10/10 |
| Efm_R | | | | | | | |
| 31_Kp_1-2x_PRO-00904- | Kp | 364 | 256 | 539 | 99 | 100 | 10/10 |
| Kp_F | | | | | | | |
| 31_Kp_1-2x_PRO-00904- | Kp | 355 | 260 | 539 | 99 | 100 | 10/10 |
| Kp_R | | | | | | | |
| 32_Kp_2-3x_PRO-00904- | Kp | 360 | 260 | 624 | 99 | 99 | 10/10 |
| Kp_F | | | | | | | |
| 32_Kp_2-3x_PRO-00904- | Kp | 356 | 260 | 624 | 99 | 100 | 10/10 |
| Kp_R | | | | | | | |
| 37_Kp_1-2x_PRO-00904- | Kp | 397 | 258 | 615 | 99 | 100 | 10/10 |
| Kp_F | | | | | | | |
| 37_Kp_1-2x_PRO-00904- | Kp | 391 | 231 | 615 | 99 | 99 | 10/10 |
| Kp_R | | | | | | | |
| 38_Kp_2-3x_PRO-00904- | Kp | 355 | 258 | 416 | 99 | 100 | 10/10 |
| Kp_F | | | | | | | |
| 38_Kp_2-3x_PRO-00904- | Kp | 284 | 243 | 416 | 99 | 100 | 10/10 |
| Kp_R | | | | | | | |
| 43_Kp_1-2x_PRO-00904- | Kp | 435 | 264 | 619 | 99 | 99 | 10/10 |
| Kp_F | | | | | | | |
| 43_Kp_1-2x_PRO-00904- | Kp | 358 | 260 | 619 | 100 | 100 | 10/10 |
| Kp_R | | | | | | | |
| 44_Kp_2-3x_PRO-00904- | Kp | 362 | 256 | 393 | 99 | 100 | 10/10 |
| Kp_F | | | | | | | |
| 44_Kp_2-3x_PRO-00904- | Kp | 360 | 260 | 393 | 99 | 100 | 10/10 |
| Kp_R | | | | | | | |
| 01_Ab_Neg_PRO-00904- | Neg | 650 | 2 | 29 | n/a | n/a | n/a |
| Ab_F | | | | | | | |
| 01_Ab_Neg_PRO-00904- | Neg | 643 | 2 | 29 | n/a | n/a | n/a |
| Ab_R | | | | | | | |
| 02_Ab_Neg_PRO-00904- | Neg | 630 | 25 | 28 | 100 | 76 | 6/10 |
| Ab_F | | | | | | | |
| 02_Ab_Neg_PRO-00904- | Neg | 710 | 2 | 28 | n/a | n/a | n/a |
| Ab_R | | | | | | | |
| 03_Kp_Neg_PRO-00904- | Neg | 476 | 344 | 38 | n/a | n/a | 0/10 |
| Kp_F | | | | | | | |
| 03_Kp_Neg_PRO-00904- | Neg | 467 | 84 | 38 | n/a | n/a | 0/10 |
| Kp_R | | | | | | | |
| 04_Kp_Neg_PRO-00904- | Neg | 396 | 36 | 37 | n/a | n/a | 0/10 |
| Kp_F | | | | | | | |
| 04_Kp_Neg_PRO-00904- | Neg | 393 | 2 | 37 | n/a | n/a | n/a |
| Kp_R | | | | | | | |
| 05_Eci_Neg_PRO-00904- | Neg | 366 | 246 | 31 | n/a | n/a | 0/10 |
| Eci_F | | | | | | | |
| 05_Eci_Neg_PRO-00904- | Neg | 365 | 341 | 31 | n/a | n/a | 0/10 |
| Eci_R | | | | | | | |
| 06_Eci_Neg_PRO-00904- | Neg | 267 | 22 | 30 | n/a | n/a | 0/10 |
| Eci_F | | | | | | | |
| 06_Eci_Neg_PRO-00904- | Neg | 256 | 24 | 30 | 100 | 83 | 10/10 |
| Eci_R | | | | | | | |
| 07_Pa_Neg_PRO-00904- | Neg | 210 | 2 | 28 | n/a | n/a | n/a |
| Pa_F | | | | | | | |
| 07_Pa_Neg_PRO-00904- | Neg | 282 | 2 | 28 | n/a | n/a | n/a |
| Pa_R | | | | | | | |
| 08_Pa_Neg_PRO-00904- | Neg | 210 | 19 | 30 | n/a | n/a | 0/10 |
| Pa_F | | | | | | | |
| 08_Pa_Neg_PRO-00904- | Neg | 270 | 2 | 30 | n/a | n/a | n/a |
| Pa_R | | | | | | | |
| 09_Efm_Neg_PRO-00904- | Neg | 321 | 233 | 31 | n/a | n/a | 0/10 |
| Efm_F | | | | | | | |
| 09_Efm_Neg_PRO-00904- | Neg | 327 | 175 | 31 | 94 | 17 | 4/10 |
| Efm_R | | | | | | | |
| 10_Efm_Neg_PRO-00904- | Neg | 330 | 232 | 31 | n/a | n/a | 0/10 |
| Efm_F | | | | | | | |
| 10_Efm_Neg_PRO-00904- | Neg | 330 | 230 | 31 | 100 | 13 | 4/10 |
| Efm_R | | | | | | | |
| 11_Sa_Neg_PRO-00904- | Neg | 277 | 225 | 35 | n/a | n/a | 0/10 |
| SaA_F | | | | | | | |
| 11_Sa_Neg_PRO-00904- | Neg | 605 | 186 | 35 | n/a | n/a | 0/10 |
| SaA_R | | | | | | | |
| 12_Sa_Neg_PRO-00904- | Neg | 280 | 2 | 39 | n/a | n/a | n/a |
| SaA_F | | | | | | | |
| 12_Sa_Neg_PRO-00904- | Neg | 346 | 2 | 39 | n/a | n/a | n/a |
| SaA_R | | | | | | | |
| 33_Pa_1-2x_PRO-00904- | Pa | 395 | 107 | 525 | 100 | 100 | 10/10 |
| Pa_F | | | | | | | |
| 33_Pa_1-2x_PRO-00904- | Pa | 282 | 30 | 525 | 100 | 80 | 10/10 |
| Pa_R | | | | | | | |
| 34_Pa_2-3x_PRO-00904- | Pa | 282 | 93 | 477 | 96 | 100 | 10/10 |
| Pa_F | | | | | | | |
| 34_Pa_2-3x_PRO-00904- | Pa | 282 | 30 | 477 | 100 | 80 | 10/10 |
| Pa_R | | | | | | | |
| 39_Pa_1-2x_PRO-00904- | Pa | 305 | 106 | 495 | 100 | 100 | 10/10 |
| Pa_F | | | | | | | |
| 39_Pa_1-2x_PRO-00904- | Pa | 299 | 25 | 495 | 100 | 92 | 10/10 |
| Pa_R | | | | | | | |
| 40_Pa_2-3x_PRO-00904- | Pa | 318 | 102 | 504 | 100 | 98 | 10/10 |
| Pa_F | | | | | | | |
| 40_Pa_2-3x_PRO-00904- | Pa | 311 | 27 | 504 | 100 | 66-81 | 0/10 |
| Pa_R | | | | | | | |
| 45_Pa_1-2x_PRO-00904- | Pa | 358 | 98 | 480 | 99 | 100 | 10/10 |
| Pa_F | | | | | | | |
| 45_Pa_1-2x_PRO-00904- | Pa | 283 | 17 | 480 | 100 | 100 | 4/10 |
| Pa_R | | | | | | | |
| 46_Pa_2-3x_PRO-00904- | Pa | 367 | 95 | 508 | 99-100 | 97-100 | 10/10 |
| Pa_F | | | | | | | |
| 46_Pa_2-3x_PRO-00904- | Pa | 283 | 27 | 508 | 100 | 100 | 10/10 |
| Pa_R | | | | | | | |
| 35_Sa_1-2x_PRO-00904- | Sa | 274 | 149 | 344 | 99 | 100 | 10/10 |
| SaA_F | | | | | | | |
| 35_Sa_1-2x_PRO-00904- | Sa | 291 | 152 | 344 | 99 | 100 | 10/10 |
| SaA_R | | | | | | | |
| 36_Sa_2-3x_PRO-00904- | Sa | 272 | 157 | 329 | 99 | 99 | 10/10 |
| SaA_F | | | | | | | |
| 36_Sa_2-3x_PRO-00904- | Sa | 281 | 157 | 329 | 99 | 100 | 10/10 |
| SaA_R | | | | | | | |
| 41_Sa_1-2x_PRO-00904- | Sa | 273 | 159 | 279 | 99 | 98 | 10/10 |
| SaA_F | | | | | | | |
| 41_Sa_1-2x_PRO-00904- | Sa | 272 | 155 | 279 | 99 | 100 | 10/10 |
| SaA_R | | | | | | | |
| 42_Sa_2-3x_PRO-00904- | Sa | 346 | 157 | 322 | 100 | 100 | 10/10 |
| SaA_F | | | | | | | |
| 42_Sa_2-3x_PRO-00904- | Sa | 347 | 158 | 322 | 99 | 98 | 10/10 |
| SaA_R | | | | | | | |
| 47_Sa_1-2x_PRO-00904- | Sa | 274 | 149 | 345 | 99 | 100 | 10/10 |
| SaA_F | | | | | | | |
| 47_Sa_1-2x_PRO-00904- | Sa | 281 | 152 | 345 | 99 | 100 | 10/10 |
| SaA_R | | | | | | | |
| 48_Sa_2-3x_PRO-00904- | Sa | 315 | 157 | 308 | 100 | 100 | 10/10 |
| SaA_F | | | | | | | |
| 48_Sa_2-3x_PRO-00904- | Sa | 344 | 156 | 308 | 99 | 100 | 10/10 |
| SaA_R | | | | | | | |

### Example 3

### Detection of Bacterial Species in Clinical Samples by Sanger Sequencing and T2MR

The T2BACTERIA® panel, performed using the T2DX® instrument, is a qualitative T2 magnetic resonance in vitro diagnostic test for the detection and identification of A. baumannii, E. coli, E. faecium, K. pneumoniae, P. aeruginosa, and S. aureus. To further characterize whole blood samples that have been run on the T2DX® instrument with the T2BACTERIA® panel, a manual singleplex amplification assay was developed to detect the T2BACTERIA® panel targets by T2MR and confirm the presence of a species by bidirectional Sanger sequencing.

During the T2Bacteria Panel Pivotal Study, 3×4 mL whole blood samples were drawn directly after blood culture draws. The first tube, tube A, was to be used for T2BACTERIA® panel testing on the T2Dx and the remaining 2 whole blood samples, tubes B and C, were stored at −70° C. to −80° C. for discordant analysis. This Example summarizes results from using the manual singleplex amplification assay on available tubes B and C. At some sites tubes B or C were either not collected or not supplied to T2 Biosystems. A total of 112 results were determined to have T2+/BC− discordant results that were not resolved by evaluating additional culture results. Patient blood samples were not available for testing from 6 subjects, resulting in 106 T2+/BC− results that were tested using the independent method.

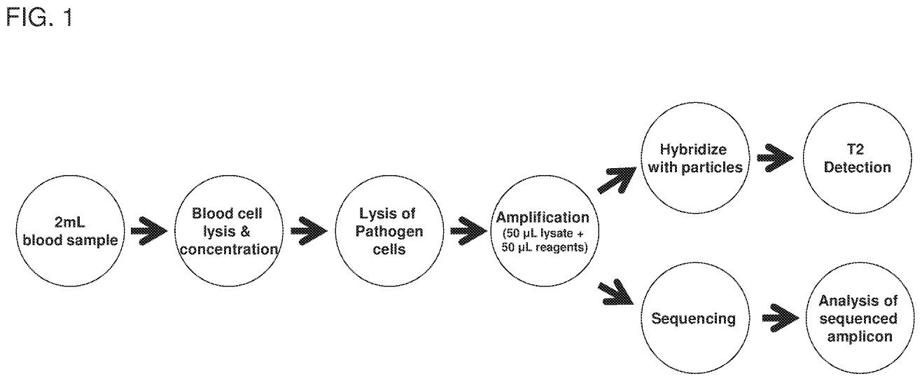

These samples were evaluated using the methods described in Examples 1 and 2 above, in which whole blood samples are processed and targets are amplified in singleplex with the T2BACTERIA® primers and detected by both T2MR and Sanger sequencing. See FIG. 1.

A. Materials and Methods

Samples were only tested for the target species that was identified as positive by T2MR when compared to a concomitant negative blood culture during the T2BACTERIA® prospective clinical trial. If a sample was positive by T2MR and negative by sequencing, sequencing was repeated with the stored amplicon. If the discordant result was consistent, sample processing, T2MR, and sequencing was repeated with a second aliquot of the sample or another tube of the same sample. If a sample was negative by T2MR and positive by sequencing for a species on the T2BACTERIA® panel, sequencing was repeated with the stored amplicon. If the discordant result was consistent, sample processing, T2MR, and sequencing was repeated with a second aliquot of the sample or another tube of the same sample.

For the test result for a sample to be considered T2MR positive, the T2MR signal for the bacterial target of interest must be greater than the defined cutoff. For the test result for a sample to be considered negative, the T2MR signal for the species of interest must be below the defined cutoff.

Sequencing analysis was performed as described in Examples 1 and 2.

Sample preparation, amplification, T2MR detection, and sequencing were performed as described in Examples 1 and 2.

B. Results

A total of 103 patient blood samples with 106 T2+/BC− results were tested, 3 samples had 2 T2+/BC− results. 33 results were identified as sequencing positive for the same species identified by the T2BACTERIA® panel in the clinical trial, and 73 results were identified as sequencing negative. Samples from 101 patients were concordant between T2MR and sequencing.

For 31 of the 32 sequencing positive samples, the top BLAST hits were concordant with the expected species. One sample for K. pneumoniae had E. aerogenes as the most prevalent result. The K. pneumoniae target is in the 23S ribosomal DNA region which is highly conserved between K. pneumoniae and E. aerogenes, in particular in the region that was sequenced and subject to BLASTn search. Although these two species have high sequence homology in the amplified region, exclusivity testing showed E. aerogenes to not be cross-reactive with T2BACTERIA® panel detection probes. Therefore, the amplified product was evaluated further by purification with gel electrophoresis, analyzed by qPCR, and subsequent Sanger Sequencing. qPCR showed that the Tm of the gel purified amplicon matched that of a K. pneumoniae control and sequencing this purified amplicon resulted in the top BLASTn results being K. pneumoniae. Several E. coli samples had BLAST hits for Shigella spp., as expected because of the known 100% sequence homology between E. coli and Shigella spp. for the amplified region. Shigella spp. has been tested in exclusivity testing and shown to be cross-reactive with the T2BACTERIA® E. coli channel. A total of 3 samples (2 Kp and 1 Ab) were initially identified as T2MR positive and sequencing negative. All three samples were concordant when retested.

These data demonstrate that the present methods can be used to perform sequencing of target nucleic acids in lysates of clinical blood sampels containing concentrated amount (estimated at approximately 10-fold) of cell debris and subject cell nucleic acids (e.g., DNA).

### Example 4

### Identification of Targets Using Next Generation Sequencing (NGS) and 16S rRNA Primers

A study was performed to determine whether NGS can be performed on amplicon produced using 16S primers and genomic DNA (g DNA) spiked into whole blood lysate and to further determine whether purification is necessary.

The targets in this study included panel members of the T2BACTERIA® panel, as shown in Table 9 below.

TABLE 9
Targets and strains used

| Target | gDNA Strain ID |
|---|---|
| Acinetobacter baumannii | ATCC BAA-1710 |
| Escherichia coli | ATCC 8739 |
| Enterococcus faecium | ATCC BAA-472 |
| Klebsiella pneumoniae | ATCC BAA-1705 |
| Pseudomonas aeruginosa | ATCC 47085 |
| Staphylococcus aureus | ATCC BAA-1718 |

A. Sample Preparation, Amplification, and Sequencing

The PCR setup was performed as follows. For each target, gDNA was spiked into negative whole blood lysate at 1,000 copies per reaction. Spiked samples were amplified with 16S primers on a Roche 480 LIGHTCYCLER® using the following primers:

(SEQ ID NO: 60)
Forward Primer - Bac16-1088
5′-GGTTAAGTCCCGCAACGAGCGC-3′
(SEQ ID NO: 61)
Reverse Primer - Bac16 R1540
5′-AGGAGGTGATCCAACCGCA-3′

Sample preparation was performed as follows. Lysate with gDNA and reaction buffer were combined in PCR strips, denatured for 5 minutes at 95° C., and spun down in a centrifuge at 8000×g for 5 min. The supernatant was pulled off and placed into a PCR plate. Enzyme with SYBR® Green was added to the wells. The plate was loaded onto a Roche 480 LIGHTCYCLER®. The LIGHTCYCLER® data were analyzed only for confirmation of the production of the amplicon. The PCR cycles included 1 cycle of 95° C. for 5 min, 45 cycles of 95° C. for 20 sec, 58° C. for 30 sec, and 68° C. for 30 sec, followed by 1 cycle of 95° C. for 5 sec and 65° C. for 1 min.

After amplification, each sample was split in half. One half was purified using the AGENCOURT AMPURE XP® PCR purification kit (Cat. No. A63880). The other half was left as an unpurified amplicon. Both unpurified and purified amplicon for each target was submitted to GENEWIZ for their AMPLICON EZ NGS service, which was performed using an ILLUMINA® 2×150PE platform configuration. Briefly, barcode sequences and standard ILLUMINA® adaptors were ligated to full-length amplicon (sequencing length was 100-250 bp). Sequencing was performed from both ends of the amplicon. Three samples were lost during the GENEWIZ service and no data is available for these (Acinetobacter baumannii (Ab) purified, Escherichia coli (Eci) unpurified, Enterococcus faecium (Efm) unpurified). Sequencing results were analyzed using BLASTn taxonomic output.

B. Results

Table 10 shows the results in terms of sequencing quality. A high number of reads and mean quality score over 35 for both unpurified and purified amplicon were obtained. These results indicate that omitting purification of the amplicon does not adversely affect the quality of sequencing result. A table with the total number of reads of each taxonomic ID without any cutoffs is shown in Table 11. Dark gray cells in Table 11 indicate positives for the correct genus and species, and light gray indicates positives for the correct genus only.

TABLE 10
NGS reads and quality

| | Raw Amplicon | | | | Purified Amplicon | | | |
|---|---|---|---|---|---|---|---|---|
| | | | Mean | % | | | Mean | % |
| | Number | Yield | Quality | Bases >= | Number | Yield | Quality | Bases >= |
| | of Reads | (Mbases) | Score | Q30 | of Reads | (Mbases) | Score | Q30 |
| Ab | 178,404 | 54 | 36.86 | 95.52 | | | | |
| Kp | 118,935 | 36 | 37.38 | 96.76 | 130,852 | 40 | 37.56 | 97.45 |
| Eci | | | | | 150,308 | 45 | 37.38 | 96.75 |
| Efm | | | | | 121,980 | 37 | 37.52 | 97.32 |
| Pa | 154,223 | 47 | 37.42 | 96.9 | 116,190 | 35 | 37.33 | 96.57 |
| Sa | 114,012 | 34 | 37.33 | 96.59 | 117,938 | 36 | 37.26 | 96.31 |

TABLE 11
Total number of reads by taxonomic ID

| | | Eci_Puri- | Efm_Puri- | Kp_Puri- | | Pa_Puri- | | Sa_Puri- | |
|---|---|---|---|---|---|---|---|---|---|
| Taxonomy ID | Ab_Raw | fied | fied | fied | Kp_Raw | fied | Pa_Raw | fied | Sa_Raw |
| Acinetobacter baumannii | 240156 | 104402 | 131205 | 94128 | 83743 | 0 | 0 | 0 | 0 |
| Acinetobacter baumannii BJAB0868 | 105 | 0 | 0 | 0 | 0 | 0 | 0 | 0 | 0 |
| Acinetobacter baumannii Naval-17 | 74 | 0 | 0 | 0 | 0 | 0 | 0 | 0 | 0 |
| Acinetobacter baumannii OIFC143 | 82 | 0 | 0 | 0 | 0 | 0 | 0 | 0 | 0 |
| Acinetobacter calcoaceticus | 74 | 0 | 0 | 0 | 0 | 0 | 0 | 0 | 0 |
| Acinetobacter junii | 396 | 0 | 0 | 0 | 0 | 0 | 0 | 0 | 0 |
| Acinetobacter pittii | 69 | 0 | 0 | 0 | 0 | 0 | 0 | 0 | 0 |
| Acinetobacter soli | 325 | 0 | 0 | 0 | 0 | | 0 | 0 | 0 |
| Acinetobacter sp. RMRCBF19 | 187 | 0 | 0 | 0 | 0 | 0 | 0 | 0 | 0 |
| Alcanivorax sp. | 0 | 56 | 0 | 59 | 0 | 0 | 0 | 0 | 0 |
| Arthrobacter sp. PD9 | 523 | 0 | 0 | 0 | 0 | 0 | 0 | 0 | 0 |
| bacterium 19(2013) | 0 | 0 | 0 | 0 | 0 | 148 | 182 | 0 | 0 |
| bacterium 26(2013) | 0 | 0 | 0 | 0 | 0 | 120 | 0 | 0 | 0 |
| bacterium endosymbiont of | 0 | 179 | 0 | 173 | 151 | 0 | 0 | 0 | 0 |
| Onthophagus Taurus | | | | | | | | | |
| bacterium MCF37(2011) | 0 | 54 | 0 | 0 | 0 | 0 | 0 | 0 | 0 |
| bacterium NLAE-zl-P23 | 0 | 0 | 0 | 54 | 0 | 0 | 0 | 0 | 0 |
| Brenneria goodwinii | 0 | 61 | 0 | 57 | 50 | 0 | 0 | 0 | 0 |
| Candidatus Accumulibacter | 163 | 0 | 0 | 0 | 0 | 0 | 0 | 0 | 0 |
| phosphatis clade IIA str. UW-1 | | | | | | | | | |
| Citrobacter sp. PSB2 | 0 | 69 | 0 | 60 | 64 | 0 | 0 | 0 | 0 |
| Comamonadaceae bacterium | 5864 | 0 | 0 | 67 | 130 | 0 | 0 | 0 | 0 |
| Comamonadaceae bacterium MPsc | 0 | 0 | 0 | 55 | 117 | 0 | 0 | 0 | 0 |
| Enterobacter cloacae | 0 | 107 | 0 | 50 | 0 | 0 | 0 | 0 | 0 |
| Enterobacter hormaechei subsp. | 0 | 93823 | 0 | 101048 | 88445 | 0 | 0 | 0 | 0 |
| steigerwaltii | | | | | | | | | |
| Enterococcus faecalis | 0 | 0 | 1139 | 0 | 0 | 0 | 0 | 0 | 0 |
| Enterococcus faecium | 0 | 0 | 87440 | 0 | 0 | 0 | 0 | 0 | 0 |
| Enterococcus mundtii | 0 | 0 | 38435 | 0 | 0 | 0 | 0 | 0 | 0 |
| Enterococcus sp. DGM UTI3a | 0 | 0 | 280 | 0 | 0 | 0 | 0 | 0 | 0 |
| Escherichia coli | 0 | 1262 | 0 | 1383 | 1088 | 0 | 0 | 0 | 0 |
| Escherichia coli Nissle 1917 | 0 | 106 | 0 | 0 | 0 | 0 | 0 | 0 | 0 |
| Escherichia coli O114:1-149 | 0 | 59 | 0 | 63 | 62 | 0 | 0 | 0 | 0 |
| Escherichia coli O128:H27 | 0 | 124 | 0 | 56 | 0 | 0 | 0 | 0 | 0 |
| Escherichia hermannii | 0 | 63 | 0 | 0 | 0 | 0 | 0 | 0 | 0 |
| Escherichia sp. | 0 | 57 | 0 | 55 | 0 | 0 | 0 | 0 | 0 |
| Homo sapiens | 12325 | 0 | 297 | 0 | 252 | 12439 | 71984 | 785 | 2498 |
| Klebsiella oxytoca | 0 | 0 | 0 | 2223 | 1274 | 0 | 0 | 0 | 0 |
| Klebsiella pneumoniae | 0 | 11821 | 0 | 26818 | 24081 | 0 | 0 | 0 | 0 |
| Klebsiella pneumoniae subsp. | 0 | 225 | 0 | 257 | 212 | 0 | 0 | 0 | 0 |
| pneumoniae | | | | | | | | | |
| Klebsiella sp. PRW-1 | 0 | 119 | 0 | 0 | 0 | 0 | 0 | 0 | 0 |
| Klebsiella sp. Y3 | 0 | 129 | 0 | 52 | 63 | 0 | 0 | 0 | 0 |
| Klebsiella variicola At-22 | 0 | 0 | 0 | 54 | 0 | 0 | 0 | 0 | 0 |
| Lactobacillus sakei subsp. | 0 | 0 | 69 | 0 | 0 | 0 | 0 | 0 | 0 |
| sakei | | | | | | | | | |
| Methylobacterium populi BJ001 | 0 | 0 | 0 | 0 | 1146 | 0 | 0 | 0 | 0 |
| Pantoea sp. At-9b | 0 | 56 | 0 | 0 | 56 | 0 | 0 | 0 | 0 |
| Providencia stuartii | 0 | 133 | 0 | 64 | 126 | 0 | 0 | 0 | 0 |
| Pseudomonas aeruginosa | 0 | 0 | 0 | 0 | 0 | 184870 | 188551 | 0 | 0 |
| Pseudomonas knackmussii | 0 | 0 | 0 | 0 | 0 | 976 | 1023 | 0 | 0 |
| Pseudomonas sp. 9_2c_3 | 0 | 0 | 0 | 0 | 0 | 164 | 0 | 0 | 0 |
| Pseudomonas sp. BRRh-6 | 0 | 0 | 0 | 0 | 0 | 94 | 0 | 0 | 0 |
| Pseudomonas sp. GLY-3102 | 0 | 0 | 0 | 0 | 0 | 46 | 0 | 0 | 0 |
| Pseudomonas sp. J16 | 0 | 0 | 0 | 0 | 0 | 398 | 447 | 0 | 0 |
| Pseudomonas sp. TB23 | 0 | 0 | 0 | 0 | 0 | 0 | 0 | 48 | 0 |
| Pseudomonas sp. ZJY-733 | 0 | 0 | 0 | 0 | 0 | 1684 | 1741 | 0 | 0 |
| Pseudomonas stutzeri | 0 | 123 | 0 | 52 | 49 | 0 | 0 | 0 | 0 |
| Salmonella enterica | 0 | 66 | 0 | 60 | 63 | 0 | 0 | 0 | 0 |
| Salmonella enterica subsp. | 0 | 147 | 0 | 59 | 64 | 0 | 0 | 0 | 0 |
| enterica serovar Derby | | | | | | | | | |
| Shigella boydii | 0 | 529 | 0 | 360 | 266 | 0 | 0 | 0 | 0 |
| Shigella dysenteriae | 0 | 465 | 0 | 452 | 402 | 0 | 0 | 0 | 0 |
| Shigella flexneri | 0 | 461 | 0 | 390 | 461 | 0 | 0 | 0 | 0 |
| Shigella sonnei 53G | 0 | 109 | 0 | 196 | 195 | 0 | 0 | 0 | 0 |
| Sodalis sp. HS1 | 0 | 729 | 0 | 656 | 623 | 0 | 0 | 0 | 0 |
| Staphylococcus aureus | 0 | 0 | 0 | 0 | 0 | 0 | 0 | 102686 | 98377 |
| Staphylococcus aureus subsp. | 0 | 0 | 0 | 0 | 0 | 0 | 0 | 88 | 85 |
| aureus | | | | | | | | | |
| Staphylococcus capitis subsp. | 0 | 0 | 0 | 0 | 0 | 0 | 0 | 223 | 191 |
| capitis | | | | | | | | | |
| Staphylococcus epidermidis | 0 | 0 | 0 | 0 | 0 | 0 | 0 | 578 | 540 |
| Staphylococcus hominis | 0 | 0 | 0 | 0 | 0 | 0 | 0 | 51 | 51 |
| Staphylococcus pasteuri | 0 | 0 | 0 | 0 | 0 | 0 | 0 | 99583 | 95960 |
| Staphylococcus sciuri | 0 | 0 | 0 | 0 | 0 | 0 | 0 | 66 | 0 |
| Staphylococcus sp. cTPY-19 | 0 | 0 | 0 | 0 | 0 | 0 | 0 | 196 | 191 |
| Staphylococcus sp. PCA17 | 0 | 0 | 0 | 0 | 0 | 0 | 0 | 265 | 252 |
| Staphylococcus sp. sen1039 | 0 | 0 | 0 | 0 | 0 | 0 | 0 | 235 | 234 |
| Staphylococcus sp. Tp10 | 0 | 0 | 0 | 0 | 0 | 0 | 0 | 192 | 157 |
| Vagococcus sp. | 0 | 0 | 139 | 0 | 0 | 0 | 0 | 0 | 0 |
| Variovorax paradoxus | 5776 | 0 | 0 | 0 | 0 | 0 | 0 | 0 | 0 |
| Grand Total | 266119 | 215534 | 259004 | 229001 | 203183 | 200939 | 263928 | 204996 | 198536 |

TABLE 12
NGS results

| | Ab Raw | | Eci Purified | | Efm Purified | | Kp Purified | | Kp Raw | |
|---|---|---|---|---|---|---|---|---|---|---|
| | Sum of | Percent | Sum of | Percent | Sum of | Percent | Sum of | Percent | Sum of | Percent |
| Tax ID | Reads | of Reads | Reads | of Reads | Reads | of Reads | Reads | of Reads | Reads | of Reads |
| Acinetobacter baumannii | 238,315 | 95.34% | 104,402 | 48.90% | 131,205 | 50.81% | 94,128 | 41.37% | 83,743 | 41.76% |
| Comamonadaceae bacterium | 5,864 | 2.35% | | | | | | | | |
| Enterobacter hormaechei | | | 93,823 | 43.95% | | | 101,048 | 44.41% | 88,445 | 44.11% |
| subsp. steigerwaltii | | | | | | | | | | |
| Enterococcus faecalis | | | | | 1,139 | 0.44% | | | | |
| Enterococcus faecium | | | | | 87,440 | 33.86% | | | | |
| Enterococcus mundtii | | | | | 38,435 | 14.88% | | | | |
| Escherichia coli | | | 1,262 | 0.59% | | | 1,383 | 0.61% | 1,088 | 0.54% |
| Klebsiella oxytoca | | | | | | | 2,223 | 0.98% | 1,274 | 0.64% |
| Klebsiella pneumoniae | | | 11,821 | 5.54% | | | 26,818 | 11.79% | 24,081 | 12.01% |
| Pseudomonas aeruginosa | | | | | | | | | | |
| Pseudomonas knackmussii | | | | | | | | | | |
| Pseudomonas sp. ZJY-733 | | | | | | | | | | |
| Staphylococcus aureus | | | | | | | | | | |
| Staphylococcus pasteuri | | | | | | | | | | |
| Variovorax paradoxus | 5,776 | 2.31% | | | | | | | | |
| Grand Total | 249,955 | 100.00% | 213,492 | 100.00% | 258,219 | 100.00% | 227,525 | 100.00% | 200,513 | 100.00% |

| | Pa Purified | | Pa Raw | | Sa Purified | | Sa Raw | |
|---|---|---|---|---|---|---|---|---|
| | Sum of | Percent | Sum of | Percent | Sum of | Percent | Sum of | Percent |
| Tax ID | Reads | of Reads | Reads | of Reads | Reads | of Reads | Reads | of Reads |
| Acinetobacter baumannii | | | | | | | | |
| Comamonadaceae bacterium | | | | | | | | |
| Enterobacter hormaechei | | | | | | | | |
| subsp. steigerwaltii | | | | | | | | |
| Enterococcus faecalis | | | | | | | | |
| Enterococcus faecium | | | | | | | | |
| Enterococcus mundtii | | | | | | | | |
| Escherichia coli | | | | | | | | |
| Klebsiella oxytoca | | | | | | | | |
| Klebsiella pneumoniae | | | | | | | | |
| Pseudomonas aeruginosa | 184,870 | 98.58% | 188,551 | 98.56% | | | | |
| Pseudomonas knackmussii | | | 1,023 | 0.53% | | | | |
| Pseudomonas sp. ZJY-733 | 1,684 | 0.90% | 1,741 | 0.91% | | | | |
| Staphylococcus aureus | | | | | 101,987 | 50.45% | 97,585 | 50.28% |
| Staphylococcus pasteuri | | | | | 99,583 | 49.26% | 95,960 | 49.44% |
| Variovorax paradoxus | | | | | | | | |
| Grand Total | 187,530 | 100.00% | 191,315 | 100.00% | 202,148 | 100.00% | 194,085 | 100.00% |

Table 12 shows the results of the NGS analysis; dark gray results indicate positives for the correct genus and species, while light gray indicates positives for the correct genus only. Ab, Pseudomonas aeruginosa (Pa), and Staphylococcus aureus (Sa) performed very well, with >90% of reads for each sample coming back as the correct species or genus. Efm had comparatively worse results but still had correct genus or species identification in 49.18% of reads. Eci and Klebsiella pneumoniae (Kp) did not perform as well in this experiment; these samples had multiple Enterobacteriaceae hits, and it is believed that the 16s region sequenced was not variable enough to distinguish between different species within the family Enterobacteriaceae. It is believed that the identification of Ab in many samples is due to contamination. Out of the three samples that had both unpurified and purified amplicons, only Pa had a difference in the overall percentage of reads on target, which suggests that omitting PCR purification prior to sequencing does not inhibit the sequencing of targets.

In summary, these data demonstrate that surprisingly, NGS can be performed to sequence amplicons produced in a concentrated and “dirty” whole blood lysate using 16S rRNA primers. Unexpectedly, these data further show that purification of amplicons prior to sequencing is not essential. These data indicate that NGS sequencing in whole blood samples or other complex samples can be used directly after amplification to result in high-quality sequencing information that can be used to detect and sequence target nucleic acids.

### Example 5

### NGS Identification of Pathogen Genus/Species with 16S Amplification of DNA Isolated from Cells Spiked into Blood

A study was performed to evaluate and compare the sensitivity of NGS identification of bacterial genus/species using 16S amplification of DNA isolated from bacterial cells spiked into whole blood and processed using sample processing approach described below.

The targets of this study are shown in Table 13.

TABLE 13
Samples used

| Target | Strain ID | Concentration |
|---|---|---|
| Negative | n/a | 0 CFU/mL |
| Acinetobacter baumannii | ATCC 17978 | 10 CFU/mL, |
| | | 100 CFU/mL, |
| | | 1000 CFU/mL |
| Staphylococcus aureus | ATCC 29213 | 10 CFU/mL, |
| | | 100 CFU/mL, |
| | | 1000 CFU/mL |

A. Sample Preparation and Sequencing

Bacterial spiked samples were prepared by spiking bacteria from frozen cell aliquots into human whole blood.

The sample processing was performed as follows, essentially as described in Example 1. Lysis tubes containing a red blood cell lysis agent were spun down and the foil was removed. 2 mL of blood (spiked or control) was pipetted into each tube, and then mixed by pipetting up and down. The sample was allowed to lyse red blood cells for 5 min, and then the sample was centrifuged for 5 min at 6000×g. The supernatant was then removed, and 150 μL of TE was added to the pellet, which was then pulse vortexed briefly and centrifuged for 5 min at 6000×g. The supernatant was removed, and 100 μL of TE was mixed with the pellet, followed by lysis of pathogen cells and white blood cells by bead beating for 5 min on a vortexer. The sample was then centrifuged for 2 min at 6000×g, and the lysate was used for PCR testing.

The PCR reaction was set up as follows. T₂ BacPan 16S Primers as shown below were used to amplify 16S target nucleic acids using Roche 480 LIGHTCYCLER®

(SEQ ID NO: 60)
Forward Primer - Bac16-1088
5′-GGTTAAGTCCCGCAACGAGCGC-3′
(SEQ ID NO: 61)
Reverse Primer - Bac16 R1540
5′-AGGAGGTGATCCAACCGCA-3′

50 μL of lysate was mixed with reaction buffer in PCR strips, denatured for 5 min at 95° C., and spun down in a centrifuge at 8000×g for 5 min. The supernatant was removed and put into a PCR plate. 50 μL of the eluted QIAGEN®-purified DNA was also added to the plate. Enzyme with SYBR® Green was added to the wells. The plate was loaded onto a Roche 480 LIGHTCYCLER®. LIGHTCYCLER® data were analyzed for confirmation of production of amplicon. The PCR cycles included 1 cycle of 95° C. for 5 min, 40-45 cycles of 95° C. for 20 sec, 58° C. for 30 sec, and 68° C. for 30 sec, followed by 1 cycle of 95° C. for 5 sec and 65° C. for 1 min. Amplicon for each target was submitted to GENEWIZ for their AMPLICON EZ NGS service, which was performed using an ILLUMINA® 2×150PE platform configuration.

B. Results

Table 14 shows the NGS reads and quality. Negative results are shown in Table 15 (the results in the gray-shaded cells indicate greater than 5% of reads). Detection of negative samples resulted mostly from environmental organisms, and no false positives for targets tested.

TABLE 14
NGS reads and quality

| | | | Mean | |
|---|---|---|---|---|
| | Number | Yield | Quality | % |
| Sample ID | of Reads | (Mbases) | Score | Bases ≥30 |
| Negative | 78,181 | 39 | 35.78 | 90.84 |
| 10 CFU/mL Ab | 100,038 | 50 | 34.97 | 86.76 |
| 100 CFU/mL Ab | 82,955 | 42 | 35.66 | 90.09 |
| 1000 CFU/mL Ab | 69,197 | 35 | 36.45 | 93.94 |
| 10 CFU/mL Sa | 103,066 | 52 | 34.32 | 83.75 |
| 100 CFU/mL Sa | 81,090 | 41 | 35.64 | 90.22 |
| 1000 CFU/mL Sa | 68,860 | 35 | 36.61 | 94.67 |
| 100 CFU/mL Efm | 91,348 | 46 | 35.18 | 87.83 |
| 9 CFU/mL KPC-3 | 168,882 | 85 | 36.28 | 93.33 |

TABLE 15
Negative Results

T2Prep

| Negative | Sum of | Percent |
|---|---|---|
| TaxID | Reads | of Reads |
| Alcaligenes faecalis | 6666 | 9.91% |
| alpha proteobacterium SK50-23 | 1691 | 2.51% |
| bacterium PG | | |
| Bradyrhizobium sp. | 3429 | 5.10% |
| Bradyrhizobium sp. ‘SH 283012’ | 1646 | 2.45% |
| Halobacillus sp. LS27 | 2046 | 3.04% |
| Mesorhizobium loti | | |
| Methylobacterium sp. 4-46 | | |
| Pelomonas sp. 7B-406 | | |
| Phenylobacterium sp. | | |
| Pseudogracilibacillus sp. DT 7-02 | 2212 | 3.29% |
| Pseudomonas aeruginosa | | |
| Pseudomonas sp. | | |
| Pseudomonas sp. MBR | | |
| Ralstonia eutropha JMP134 | 10307 | 15.32% |
| Ralstonia mannitolilytica | 9842 | 14.63% |
| Rhizobium tubonense | | |
| Stenotrophomonas maltophilia | 9448 | 14.05% |
| Stenotrophomonas pavanii | 6230 | 9.26% |
| Stenotrophomonas sp. | 2590 | 3.85% |
| Streptococcus agalactiae | | |
| Streptococcus sp. LMG 27685 | | |
| Variovorax paradoxus | 11152 | 16.58% |
| Grand Total | 67259 | 100.00% |

Table 16 shows the results of Ab titration. No Ab was detected in samples processed using the sample preparation procedure described above. It should be noted that these studies utilized a different strain of Ab than was used in the study described in Example 4. Acinetobacter junii and Acinetobacter pittii were detected in both sample types. These species are homologous to Ab (about 98% for A. pittii and about 94% for A. junii). NGS was able to identify Acinetobacter spp. in samples that contained 10 CFU/mL of Ab.

TABLE 16
Results of Ab Titration

| | T2Prep | | | | | |
|---|---|---|---|---|---|---|
| | 10 CFU/mL | | 100 CFU/mL | | 1000 CFU/mL | |
| Ab Titration | Sum of | Percent of | Sum of | Percent of | Sum of | Percent of |
| Tax ID | Reads | Reads | Reads | Reads | Reads | Reads |
| Acinetobacter junii | 9592 | 14.77% | 6602 | 10.48% | 25367 | 35.79% |
| Acinetobacter pittii | 10465 | 16.11% | 6870 | 10.91% | 26536 | 37.43% |
| Bacillus pumilus | 3028 | 4.66% | | | | |
| bacterium MNFS-9 | 3324 | 5.12% | 1220 | 1.94% | | |
| bacterium PG | | | | | | |
| Bradyrhizobium sp. ‘SH 283012’ | | | 1481 | 2.35% | | |
| Clostridium thermobutyricum | | | | | 1138 | 1.61% |
| Haemophilus influenzae | 2517 | 3.88% | | | | |
| Haemophilus parainfluenzae | 2409 | 3.71% | | | | |
| T3T1 | | | | | | |
| marine bacterium AS-39 | 2178 | 3.35% | | | | |
| Methylobacterium isbiliense | | | | | | |
| Methylobacterium sp. 4-46 | | | | | | |
| Pelomonas sp. 7B-406 | | | | | | |
| Phenylobacterium sp. | | | | | | |
| Pseudomonas sp. | 3248 | 5.00% | 1217 | 1.93% | | |
| Ralstonia eutropha JMP134 | 5186 | 7.99% | 7868 | 12.49% | 3489 | 4.92% |
| Ralstonia mannitolilytica | 4739 | 7.30% | 7607 | 12.08% | 3264 | 4.60% |
| Staphylococcus epidermidis | | | | | | |
| Stenotrophomonas maltophilia | | | 4397 | 6.98% | 1425 | 2.01% |
| Stenotrophomonas pavanii | | | 1424 | 2.26% | | |
| Streptococcus agalactiae | | | | | | |
| Streptococcus gordonii | | | 3123 | 4.96% | | |
| Streptococcus sp. HTS2 | | | 2804 | 4.45% | | |
| Streptococcus sp. LMG 27685 | | | | | | |
| Variovorax paradoxus | 18255 | 28.11% | 18377 | 29.17% | 9668 | 13.64% |
| Grand Total | 64941 | 100% | 62990 | 100% | 70887 | 100% |

Table 17 shows the results of Sa titration. NGS was able to identify Sa at 10 CFU/mL.

TABLE 17
Results of Sa titration

| | T2Prep | | | | | |
|---|---|---|---|---|---|---|
| | 10 CFU/mL | | 100 CFU/mL | | 1000 CFU/mL | |
| Sa Titration | Sum of | Percent of | Sum of | Percent of | Sum of | Percent of |
| Tax ID | Reads | Reads | Reads | Reads | Reads | Reads |
| Acinetobacter sp. | | | | | | |
| bacterium | | | | | | |
| bacterium MNFS-9 | 1822 | 2.91% | | | | |
| bacterium PG | | | | | | |
| Bradyrhizobium sp. ‘SH 283012’ | 1046 | 1.67% | | | | |
| Escherichia coli | | | | | | |
| Methylobacterium isbiliense | | | | | | |
| Methylobacterium sp. 4-46 | | | | | | |
| Pelomonas sp. 7B-406 | | | | | | |
| Phenylobacterium sp. | | | | | | |
| Pseudomonas sp. | 1836 | 2.93% | | | | |
| Ralstonia eutropha JMP134 | 3538 | 5.65% | 3918 | 11.42% | 3182 | 4.18% |
| Ralstonia mannitolilytica | 3322 | 5.30% | 2710 | 7.90% | 2687 | 3.53% |
| Staphylococcus aureus | 12540 | 20.02% | 10405 | 30.32% | 37044 | 48.69% |
| Staphylococcus hominis | 9611 | 15.35% | 8163 | 23.79% | 24661 | 32.41% |
| Stenotrophomonas maltophilia | 3758 | 6.00% | 3090 | 9.00% | 2210 | 2.90% |
| Stenotrophomonas pavanii | 1089 | 1.74% | | | | |
| Streptococcus agalactiae | | | | | | |
| Streptococcus sp. LMG 27685 | | | | | | |
| Variovorax paradoxus | 24062 | 38.42% | 6030 | 17.57% | 6295 | 8.27% |
| Grand Total | 62624 | 100% | 34316 | 100% | 76079 | 100% |

C. Conclusions

The Ab and Sa data show that in all cases that sequencing of targets prepared using the sample processing procedure described herein detected the target at low titers. These data indicate that sequencing (e.g., NGS) can be used to detect pathogens at low titer in blood or other complex samples, e.g., about 1-10 CFU/mL.

### Example 6

### NGS Identification of Antibiotic Resistance Targets with Amplification of Plasmid-Based DNA Isolated from Cells Spiked into Blood Lysates

A study was performed to determine whether NGS can detect alternate targets such as antibiotic resistance targets in whole blood lysates. The targets included those listed in Table 18.

TABLE 18
Samples used

| Target | Strain ID | Concentration |
|---|---|---|
| Negative | n/a | 0 CFU/mL |
| bla_{KPC-3} resistance gene | AR-061 (Eci) | 9.0 CFU/mL |

A. Sample Preparation

The bla_{KPC-3} resistance gene was spiked into negative human whole blood. The sample processing was performed as follows, essentially as described in Example 1. Lysis tubes containing a red blood cell lysis agent were spun down and the foil was removed. 2 mL of blood (spiked or negative control) was pipetted into each tube, and then mixed by pipetting up and down. The sample was allowed to lyse red blood cells for 5 min, and then the sample was centrifuged for 5 min at 6000×g. The supernatant was then removed, and 150 μL of TE was added to the pellet, which was then pulse vortexed briefly and centrifuged for 5 min at 6000×g. The supernatant was removed, and 100 μL of TE was mixed with the pellet, followed by lysis of pathogen cells and white blood cells by bead beating for 5 min on a vortexer. The sample was then centrifuged for 2 min at 6000×g, and the lysate was used for PCR testing.

The PCR reaction was set up using a primer pair to amplify the bla_{kpc-3} target in a symmetric (200 nM) reaction buffer.

50 μL of lysate was mixed with reaction buffer in PCR strips, denatured for 5 min at 95° C., and spun down in a centrifuge at 8000×g for 5 min. The supernatant was removed and put into a PCR plate. Enzyme with SYBR® Green was added to the wells. The plate was loaded onto a Roche 480 LIGHTCYCLER®. LIGHTCYCLER® data was analyzed for confirmation of production of amplicon. Amplicon for each target was submitted to GENEWIZ for their Amplicon EZ NGS service. Sequencing results were analyzed using BLASTn taxonomic output.

B. Results

Table 14 above shows the NGS reads and quality. Detection of negative samples resulted mostly in environmental organisms, and there were no false positives for targets tested. Table 19 shows the results for detection of KPC-3. These data, demonstrating detection of KPC-3, show the method applies to detecting alternate targets such as antimicrobial resistance target genes. This approach can be used to identify particular antimicrobial resistance target genes as well as variants thereof.

TABLE 19
KPC-3 results

| ID | |
|---|---|
| Klebsiella pneumoniae | Ar-061 T2Prep |
| KPC-3 | 100.00% |

### Example 7

### Comparison of Sanger Sequencing Versus NGS

A study was performed to compare Sanger sequencing and NGS with both single target and double target spikes. Table 20 shows the targets and strain ID.

TABLE 20
Targets and strain ID

| Target | gDNA Strain ID |
|---|---|
| Enterococcus faecium | ATCC BAA-472 |
| Enterococcus faecalis | ATCC 49533 |
| Enterococcus faecium and | ATCC BAA-472 and ATCC 49533 |
| Enterococcus faecalis | |

A. Sample Preparation

The PCR reaction was prepared as follows. The gDNA as described in Table 20 was spiked in negative whole blood lysate prepared according to the sample processing procedure described in Examples 1-6. 1000 copies of gDNA were spiked per PCR reaction. T2BACTERIA® Efm Primers were used in an asymmetric master mix using an optimized sequencing ratio, e.g., as described in International Patent Application Publication No. WO 2017/127731, which is incorporated herein by reference in its entirety.

(SEQ ID NO: 96)
Efm Forward Primer -
5′-CTA TGT AGG GAA GGG ATA AAC GCT GA-3′
(SEQ ID NO: 97)
Efm Reverse Primer -
5′-GCG CTA AGG AGC TTA ACT TCT GTG TTC G-3′

Lysate and reaction buffer were combined in PCR strips, denatured for 5 min at 95° C., and spun down in a centrifuge at 8000×g for 5 min. Enzyme was added to each sample, and placed on a MASTERCYCLER® thermal cycler. The PCR cycling included 1 cycle of 95° C. for 3 min, followed by 40-46 cycles of 95° C. for 20 sec, 58° C. for 30 sec, and 68° C. for 30 sec, followed by 1 cycle of 68° C. for 3 min.

Each sample was detected with T₂ magnetic resonance (see, e.g., WO 2017/127731) to confirm amplicon production. Each sample was split in half. One half was sequenced with Sanger sequencing, and the other half was sequenced with NGS. Both amplicons were submitted to GENEWIZ for their Amplicon EZ NGS service and traditional Sanger sequencing.

B. Results

Table 21 shows the T2 signal of the amplicon as detected using a magnetic particle coated with Efm-specific probes. These results show that amplicon was produced.

TABLE 21
T2 signal of the amplicon

| Sample | Efm Particle (ms) |
|---|---|
| Efm | 233 |
| Efs | 28 |
| Efm/Efs double spike | 175 |

Table 22 shows the results of the Sanger sequencing. Sequencing of both of the single spikes worked very well, demonstrating that Sanger sequencing can be used for sequencing of amplicons in whole blood lysates, including those made using the sample processing procedure described in the preceding Examples. However, because Sanger can only detect one sequence, the Efm was masked by the Efs in the dual spike. Without wishing to be bound by theory, this could be because there was more Efs amplicon than Efm amplicon in the sample.

TABLE 22
Sanger Results

| Sample | Efm Hits | Efs Hits | Ident | Coverage |
|---|---|---|---|---|
| 1_Efm_F | 10/10 | | 100 | 100 |
| 1_Efm_R | 10/10 | | 100 | 100 |
| 1_Efm_consensus | 10/10 | | 100 | 100 |
| 2_Efs_F | | 10/10 | 99 | 100 |
| 2_Efs_R | | 10/10 | 99 | 100 |
| 2_Ef_consensus | | 10/10 | 99 | 100 |
| 3_Dual_F | | 10/10 | 99 | 100 |
| 3_Dual_R | | 10/10 | 95 | 99 |
| 3_Dual_consensus | | 10/10 | 98 | 100 |

Table 23 shows the results of the NGS. Sequencing of both single spikes worked well, confirming that NGS sequencing can be used to detect amplicons in whole blood lysates, including those made using the sample processing procedure described in the preceding Examples. Surprisingly, the results of the double spike show that coinfections can be detected by the NGS method. The NGS percent of reads further suggest that the Efs amplicon was dominant. The Enterococcus dispar and Enterococcus sp. CCM4360 BLAST results are due to a one base pair mismatch from the Efm sequence. These data indicate that if a patient has a coinfection with two pathogen species, NGS is able to identify each species even if the species are closely related.

TABLE 23
NGS Blast Results

| | Efm | | Efs | | Dual | |
|---|---|---|---|---|---|---|
| | Number | Percent | Number | Percent | Number | Percent |
| Blast Result | of reads | of Reads | of reads | of Reads | of reads | of Reads |
| Enterococcus dispar | 268 | 0.11% | | | | |
| ATCC 51266 | | | | | | |
| Enterococcus | | | 235,351 | 100% | 149,527 | 72% |
| faecalis | | | | | | |
| Enterococcus | 252,504 | 99.89% | | | 58,804 | 28% |
| faecium | | | | | | |
| Enterococcus sp. | | | | | 127 | 0.06% |
| CCM4360 | | | | | | |

### Example 8

### Exemplary Targets and Combined Use of T2MR and Sequencing

Table 24 shows exemplary targets that can be detected using the sequencing methods described herein, including Sanger, massively-parallel, and/or single-molecule sequencing. These targets can be detected using direct sequencing approaches, as described above. In other examples, these targets can be detected using a combined T2MR/sequencing detection approach.

For example, T2MR detection, e.g., as described above, can be used to provide group-level (e.g., genus-level) identification of target nucleic acids in complex samples such as blood, which can be followed by sequencing to provide more detailed species-level or variant-level information. The “Pan” channels would encompass any bacterial result from other channels that fall within the appropriate group-level categorization. Additionally, sequencing could detect species not listed on the matrix in Table 24. The other genus channels would be similar. For example, a sample containing an E. coli target nucleic acid would be identified as Pan Gram negative (e.g., by T2MR), Enterobacter spp.-positive, and E. coli-positive (e.g., by sequencing). Sequencing can provide additional information regarding antimicrobial resistance or identify the particular strain or subspecies to which the E. coli belongs. A Gram negative bacterium not on this panel could be identified as Pan Gram negative, Enterobacter spp.-positive and negative for all the other channels on the panel. Sequencing can then be used search for sequences that are in the Enterobacter genus. Thus, T2MR detection can be used to narrow the types of sequences that are analyzed. Sequencing can also be used to confirm or validate T2MR detection.

TABLE 24
Exemplary Targets

| | | Gram Positive | Gram Negative | Resistance | |
|---|---|---|---|---|---|
| Result | Gram & Genus | Species | Species | Genes | Fungal Species |
| No. | Channels | Channels | Channels | Channels | Channels |
| 1 | Pan Gram positive | E. faecium | A. baumanii | KPC | C. albicans |
| 2 | Pan Gram negative | E. faecalis | E. coli | NDM | C. tropicalis |
| 3 | Enterobacteriaceae | S. pneumoniae | H. influenzae | VIM | C. dublinensis |
| 4 | Enterobacter spp. | S. pyogenes | K. pneumoniae | IMP | C. parapsilosis, |
| | | | | | C. metapsilosis, |
| | | | | | C. orthopsilosis |
| 5 | Enterobacter | S. viridans | P. aeruginosa | OXA | C. krusei & |
| | cloacae complex | | | | C. glabrata |
| 6 | Citrobacter spp. | S. aureus | | mecA | C. auris |
| 7 | Enterococcus spp. | | | vanA/B | |
| 8 | Streptococcus spp. | | | CTX-M | |
| | | | | 14/15 | |
| 9 | Staphylococcus | | | mefA/E | |
| | spp. | | | | |
| 10 | Coagulase- | | | ermA/B | |
| | negative | | | | |
| | Staphylococcus | | | | |
| | spp. | | | | |
| 11 | Acinetobacter spp. | | | SHV | |
| 12 | Corynebacterium | | | TEM | |
| | spp. | | | | |
| 13 | Mycobacterium | | | CMY/DHA | |
| | spp. | | | | |
| 14 | Pan Fungal | | | FKS | |
| 15 | Candida spp. | | | Pdr1 & | |
| | | | | Erg11 | |

In some examples, the method may include using T2MR to obtain group-level information regarding a biothreat species, e.g., one or more of Bacillus anthracis, Francisella tularensis, Burkholderia spp. (e.g., B. mallei or B. pseudomallei), Yersinia pestis, and Rickettsia prowazekii, which can be followed by sequencing to obtain species-level information. In other examples, the method may include using T2MR to obtain group-level information regarding a toxin gene, e.g., Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BoNT/E, BoNT/F, and BoNT/G, which can be followed by sequencing to obtain species- or variant-level information.

### Example 9

### T2MR+NGS Identifies Species and Resistance Gene Identity Directly from Blood Lysate

A feasibility study for direct species identification using non-optimized 16S primers was conducted using NGS. The targets shown in Table 25 were spiked into blood lysate at 1,000 copies per single-spike blood lysate reaction for multi-spike samples. Only non-human reads were evaluated. The 16S primers used in this experiment differentiated between the species that were spiked, with percentages of reads ranging from 96.5% to 100%. In this experiment, the indicated species were not distinguishable in the amplified region (500 bp): (i) A. baumanii, A. calcoaceticus, A. generi; (ii) E. aerogenes, Raoultella spp., (iii) E. coli, E. fergosonii, Salmonella enterica, Shigella spp., (iv) K. pneumoniae, K. quasipneumoniae, K. variicola; and (v) S. aureus, S. argenteus.

TABLE 25
Species Detection using 16S primers and NGS sequencing

| | | Sum of | Percent of |
|---|---|---|---|
| Single Spiked Target | Detected? | Reads | Reads |
| Acinetobacter baumannii | + | 21069 | 99.5* |
| Enterobacter aerogenes | + | 17077 | 100.0 |
| Enterobacter cloacae | + | 11288 | 100.0 |
| Enterococcus faecium | + | 14539 | 100.0 |
| Escherichia coli | + | 20352 | 100.0 |
| Klebsiella pneumoniae | + | 16544 | 99.3* |
| Pseudomonas aeruginosa | + | 12375 | 96.5** |
| Staphylococcus aureus | + | 17092 | 100.0 |

*V. paradoxus detected at <1% of reads
**S. maltophilia, V. paradoxus, V. atypica detected at <2% of reads each

To assess whether T2MR+NGS can deliver both species and resistance gene identity, seven isolates containing resistance genes were spiked at 100 CFU/mL in whole blood:
Klebsiella pneumoniae AR-0079 (ctx-m-14, dha-1, tem-1B)
Morganella morganii AR-0057 (ctx-m-15, ndm-1)
Pluralibacter gergoviae JMI 968517 (dha-7, kpc-2, shv-5, tem)
Klebsiella ozaenae AR-0051 (ctx-m-15, oxa-181)
Enterobacter cloacae AR-0154 (vim-1, tem-1B)
Enterobacter aerogenes AR-0161 (imp-4, tem-1B)
Candida albicans

A non-optimized 16S/Resistance Gene multiplexed primer set (15 primer pairs) was tested using T2MR and NGS. Table 26 shows the sequences of the forward and reverse 16S primers used for sequencing; the “gramPA” primers contain the added ILLUMINA® partial adapter sequence on the 5′ ends. As is shown in Table 27, T2MR+NGS delivered highly multiplexed species and resistance gene identification. 12/13 targets were identified by T2MR, and 13/13 targets were identified by sequencing. Further optimization has been completed for SHV T2MR detection. Only non-human reads were evaluated. The T2MR 16S detects species, and NGS identifies them. NGS enabled identification in highly multiplexed samples.

TABLE 26
16S Primer Sequences

| Name | Sequence |
|---|---|
| gram_F | 5′-CTCCTACGGGAGGCAGCAGT-3′ |
| | (SEQ ID NO: 98) |
| gram_R | 5′-GTATTACCGCGGCTGCTGGCA-3′ |
| | (SEQ ID NO: 99) |
| gramPA_F | 5′-TCGTCGGCAGCGTCAGATGTGTATAAGAGACAGCTCC |
| | TACGGGAGGCAGCAGT-3′ (SEQ ID NO: 100) |
| gramPA_R | 5′-GTCTCGTGGGCTCGGAGATGTGTATAAGAGACAGGTA |
| | TTACCGCGGCTGCTGGCA-3′ (SEQ ID NO: 101) |

TABLE 27
T2MR and NGS Detection of Species and Resistance Gene

| Target | T2MR | NGS |
|---|---|---|
| 16S Pan Bacteria K. pneumoniae/ | + | + |
| K. ozaenae | | |
| 16S Pan Bacteria M. morganii | | + |
| 16S Pan Bacteria E. cloacae/ | | + |
| P. gergoviae | | |
| 16S Pan Bacteria E. aerogenes | | + |
| 23S Enterobacter/Klebsiella | + | + |
| DHA | + | + |
| KPC | + | + |
| NDM | + | + |
| OXA-48 | + | + |
| IMP | + | + |
| VIM | + | + |
| CTXM15 | + | + |
| CTXM14 | + | + |
| SHV | − | + |
| TEM | + | + |
| Candida albicans | + | + |
| IC | + | + |

NGS Contaminants 0.63% of non-human reads: S. cerevisiae, Citrobacter spp., CMY, unidentified fungal.

In another experiment, 10 CFU/mL blood samples spiked with multiple resistant organisms were run on the automated T2DX® instrument with the T2Carba Resistance+ panel and tested either by T2MR or by NGS. 100% concordance was obtained between T2MR and sequencing (Table 28). Samples were run either with or without amplicon purification prior to sequencing; both had 100% concordance, and amplicon purification only impacted the background level of human sequences. These results demonstrate that T2MR provides a therapeutically meaningful rapid result prior to sequencing and enables reflex to sequencing based on T2MR positivity to enable typing and higher resolution analyses. Moreover, T2DX® cellular pathogen detection provides higher specificity (about 98%) compared to cell-free DNA methods (about 40-63%). Further, these data demonstrate that NGS of cellular pathogen targets can be performed from automated T2MR sample preparation approaches.

TABLE 28
Concordance between T2MR and NGS Detection

| 10 CFU/mL blood spike | T2DX ® | NGS |
|---|---|---|
| Enterobacter spp./ | + | + |
| Klebsiella spp. | | |
| OXA | − | − |
| NDM | + | + |
| KPC | − | − |
| VIM | + | + |
| IMP | + | + |
| AmpC | − | − |
| Internal Control | + | + |

In another example, blood samples were spiked with four species that contained the targets Enterococcus and Klebsiella spp. (“EK”), NDM, VIM, IMP, DHA, and OIC. The spiked blood samples were processed on the T2DX® instrument and tested by T2MR. In this experiment, all of the expected targets except DHA were detected by T2MR. Remaining amplicon from the T2DX® cartridges was pooled and prepared for NGS sequencing. The NGS results agreed with the T2MR detections. All targets were found within the top 25 sequences in NGS results.

One exemplary panel to combine T2MR and Sequencing is as follows. T2MR results, which are available in approximately 3.5 h, can give results akin to those available soon after a blood culture goes positive, e.g., a Gram positive organism, Gram negative organism, or a fungal organism. The panel may contain selected, high prevalence genus or species results (e.g., S. aureus, E. coli, and Streptococcus species) as well as selected virulent species that require rapid treatment. The sequencing results, which may be available in approximately 6 to 24 h (depending on the sequencer used) can provide species information and resistance gene identification (e.g., differentiating variants and SNPs (e.g., FSK gene, ESBL variants, and the like). Sequencing can also identify whether a skin contaminant is present (e.g., coagulase negative Staphylococcus+mecA versus S. aureus+mecA.

Conclusions

T2MR+sequencing can bring sequencing to direct-from-blood sample detection. The rapid, lower cost T2MR result can be used to screen for samples from which more information can be obtained by sequencing. Pre-screening with T2MR allows for avoiding testing of negative samples, pooling of samples on sequencing runs to reduce cost-per-sample sequenced, and narrows the window of data analysis. Sequencing provides higher resolution information to expand on the T2MR menu and to detect, for example, SNPs and resistance variants that cannot be distinguished by molecular diagnostics. The multi-staged results from T2MR and sequencing provide information equivalent to existing laboratory methods but on an accelerated, clinically meaningful timeline. T2MR′s sensitivity and specificity of 90%-100% for bloodstream infections facilitates appropriate sequencing and aids in data analysis. T2MR also can provide fully-automated, reproducible sample processing (e.g., as in the T2DX® instrument) for high sensitivity detection from blood for sequencing. This approach is broadly applicable, e.g., for bacterial and fungal testing in whole blood and other complex matrices for a number of indications, including, without limitation, sepsis, transplant, endocarditis, and Lyme disease.

### Example 10

### Combined T2Bacteria/NGS Panel

Sequencing primers were prepared by adding ILLUMINA NEXTERA® partial adapter sequences to the 5′ end of forward and reverse primers for a consensus region of the 16S ribosomal RNA (see Table 26). The sequencing primers were titrated into T2BACTERIA® Panel reaction buffers, and the highest concentration of sequencing primers that did not inhibit T2MR signal for T2BACTERIA® Panel species was selected to formulate a T2BACTERIA® Panel/NGS reaction buffer. Species detected by the T2BACTERIA® Panel (Acinetobacter baumannii, Enterococcus faecium, Escherichia coli, Klebsiella pneumoniae, Pseudomonas aeruginosa, and Staphyococcus aureus) were spiked at 2-3× limit of detection (LoD) into K2EDTA anticoagulated whole human blood. Off-panel species, including Bacillus subtilis, Borrelia burgdorferi, Raoultella ornithinolytica, Streptococcus pneumoniae, and Yersinia pseudotuberculosis were spiked at 20 CFU/mL into K2EDTA anticoagulated whole human blood. Each sample was processed with a T2DX® Instrument and detected with the T2BACTERIA® Panel software, and the sequencing sample was removed from the cartridges after the T2DX® Instrument was finished.

Sequencing libraries were prepared from the sequencing sample in the following manner. The sequencing samples were first removed from the T2DX® cartridge and purified with AMPure XP® beads. Unique NEXTERA® DNA CD indexes were added to each library via PCR amplification, and the libraries were purified with AMPure XP® beads and quantified with a QUBIT™ 4 fluorimeter. Libraries were normalized to the same concentration and mixed together. Multiplex libraries were paired end sequenced on an ILLUMINA® iSeq sequencing system. The demultiplexed fastq result files were analyzed using a combination of open source bioinformatics tools and custom Python-based analysis scripts. Short primer dimer or chimeric sequences were filtered from results, and remaining sequences were aligned to a 16s database with BLASTn.

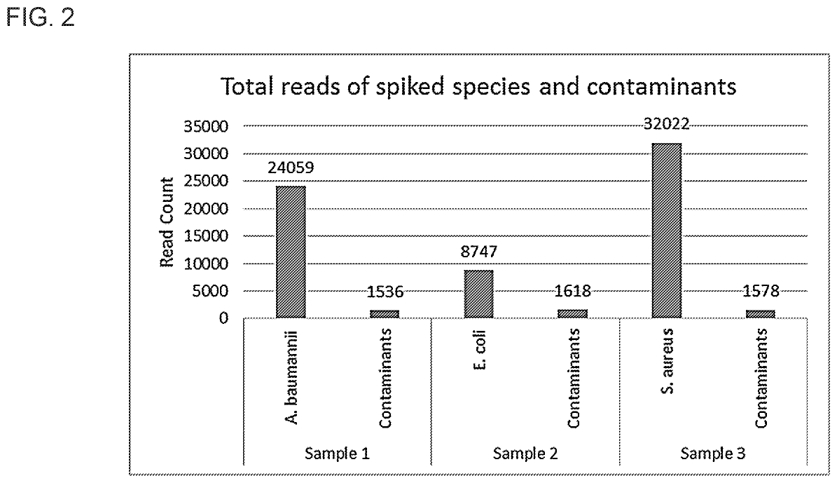

Table 29 shows T2MR and NGS results for the on- and off-panel species. These results demonstrate that on-panel species are detected by T2MR and off-panel species do not cross-react. Because the sequenced region was a short piece of conserved 16S rRNA DNA, multiple closely related species were aligned to each sequence. In most cases, the spiked species is represented as one of the best NGS matches. The Klebsiella pneumoniae spike more closely matched with K. pneumoniae subsp. ozaenae than K. pneumoniae. Borrelia burgdorferi was not represented as a top match with a Ralstonia insidiosa, a contaminating background species, coming in as the best match; this error was due to amplification efficiency of the B. burgdorferi target. In most cases, the spiked species have higher read counts compared to background species (FIG. 2).

TABLE 29
Comparison of T2MR and top NGS matches for whole blood samples
containing low titers of on- and off-panel species

| | | T2MR | |
|---|---|---|---|
| Sample | Titer (CFU/mL) | Detection | Best NGS Matches |
| Acinetobacter baumannii | 6 | A. baumanii | A. baumannii |
| | | | A. seifertii |
| Enterococcus faecium | 15 | E. faecium | E. faecium |
| | | | E. durans |
| | | | E. hirae |
| | | | E. thailandicus |
| | | | E. rattii |
| | | | E. villorum |
| | | | Vagococcus entomophilus |
| Escherichia coli | 15 | E. coli | E. coli |
| | | | E. albertii |
| | | | E. fergusonii |
| | | | Shigella flexneri |
| | | | S. dysenteriae |
| | | | S. sonnei |
| | | | Brenneria alni |
| Klebsiella pneumoniae | 6 | K. pneumoniae | K. pneumoniae subsp. ozaenae |
| Pseudomonas aeruginosa | 12 | P. aeruginosa | P. aeruginosa |
| | | | P citronellolis |
| | | | P. stutzeri |
| | | | P. nitroreducens |
| | | | P. delhiensis |
| | | | P. alcaligenes |
| | | | P. knackmussii |
| | | | P. guezennei |
| | | | P. multiresinivorans |
| | | | P. fluvialis |
| Staphylococcus aureus | 15 | S. aureus | S. aureus |
| | | | S. aureus subsp. anaerobius |
| | | | S. simiae |
| Borreliella burgdorferi | 20 | Not Detected | Ralstonia insidiosa (contaminant) |
| Bacillus subtilis | 20 | Not Detected | B. subtilis |
| | | | B. subtilis subsp. spizizenii |
| | | | B. subtilis subsp. inaquosorum |
| | | | B. pseudalcaliphilius |
| | | | B. mojavensis |
| | | | B. aquimaris |
| | | | B. tequilensis |
| | | | B. halotolerans |
| | | | B. swezeyi |
| Raoultella ornithinolytic | 20 | Not Detected | R. ornithinolytica |
| | | | R. planticola |
| | | | Kluyvera ryocrescens |
| | | | Cedecea lapagei |
| | | | Citrobacter europaeus |
| Streptococcus pneumoniae | 20 | Not Detected | S. pneumoniae |
| | | | S. pseudopneumoniae |
| | | | S. australis |
| | | | S. oralis |
| | | | S. oralis subsp. dentisani |
| | | | S. oralis subsp. tigurinus |
| | | | S. infantis |
| | | | S. rubneri |
| | | | S. mitis |
| | | | S. cristatus |
| Yersinia pseudotuberculosis | 20 | Not Detected | Y. pseudotuberculosis |
| | | | Y. pestis |
| | | | Y. frederikensenii |
| | | | Y. wautersii |

These data demonstrate that sequencing (e.g., using a 16S primer set) can be used in combination with T2MR-based panel assays, e.g., the T2CANDIDA®, T2BACTERIA®, T2LYME®, and other panels to provide additional breadth of detection and species identification.

### Example 11

### Synthetic IC Setting Cutoff

We designed a process for setting a read count cutoff to differentiate background contamination from spiked or clinically relevant species. A baseline for contaminating species was established by determining the average normalized read counts from contaminating species in negative samples. Negative samples were processed using a manual assay and amplified with partial adapter primers. A synthetic control DNA with partial adapter sequences was added to the amplicon from the negative samples at controlled copy numbers. These experiments were tested with 1e10 and 1e12 copies per reaction of synthetic IC. It is expected that the concentration of synthetic IC can be set lower, because the number of reads from the 1e10 copies/reaction sample was 5-10× higher than spiked samples. The control level could be set to a static concentration, but would be affected by degree of NGS multiplexing and dilution of the libraries. A range of 1e8 copies/reaction-1e12 copies/reaction may be used for the synthetic IC. Libraries were prepared using adapter PCR as described above (e.g., in Example 10). The negative samples were sequenced on an ILLUMINA® iSeq sequencing system, and the read counts for each taxonomic call were normalized by the read count of the synthetic control. A cutoff was set 3 standard deviations above the average normalized read count of the highest contaminating sequence from the negative samples. This cutoff was a good compromise between eliminating background species from the result and obtaining sensitive detection of spiked signals. It is expected that cutoffs in the range of 1-6SD above the average normalized read count of the highest contaminating sequence from negative samples could be used.

Enterococcus faecium cells were spiked in whole blood at 5, 10, 25, and 50 CFU/mL, and as above, a library containing the synthetic control was prepared for sequencing from each sample. The number of reads for all taxonomic calls was normalized by the number of reads of the synthetic control and compared to the cutoff. For all tested spikes, the Enterococcus normalized reads were above the cutoff and no contaminating species surpassed the cutoff (FIG. 3; triangles below cutoff). The putative limit of detection for sequencing of Enterococcus faecium using this method is 5 CFU/mL and normalized read counts increased linearly with the spike concentration (FIG. 3). Without wishing to be bound by theory, the low levels of contaminating DNA below the cutoff is thought to be due to the use of whole pathogen cell DNA rather than cfDNA, in part because a technique that sequences the small pieces of cfDNA will detect any incidental contamination found in the environment or reagents.

These data demonstrate that a read count cutoff to differentiate background contamination from spiked or clinically relevant species in complex samples such as blood can be set using synthetic control DNAs at a controlled concentration. Additionally, it is expected that quantification of pathogen load may be performed using normalization controls.

### Example 12

### T2MR and NGS Testing Panel

A panel that combines T2MR-based detection (e.g., using a T2DX® instrument) and NGS was developed for high-sensitivity, direct from sample amplicon preparation for targeted NGS. The panel includes bacterial and fungal species using 16S and internal transcribed spacer (ITS) primers, respectively, as well as resistance genes (an exemplary, non-limiting list is shown in Table 30).

TABLE 30
Exemplary T2MR-NGS Panel

| Target | Description |
|---|---|
| 16S | Bacterial genus/species identification |
| ITS | Fungal species identification |
| mecA/C | Methicillin resistance genes |
| vanA/B | Vancomycin resistance genes |
| KPC | Carbapenemase gene |
| OXA-48 | Carbapenemase gene |
| VIM | Carbapenemase gene |
| IMP | Carbapenemase gene |
| NDM | Carbapenemase gene |
| CMY | AmpC beta-lactamase gene |
| DHA | AmpC beta-lactamase gene |
| CTX-M | Extended spectrum beta-lactamase gene |

Following amplicon generation (e.g., using the T2DX® instrument), the sample can be prepared for sequencing on benchtop, for example, using commercially available NGS kits. Samples can be sequenced with an ILLUMINA® NGS system or other NGS systems.

In one optional example, rapid T2MR Pan-Bacteria/Pan-Fungal screening can be performed using T2MR to rapidly (3-5 h) determine whether bacteria or fungi are present in the sample (e.g., whole blood), and NGS can be used on positive samples to provide a comprehensive pathogen detection panel. Initial rapid T2MR Pan-Bacteria/Pan-Fungal testing will identify positive cases that should move to NGS for species identification, thereby saving time and costs.

### Sequence Listing

The following sequences are used throughout the application. “/i6diPr/” indicates 2,6-Diaminopurine, and “NitInd” indicates 5′ 5-Nitroindole.

| | SEQ ID |
|---|---|
| Sequence | NO: |
| 5′-CGT TTT CCA AAT CTG TAA CAG ACT GGG-3′ | 1 |
| 5′-AGG ACG TTG ATA GG TTG GAT GTG GA-3′ | 2 |
| 5′-GGT AGC TAT GTA GGG AAG GGA TAA ACG CTG A-3′ | 3 |
| 5′-GCG CTA AGG AGC TTA ACT TCT GTG TTC G-3′ | 4 |
| 5′-GAC GGT TGT CCC GGT TTA AGC A-3′ | 5 |
| 5′-GCT GGT ATC TTC GAC TGG TCT-3′ | 6 |
| 5′-AGG CTG GGT GTG TAA GCG TTG T-3′ | 7 |
| 5′-CAA GCA ATT CGG TTG GAT ATC CGT T-3′ | 8 |
| 5′-GGT AAT GAATTA CCT /i6diPr/TC TCT GCT GGTTTC TTC TT-3′ | 9 |
| 5′-ACC AGC ATC TTC /i6diPr/GC ATC TTC TGT AAA-3′ | 10 |
| 5′-GAA GTT ATG TTT /i6diPr/CT ATT CGA ATC GTG GTC CAGT-3′ | 11 |
| 5′-GTT GTA AAG CCA TGA TGC TCG TAA CCA-3′ | 12 |
| 5′-GGC ATG CCT GTT TGA GCG TC-3′ | 13 |
| 5′-GCT TAT TGA TAT GCT TAA GTT CAG CGG GT-3′ | 14 |
| 5′-TGA GGC TTG ACT ATA CAA CAC C-3′ | 15 |
| 5′-CTA AAA TGA ACA GAT AAA GTA AGA TTC AA-3′ | 16 |
| 5′-AAA ACT TAT ATG ACT TCA AAT CCA GTT TT-3′ | 17 |
| 5′-TTT ACT CAA TAA AAG ATA ACA CCA CAG-3′ | 18 |
| 5′-TGG ATA AGT AAA AGC AAC TTG GTT-3′ | 19 |
| 5′-AAT GAA GAT TCA ACT CAA TAA GAA ACA ACA-3′ | 20 |
| 5′-TAC CAA GGC GCT TGA GAG AAC TC-3′ | 21 |
| 5′-CTG GTG TGT AGG TGA AGT C-3′ | 22 |
| 5′-GTG TGT TGT AGG GTG AAG TCG AC-3′ | 23 |
| 5′-CAC CTT GAA ATC ACA TAC CTG A-3′ | 24 |
| 5′-CCA TTT GAA GTT GTT TAT TAT GC-3′ | 25 |
| 5′-GGG AAA TGA TTA ATT ATG CAT TAA ATC-3′ | 26 |
| 5′-TT TTT CAG ATT TAG GAT TAG TTG ATT-3′ | 27 |
| 5′-GAT CCG TAT TGG TTA TAT CAT C-3′ | 28 |
| 5′-GG AAT AAT ACG CCG ACC AGC-3′ | 29 |
| 5′-AAG GAT CTA TTT CAG TAT GAT GCA G-3′ | 30 |
| 5′- | 31 |
| TGCCGAAGCGTTTTCCAAATCTGTAACAGACTGGGCTGATTGAATCTTACTTT | |
| ATCTGTTCATTTTAGCTAGAGGTATAACTAAATCAAGTTGTCTTGCATATTTAA | |
| GAATCGATTGATGCTTTATATACAACTGCTTGGGTGTTGTATAGTCAAGCCTCA | |
| CGAGCAATTAGTATTGGTCAGCTTCACATATCACTATGC-3′ | |
| 5′- | 32 |
| GCATGGGAACAGGTGTATCCTTCTCGCTATCGCCACCACACTGGGTGTTGTTT | |
| CTTATTGAGTTGAATCTTCATTCACTCAAAACTGGATTGAAGTTTGAATCAAAA | |
| TAACCAAGTTGCTTTTACTTATCCATTCTTTGGTTAAGTCCTCGACCGATTAGT | |
| ATTGGTCCGCTCCAACTATCACTAGCCTTCCACTTCCAA-3′ | |
| 5′- | 33 |
| GCATGGTTACAGGTGTATCCTTCTCGCTATCGCCACCACACTGTGGTGTTATC | |
| TTTTATTGAGTAAATTTTGTTCACTCAAAACTGGATTTGAAGTCATATAAGTTTT | |
| TTTCCGAGTTCTTTTCTTTTAACCTATTGGTTAAGTCCTCGATCGATTAGTATCA | |
| GTCCGCTCCATACATCACTGTACTTCCACTCCTGACC-3′ | |
| 5′- | 34 |
| CAGCTCCATCCGCAGGGACTTCACCTACACACCAGCGTGCCTTCTCCCGAAG | |
| TTACGGCACCATTTTGCCTAGTTCCTTCACCCGAGTTCTCTCAAGCGCCTTGG | |
| TATTCTCTACCTGACCACCTGTGTCGGTTTGGGGTACGATTTGATGTTACCTG | |
| ATGCTTAGAGGCTTTTCCTGGAAGCAGGGCATTTGTTACTTC-3′ | |
| 5′- | 35 |
| CGCTTGGGCTTACGTCTATCCGGATTCAGGTATGTGATTTCAAGGTGTTTTGC | |
| GGTTCATGCGAACTTTCGGTTCGTCGACTTCACCTTACAACACACAATCGTCA | |
| GATTGTTTGGGTGTTATATGGTCAAGCCTCACGGGCAATTAGTACTGGTTAGC | |
| TCAACGCCTC-3′ | |
| 5′- | 36 |
| TTTACCACTAACACCATAGAAATTATAACGGTCAATGCCATGATTTAATGCATA | |
| ATTAATCATTTCCCATTGCACTGCATAACTTCCGGCAAAATGACGGAATGCATT | |
| TGATGTACCACCAGCATAATAAACAACTTCAAATGGGTTGATA-3′ | |
| 5′- | 37 |
| TGTGATTTAAACAAGTTTACTAAGGCATCATTTTTCTCGCGACCTTCAAATGGC | |
| ACGATATCTTTATCATATAGATGATATAACCAATACGGATCTAATTTAACATATA | |
| AACATTGATGTTGCTGTAAATATTTATCTAACTCTTTTAAATAATAATCAACTAA | |
| TCCTAAATCTGAAAAATCCATT-3′ | |
| 5′-GCA TTA ATC GAC GGT ATG GTT GAC C-3′ | 38 |
| 5′-CCT GCT GAA ACA GGT TTT CCC ACA TA-3′ | 39 |
| 5′-AGT GAT GAT GAG TTG TTT GCC AGT G-3′ | 40 |
| 5′-TGA ATT GTC GCC GCG TGA CCA G-3′ | 41 |
| 5′-ACC CAG CGG TTT GAG GGA GAA AC-3′ | 42 |
| 5′-AAA GTT TGA AGA TAT ACG TGG TGG ACG TTA-3′ | 43 |
| 5′-CGC ACG CGC AAG ATG GAA ACG-3′ | 44 |
| 5′-AAG TTC AGC GGG TAT TCC TAC CT-3′ | 45 |
| 5′-AGC TTT TTG TTG TCT CGC AAC ACT CGC-3′ | 46 |
| 5′-CTA CCA AAC ACA ATG TGT TTG AGA AG-3′ | 47 |
| 5′-CCT GAT TTG AGG TCA AAC TTA AAG ACG TCT G-3′ | 48 |
| 5′-AGT CCT ACC TGA TTT GAG GTC NitInd AA-3′ | 49 |
| 5′-CCG NitInd GG GTT TGA GGG AGA AAT-3′ | 50 |
| 5′-AAA GTT ATG AAATAA ATT GTG GTG GCC ACT AGC-3′ | 51 |
| 5′-ACC CGG GGGTTT GAG GGA GAA A-3′ | 52 |
| 5′-AGT CCT ACC TGA TTT GAG GTC GAA-3′ | 53 |
| 5′-CCG AGG GTT TGA GGG AGA AAT-3′ | 54 |
| 5′-GGA AGG GAT CAG GTG GTT CAC TCT T-3′ | 55 |
| 5′-AAA ACT TAT GTG ACT TCA AAT CCA GTT TT-3′ | 56 |
| 5′-TCT GAC GAT TGT GTG TTG TAA GG-3′ | 57 |
| 5′-GGA TAG ACG TAA GCC CAA GC-3′ | 58 |
| 5′-GCA TGG TTA CAG GTG TAT CCT TCT CGC TAT CGC CAC CAC ACT GTG | 59 |
| GTG TTA TCT TTT ATT GAG TAA ATT TTG TTC ACT CAA AAC TGG ATT TGA | |
| AGT CAT ATA AGT TTT TTT CCG AGT TCT TTT CTT TTA ACC TAT TGG TTA | |
| AGT CCT CGA TCG ATT AGT ATC AGT CCG CTC CAT ACA TCA CTG TAC | |
| TTC CAC TCC TGA-3′ | |
| 5′-GGTTAAGTCCCGCAACGAGCGC-3′ | 60 |
| 5′-AGGAGGTGATCCAACCGCA-3′ | 61 |
| 5′-GGG CAT GCC TGT TTG AGC GT-3′ | 62 |
| 5′-CTA CCT GAT TTG AGG CGA CAA CAA AAC-3′ | 63 |
| 5′-CCG CGA AGA TTG GTG AGA AGA CAT-3′ | 64 |
| 5′-CCT ACC TGA TTT GAG GGC GAA ATG TC-3′ | 65 |
| 5′-GGA GCA ACG CCT AAC CGG G-3′ | 66 |
| 5′-GTC CTA CCT GAT TTG AGG GGA AAA AGC-3′ | 67 |
| 5′-AAC AAA TCC ACC AAC GGT GAG AAG ATA T-3′ | 68 |
| 5′-GCG TAG ACT TCG CTG CGG AT-3′ | 69 |
| 5′-CGT AGA CTT CGC TGC GGA T-3′ | 70 |
| 5′-CTG GGC GGT GAG AAG AAA TC-3′ | 71 |
| 5′-GCG TAG ACT TCG CTG CTG GAA-3′ | 72 |
| 5′-CCG TGC GGT GAG AAG AAA TC-3′ | 73 |
| 5′-TCC TAC CTG ATT TGA GGA AAT AGC ATG G-3′ | 74 |
| 5′-ATT TAG CGG ATG CAA AAC CAC C-3′ | 75 |
| 5′-GGAAATCTAACGAGAGAGCATGCT-3′ | 76 |
| 5′-CGATGCGTGACACCCAGGC-3′ | 77 |
| 5′-CAAGGTGCAATGACTTTGTTTGGGCA-3′ | 78 |
| 5′-GCAACTTCAAAGTGTACAGTATTGGTATCCC-3′ | 79 |
| 5′-AGCTGTAGTTTAAGGCAAATGTTGG-3′ | 80 |
| 5′-AGGATCGCAAAATCAACCACAAACA-3′ | 81 |
| 5′-CCTAAATGTTAAACCCCTTGACAACCCA-3′ | 82 |
| 5′-CCCATCAGGATATCCAGCTTCGG-3′ | 83 |
| 5′-CTTTACCGATACTTCAATTTCACCGAGCTCCA-3′ | 84 |
| 5′-CACAGGTCTCTGCAAATCTGTAAAGAGAA-3′ | 85 |
| 5′-CGATGGTTCACGGGATTCTGCAATTC-3′ | 86 |
| 5′-GAGACGTTTTGGATACATGTGAAAGAAGGC-3′ | 87 |
| 5′-TTGTAGAACAATCTGGGCTTTTTG G-3′ | 88 |
| 5′-GGAGAACTCATATCAGGAGCACAA-3′ | 89 |
| 5′-GCTATTTCTGCTGTTAAAAGTTCTTG T-3′ | 90 |
| 5′-CTAAAACTTAAGCTTTGCAATTGTGG-3′ | 91 |
| 5′-TCTAGCGGTTGACAGAGAAACATTG-3′ | 92 |
| 5′-AAAAAATTAAAACCATATAACCCACGAA-3′ | 93 |
| 5′-GAGACAGCGTCCAAATCGTTACACC-3′ | 94 |
| 5′-TCTTAACCTTCCAGCACCGGGCA-3′ | 95 |
| 5′-CTA TGT AGG GAA GGG ATA AAC GCT GA-3′ | 96 |
| 5′-GCG CTA AGG AGC TTA ACT TCT GTG TTC G-3′ | 97 |
| 5′-CTCCTACGGGAGGCAGCAGT-3′ | 98 |
| 5′-GTATTACCGCGGCTGCTGGCA-3′ | 99 |
| 5′- | 100 |
| TCGTCGGCAGCGTCAGATGTGTATAAGAGACAGCTCCTACGGGAGGCAGCA | |
| GT-3′ | |
| 5′- | 101 |
| GTCTCGTGGGCTCGGAGATGTGTATAAGAGACAGGTATTACCGCGGCTGCTG | |
| GCA-3′ | |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method for detecting a target nucleic acid in a biological sample obtained from a subject, wherein the biological sample comprises subject-derived cells or cell debris, the method comprising:
(a) amplifying a target nucleic acid in the biological sample to form an amplified solution comprising an amplified target nucleic acid; and
(b) sequencing the amplified target nucleic acid to detect whether the target nucleic acid is present in the biological sample,
wherein the method is capable of detecting a concentration of about 10 copies/mL of the target nucleic acid in the biological sample.

2. The method of claim 1, wherein:
(i) the biological sample has a volume of about 0.2 mL to about 5 mL;
(ii) the biological sample is selected from the group consisting of blood, bloody fluids, tissue samples, urine, cerebrospinal fluid (CSF), synovial fluid (SF), and sputum;
(iii) the target nucleic acid is characteristic of a pathogen; and/or
(iv) step (a) comprises amplifying the target nucleic acid in a lysate produced by lysing cells in the biological sample.

3-4. (canceled)

5. The method of claim 2, claim wherein:
(i) the blood is whole blood, a crude blood lysate, serum, or plasma;
(ii) the bloody fluid is wound exudate, phlegm, or bile;
(iii) the tissue sample is a tissue biopsy; or
(iv) the lysate has at least about a 2:1 higher concentration of cell debris relative to the biological sample.

6-19. (canceled)

20. A method for detecting a target pathogen nucleic acid in a whole blood sample, the method comprising:
(a) contacting a whole blood sample suspected of containing one or more pathogen cells with an erythrocyte lysis agent, thereby lysing red blood cells;
(b) centrifuging the product of step (a) to form a supernatant and a pellet;
(c) discarding some or all of the supernatant of step (b) and resuspending the pellet to form an extract, optionally washing the pellet one or more times prior to resuspending the pellet;
(d) lysing the remaining cells in the extract of step (c) to form a lysate, the lysate containing both subject cell nucleic acid and pathogen nucleic acid;
(e) amplifying pathogen nucleic acids in the lysate of step (d) to form an amplified lysate solution comprising an amplified target pathogen nucleic acid; and
(f) sequencing the amplified target pathogen nucleic acid, thereby detecting the target pathogen nucleic acid in the sample.

21. The method of claim 20, wherein:
(i) step (c) comprises washing the pellet one time prior to resuspending the pellet;
(ii) step (a) further comprises adding a total process control (TPC) to the whole blood sample;
(iii) the lysate or the amplified lysate solution has at least about a 2:1 higher concentration of subject cell DNA and/or cell debris relative to the whole blood sample;
(iv) the whole blood sample has a volume of about 0.2 mL to about 5 mL; and/or
(v) the lysate produced from the whole blood sample has a volume of about 10 μL to about 500 μL.

22-30. (canceled)

31. A method for detecting a target pathogen nucleic acid in a whole blood sample, the method comprising:
(a) providing an amplified lysate solution that has been produced by:
(i) contacting a whole blood sample suspected of containing one or more pathogen cells with an erythrocyte lysis agent, thereby lysing red blood cells;
(ii) centrifuging the product of step (a)(i) to form a supernatant and a pellet;
(iii) discarding some or all of the supernatant of step (a)(ii) and resuspending the pellet to form an extract, optionally washing the pellet one or more times prior to resuspending the pellet;
(iv) lysing the remaining cells in the extract of step (a)(iii) to form a lysate, the lysate containing both subject cell nucleic acid and pathogen nucleic acid;
(v) amplifying pathogen nucleic acids in the lysate of step (a)(iv) to form an amplified lysate solution comprising an amplified target pathogen nucleic acid; and
(b) sequencing the amplified target pathogen nucleic acid, thereby detecting the target pathogen nucleic acid in the sample.

32-41. (canceled)

42. A method for detecting a target pathogen nucleic acid in a whole blood sample, the method comprising:
(a) contacting a whole blood sample suspected of containing one or more pathogen cells with an erythrocyte lysis agent, thereby lysing red blood cells;
(b) centrifuging the product of step (a) to form a supernatant and a pellet;
(c) discarding some or all of the supernatant of step (b) and washing the pellet once;
(d) centrifuging the product of step (c) to form a supernatant and a pellet;
(e) discarding some or all of the supernatant of step (d) and mixing the pellet of (d) with a buffer solution;
(f) combining the product of step (e) with beads to form a mixture and agitating the mixture to form a lysate, said lysate containing both subject cell nucleic acid and pathogen nucleic acid;
(g) amplifying pathogen nucleic acids in the lysate of step (f) to form an amplified lysate solution comprising an amplified target pathogen nucleic acid; and
(h) sequencing the amplified target pathogen nucleic acid, thereby detecting the target pathogen nucleic acid in the sample.

43-59. (canceled)

60. The method of claim 1, wherein:
(i) the amplifying is performed in the presence of from about 0.5 μg to about 100 μg of subject cell DNA; and/or
(ii) the amplifying comprises polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, or ramification amplification (RAM).

61-64. (canceled)

65. The method of claim 1, wherein the amplifying comprises PCR.

66. (canceled)

67. The method of claim 2, wherein:
(a) the amplifying comprises:
(i) adding to the lysate an amplification buffer solution comprising a buffering agent to form a reaction mixture;
(ii) heating the reaction mixture to form a denatured reaction mixture; and
(iii) adding a thermostable nucleic acid polymerase to the denatured reaction mixture under conditions and for a time sufficient for amplification of the target nucleic acid;
(b) the amplifying comprises:
(i) adding to the lysate an amplification buffer solution comprising a buffering agent and a thermostable nucleic acid polymerase to form a reaction mixture under conditions and for a time sufficient for amplification of the target nucleic acid;
(ii) heating the reaction mixture to form a denatured reaction mixture; and
(iii) centrifuging the denatured reaction mixture to form a pellet and a supernatant;
(c) the thermostable nucleic acid polymerase is a thermostable DNA polymerase; or
(d) the thermostable nucleic acid polymerase is inhibited by the presence of subject-derived cells or cell debris under normal reaction conditions.

68-104. (canceled)

105. The method of claim 1, wherein:
(i) the method does not include extracting or purifying the amplified target nucleic acid prior to the sequencing;
(ii) the method further comprises cleaning up the amplified target nucleic acid prior to the sequencing;
(iii) the sequencing comprises massively parallel sequencing, Sanger sequencing, or single-molecule sequencing;
(iv) the method further comprises amplifying one or more additional target nucleic acids in a multiplexed amplification reaction to generate one or more additional amplicons;
(v) the target nucleic acid is characteristic of a pathogen, and the method identifies the genus of the pathogen; or
(vi) the method further comprises detecting the amplified target nucleic acid using T2 magnetic resonance (T2MR) prior to the sequencing.

106-109. (canceled)

110. The method of claim 105, wherein:
(i) the massively parallel sequencing comprises sequencing by synthesis or sequencing by ligation; and/or
(ii) the massively parallel sequencing comprises use of a synthetic control DNA to normalize read counts, wherein the target nucleic acid is detected in the sample if the normalized read count for the target nucleic acid is at or above a reference read count.

111-121. (canceled)

122. The method of claim 105, wherein:
(a) the method comprises the following steps:
(i) adding magnetic particles to a portion of the amplified solution or amplified lysate solution to form a detection mixture, wherein the magnetic particles have binding moieties on their surface, the binding moieties operative to alter aggregation of the magnetic particles in the presence of the amplified target nucleic acid, and
(ii) detecting the presence of the amplified target nucleic acid by measuring the aggregation of the magnetic particles using T2MR; and/or
(b) detecting the amplified target nucleic acid using T2MR results in a species or group-level identification of the target nucleic acid by T2MR.

123. The method of claim 122, wherein:
step (ii) comprises the following steps:
(a) providing the detection mixture in a detection tube within a device, the device comprising a support defining a well for holding the detection tube comprising the mixture, and having an RF coil configured to detect a signal produced by exposing the mixture to a bias magnetic field created using one or more magnets and an RF pulse sequence;
(b) exposing the detection mixture to a bias magnetic field and an RF pulse sequence;
(c) following step (b), measuring the signal from the detection tube; and
(d) on the basis of the result of step (c), detecting the amplified target nucleic acid; and/or
the magnetic particles comprise a first population of magnetic particles conjugated to a first probe, and a second population of magnetic particles conjugated to a second probe, the first probe operative to bind to a first segment of the amplified target nucleic acid and the second probe operative to bind to a second segment of the amplified target nucleic acid, wherein the magnetic particles form aggregates in the presence of the amplified target nucleic acid.

124-136. (canceled)

137. The method of claim 105, wherein:
(i) sequencing the amplified target nucleic acid results in a species-level or variant-level identification of the target nucleic acid; and/or
(ii) the detecting by T2MR is completed within 5 hours of amplifying the target nucleic acid.

138-148. (canceled)

149. The method of claim 2, wherein:
(i) the pathogen is a fungal pathogen, a bacterial pathogen, a protozoan pathogen, or a viral pathogen;
(ii) the method is capable of detecting a concentration of about 10 colony-forming units (CFU)/mL of the pathogen species in the whole blood sample or lower; or
(iii) the method further comprises diagnosing the subject based on the detection of the target nucleic acid, wherein the presence of the target nucleic indicates that the subject is suffering from a disease associated with the pathogen.

150. (canceled)

151. The method of claim 149, wherein:
(i) the fungal pathogen is a Candida spp., an Aspergillus spp., or a Cryptococcus spp.;
(ii) the bacterial pathogen is a Gram positive bacterium, a Gram negative bacterium, an Enterobacteriaceae family bacterium, an Enterobacter spp., a Citrobacter spp., a Enterococcus spp., a Streptococcus spp., a Staphylococcus spp., an Acinetobacter spp., a Corynebacterium spp., Enterobacter cloacae complex, or a Mycobacterium spp.; or
(iii) the protozoan pathogen is Babesia microti or Babesia divergens.

152-166. (canceled)

167. The method of claim 1, wherein the target nucleic acid is an antimicrobial resistance gene.

168. The method of claim 167, wherein the antimicrobial resistance gene is bla_{KPC}, blaZ, bla_{NDM}, bla_{IMP}, bla_{VIM}, bla_{OXA}, bla_{CMY}, bla_{DHA}, bla_{TEM}, bla_{SHV}, bla_{CTX-M}, bla_{SME}, bla_{FOX}, bla_{MIR}, femA, femB, mecA, mecC, macB, fosA, vanA, vanB, vanC, vanD, vanE, vanG, mefA, mefE, ermA, ermB, tetA, tetB, tetX, tetR, qnrA, qnrB, qnrS, FKS1, FKS2, ERG11, or PDR1.

169-170. (canceled)

171. A method for detecting a target nucleic acid in a biological sample obtained from a subject, wherein the biological sample comprises subject-derived cells or cell debris, the method comprising:
(a) amplifying a target nucleic acid in the biological sample to form an amplified solution comprising an amplified target nucleic acid;
(b) detecting the amplified target nucleic acid using T2MR to provide a group-level identification of the target nucleic acid; and
(c) sequencing the amplified target nucleic acid to provide a species-level or variant-level identification of the target nucleic acid,
wherein the method is capable of detecting a concentration of about 10 copies/mL of the target nucleic acid in the biological sample.
